(19) United States
(12) Patent Application Publication
(10) Pub. No.: US 2024/0261393 A1
(43) Pub. Date: Aug. 8, 2024

(54) SARS-COV-2 RNA VACCINE COMPOSITIONS AND METHODS OF USE

(71) Applicant: HDT Bio Corp., Seattle, WA (US)

(72) Inventors: Steven Gregory Reed, Bellevue, WA (US); Darrick Albert Carter, Seattle, WA (US); Amit Praful Khandhar, Issaquah, WA (US); Jacob Freeman Archer, Seattle, WA (US); Lars Peter Aksel Berglund, Seattle, WA (US); Jesse Erasmus, Port Orchard, WA (US); Bryan Berube, Issaquah, WA (US); Malcolm S. Duthie, Sammamish, WA (US)

(21) Appl. No.: 18/612,698

(22) Filed: Mar. 21, 2024

Related U.S. Application Data
(63) Continuation of application No. PCT/US2022/013513, filed on Jan. 24, 2022.
(60) Provisional application No. 63/247,169, filed on Sep. 22, 2021.
(60) Provisional application No. 63/297,397, filed on Jan. 7, 2022.

(51) Int. Cl. A61K 39/215; A61K 9/107; A61K 9/16; A61K 9/19; A61K 39/00; A61P 37/04

(52) U.S. Cl. CPC A61K 39/215; A61K 9/1075; A61K 9/1617; A61K 9/1623; A61K 9/19; A61P 37/04; A61K 2039/53; A61K 2039/55505; A61K 2039/55516

(57) ABSTRACT

The disclosure provides compositions, methods of treatment, and methods of making and using compositions to deliver a nucleic acid to a subject. Methods of using the compositions as a COVID-19 vaccine for the treatment of a coronavirus infection are also provided.

### CROSS-REFERENCE

This application is a continuation of International Application No. PCT/US2022/013513, filed Jan. 24, 2022, which claims the benefit of priority to U.S. Provisional Patent Application No. 63/247,169, filed Sep. 22, 2021, and U.S. Provisional Patent Application No. 63/297,397, filed Jan. 7, 2022, the contents of each of which is incorporated herein by reference in their entirety.

### STATEMENT AS TO FEDERALLY SPONSORED RESEARCH

This invention was made with government support under contracts 75N93020C00052 and 75N93019C00037 awarded by the National Institute of Allergy and Infectious Diseases, Division of Microbiology and Infectious Diseases. The government has certain rights in the invention.

### SEQUENCE LISTING

The instant application contains a Sequence Listing which has been submitted electronically in xml format and is hereby incorporated by reference in its entirety. Said xml copy, created on Apr. 16, 2024, is named 201953-713301-SL.xml and is 258,048 bytes in size.

### BACKGROUND

COVID-19 is an infectious respiratory illness caused by the severe acute respiratory syndrome-corona virus 2 (SARS-CoV-2), which has spread across the world. Commercially available mRNA vaccines provide some protection against COVID-19 disease in individuals infected with SARS-CoV-2, the virus is still highly transmissible and can cause a wide array of symptoms even in vaccinated individuals that become infected. Furthermore, SARS-CoV-2 is known to mutate and the effectiveness of vaccines against current and new variants of SARS-CoV-2 are still being determined. Thus, there is a great need for vaccine compositions that are highly effective against SARS-CoV-2 infections.

### BRIEF SUMMARY

Provided herein are compositions, wherein the compositions comprise a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the at least one nucleic acid comprises a sequence at least 85% identical to SEQ ID NOS: 1-8.

Further provided herein are compositions, wherein the compositions comprise a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the nucleic acid comprises a sequence coding a SARS-CoV-2 omicron variant spike protein antigen sequence or functional variant thereof.

Further provided herein are compositions, wherein the compositions comprise a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the nucleic acid comprises a sequence coding a S2 region, optionally a stem helix, of SARS-CoV-2 spike protein antigen sequence or functional variant thereof.

Further provided herein are compositions, wherein the compositions comprise a lipid carrier, wherein the lipid carrier comprises: liquid oil; an inorganic nanoparticle, wherein the inorganic nanoparticle comprises iron oxide present in an amount of about 0.2 mg/ml 12 nm iron oxide; and surfactants, wherein the surfactants comprise a cationic lipid; and at least one nucleic acid, wherein the nucleic acid comprises a sequence coding a SARS-CoV-2 spike protein antigen sequence or functional variant thereof.

Further provided herein are compositions, wherein the compositions comprise a lipid carrier, wherein the lipid carrier is a nanoemulsion comprising: about 30 mg/mL DOTAP chloride; about 37.5 mg/mL squalene; about 37 mg/ml sorbitan monostearate; about 37 mg/ml polysorbate 80; about 10 mM sodium citrate; and about 0.2 mg Fe/ml 12 nm oleic acid-coated iron oxide nanoparticles; and at least one nucleic acid, wherein the at least one nucleic acid comprises a sequence coding a SARS-CoV-2 spike protein antigen sequence or functional variant thereof.

Further provided herein are compositions, wherein the compositions comprise a lipid carrier, wherein the lipid carrier is a nanoemulsion comprising: DOTAP chloride present in an amount of about 0.75 mg; squalene present in an amount of about 0.94 mg; sorbitan monostearate present in an amount of about 0.93 mg; polysorbate 80 present in an amount of about 0.93 mg; citric acid monohydrate present in an amount of about 1.05 mg; oleic acid-coated iron oxide nanoparticles present in an amount of about 0.005 mg; and at least one nucleic acid, wherein the at least one nucleic acid comprises a sequence coding a SARS-CoV-2 spike protein antigen sequence or functional variant thereof.

Further provided herein are compositions, wherein the compositions comprise: a lipid carrier, wherein the lipid carrier is a nanoemulsion comprising a hydrophobic core, one or more inorganic nanoparticles and one or more lipids, and at least one nucleic acid sequence, wherein the nucleic acid sequence comprises a sequence which encodes an antigen, wherein the antigen is a SARS-CoV-2 spike protein. Further provided herein are vaccines comprising: a lipid carrier, wherein the lipid carrier is a nanoemulsion comprising a hydrophobic core, one or more inorganic nanoparticles and one or more lipids, and at least one nucleic acid sequence, wherein the nucleic acid sequence comprises a sequence which encodes an antigen, wherein the antigen is a SARS-CoV-2 spike protein.

Further provided herein are methods of generating an immune response in a subject, the methods comprising: administering to a subject a composition provided herein thereby generating an immune response to an antigen.

Further provided herein are methods of reducing the severity of a SARS-CoV-2 infection, the methods comprising: administering prior to infection a composition provided herein.

Further provided herein are methods of augmenting an immune response in a subject, the method comprising: administering to a subject the composition provided herein, thereby augmenting an immune response to an antigen.

Further provided herein are methods of treating a coronavirus infection, the method comprising: administering to a subject the composition provided herein, thereby treating the coronavirus infection.

Further provided herein are methods of immunoprotecting a subject, the methods comprise administering to a subject a composition or a vaccine provided herein.

Further provided herein are compositions for immunoprotecting a subject, the compositions comprise: a lipid carrier, wherein the lipid carrier is a nanoemulsion comprising a hydrophobic core, one or more inorganic nanoparticles and one or more lipids, and at least one nucleic acid sequence, wherein the nucleic acid sequence comprises a sequence which encodes an antigen, wherein the antigen is a SARS-CoV-2 spike protein.

Provided herein are dried compositions, the dried compositions comprising: a lipid carrier, wherein the lipid carrier is a nanoemulsion comprising a hydrophobic core, one or more inorganic nanoparticles and one or more lipids, at least one nucleic acid sequence, wherein the nucleic acid sequence comprises a sequence which encodes an antigen, wherein the antigen is a SARS-CoV-2 spike protein, and at least one cryoprotectant.

Further provided herein are methods for preparing a lyophilized composition, the methods comprising: obtaining a lipid carrier, wherein the lipid carrier is a nanoemulsion comprising a hydrophobic core, one or more inorganic nanoparticles and one or more lipids, incorporating at least one nucleic acid into the lipid carrier to form a lipid carrier-nucleic acid complex, adding at least one cryoprotectant to the lipid carrier-nucleic acid complex to form a formulation, and lyophilizing the formulation to form a lyophilized composition.

Further provided herein are methods for preparing a spray-dried composition, the methods comprising: obtaining a lipid carrier, wherein the lipid carrier is a nanoemulsion comprising a hydrophobic core, one or more inorganic nanoparticles and one or more lipids, incorporating at least one nucleic acid into the lipid carrier to form a lipid carrier-nucleic acid complex, adding at least one cryoprotectant to the lipid carrier-nucleic acid complex to form a formulation, and spray drying the formulation to form a spray-dried composition.

Further provided herein are methods for reconstituting a lyophilized composition, the methods comprising: (a) obtaining a lipid carrier, wherein the lipid carrier is a nanoemulsion comprising a hydrophobic core, one or more inorganic nanoparticles, and one or more lipids; (b) incorporating at least one nucleic acid into the said lipid carrier to form a lipid carrier-nucleic acid complex, adding at least one cryoprotectant to the lipid carrier-nucleic acid complex to form a formulation; (c) lyophilizing the formulation to form a lyophilized composition, and reconstituting the lyophilized composition in a suitable diluent.

Further provided herein are methods for reconstituting a spray-dried composition, the methods comprising: (a) obtaining a lipid carrier, wherein the lipid carrier is a nanoemulsion comprising a hydrophobic core, one or more inorganic nanoparticles, and one or more lipids; (b) incorporating at least one nucleic acid into the said lipid carrier to form a lipid carrier-nucleic acid complex; (c) adding at least one cryoprotectant to the lipid carrier-nucleic acid complex to form a formulation; (d) spray drying the formulation to form a spray-dried composition; and (e) reconstituting the spray-dried composition in a suitable diluent.

Further provided herein are compositions for prophylaxis of SARS-CoV-2, wherein the compositions comprise: (a) a sorbitan fatty acid ester; (b) an ethoxylated sorbitan ester; (c) a cationic lipid; (d) an immune stimulant; and (e) at least one RNA molecule.

Further provided herein are compositions for prophylaxis of SARS-CoV-2 wherein the compositions comprise: (a) sorbitan monostearate (e.g., SPAN-60®); (b) polysorbate 80 (e.g., TWEEN-80®); (c) DOTAP; (d) an immune stimulant; and (e) at least one RNA molecule.

Further provided herein are dried compositions comprising: a lipid carrier, wherein the lipid carrier is a nanoemulsion comprising a hydrophobic core, and one or more lipids; at least one nucleic acid that comprises a sequence which encodes a sequence capable of expressing an antigen, optionally wherein the antigen is a SARS-CoV-2 spike protein or a functional variant thereof; and at least one sugar present in amount of (i) at least about 50% by weight of the dried composition, or (ii) present in an amount of least 50 mg.

Further provided herein are kits comprising a composition provided herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which.

FIG. 1 shows repRNA constructs disclosed herein. Schematic representations of the SARS-CoV-2 spike are shown with the signal peptide (sp), S1 and S2 domains, transmembrane domain (TM), and cytoplasmic domain (CD) indicated. The repRNAs are identified as SEQ ID NO: 1 encoding the A.1 spike, B.1 spike, SEQ ID NO: 2 encoding the A.1. pre-fusion stabilized spike, B.1 pre-fusion stabilized spike, SEQ ID NO: 5 encoding the B.1.1.7 pre-fusion stabilized spike; and SEQ ID NO: 4 encoding the B.1.351 pre-fusion stabilized spike. Mutations are shown relative to the A.1 strain (SEQ ID NO: 1).

FIG. 2 shows the effect of spike modifications on binding antibody responses in mice. C57BL/6 mice (n=5/group) were immunized with lipid carrier-formulated repRNA encoding the wild-type (WT), the prefusion-stabilized (PreF), the furin cleavage site-deleted (Furₘᵤₜ), or a combination of the PreF and Furₘᵤₜ modifications of the full-length spike of A.1 lineage SARS-CoV-2. Vaccinations were administered on days 0 and 28, and serum collected on days 14, 28, and 38 assayed for anti-spike binding IgG by enzyme linked immunosorbent assay.

FIGS. 3A-3C show schematic representations of exemplary nanoparticle (NP) carriers. FIG. 3A shows an oil-in-water emulsion. FIG. 3B shows a nanostructured lipid carrier (NLC). FIG. 3C shows a nanoparticle having an inorganic nanoparticle in liquid oil.

FIGS. 4A-4B show the increased protein production induced by the Miglyol lipid carrier formulation, NP-3. FIG. 4A shows the first assay. FIG. 4B shows the second assay.

FIGS. 5A-5B show the decreased immune response induced by the Miglyol lipid carrier formulation, NP-3. FIG. 5A shows the first assay. FIG. 5B shows the second assay.

FIGS. 6A-6B show the correlation between enhanced protein production and low TNF (e.g., TNF alpha) stimulation observed with NP-3 as a result of the first and second assays. FIG. 6A shows the first assay. FIG. 6B shows the second assay.

FIGS. 7A-7F show SEAP levels in BALB/c mice injected intramuscularly with various embodiments of lipid carrier formulations described herein. FIG. 7A shows SEAP levels on day 4 post-injection. FIG. 7B shows SEAP levels on day 6 post-injection. FIG. 7C shows SEAP levels on day 8 post-injection. FIG. 7D shows SEAP levels on day 4 post-injection.

FIG. 7E shows SEAP levels on day 6 post-injection. FIG. 7F shows SEAP levels on day 8 post-injection. X-axis: Condition, Y-axis: Relative light units (RLU).

FIG. 8 is a bar chart with measurements of Z-average measurement and polydispersity index (PDI) on the Y-axis and group number on the X-axis for conditions 1 to 14.

FIGS. 9A-9B are dot charts with IgG (μg/ml) on the Y-axis, group number on the X-axis for conditions 1 to 14, and recordings shown for measurements at day 14 anti-D614G (1:40 dilution) and day 28 anti-D614G (1:200 dilution), respectively.

FIG. 10 shows a dot chart at day 28 with anti-D614G (1:200 dilution) IgG (ug/ml) measurements on the Y-axis and indications of storage conditions on the X-axis.

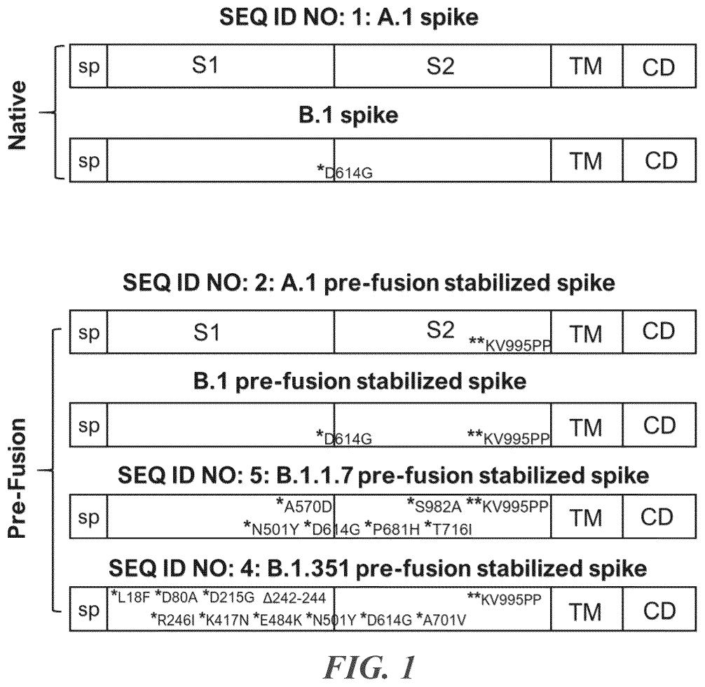

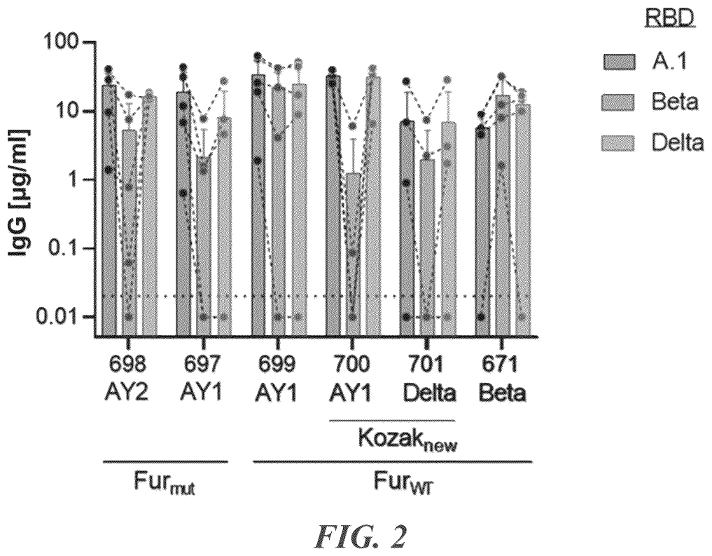

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### DETAILED DESCRIPTION OF THE INVENTION

Provided herein are compositions, kits, methods, and uses thereof for inducing an immune response to a coronavirus. Briefly, further described herein are (1) nanoparticle carrier systems; (2) nucleic acids encoding for coronavirus antigens and RNA polymerases; (3) combination compositions; (4) thermally stable, dried, and lyophilized SARS-CoV-2 RNA vaccines; (5) pharmaceutical compositions; (6) dosing; (7) administration; (8) therapeutic applications; and (9) kits.

### Definitions

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document. All references disclosed herein, including patent references and non-patent references, are hereby incorporated by reference in their entirety as if each was incorporated individually. However, where a patent, patent application, or publication containing express definitions is incorporated by reference, those express definitions should be understood to apply to the incorporated patent, patent application, or publication in which they are found, and not necessarily to the text of this application, in particular the claims of this application, in which instance, the definitions provided herein are meant to supersede.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, “optional” or “optionally” means that the subsequently described circumstance may or may not occur, so that the description includes instances where the circumstance occurs and instances where it does not.

As used herein, the term “about” or “approximately” means a range of up to +20%, of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “effective amount” or “therapeutically effective amount” refers to an amount that is sufficient to achieve or at least partially achieve the desired effect.

### Nanoparticle Carrier Systems

Provided herein are various compositions comprising a nanoparticle or a plurality of nanoparticles. Nanoparticles also referred to herein as carriers or abbreviated as NPs. Nanoparticles provided herein may be an organic, inorganic, or a combination of inorganic and organic materials that are less than about 1 micrometer (μm) in diameter. In some embodiments, nanoparticles provided herein are used as a delivery system for a bioactive agent provided herein.

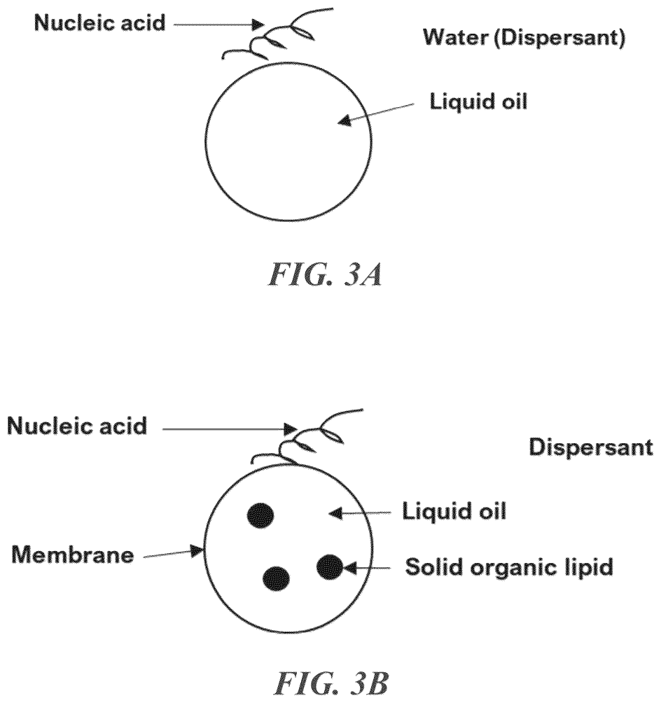

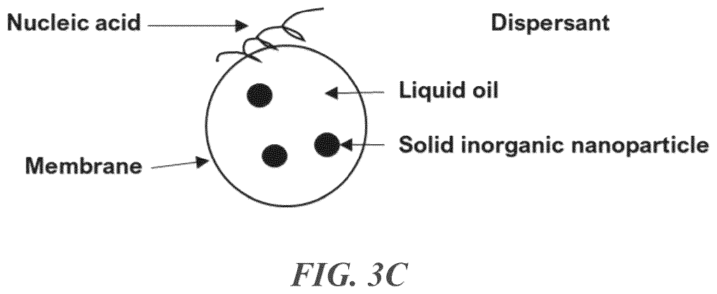

Various nanoparticles and formulations of nanoparticles (i.e., nanoemulsions) are employed. Exemplary nanoparticles are illustrated in FIGS. 3A-3C. Nanoparticles provided herein can include but are not limited to: oil in water emulsions, nanostructured lipid carriers (NLCs), cationic nanoemulsions (CNEs), vesicular phospholipid gels (VPG), polymeric nanoparticles, cationic lipid nanoparticles, liposomes, gold nanoparticles, solid lipid protein nanoparticles, polyethylenimine nanoparticles (LNPs or SLNs), mixed phase core NLCs, ionizable lipid carriers, magnetic carriers, polyethylene glycol (PEG)-functionalized carriers, cholesterol-functionalized carriers, polylactic acid (PLA)-functionalized carriers, and polylactic-co-glycolic acid (PLGA)-functionalized lipid carriers.

Oil in water emulsions, as illustrated in FIG. 3A (not to scale), are stable, immiscible fluids containing an oil droplet dispersed in water or aqueous phase. FIG. 3B (not to scale) illustrates a nanostructured lipid carrier (NLCs) which can comprise a blend of solid organic lipids (e.g., trimyristin) and liquid oil (e.g., squalene). In NLCs, the solid lipid is dispersed in the liquid oil. The entire nanodroplet is dispersed in the aqueous (water) phase. In some embodiments, the nanoparticle comprises inorganic nanoparticles, as illustrated in FIG. 3C (not to scale), as solid inorganic nanoparticles (e.g., iron oxide nanoparticles) dispersed in liquid oil. The entire nanodroplet is then dispersed as a colloid in the aqueous (water) phase. In some embodiments, the nanoparticles provided herein are dispersed in an aqueous solution. Non-limiting examples of aqueous solutions include water (e.g., sterilized, distilled, deionized, ultrapure, RNase-free, etc.), saline solutions (e.g., Kreb's, Ascaris, Dent's, Tet's saline), or 1% (w/v) dimethyl sulfoxide (DMSO) in water.

In some embodiments, the nanoparticles provided herein comprise a hydrophilic surface. In some embodiments, the hydrophilic surface comprises a cationic lipid. In some embodiments, the hydrophilic surface comprises an ionizable lipid. In some embodiments, the nanoparticle comprises a membrane. In some embodiments, the membrane comprises a cationic lipid. In some embodiments, the nanoparticles provided herein comprise a cationic lipid. Exemplary cationic lipids for inclusion in the hydrophilic surface include, without limitation: 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[l-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxy-dodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″, 9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9′″Z,12Z,12′,12″Z,12′″Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Other examples for suitable classes of lipids include, but are not limited to, the phosphatidylcholines (PCs), phosphatidylethanolamines (PEs), phosphatidylglycerol (PGs); and PEGylated lipids including PEGylated version of any of the above lipids (e.g., DSPE-PEGs). In some embodiments, the nanoparticle provided herein comprises DOTAP.

In some embodiments, the nanoparticle provided herein comprises an oil. In some embodiments, the oil is in liquid phase. Non-limiting examples of oils that can be used include α-tocopherol, coconut oil, dihydroisosqualene (DHIS), farnesene, grapeseed oil, lauroyl polyoxylglyceride, mineral oil, monoacylglycerol, palmkemal oil, olive oil, paraffin oil, peanut oil, propolis, squalene, squalane, solanesol, soy lecithin, soybean oil, sunflower oil, a triglyceride, or vitamin E. In some embodiments, the nanoparticle provided herein comprises a triglyceride. Exemplary triglycerides include but are not limited to: capric triglycerides, caprylic triglycerides, a caprylic and capric triglycerides, triglyceride esters, and myristic acid triglycerins.

In some embodiments, the nanoparticles provided herein comprise a liquid organic material and a solid inorganic material. In some embodiments, the nanoparticle provided herein comprises an inorganic particle. In some embodiments, the inorganic particle is a solid inorganic particle. In some embodiments, the nanoparticle provided herein comprises the inorganic particle within the hydrophobic core.

In some embodiments, the nanoparticle provided herein comprises a metal. In some embodiments, the nanoparticle provided herein comprises a metal within the hydrophobic core. The metal can be without limitation, a metal salt such as a transition metal salt, a metal oxide such as a transition metal oxide, a metal hydroxide such as a transition metal hydroxide, a metal phosphate such as a transition metal phosphate, or a metalloid (e.g., silicon and silicon-based compounds or alloys). In some embodiments, the nanoparticle provided herein comprises aluminum oxide (Al₂O₃), aluminum oxyhydroxide, iron oxide (Fe₃O₄, Fe₂O₃, FeO, or combinations thereof), titanium dioxide, silicon dioxide (SiO₂), aluminum hydroxyphosphate (Al(OH)ₓ(PO₄)_{y}), calcium phosphate (Ca₃(PO₄)₂), calcium hydroxyapatite (Ca₁₀(PO₄)₆(OH)₂), iron gluconate, or iron sulfate. The inorganic particles may be formed from one or more same or different metals (any metals including transition metal). In some embodiments, the inorganic particle is a transition metal oxide. In some embodiments, the transition metal is magnetite (Fe₃O₄), maghemite (y-Fe₂O₃), wüstite (FeO), or hematite (alpha (α)-Fe₂O₃). In some embodiments, the metal is aluminum hydroxide or aluminum oxyhydroxide, and a phosphate-terminated lipid or a surfactant, such as oleic acid, oleylamine, SDS, TOPO or DSPA is used to coat the inorganic solid nanoparticle before it is mixed with the liquid oil to form the hydrophobic core. In some embodiments, the metal can comprise a paramagnetic, a superparamagnetic, a ferrimagnetic or a ferromagnetic compound. In some embodiments, the metal is a superparamagnetic iron oxide (Fe₃O₄).

In some embodiments, the nanoparticle provided herein comprises a cationic lipid, an oil, and an inorganic particle. In some embodiments, the nanoparticle provided herein comprises DOTAP; squalene and/or glyceryl trimyristate-dynasan; and iron oxide. In some embodiments, the nanoparticle provided herein further comprises a surfactant. Thus, in some embodiments, the nanoparticles provided herein comprise a cationic lipid, an oil, an inorganic particle, and a surfactant.

Surfactants are compounds that lower the surface tension between two liquids or between a liquid and a solid component of the nanoparticles provided herein. Surfactants can be hydrophobic, hydrophilic, or amphiphilic. In some embodiments, the nanoparticle provided herein comprises a hydrophobic surfactant. Exemplary hydrophobic surfactants that can be employed include but are not limited to: sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), and sorbitan trioleate (SPAN® 85). Suitable hydrophobic surfactants include those having a hydrophilic-lipophilic balance (HLB) value of 10 or less, for instance, 5 or less, from 1 to 5, or from 4 to 5. For instance, the hydrophobic surfactant can be a sorbitan ester having an HLB value from 1 to 5, or from 4 to 5.

In some embodiments, the nanoparticle provided herein comprises a hydrophilic surfactant, also called an emulsifier. In some embodiments, the nanoparticle provided herein comprises polysorbate. Polysorbates are oily liquids derived from ethoxylated sorbitan (a derivative of sorbitol) esterified with fatty acids. Exemplary hydrophilic surfactants that can be employed include but are not limited to: polysorbates such as Tween, Kolliphor, Scattics, Alkest, or Canarcel; polyoxyethylene sorbitan ester (polysorbate); polysorbate 80 (polyoxyethylene sorbitan monooleate, or Tween 80); polysorbate 60 (polyoxyethylene sorbitan monostearate, or Tween 60); polysorbate 40 (polyoxyethylene sorbitan monopalmitate, or Tween 40); and polysorbate 20 (polyoxyethylene sorbitan monolaurate, or Tween 20). In one embodiment, the hydrophilic surfactant is polysorbate 80.

Nanoparticles provided herein comprises a hydrophobic core surrounded by a lipid membrane (e.g., a cationic lipid such as DOTAP). In some embodiments, the hydrophobic core comprises: one or more inorganic particles; a phosphate-terminated lipid; and a surfactant.

Inorganic solid nanoparticles described herein may be surface modified before mixing with the liquid oil. For instance, if the surface of the inorganic solid nanoparticle is hydrophilic, the inorganic solid nanoparticle may be coated with hydrophobic molecules (or surfactants) to facilitate the miscibility of the inorganic solid nanoparticle with the liquid oil in the “oil” phase of the nanoemulsion particle. In some embodiments, the inorganic particle is coated with a capping ligand, the phosphate-terminated lipid, and/or the surfactant. In some embodiments the hydrophobic core comprises a phosphate-terminated lipid. Exemplary phosphate-terminated lipids that can be employed include but are not limited to: trioctylphosphine oxide (TOPO) or distearyl phosphatidic acid (DSPA). In some embodiments, the hydrophobic core comprises a surfactant such as a phosphorous-terminated surfactant, a carboxylate-terminated surfactant, a sulfate-terminated surfactant, or an amine-terminated surfactant. Typical carboxylate-terminated surfactants include oleic acid. Typical amine terminated surfactants include oleylamine. In some embodiments, the surfactant is distearyl phosphatidic acid (DSPA), oleic acid, oleylamine or sodium dodecyl sulfate (SDS). In some embodiments, the inorganic solid nanoparticle is a metal oxide such as an iron oxide, and a surfactant, such as oleic acid, oleylamine, SDS, DSPA, or TOPO, is used to coat the inorganic solid nanoparticle before it is mixed with the liquid oil to form the hydrophobic core.

In some embodiments, the hydrophobic core comprises: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate.

In some embodiments, the hydrophobic core comprises: one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80.

In some embodiments, the hydrophobic core consists of: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate. In some embodiments, the hydrophobic core consists of: one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80. In some embodiments, the nanoparticle provided herein can comprise from about 0.2% to about 40% w/v squalene, from about 0.001% to about 10% w/v iron oxide nanoparticles, from about 0.2% to about 10% w/v DOTAP, from about 0.25% to about 5% w/v sorbitan monostearate, and from about 0.5% to about 10% w/v polysorbate 80. In some embodiments the nanoparticle provided herein from about 2% to about 6% w/v squalene, from about 0.01% to about 1% w/v iron oxide nanoparticles, from about 0.2% to about 1% w/v DOTAP, from about 0.25% to about 1% w/v sorbitan monostearate, and from about 0.5%) to about 5% w/v polysorbate 80. In some embodiments, the nanoparticle provided herein can comprise from about 0.2% to about 40% w/v squalene, from about 0.001% to about 10% w/v aluminum hydroxide or aluminum oxyhydroxide nanoparticles, from about 0.2% to about 10% w/v DOTAP, from about 0.25% to about 5% w/v sorbitan monostearate, and from about 0.5% to about 10% w/v polysorbate 80. In some embodiments, the nanoparticle provided herein can comprise from about 2% to about 6% w/v squalene, from about 0.01% to about 1% w/v aluminum hydroxide or aluminum oxyhydroxide nanoparticles, from about 0.2% to about 1% w/v DOTAP, from about 0.25% to about 1% w/v sorbitan monostearate, and from about 0.5%) to about 5% w/v polysorbate 80.

In some embodiments, a composition described herein comprises at least one nanoparticle formulations as described in Table 1. In some embodiments, a composition described herein comprises any one of NP-1 to NP-30. In some embodiments, a composition
described herein comprises any one of NP-1 to NP-31. In some embodiments, the nanoparticles provided herein are admixed with a nucleic acid provided herein. In some embodiments, nanoparticles provided herein are made by homogenization and ultrasonication techniques.

TABLE 1
Nanoparticle Formulations.

| | | | | Additional |
|---|---|---|---|---|
| | | | | Ingredients |
| | Cationic Lipid(s) | Oil(s) | Surfactant(s) | % (w/v), |
| Name | % (w/v) or mg/ml | % (w/v) or mg/ml | % (w/v) or mg/ml | mg/ml, or mM |
| NP-1 | 30 mg/ml 1,2- | 37.5 mg/ml squalene | 37 mg/ml sorbitan | 0.2 mg Fe/ml |
| | dioleoyl-3- | | monostearate, | 12 nm oleic |
| | trimethylammonium- | | (2R)-2- | acid-coated iron |
| | propane (DOTAP) | | [(2R,3R,4S)-3,4- | oxide |
| | chloride | | Dihydroxyoxolan- | nanoparticles |
| | | | 2-yl]-2- | 10 mM sodium |
| | | | hydroxyethyl | citrate |
| | | | octadecenoate, | dihydrate. |
| | | | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN 80 ®) | |
| NP-2 | 30 mg/ml 1,2- | 37.5 mg/ml squalene | 37 mg/ml sorbitan | 1 mg Fe/ml 15 |
| | dioleoyl-3- | | monostearate | nm oleic acid- |
| | trimethylammonium- | | (2R)-2- | coated iron |
| | propane (DOTAP) | | [(2R,3R,4S)-3,4- | oxide |
| | chloride | | Dihydroxyoxolan- | nanoparticles |
| | | | 2-yl]-2- | 10 mM sodium |
| | | | hydroxyethyl | citrate dihydrate |
| | | | octadecenoate | |
| | | | C₂₄H₄₆O₆ | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN 80 ®) | |
| NP-3 | 30 mg/ml 1,2- | 37.5 mg/ml Miglyol | 37 mg/ml sorbitan | 0.2 mg Fe/ml |
| | dioleoyl-3- | 812N | monostearate, | 15 nm oleic |
| | trimethylammonium- | (triglyceride ester of | (2R)-2- | acid-coated iron |
| | propane (DOTAP) | saturated | [(2R,3R,4S)-3,4- | oxide |
| | chloride | coconut/palmkernel oil | Dihydroxyoxolan- | nanoparticles |
| | | derived caprylic and | 2-yl]-2- | 10 mM sodium |
| | | capric fatty acids and | hydroxyethyl | citrate dihydrate |
| | | plant derived glycerol) | octadecenoate | |
| | | | C₂₄H₄₆O₆ | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN 80 ®) | |
| NP-4 | 30 mg/ml 1,2- | 37.5 mg/ml Miglyol | 37 mg/ml sorbitan | 1 mg Fe/ml 15 |
| | dioleoyl-3- | 812N | monostearate, | nm oleic acid- |
| | trimethylammonium- | (triglyceride ester of | (2R)-2- | coated iron |
| | propane (DOTAP) | saturated | [(2R,3R,4S)-3,4- | oxide |
| | chloride | coconut/palmkernel oil | Dihydroxyoxolan- | nanoparticles |
| | | derived caprylic and | 2-yl]-2- | 10 mM sodium |
| | | capric fatty acids and | hydroxyethyl | citrate |
| | | plant derived glycerol) | octadecenoate, | dihydrate. |
| | | | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN 80 ®) | |
| NP-5 | 30 mg/ml DOTAP | 37.5 mg/ml squalene | 37 mg/ml sorbitan | 1 mg/ml |
| | chloride | | monostearate | trioctylphosphine |
| | | | (SPAN ® 60) | oxide |
| | | | 37 mg/ml | (TOPO)-coated |
| | | | polysorbate 80 | aluminum |
| | | | (TWEEN ® 80) | hydroxide |
| | | | | (Alhydrogel ® |
| | | | | 2%) particles |
| | | | | 10 mM sodium |
| | | | | citrate dihydrate |
| NP-6 | 30 mg/ml DOTAP | 37.5 mg/ml Solanesol | 37 mg/ml sorbitan | 0.2 mg Fe/ml |
| | chloride | (Cayman chemicals) | monostearate | oleic acid- |
| | | | (SPAN ® 60) | coated iron |
| | | | 37 mg/ml | oxide |
| | | | polysorbate 80 | nanoparticles |
| | | | (TWEEN ® 80) | 10 mM sodium |
| | | | | citrate |
| NP-7 | 30 mg/ml DOTAP | 37.5 mg/ml squalene | 37 mg/ml sorbitan | 10 mM sodium |
| | chloride | 2.4 mg/ml Dynasan | monostearate | citrate |
| | | 114 | (SPAN ® 60) | |
| | | | 37 mg/ml | |
| | | | polysorbate 80 | |
| | | | (TWEEN ® 80 | |
| NP-8 | 4 mg/ml DOTAP | 43 mg/ml squalene | 5 mg/ml Span ® | 10 mM sodium |
| | chloride | | 85 | citrate |
| | | | 5 mg/ml Tween ® | |
| | | | 80 | |
| NP-9 | 7.5 mg/ml 1,2- | 9.4 mg/ml squalene | 9.3 mg/ml | 0.05 mg/ml 15 |
| | dioleoyl-3- | ((6E,10E,14E,18E)- | sorbitan | nanometer |
| | trimethylammonium- | 2,6,10,15,19,23- | monostearate | superparamagnetic |
| | propane (DOTAP) | Hexamethyltetracosa- | (2R)-2- | iron oxide |
| | chloride | 2,6,10,14,18,22- | [(2R,3R,4S)-3,4- | (Fe₃O₄) |
| | | hexaene, C₃₀H₅₀) | Dihydroxyoxolan- | 10 mM sodium |
| | | 0.63 mg/ml glyceryl | 2-yl]-2- | citrate dihydrate |
| | | trimyristate-dynasan | hydroxyethyl | |
| | | (DYNASAN 114 ®) | octadecenoate, | |
| | | | C₂₄H₄₆O₆) | |
| | | | (SPAN ® 60) | |
| | | | 9.3 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN 80 ®) | |
| NP-10 | 0.4% DOTAP | 0.25% glyceryl | 0.5% sorbitan | |
| | | trimyristate-dynasan | monostearate | |
| | | (DYNASAN 114 ®) | (SPAN ® 60) | |
| | | 4.75% Squalene | 0.5% polysorbate | |
| | | | 80 (TWEEN 80 ®) | |
| NP-11 | 3.0% DOTAP | 0.25% glyceryl | 3.7% sorbitan | |
| | | trimyristate-dynasan | monostearate | |
| | | (DYNASAN 114 ®) | (SPAN ® 60) | |
| | | 3.75% Squalene | 3.7% polysorbate | |
| | | | 80 (TWEEN 80 ®) | |
| NP-12 | 0.4% DOTAP | 4.3% Squalene | 0.5% sorbitan | |
| | | | trioleate (SPAN ® | |
| | | | 85) | |
| | | | 0.5% polysorbate | |
| | | | 80 (TWEEN ® 80) | |
| NP-13 | 0.4% DOTAP | 0.25% glyceryl | 2.0% polysorbate | |
| | | trimyristate-dynasan | 80 (TWEEN ® 80) | |
| | | (DYNASAN 114 ®) | | |
| | | 4.08% squalene | | |
| NP-14 | 0.4% DOTAP | 0.25% glyceryl | 0.5% sorbitan | |
| | | trimyristate-dynasan | trioleate (SPAN ® | |
| | | (DYNASAN 114 ®) | 85) | |
| | | 4.08% squalene | 2.0% polysorbate | |
| | | | 80 (TWEEN ® 80) | |
| NP-15 | 0.4% DOTAP | 0.25% glyceryl | 0.25% sorbitan | |
| | | trimyristate-dynasan | trioleate (SPAN ® | |
| | | (DYNASAN 114 ®) | 85) | |
| | | 4.08% squalene | 2.0% polysorbate | |
| | | | 80 (TWEEN ® 80) | |
| NP-16 | 0.4% DOTAP | 5% squalene | 0.5% sorbitan | |
| | | | trioleate (SPAN ® | |
| | | | 85) | |
| | | | 2.0% polysorbate | |
| | | | 80 (TWEEN ® 80) | |
| NP-17 | 0.4% DOTAP | 5% squalene | 0.5% sorbitan | |
| | | | monostearate | |
| | | | (SPAN ® 60) | |
| | | | 2% polysorbate | |
| | | | 80 (TWEEN ® 80) | |
| NP-18 | 0.4% DOTAP | 0.25% glyceryl | 2% sorbitan | |
| | | trimyristate-dynasan | trioleate (SPAN ® | |
| | | (DYNASAN 114 ®) | 85) | |
| | | 4.08% squalene | | |
| | | | 2% polysorbate | |
| | | | 80 (TWEEN ® 80) | |
| NP-19 | 0.4% DOTAP | 0.25% glyceryl | 0.5% sorbitan | 1% aluminum |
| | | trimyristate-dynasan | monostearate | hydroxide |
| | | (DYNASAN 114 ®) | (SPAN ® 60) | |
| | | 4.75% Squalene | 0.5% polysorbate | |
| | | | 80 (TWEEN 80 ®) | |
| NP-20 | 3.0% DOTAP | 0.25% glyceryl | 3.7% sorbitan | 1% aluminum |
| | | trimyristate-dynasan | monostearate | hydroxide |
| | | (DYNASAN 114 ®) | (SPAN ® 60) | |
| | | 3.75% Squalene | 3.7% polysorbate | |
| | | | 80 (TWEEN ® 80) | |
| NP-21 | 0.4% DOTAP | 4.3% Squalene | 0.5% sorbitan | 1% aluminum |
| | | | trioleate (SPAN ® | hydroxide |
| | | | 85) | |
| | | | 0.5% polysorbate | |
| | | | 80 (TWEEN ® 80 | |
| NP-22 | 0.4% DOTAP | 0.25% glyceryl | 2.0% polysorbate | 1% aluminum |
| | | trimyristate-dynasan | 80 (TWEEN ® 80) | hydroxide |
| | | (DYNASAN 114 ®) | | |
| | | 4.08% squalene | | |
| NP-23 | 0.4% DOTAP | 0.25% glyceryl | 0.5% sorbitan | 1% aluminum |
| | | trimyristate-dynasan | trioleate (SPAN ® | hydroxide |
| | | (DYNASAN 114 ®) | 85) | |
| | | 4.08% squalene | 2.0% polysorbate | |
| | | | 80 (TWEEN ® 80 | |
| NP-24 | 0.4% DOTAP | 0.25% glyceryl | 0.25% sorbitan | 1% aluminum |
| | | trimyristate-dynasan | trioleate (SPAN ® | hydroxide |
| | | (DYNASAN 114 ®) | 85) | |
| | | 4.08% squalene | 2.0% polysorbate | |
| | | | 80 (TWEEN ® 80 | |
| NP-25 | 0.4% DOTAP | 5% squalene | 0.5% sorbitan | 1% aluminum |
| | | | trioleate (SPAN ® | hydroxide |
| | | | 85) | |
| | | | 2.0% polysorbate | |
| | | | 80 (TWEEN ® 80 | |
| NP-26 | 0.4% DOTAP | 5% squalene | 0.5% sorbitan | 1% aluminum |
| | | | monostearate | hydroxide |
| | | | (SPAN ® 60) | |
| | | | 2% polysorbate | |
| | | | 80 (TWEEN ® 80) | |
| NP-27 | 0.4% DOTAP | 0.25% glyceryl | 2% sorbitan | 1% aluminum |
| | | trimyristate-dynasan | trioleate (SPAN ® | hydroxide |
| | | (DYNASAN 114 ®) | 85) | |
| | | 4.08% squalene | 2% polysorbate | |
| | | | 80 (TWEEN ® 80) | |
| NP-28 | 0.5-5.0 mg/ml | 0.2-10% (v/v) | 0.01-2.5% (v/v) | |
| | DOTAP | squalene | polysorbate 80 | |
| | | | (TWEEN ® 80) | |
| NP-29 | 0.4% (w/w) DOTAP | 4.3% (w/w) squalene | 0.5% (w/w) | |
| | | | sorbitan trioleate | |
| | | | (SPAN ® 85) | |
| | | | 0.5% (w/w) | |
| | | | polysorbate 80 | |
| | | | (TWEEN ® 80) | |
| NP-30 | 30 mg/ml DOTAP | 37.5 mg/ml squalene | 37 mg/ml sorbitan | 10 mM sodium |
| | chloride | | monostearate | citrate |
| | | | (SPAN ® 60) | |
| | | | 37 mg/ml | |
| | | | polysorbate 80 | |
| | | | (TWEEN ® 80) | |
| NP-31 | 30 mg/ml DOTAP | 37.5 mg/ml squalene | 37 mg/ml sorbitan | 0.4 mg Fe/ml |
| | chloride | | monostearate | 5 nm oleic acid- |
| | | | (SPAN ® 60) | coated iron |
| | | | 37 mg/ml | oxide |
| | | | polysorbate 80 | nanoparticles |
| | | | (TWEEN ® 80) | 10 mM sodium |
| | | | | citrate dihydrate |

In some embodiments, nanoparticles provided herein comprise: sorbitan monostearate (e.g., SPAN-60), polysorbate 80 (e.g., TWEEN-80), DOTAP, squalene, and no solid particles. In some embodiments, nanoparticles provided herein comprise: sorbitan monostearate (e.g., SPAN-60), polysorbate 80 (e.g., TWEEN-80), DOTAP, squalene, and iron oxide particles. In some embodiments, nanoparticles provided herein comprise an immune stimulant. In some embodiments, the immune stimulant is squalene. In some embodiments, the immune stimulant is Miglyol 810 or Miglyol 812. In some embodiments, the immune stimulant can decrease the total amount of protein produced, but can increase the immune response to a composition provided herein (e.g., when delivered as a vaccine). In some embodiments, the immune stimulant can increase the total amount of protein produced, but can decrease the immune response to a composition provided herein.

Nanoparticles provided herein can be of various average diameters in size. In some embodiments, nanoparticles provided herein have an average diameter (z-average hydrodynamic diameter, measured by dynamic light scattering) ranging from about 20 nm to about 200 nm. In some embodiments, the z-average diameter of the nanoparticle ranges from about 20 nm to about 150 nm, from about 20 nm to about 100 nm, from about 20 nm to about 80 nm, from about 20 nm to about 60 nm. In some embodiments, the z-average diameter of the nanoparticle) ranges from about 40 nm to about 200 nm, from about 40 nm to about 150 nm, from about 40 nm to about 100 nm, from about 40 nm to about 90 nm, from about 40 nm to about 80 nm, or from about 40 nm to about 60 nm. In one embodiment, the z-average diameter of the nanoparticle is from about 40 nm to about 80 nm. In some embodiments, the z-average diameter of the nanoparticle is from about 40 nm to about 60 nm. In some embodiments, the nanoparticle is up to 100 nm in diameter. In some embodiments, the nanoparticle is 50 to 70 nm in diameter. In some embodiments, the nanoparticle is 40 to 80 nm in diameter. In some embodiments, the inorganic particle (e.g., iron oxide) within the hydrophobic core of the nanoparticle can be an average diameter (number weighted average diameter) ranging from about 3 nm to about 50 nm. For instance, the inorganic particle can have an average diameter of about 5 nm, about 10 nm, about 15 nm, about 20 nm, about 25 nm, about 30 nm, about 35 nm, about 40 nm, about 45 nm, or about 50 nm. In some embodiments, the ratio of esters and lipids yield a particle size between 30 nm and 200 nm. In some embodiments, the ratio of esters and lipids yield a particle size between 40 nm and 70 nm.

Nanoparticles provided herein may be characterized by the polydispersity index (PDI), which is an indication of their quality with respect to size distribution. In some embodiments, average polydispersity index (PDI) of the nanoparticles provided herein ranges from about 0.1 to about 0.5. In some embodiments, the average PDI of the nanoparticles can range from about 0.2 to about 0.5, from about 0.1 to about 0.4, from about 0.2 to about 0.4, from about 0.2 to about 0.3, or from about 0.1 to about 0.3.

In some embodiments, the nanoparticles provided herein comprise an oil-to-surfactant molar ratio ranging from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about 1:1. In some embodiments, the nanoparticles provided herein comprise a hydrophilic surfactant-to-lipid ratio ranging from about 0.1:1 to about 2:1, from about 0.2:1 to about 1.5:1, from about 0.3:1 to about 1:1, from about 0.5:1 to about 1:1, or from about 0.6:1 to about 1:1. In some embodiments, the nanoparticles provided herein comprise a hydrophobic surfactant-to-lipid ratio ranging from about 0.1:1 to about 5:1, from about 0.2:1 to about 3:1, from about 0.3:1 to about 2:1, from about 0.5:1 to about 2:1, or from about 1:1 to about 2:1.

In some embodiments, the nanoparticles provided herein comprise from about 0.2% to about 40% w/v liquid oil, from about 0.001% to about 10% w/v inorganic solid nanoparticle, from about 0.2% to about 10% w/v lipid, from about 0.25% to about 5% w/v hydrophobic surfactant, and from about 0.5% to about 10% w/v hydrophilic surfactant. In some embodiments, the lipid comprises a cationic lipid, and the oil comprises squalene, and/or the hydrophobic surfactant comprises sorbitan ester.

In some embodiments, nanoparticles provided herein comprise a ratio of the esters that yields a hydrophilic-lipophilic balance between 8 and 11.

In some embodiments, nucleic acids provided herein are incorporated, associated with, or complexed a lipid carrier provided herein to form a lipid carrier-nucleic acid complex. The lipid carrier-nucleic acid complex is formed via non-covalent interactions or via reversible covalent interactions.

### Nucleic Acids

Provided herein is a composition comprising a nucleic acid. In some embodiments, the nucleic acid is in complex with the nanoparticle. In some embodiments, the nucleic acid is in complex with the membrane of the nanoparticle. In some embodiments, the nucleic acid is in complex with the hydrophilic surface of the nanoparticle. In some embodiments, the nucleic acid is within the nanoparticle. In some embodiments, the nucleic acid is within the hydrophobic core.

In some embodiments, the nucleic acid is deoxyribonucleic acid (DNA) or ribonucleic acid (RNA). The nucleic acid may be linear or include a secondary structure (e.g., a hair pin). In some embodiments, the nucleic acid is a polynucleotide comprising modified nucleotides or bases, and/or their analogs. A polynucleotide may comprise modified nucleotides, such as methylated nucleotides and their analogs. If present, modification to the nucleotide structure may be imparted before or after assembly of compositions provided herein. Modified nucleobases which can be incorporated into modified nucleosides and nucleotides and be present in the RNA molecules include: m5C (5-methylcytidine), m5U (5-methyluridine), m6A (N6-methyladenosine), s2U (2-thiouridine), Um (2′-O-methyluridine), m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A (N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); m1I (1-methylinosine); m′Im (1,2′-O-dimethylinosine); m3C (3-methylcytidine); Cm (2T-O-methylcytidine); s2C (2-thiocytidine); ac4C (N4-acetylcytidine); f5C (5-fonnylcytidine); m5Cm (5,2-O-dimethylcytidine); ac4Cm (N4acetyl2TOmethylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); hoSU (5-hydroxyuridine); moSU (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethylaminomethyl-2-L-Omethyluridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,T-O-dimethyladenosine); rn62Am (N6,N6,O-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); f5Cm (5-formyl-2′-O-methylcytidine); m1Gm (1,2′-O-dimethylguanosine); m′Am (1,2-O-dimethyl adenosine) irinomethyluridine); tm5s2U (S-taurinomethyl-2-thiouridine)); imG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-methylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Any one or any combination of these modified nucleobases may be included in the self-replicating RNA of the invention. Many of these modified nucleobases and their corresponding ribonucleosides are available from commercial suppliers. If desired, the nucleic acid can contain phosphoramidate, phosphorothioate, and/or methylphosphonate linkages. The RNA sequence can be modified with respect to its codon usage, for example, to increase translation efficacy and half-life of the RNA. A poly A tail (e.g., of about 30 adenosine residues or more) may be attached to the 3′ end of the RNA to increase its half-life. The 5′ end of the RNA may be capped with a modified ribonucleotide with the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methyltransferase, which catalyzes the construction of N7-monomethylated cap 0 structures). Cap structure can provide stability and translational efficacy to the RNA molecule. The 5′ cap of the RNA molecule may be further modified by a 2′-O-Methyltransferase which results in the generation of a cap 1 structure (m7Gppp [m2′-O] N), which may further increase translation efficacy. A cap 1 structure may also increase in vivo potency.

In some embodiments, compositions provided herein comprise one or more nucleic acids. In some embodiments, compositions provided herein comprise two or more nucleic acids. In some embodiments, compositions provided herein comprise at least one DNA. In some embodiments, compositions provided herein comprise at least one RNA. In some embodiments, compositions provided herein comprise at least one DNA and at least one RNA. In some embodiments, nucleic acids provided herein are present in an amount of above 5 ng to about 1 mg. In some embodiments, nucleic acids provided herein are present in an amount of up to about 25, 50, 75, 100, 150, 175 ng. In some embodiments, nucleic acids provided herein are present in an amount of up to about 1 mg. In some embodiments, nucleic acids provided herein are present in an amount of about 0.05 μg, 0.1 μg, 0.2 μg, 0.5 μg, 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, 1 mg. In some embodiments, nucleic acids provided herein are present in an amount of 0.05 μg, 0.1 μg, 0.2 μg, 0.5 μg, 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, 1 mg. In some embodiments, the nucleic acid is at least about 200, 250, 500, 750, 1000, 2000, 3000, 4000, 5000, 6000, 7000, 8000, 9000, 10,000, 11,000, 12,000, 13,000, 14,000, 15,000, 16,000, 17,000, 18,000, 19,000, or 20,000 nucleotides in length. In some embodiments, the nucleic acid is up to about 7000, 8000, 9000, 10,000, 11,000, 12,000, 13,000, 14,000, 15,000, 16,000, 17,000, 18,000, 19,000, or 20,000 nucleotides in length. In some embodiments, the nucleic acid is about 7500, 10,000, 15,000, or 20,000 nucleotides in length.

In some embodiments, compositions provided herein comprise at least one nucleic acid sequence comprising a sequence which encodes a viral antigen. In some embodiments, the viral antigen is a surface protein or a transmembrane protein. In some embodiments, the viral antigen is a spike protein, a glycoprotein, or an envelope protein. In some embodiments, the viral antigen is a coronavirus antigen or a fragment thereof. In some embodiments, the antigen is a SARS-CoV antigen, a SARS-CoV-2 antigen, a Middle East Respiratory Syndrome (MERS) coronavirus antigen, a fragment or a variant thereof.

### SARS-CoV-2 Spike Protein Antigens

Severe acute respiratory syndrome coronavirus 2 (SARS-CoV-2) is a virus that causes the infectious disease called COVID-19 (coronavirus disease 2019). Variants of the SARS-CoV-2 virus include the alpha, beta, delta, mu, and omicron variants. The Alpha (B.1.1.7), Beta (B.1.351, B.1.351.2, B.1.351.3), Delta (B.1.617.2, AY.1, AY.2, AY.3), Omicron (B.1.1.529), and Gamma (P.1, P.1.1, P.1.2) variants circulating in the United States are classified as variants of concern. SARS-CoV-2 structure, its components, along with variants and their various features are described below.

Coronaviruses are single-stranded RNA-enveloped viruses that have four structural proteins, known as the S (spike), E (envelope), M (membrane), and N (nucleocapsid) proteins. The N protein holds the RNA genome, and the S, E, and M proteins together create the viral envelope. In SARS-CoV-2, the spike (S) protein facilitates viral attachment and fusion with the membrane of a host cell via the host cell receptor, angiotensin-converting enzyme 2 (ACE2). The S protein mediates viral cell entry into the host cell. The total length of SARS-CoV-2 spike is generally 1273 amino acids and includes a signal peptide (amino acids 1-13) located at the N-terminus, the S1 subunit (residues 14-685), and the S2 subunit (residues 686-1273); the last two regions are responsible for receptor binding and membrane fusion, respectively. In the S1 subunit, there is an N-terminal domain (14-305 residues) and a receptor-binding domain (RBD, 319-541 residues). The fusion peptide (FP) (788-806 residues), heptapeptide repeat sequence 1 (HR1) (912-984 residues), HR2 (1163-1213 residues), TM domain (1213-1237 residues), and cytoplasm domain (1237-1273 residues) comprise the S2 subunit. Specifically, the HR1 and HR2 are composed of a repetitive heptapeptide: HPPHCPC, where H is a hydrophobic or traditionally bulky residue, P is a polar or hydrophilic residue, and C is another charged residue. HR1 and HR2 form a six-helical bundle (6-HB) referred to as the “stem helix” domain of the S2 protein. When the RBD binds to ACE2 on a host cell membrane, S2 changes conformation by inserting FP into the target cell membrane, exposing the pre-hairpin coiled-coil of the HR1 domain and triggering interaction between the HR2 domain and HR1 trimer to form the stem helix (6-HB), thus bringing the viral envelope and cell membrane into proximity for viral fusion and entry. The wild-type spike protein amino acid sequence is provided in SEQ ID NO: 17. The amino acid sequence corresponding to the stem helix is provided in SEQ ID NO: 18.

Coronaviruses regularly undergo antigenic drift, a type of genetic variation in viruses, arising from the accumulation of mutations in viral genes that encode for virus-surface proteins that host antibodies recognize. Several SARS-CoV-2 variants have been identified by public health agencies and are provided in Table 2 below.

TABLE 2
SARS-CoV-2 Variants.

| | | | | Additional spike | Earliest | |
|---|---|---|---|---|---|---|
| WHO | Pango | GISAID | Nextstrain | amino acid | documented | Date of |
| label | lineage* | clade | clade | changes monitored ° | samples | designation |
| Alpha | B.1.1.7 ^{#} | GRY | 20I (V1) | N501Y | United | 18 Dec. |
| | | | | A570D | Kingdom, | 2020 |
| | | | | P681H | September 2020 | |
| | | | | T716I | | |
| | | | | S982A | | |
| | | | | D1118H | | |
| Beta | B.1.351 | GH/501Y.V2 | 20H (V2) | D80A | South | 18 Dec. |
| | | | | D215G | Africa, | 2020 |
| | | | | K417N | May 2020 | |
| | | | | A701V | | |
| | | | | N501Y | | |
| | | | | E484K | | |
| Gamma | P.1 | GR/501Y.V3 | 20J (V3) | L18F | Brazil, | 11 Jan. |
| | | | | T20N | November 2020 | 2021 |
| | | | | P26S | | |
| | | | | D138Y | | |
| | | | | R190S | | |
| | | | | K417T | | |
| | | | | E484K | | |
| | | | | N501Y | | |
| | | | | H655Y | | |
| | | | | T1027I | | |
| Delta | B.1.617.2^{§} | G/478K.V1 | 21A | T19R | India, | VOI: 4 |
| | | | | L452R | October 2020 | Apr. 2021 |
| | | | | T478K | | VOC: 11 |
| | | | | P681R | | May 2021 |
| | | | | D950N | | |
| Eta | B.1.525 | G/484K.V3 | 21D | | Multiple | 17 Mar. |
| | | | | | countries, | 2021 |
| | | | | | December 2020 | |
| Iota | B.1.526 | GH/253G.V1 | 21F | | United | 24 Mar. |
| | | | | | States of America, | 2021 |
| | | | | | November 2020 | |
| Kappa | B.1.617.1 | G/452R.V3 | 21B | | India, | 4 Apr. 2021 |
| | | | | | October 2020 | |
| Lambda | C.37 | GR/452Q.V1 | 21G | | Peru, December | 14 Jun. |
| | | | | | 2020 | 2021 |
| Mu | B.1.621 | GH | 21H | | Colombia, | 30 Aug. |
| | | | | | January 2021 | 2021 |
| Omicron | B.1.1.529 | GR/484A | 21K | A67V, Δ69-70, | November 2021 | VOC: 26 |
| | | | | T95I, G142D, Δ143- | South | Nov. 2021 |
| | | | | 145, Δ211, L212I, | Africa, Hong | |
| | | | | ins214EPE, G339D, | Kong, | |
| | | | | S371L, S373P, | Belgium, | |
| | | | | S375F, K417N, | Israel | |
| | | | | N440K, G446S, | | |
| | | | | S477N, T478K, | | |
| | | | | E484A, Q493R, | | |
| | | | | G496S, Q498R, | | |
| | | | | N501Y, Y505H, | | |
| | | | | T547K, D614G, | | |
| | | | | H655Y, N679K, | | |
| | | | | P681H, N764K, | | |
| | | | | D796Y, N856K, | | |
| | | | | Q954H, N969K, | | |
| | | | | L981F | | |

*includes all descendent lineages. The full list of Pango lineages can be found here: Error! Hyperlink reference not valid.cov-lineages.org/lineage_list.html; for FAQ, visit: pango.network/faqs/

In some embodiments, the antigen is derived from a SARS-CoV-2 variant of concern (VOC). A variant of concern is a variant for which there is evidence of an increase in transmissibility, more severe disease (e.g., increased hospitalizations or deaths), significant reduction in neutralization by antibodies generated during previous infection or vaccination, reduced effectiveness of treatments or vaccines, or diagnostic detection failures. Possible attributes of a variant of concern include, in addition to the possible attributes of a variant of interest, (i) evidence of impact on diagnostics, treatments, or vaccines, (ii) widespread interference with diagnostic test targets, (iii) evidence of substantially decreased susceptibility to one or more class of therapies, (iv) evidence of significant decreased neutralization by antibodies generated during previous infection or vaccination, (v) evidence of reduced vaccine-induced protection from severe disease, (vi) evidence of increased transmissibility, and (vii) evidence of increased disease severity.

In some embodiments, the antigen is derived from a SARS-CoV-2 variant of interest (VOI). A variant of interest is a variant with specific genetic markers that have been associated with changes to receptor binding, reduced neutralization by antibodies generated against previous infection or vaccination, reduced efficacy of treatments, potential diagnostic impact, or predicted increase in transmissibility or disease severity. Possible attributes of a variant of interest include (i) specific genetic markers that are predicted to affect transmission, diagnostics, therapeutics, or immune escape, (ii) evidence that it is the cause of an increased proportion of cases or unique outbreak clusters, and (iii) limited prevalence or expansion in the US or in other countries.

In some embodiments, nucleic acids provided herein encodes for an antigen listed in Table 3 or a fragment thereof. In some embodiments, the nucleic acid comprises at least 80%, 85%, 90%, 95%, 96%, 97%, 98%, or 99% sequence identity to a sequence which specifically binds an antigen listed in Table 3. In some embodiments, the nucleic acid provided herein comprises at least 80%, 85%, 90%, 95%, 96%, 97%, 98%, or 99% sequence similarity to an RNA sequence listed in Table 3. Percent (%) sequence identity for a given sequence relative to a reference sequence is defined as the percentage of identical residues identified after aligning the two sequences and introducing gaps if necessary, to achieve the maximum percent sequence identity. Percent identity can be calculated using alignment methods known in the art, for instance alignment of the sequences can be conducted using publicly available software such as BLAST, Align, ClustalW2. Those skilled in the art can determine the appropriate parameters for alignment, but the default parameters for BLAST are specifically contemplated. Exemplary nucleic acid sequences encoding for exemplary SARS-CoV-2 antigens are listed in Table 3.

TABLE 3
SARS CoV-2 Spike Protein Nucleic Acid and Amino Acid Sequences.

| SEQ ID NOS | Name | Variant |
|---|---|---|
| SEQ ID NO: 1 | Delta V5 RNA Sequence | A.1 |
| SEQ ID NO: 2 | K995P-V996P RNA Sequence | A.1-preF |
| SEQ ID NO: 3 | D614G RNA Sequence | B.1 |
| SEQ ID NO: 4 | B.1.351-PP-D614G RNA Sequence | Beta-preF |
| SEQ ID NO: 5 | B.1.1.7-PP-D614G RNA Sequence | Alpha-preF |
| SEQ ID NO: 6 | Delta.AY1-S2P-wtFur RNA Sequence | Delta-preF |
| SEQ ID NO: 7 | Delta.AY1-S2P-wtFur-newKozak | Delta-preF- |
| | RNA Sequence | kozak |
| SEQ ID NO: 8 | Omicron-B.1.1.529 RNA Sequence | Omicron |
| SEQ ID NO: 9 | Delta V5 Amino Acid Sequence | A.1 |
| SEQ ID NO: 10 | K995P-V996P Amino Acid Sequence | A.1-preF |
| SEQ ID NO: 11 | D614G Amino Acid Sequence | B.1 |
| SEQ ID NO: 12 | B.1.351-PP-D614G Amino | Beta-preF |
| | Acid Sequence | |
| SEQ ID NO: 13 | B.1.1.7-PP-D614G Amino | Alpha-preF |
| | Acid Sequence | |
| SEQ ID NO: 14 | Delta.AY1-S2P-wtFur | Delta-preF |
| | Amino Acid Sequence | |
| SEQ ID NO: 15 | Delta.AY1-S2P-wtFur-newKozak | Delta-preF- |
| | | kozak |
| SEQ ID NO: 16 | Omicron-B.1.1.529 Amino | Omicron |
| | Acid Sequence | |
| SEQ ID NO: 17 | Wuhan-Hu-1-Full-Length | Wild-type |
| | Wild-Type Spike Protein Amino | (WT) |
| | Acid Sequence | Spike |
| SEQ ID NO: 18 | Wuhan-Hu-1-Wild-Type Spike | Wild-type |
| | Protein-Stem Helix Amino | (WT) |
| | Acid Sequence [residues 913 to 1213] | Spike |
| SEQ ID NO: 19 | Omicron-B.1.1.529 Amino | Omicron |
| | Acid Sequence of the Stem | |
| | Helix (HR1 and HR2) | |

*Pre-F is stabilized pre-fusion spike protein

In some embodiments, compositions provided herein comprise a SARS-CoV-2 spike protein or a fragment thereof, wherein the SARS-CoV-2 spike protein amino acid sequence is encoded by any nucleic acid sequence disclosed herein. In some embodiments, compositions provided herein comprise an RNA sequence set forth in any one of SEQ ID NOS: 1 to 8. In some embodiments, the spike protein has an amino acid sequence corresponding to any of the sequences set forth in any one of SEQ ID NOS: 9 to 19.

In some embodiments, compositions provided herein comprise a nucleic acid sequence comprising (i) any vector backbone, and (ii) any of the antigen sequences set forth in SEQ ID NOS: 24-31, or any nucleic acid sequence which is at least 80%, or at least 85%, or at least 90%, or at least 93%, or at least 95%, or at least 97% identical to any of the antigen sequences set forth in SEQ ID NOS: 24-31.

In some embodiments, compositions provided herein comprise a nucleic acid sequence comprising (i) a vector backbone as set forth in one of SEQ ID NOS: 32-38, and (ii) any sequence encoding an amino acid sequence defining any SARS-CoV-2 spike protein, or any sequence encoding an amino acid sequence which is at least 80%, or at least 85%, or at least 90%, or at least 93%, or at least 95%, or at least 97% identical to the sequence defining any SARS-CoV-2 spike protein.

In some embodiments, compositions provided herein comprise a nucleic acid sequence comprising (i) a vector backbone as set forth in one of SEQ ID NOS: 32-38, or any sequence which is at least 80%, or at least 85%, or at least 90%, or at least 93%, or at least 95%, or at least 97% identical to a sequence as set forth in one of SEQ ID NOS: 32-38, and (ii) any sequence encoding an amino acid sequence defining any SARS-CoV-2 spike protein, or any sequence encoding an amino acid sequence which is at least 80%, or at least 85%, or at least 90%, or at least 93%, or at least 95%, or at least 97% identical to the sequence defining any SARS-CoV-2 spike protein.

### Self-Replicating Nucleic Acids

Provided herein are compositions comprising a self-replicating nucleic acid. The SARS-CoV-2 spike antigen provided herein or fragment thereof can be encoded as part of a self-replicating nucleic acid construct. In some embodiments, the self-replicating nucleic acid molecule comprises at least one or more genes selected from the group consisting of viral replicases, viral proteases, viral helicases and other nonstructural viral proteins, and also comprises 5′- and 3′-end cis-active replication sequences, and an antigenic sequence encoding a SARS-CoV-2 spike protein. A subgenomic promoter that directs expression of the heterologous sequence(s) can be included in the self-replicating nucleotide sequence. If desired, a heterologous sequence may be fused in frame to other coding regions in the self-replicating RNA and/or may be under the control of an internal ribosome entry site (IRES).

In some embodiments, the self-replicating nucleotide sequence is a self-replicating RNA molecule. Self-replicating RNA molecules are designed so that the self-replicating RNA molecule cannot induce production of infectious viral particles. This can be achieved, for example, by omitting one or more viral genes encoding structural proteins that are necessary for the production of viral particles in the self-replicating RNA. For example, when the self-replicating RNA molecule is based on an alphavirus, such as Sindbis virus (SIN), Semliki forest virus and Venezuelan equine encephalitis virus (VEE), one or more genes encoding viral structural proteins, such as capsid and/or envelope glycoproteins, can be omitted. If desired, self-replicating RNA molecules of the invention can be designed to induce production of infectious viral particles that are attenuated or virulent, or to produce viral particles that are capable of a single round of subsequent infection.

A self-replicating RNA molecule can, when delivered to an animal cell even without any proteins, lead to the production of multiple daughter RNAs by transcription from itself (or from an antisense copy of itself). The self-replicating RNA can be directly translated after delivery to a cell, and this translation provides an RNA-dependent RNA polymerase which then produces transcripts from the delivered RNA. Thus, the delivered RNA leads to the production of multiple daughter RNAs. These transcripts are antisense relative to the delivered RNA and may be translated themselves to provide in situ expression of encoded SARS-CoV-2 spike protein, or may be transcribed to provide further transcripts with the same sense as the delivered RNA which are translated to provide in situ expression of the encoded SARS-CoV-2 spike protein (s).

The self-replicating RNA molecules provided herein can contain one or more modified nucleotides and therefore have improved stability and be resistant to degradation and clearance in vivo, and other advantages. In some embodiments, self-replicating RNA molecules that contain modified nucleotides avoid or reduce stimulation of endosomal and cytoplasmic immune receptors when the self-replicating RNA is delivered into a cell. This permits self-replication, amplification and expression of protein to occur. This also reduces safety concerns relative to self-replicating RNA that does not contain modified nucleotides, because the self-replicating RNA that contains modified nucleotides reduce activation of the innate immune system and subsequent undesired consequences (e.g., inflammation at injection site, irritation at injection site, pain, and the like). RNA molecules produced as a result of self-replication are recognized as foreign nucleic acids by the cytoplasmic immune receptors. Thus, self-replicating RNA molecules that contain modified nucleotides provide for efficient amplification of the RNA in a host cell and expression of SARS-CoV-2 spike proteins, as well as adjuvant effects.

In some embodiments, self-replicating RNA molecules provided herein contain at least one modified nucleotide. Modified nucleotides that are not part of the 5′ cap (e.g., in addition to the modification that are part of the 5″ cap) can be used. Accordingly, the self-replicating RNA molecule can contain a modified nucleotide at a single position, can contain a particular modified nucleotide (e.g., pseudouridine, N6-methyladenosine, 5-methylcytidine, 5-methyluridine) at two or more positions, or can contain two, three, four, five, six, seven, eight, nine, ten or more modified nucleotides (e.g., each at one or more positions). Preferably, the self-replicating RNA molecules comprise modified nucleotides that contain a modification on or in the nitrogenous base, but do not contain modified sugar or phosphate moieties. In some examples, between 0.001% and 99% or 100% of the nucleotides in a self-replicating RNA molecule are modified nucleotides. For example, 0.001%-25%, 0.01%-25%, 0.1%-25%, or 1%-25% of the nucleotides in a self-replicating RNA molecule are modified nucleotides. In other examples, between 0.001% and 99% or 100% of a particular unmodified nucleotide in a self-replicating RNA molecule is replaced with a modified nucleotide. For example, about 1% of the nucleotides in the self-replicating RNA molecule that contain uridine can be modified, such as by replacement of uridine with pseudouridine. In other examples, the desired amount (percentage) of two, three, or four particular nucleotides (nucleotides that contain uridine, cytidine, guanosine, or adenine) in a self-replicating RNA molecule are modified nucleotides. For example, 0.001%-25%, 0.01%-25%, 0.1%-25%, or 1%-25% of a particular nucleotide in a self-replicating RNA molecule are modified nucleotides. In other examples, 0.001%-20%, 0.001%-15%, 0.001%-10%, 0.01%-20%, 0.01%-15%, 0.1%-25, 0.01%-10%, 1%-20%, 1%-15%, 1%-10%, or about 5%, about 10%, about 15%, about 20% of a particular nucleotide in a self-replicating RNA molecule are modified nucleotides. It is preferred that less than 100% of the nucleotides in a self-replicating RNA molecule are modified nucleotides. It is also preferred that less than 100% of a particular nucleotide in a self-replicating RNA molecule are modified nucleotides. Thus, preferred self-replicating RNA molecules comprise at least some unmodified nucleotides.

Self-replicating RNA molecules that comprise at least one modified nucleotide can be prepared using any suitable method. Several suitable methods are known in the art for producing RNA molecules that contain modified nucleotides. For example, a self-replicating RNA molecule that contains modified nucleotides can be prepared by transcribing (e.g., in vitro transcription) a DNA that encodes the self-replicating RNA molecule using a suitable DNA-dependent RNA polymerase, such as T7 phage RNA polymerase, SP6 phage RNA polymerase, T3 phage RNA polymerase, and the like, or mutants of these polymerases which allow efficient incorporation of modified nucleotides into RNA molecules. The transcription reaction will contain nucleotides and modified nucleotides, and other components that support the activity of the selected polymerase, such as a suitable buffer, and suitable salts. The incorporation of nucleotide analogs into a self-replicating RNA may be engineered, for example, to alter the stability of such RNA molecules, to increase resistance against RNases, to establish replication after introduction into appropriate host cells (“infectivity” of the RNA), and/or to induce or reduce innate and adaptive immune responses. Suitable synthetic methods can be used alone, or in combination with one or more other methods (e.g., recombinant DNA or RNA technology), to produce a self-replicating RNA molecule that contain one or more modified nucleotides.

Nucleic acid synthesis can also be performed using suitable recombinant methods that are well-known and conventional in the art, including cloning, processing, and/or expression of polynucleotides and gene products encoded by such polynucleotides. DNA shuffling by random fragmentation and PCR reassembly of gene fragments and synthetic polynucleotides are examples of known techniques that can be used to design and engineer polynucleotide sequences. Site-directed mutagenesis can be used to alter nucleic acids and the encoded proteins, for example, to insert new restriction sites, alter glycosylation patterns, change codon preference, produce splice variants, introduce mutations and the like.

In some embodiments, nucleic acids provided herein encode for an RNA polymerase. In some embodiments, nucleic acids provided herein encode for a viral RNA polymerase. In some embodiments, nucleic acids provided herein encode for: (1) a viral RNA polymerase; and (2) a protein or functional fragment thereof. In some embodiments, compositions provided herein comprise a first nucleic acid encoding for a viral RNA polymerase; and a second nucleic acid encoding for a protein or functional fragment thereof.

Provided herein are compositions comprising a self-replicating RNA. A self-replicating RNA (also called a replicon) includes any genetic element, for example, a plasmid, cosmid, bacmid, phage or virus that is capable of replication largely under its own control. Self-replication provides a system for self-amplification of the nucleic acids provided herein in mammalian cells. In some embodiments, the self-replicating RNA is single stranded. In some embodiments, the self-replicating RNA is double stranded.

An RNA polymerase provided herein can include but is not limited to: an alphavirus RNA polymerase, an Eastern equine encephalitis virus (EEEV) RNA polymerase, a Western equine encephalitis virus (WEEV), Venezuelan equine encephalitis virus (VEEV), Also, Chikungunya virus (CHIKV), Semliki Forest virus (SFV), or Sindbis virus (SINV). In some embodiments, the RNA polymerase is a VEEV RNA polymerase. In some embodiments, the nucleic acid encoding for the RNA polymerase comprises at least 85% identity to the nucleic acid sequence of SEQ ID NO: 20. In some embodiments, the nucleic acid encoding for the RNA polymerase comprises at least 90% identity to the nucleic acid sequence of SEQ ID NO: 20. In some embodiments, the nucleic acid encoding for the RNA polymerase comprises at least 95% identity to the nucleic acid sequence of SEQ ID NO: 20. In some embodiments, the nucleic acid encoding for the RNA polymerase comprises at least 99% identity to the nucleic acid sequence of SEQ ID NO: 20. In some embodiments, the nucleic acid encoding for the RNA polymerase is SEQ ID NO: 20.

In some embodiments, the amino acid sequence for VEEV RNA polymerase comprises at least 85% identity to RELPVLDSAAFNVECFKKYACNNEYWETFKENPIRLTEEN VVNYITKLKGP (SEQ ID NO: 21), TQMRELPVLDSAAFNVECFKKYACNNEYWE TFKENPIRLTE (SEQ ID NO: 22), or SEQ ID NO: 23. In some embodiments, the amino acid sequence for VEEV RNA polymerase comprises at least 90% identity to SEQ ID NO: 21, SEQ ID NO: 22, or SEQ ID NO: 23. In some embodiments, the amino acid sequence for VEEV RNA polymerase comprises at least 95% identity to SEQ ID NO: 21, SEQ ID NO: 22, or SEQ ID NO: 23. In some embodiments, the amino acid sequence for VEEV RNA polymerase comprises at least 99% identity to SEQ ID NO: 21, SEQ ID NO: 22, or SEQ ID NO: 23. In some embodiments, the amino acid sequence for VEEV RNA polymerase is SEQ ID NO: 21, SEQ ID NO: 22, or SEQ ID NO: 23.

Provided herein are compositions and methods comprising replicon RNA (repRNA) encoding one or more structural proteins from a non-enveloped virus. In some embodiments, the repRNA encodes a protease. In some embodiments, the repRNA encodes the 3CD protease. In some embodiments, the structural protein and the protease are co-expressed. In further embodiments, the repRNA comprises one or more open reading frames. In some embodiments, the open reading frames are separated by an internal ribosomal entry site (IRES). In some embodiments, the open reading frames are separated by a ribosomal skipping peptide sequence. In some embodiments the ribosomal skipping peptide sequence is from Thosea asigna virus (T2A).

### Combination Compositions

Provided herein are compositions comprising a nanoparticle described herein and a nucleic acid encoding for a coronavirus antigen. Further provided herein is a nanoemulsion comprising a plurality of nanoparticles provided herein. In some embodiments, the nucleic acid further encodes for an RNA polymerase. In some embodiments the RNA polymerase is a viral RNA polymerase. In some embodiments, the nucleic acid encoding the RNA polymerase is on the same nucleic acid strand as the nucleic acid sequence encoding the protein (e.g., cis). In some embodiments, the nucleic acid encoding the RNA polymerase is on a different nucleic acid strand as the nucleic acid sequence encoding the protein (e.g., trans). In some embodiments, the nucleic acid encoding the RNA polymerase is a DNA molecule. In some embodiments, nucleic acid sequences encoding an antigen provided herein are DNA or RNA molecules. In some embodiments, antigens provided herein are encoded by DNA. Nanoparticles for inclusion include, without limitation, any one of NP-1 to NP-30, or any one of NP-1 to NP-31. Nucleic acids for inclusion include, without limitation, comprise a region comprising any one of, or a plurality of, SEQ ID NOS: 1 to 8 and/or SEQ ID NOS: 24 to 31. In some instances, the nucleic acids further comprise a region encoding for an RNA polymerase, e.g., a region comprising a sequence of SEQ ID NO: 20.

Compositions provided herein can be characterized by an nitrogen:phosphate (N:P) molar ratio. The N:P ratio is determined by the amount of cationic lipid in the nanoparticle which contain nitrogen and the amount of nucleic acid used in the composition which contain negatively charged phosphates. A molar ratio of the lipid carrier to the nucleic acid can be chosen to increase the delivery efficiency of the nucleic acid, increase the ability of the nucleic acid-carrying nanoemulsion composition to elicit an immune response to the antigen, increase the ability of the nucleic acid-carrying nanoemulsion composition to elicit the production of antibody titers to the antigen in a subject. In some embodiments, compositions provided herein have a molar ratio of the lipid carrier to the nucleic acid can be characterized by the nitrogen-to-phosphate molar ratio, which can range from about 0.01:1 to about 1000:1, for instance, from about 0.2:1 to about 500:1, from about 0.5:1 to about 150:1, from about 1:1 to about 150:1, from about 1:1 to about 125:1, from about 1:1 to about 100:1, from about 1:1 to about 50:1, from about 1:1 to about 50:1, from about 5:1 to about 50:1, from about 5:1 to about 25:1, or from about 10:1 to about 20:1. In certain embodiments, the molar ratio of the lipid carrier to the nucleic acid, characterized by the nitrogen-to-phosphate (N:P) molar ratio, ranges from about 1:1 to about 150:1, from about 5:1 to about 25:1, or from about 10:1 to about 20:1. In one embodiment, the N:P molar ratio of the nanoemulsion composition is about 15:1. In some embodiments, the nanoparticle comprises a nucleic acid provided herein covalently attached to the membrane.

Compositions provided herein can be characterized by an oil-to-surfactant molar ratio. In some embodiments, the oil-to-surfactant ratio is the molar ratio of squalene:DOTAP, hydrophobic surfactant, and hydrophilic surfactant. In some embodiments, the oil-to-surfactant ratio is the molar ratio of squalene:DOTAP, sorbitan monostearate, and polysorbate 80. In some embodiments, the oil-to surfactant molar ratio ranges from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about 1:1. In some embodiments, the oil-to-surfactant molar ratio is at least about 0.1:1, at least about 0.2:1, at least about 0.3:1, at least about 0.4:1, at least about 0.5:1, at least about 0.6:1, at least about 0.7:1. In some embodiments, the oil-to surfactant molar ratio is at least about 0.4:1 up to 1:1.

Compositions provided herein can be characterized by hydrophilic surfactant-to-lipid (e.g., cationic lipid) ratio. In some embodiments, the hydrophilic surfactant-to-lipid ratio ranges from about 0.1:1 to about 2:1, from about 0.2:1 to about 1.5:1, from about 0.3:1 to about 1:1, from about 0.5:1 to about 1:1, or from about 0.6:1 to about 1:1. Compositions provided herein can be characterized by hydrophobic surfactant-to-lipid (e.g., cationic lipid) ratio ranging. In some embodiments, the hydrophobic surfactant-to-lipid ratio ranges from about 0.1:1 to about 5:1, from about 0.2:1 to about 3:1, from about 0.3:1 to about 2:1, from about 0.5:1 to about 2:1, or from about 1:1 to about 2:1.

Provided herein is a dried composition comprising a sorbitan fatty acid ester, an ethoxylated sorbitan ester, a cationic lipid, an immune stimulant, and an RNA. Further provided herein are dried compositions, wherein the dried composition comprises sorbitan monostearate (e.g., SPAN-60), polysorbate 80 (e.g., TWEEN-80), DOTAP, an immune stimulant, and an RNA.

### Thermally Stable, Dried, and Lyophilized SARS-COV-2 RNA Vaccines

Provided herein are dried or lyophilized compositions and vaccines. Further provided herein are pharmaceutical compositions comprising a dried or lyophilized composition provided herein that is reconstituted in a suitable diluent and a pharmaceutically acceptable carrier. In some embodiments, the diluent is aqueous. In some embodiments, the diluent is water.

A lyophilized composition is generated by a low temperature dehydration process involving the freezing of the composition, followed by a lowering of pressure, and removal of ice by sublimation. In certain cases, lyophilisation also involves the removal of bound water molecules through a desorption process. In some embodiments, compositions and vaccines provided herein are spray-dried. Spray drying is a process by which a solution is fed through an atomizer to create a spray, which is thereafter exposed to a heated gas stream to promote rapid evaporation. When sufficient liquid mass has evaporated, the remaining solid material in the droplet forms particles which are then separated from the gas stream (e.g., using a filter or a cyclone). Drying aids in the storage of the compositions and vaccines provided herein at higher temperatures (e.g., greater than 4° C.) as compared to the sub-zero temperatures needed for the storage of existing mRNA vaccines. In some embodiments, dried compositions and lyophilized compositions provided herein comprise (a) a lipid carrier, wherein the lipid carrier is a nanoemulsion comprising: (i) a hydrophobic core; (ii) one or more inorganic nanoparticles; (iii) and one or more lipids; (b) one or more nucleic acids; and (c) at least one cryoprotectant. In some embodiments, the cryoprotectant is selected from the group consisting of: sucrose, maltose, trehalose, mannitol, glucose, and any combinations thereof. Additional examples of cryoprotectants include but are not limited to: dimethyl sulfoxide (DMSO), glycerol, propylene glycol, ethylene glycol, 3-O-methyl-D-glucopyranose (3-OMG), olyethylene glycol (PEG), 1,2-propanediol, acetamide, trehalose, formamide, sugars, proteins, and carbohydrates.

In some embodiments, compositions and methods provided herein comprise at least one cryoprotectant. Exemplary cryoprotectants for inclusion are, but not limited to, sucrose, maltose, trehalose, mannitol, or glucose, and any combinations thereof. In some embodiments, additional or alternative cryoprotectant for inclusion is sorbitol, ribitol, erthritol, threitol, ethylene glycol, or fructose. In some embodiments, additional or alternative cryoprotectant for inclusion is dimethyl sulfoxide (DMSO), glycerol, propylene glycol, ethylene glycol, 3-O-methyl-D-glucopyranose (3-OMG), polyethylene glycol (PEG), 1,2-propanediol, acetamide, trehalose, formamide, sugars, proteins, and carbohydrates. In some embodiments, the cryoprotectant is present at about 1% w/v to at about 20% w/v, preferably about 10% w/v to at about 20% w/v, and more preferably at about 10% w/v. In certain aspects of the disclosure, the cryoprotectant is sucrose. In some aspects of the disclosure, the cryoprotectant is maltose. In some aspects of the disclosure, the cryoprotectant is trehalose. In some aspects of the disclosure, the cryoprotectant is mannitol. In some aspects of the disclosure, the cryoprotectant is glucose. In some embodiments, the cryoprotectant is present in an amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 450, 500 or more mg. In some embodiments, the cryoprotectant is present in an amount of about 50 to about 500 mg. In some embodiments, the cryoprotectant is present in an amount of about 200 to about 300 mg. In some embodiments, the cryoprotectant is present in an amount of about 250 mg. In some embodiments, the cryoprotectant is present in amount of a lyophilized composition by weight of at least about 50, 55, 60, 65, 70, 75, 80, 85, 90, 95 or more percent. In some embodiments, the cryoprotectant is present in amount of a lyophilized composition by weight of about 95%. In some embodiments, the cryoprotectant is present in amount of a lyophilized composition by weight of 80 to 98%, 85 to 98%, 90 to 98%, or 94 to 96%. In some embodiments, the cryoprotectant is a sugar. In some embodiments, the sugar is sucrose, maltose, trehalose, mannitol, or glucose. In some embodiments, the sugar is sucrose. In some embodiments, the sucrose is present in an amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 450, 500 or more mg. In some embodiments, the sucrose is present in an amount of about 50 to about 500 mg. In some embodiments, the sucrose is present in an amount of about 200 to about 300 mg. In some embodiments, the sucrose is present in an amount of about 250 mg. In some embodiments, the sucrose is present in amount of a lyophilized composition by weight of at least about 50, 55, 60, 65, 70, 75, 80, 85, 90, 95 or more percent. In some embodiments, the sucrose is present in amount of a lyophilized composition by weight of about 95%. In some embodiments, the sucrose is present in amount of a lyophilized composition by weight of 80 to 98%, 85 to 98%, 90 to 98%, or 94 to 96%.

In some embodiments, the cryoprotectant is sucrose. In some embodiments, the cryoprotectant is at a concentration of at least about 0.1% w/v. In some embodiments, the cryoprotectant is at a concentration of about 1% w/v to at about 20% w/v. In some embodiments, the cryoprotectant is at a concentration of about 10% w/v to at about 20% w/v. In some embodiments, the cryoprotectant is at a concentration of about 10% w/v.

In some embodiments, compositions and vaccines provided herein are thermally stable. A composition is considered thermally stable when the composition resists the action of heat or cold and maintains its properties, such as the ability to protect a nucleic acid molecule from degradation at given temperature. In some embodiments, compositions and vaccines provided herein are thermally stable at about 25° C. or standard room temperature. In some embodiments, compositions and vaccines provided herein are thermally stable at about 45° C. In some embodiments, compositions and vaccines provided herein are thermally stable at about 20° C. In some embodiments, compositions and vaccines provided herein are thermally stable at about 2° C. to about 8° C. In some embodiments, compositions and vaccines provided herein are thermally stable at a temperature of at least about −80° C., at least about −20° C., at least about 0° C., at least about 2° C., at least about 4° C., at least about 6° C., at least about 8° C., at least about 10° C., at least about 20° C., at least about 25° C., at least about 30° C., at least about 37° C., up to 45° C. In some embodiments, compositions and vaccines provided herein are thermally stable for at least about 5 day, at least about 1 week, at least about 2 weeks, at least about 1 month, up to 3 months. In some embodiments, compositions and vaccines provided herein are stored at a temperature of at least about 4° C. up to 37° C. for at least about 5 day, at least about 1 week, at least about 2 weeks, at least about 1 month, up to 3 months. In some embodiments, compositions and vaccines provided herein are stored at a temperature of at least about 20° C. up to 25° C. for at least about 5 day, at least about 1 week, at least about 2 weeks, at least about 1 month, up to 3 months.

Also provided herein are methods for preparing a lyophilized composition comprising obtaining a lipid carrier, wherein the lipid carrier is a nanoemulsion comprising a hydrophobic core, one or more inorganic nanoparticles and one or more lipids; incorporating one or more nucleic acid into the lipid carrier to form a lipid carrier-nucleic acid complex; adding at least one cryoprotectant to the lipid carrier-nucleic acid complex to form a formulation; and lyophilizing the formulation to form a lyophilized composition.

Further provided herein are methods for preparing a spray-dried composition comprising obtaining a lipid carrier, wherein the lipid carrier is a nanoemulsion comprising a hydrophobic core, one or more inorganic nanoparticles and one or more lipids; incorporating one or more nucleic acid into the lipid carrier to form a lipid carrier-nucleic acid complex; adding at least one cryoprotectant to the lipid carrier-nucleic acid complex to form a formulation; and spray drying the formulation to form a spray-dried composition.

Further provided herein are methods for reconstituting a lyophilized composition comprising: obtaining a lipid carrier, wherein the lipid carrier is a nanoemulsion comprising a hydrophobic core, one or more inorganic nanoparticles, and one or more lipids; incorporating one or more nucleic acid into the said lipid carrier to form a lipid carrier-nucleic acid complex; adding at least one cryoprotectant to the lipid carrier-nucleic acid complex to form a formulation; lyophilizing the formulation to form a lyophilized composition; and reconstituting the lyophilized composition in a suitable diluent.

Further provided herein are methods for reconstituting a spray-dried composition comprising: obtaining a lipid carrier, wherein the lipid carrier is a nanoemulsion comprising a hydrophobic core, one or more inorganic nanoparticles, and one or more lipids, incorporating one or more nucleic acid into the said lipid carrier to form a lipid carrier-nucleic acid complex; adding at least one cryoprotectant to the lipid carrier-nucleic acid complex to form a formulation; spray drying the formulation to form a spray-dried composition; and reconstituting the spray-dried composition in a suitable diluent.

### Pharmaceutical Compositions

Provided herein is a suspension comprising a composition provided herein. In some embodiments, suspensions provided herein comprise a plurality of nanoparticles or compositions provided herein. In some embodiments, compositions provided herein are in a suspension, optionally a homogeneous suspension. In some embodiments, compositions provided herein are in an emulsion form.

Also provided herein is a pharmaceutical composition comprising a composition provided herein. In some embodiments, compositions provided herein are combined with pharmaceutically acceptable salts, excipients, and/or carriers to form a pharmaceutical composition. Pharmaceutical salts, excipients, and carriers may be chosen based on the route of administration, the location of the target issue, and the time course of delivery of the drug. A pharmaceutically acceptable carrier or excipient may include solvents, dispersion media, coatings, antibacterial and antifungal agents, isotonic and absorption delaying agents, etc., compatible with pharmaceutical administration.

In some embodiments, the pharmaceutical composition is in the form of a solid, semi-solid, liquid or gas (aerosol). Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P., and isotonic sodium chloride solution. In addition, sterile, fixed oils are employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quatemary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

### Dosing

Compositions provided herein may be formulated in dosage unit form for ease of administration and uniformity of dosage. A dosage unit form is a physically discrete unit of a composition provided herein appropriate for a subject to be treated. It will be understood, however, that the total usage of compositions provided herein will be decided by the attending physician within the scope of sound medical judgment. For any composition provided herein the therapeutically effective dose can be estimated initially either in cell culture assays or in animal models, such as mice, rabbits, dogs, pigs, or non-human primates. The animal model is also used to achieve a desirable concentration range and route of administration. Such information can then be used to determine useful doses and routes for administration in humans. Therapeutic efficacy and toxicity of compositions provided herein can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies may be used in formulating a range of dosage for human use.

### Administration

Provided herein are compositions and pharmaceutical compositions for administering to a subject in need thereof. In some embodiments, pharmaceutical compositions provided here are in a form which allows for compositions provided herein to be administered to a subject.

In some embodiments, the administering is local administration or systemic administration. In some embodiments, a composition described herein is formulated for administration/for use in administration via a subcutaneous, intradermal, intramuscular, inhalation, intravenous, intraperitoneal, intracranial, or intrathecal route. In some embodiments, the administering is every 1, 2, 4, 6, 8, 12, 24, 36, or 48 hours. In some embodiments, the administering is daily, weekly, or monthly. In some embodiments, the administering is repeated at least about every 28 days or 56 days.

In some embodiments, a single dose of a composition provided herein is administered to a subject. In some embodiments, a composition or pharmaceutical composition provided herein is administered to the subject by two doses. In some embodiments, a second dose of a composition or pharmaceutical composition provided herein is administered about 28 days or 56 days after the first dose. In some embodiments, a first dose is administered, and a second dose is administered about 14 days later, or about 21 days later, or about 28 days later, or about 35 days later, or about 42 days later, or about 49 days later, or about 56 days later, or about 63 days later, or about 70 days later, or about 77 days later, or about 84 days later. In some embodiments, the second dose is administered about 10-90 days following administration of the first dose, or about 15-85 days following administration of the first dose, or about 20-80 days following administration of the first dose, or about 25-75 days following administration of the first dose, or about 30-70 days following administration of the first dose, or about 35-65 days following administration of the first dose, or about 40-60 days following administration of the first dose.

In some embodiments, a third dose of a composition or pharmaceutical composition provided herein is administered to a subject. In some embodiments, the third dose is administered about 1 month following administration of the second dose, about 2 months following administration of the second dose, about 3 months following administration of the second dose, about 4 months following administration of the second dose, about 5 months following administration of the second dose, about 6 months following administration of the second dose, about 7 months following administration of the second dose, about 8 months following administration of the second dose, about 9 months following administration of the second dose, about 10 months following administration of the second dose, about 11 months following administration of the second dose, about 12 months following administration of the second dose, about 13 months following administration of the second dose, about 14 months following administration of the second dose, about 15 months following administration of the second dose, about 16 months following administration of the second dose, about 17 months following administration of the second dose, or about 18 months following administration of the second dose.

### Therapeutic Applications

Provided herein are methods of treating or preventing a disease in a subject. In some embodiments, compositions described herein are used for the treatment of an infection. In some embodiments, compositions described herein are used for the treatment of a respiratory infection. In some embodiments, the infection is a viral infection. In some embodiments, the viral infection is from a Coronavirus. In some embodiments, the coronavirus is SARS-CoV-2. In some embodiments, the Coronavirus is MERS or SARS. In some embodiments, the Coronavirus is a SARS-CoV-2 variant.

Further provided herein are methods for immunoprotecting the subject. In some embodiments, the method reduces the severity of a SARS-CoV-2 infection. In some embodiments, the method prevents a SARS-CoV-2 infection in a subject. In some embodiments, compositions described herein are used for enhancing the immune response of a subject to a viral antigen provided herein or a variant thereof. In some embodiments, compositions described herein are used for immunizing a subject. In some embodiments, compositions described herein are used for the reduction of severity of an infection in a subject. In some embodiments, compositions described herein provide for reduction of severity or duration of symptoms associated with an infection in a subject.

In some embodiments, the subject is a mammal. In some embodiments, the subject is a human. In some embodiments, the subject has or is suspected of having a viral infection. In some embodiments, the subject has or is suspected of having a coronavirus infection. In some embodiments, the subject has or is suspected of having COVID-19 caused by the SARS-CoV-2 virus or a variant thereof. In some embodiments, the subject has COVID-19 caused by the delta variant of SARS-CoV-2. In some embodiments, the subject has COVID-19 caused by the omicron variant of SARS-CoV-2. In some embodiments, the subject is administered a composition provided herein that comprises a nucleic acid encoding a stem helix region of the SARS-CoV-2 spike protein. In some embodiments, the subject is immunocompromised. In some embodiments, the subject is immunosuppressed prior to administration (e.g., by an immunosuppressive agent). In some embodiments, the subject has received at least one dose of a SARS-CoV-2 vaccine prior to administration of a composition provided herein. In some embodiments, the subject had previously exhibited at least one symptom of an upper respiratory infection.

### Kits

Provided herein is a kit comprising a composition provided herein, a pharmaceutical composition provided herein; and optionally, a delivery system for administration to a subject.

In some embodiments, the kit further comprises one or more surfactants. In some embodiments, a formulation of a composition described herein is prepared in a single container for administration. In some embodiments, a formulation of a composition described herein is prepared two containers for administration, separating the nucleic acid from the nanoparticle carrier. As used herein, “container” includes vessel, vial, ampule, tube, cup, box, bottle, flask, jar, dish, well of a single-well or multi-well apparatus, reservoir, tank, or the like, or other device in which the herein disclosed compositions may be placed, stored and/or transported, and accessed to remove the contents. Examples of such containers include glass and/or plastic sealed or re-sealable tubes and ampules, including those having a rubber septum or other sealing means that is compatible with withdrawal of the contents using a needle and syringe. In some implementations, the containers are RNase free.

In some embodiments, the kit comprises: (a) a lipid carrier, wherein the lipid carrier is a nanoemulsion comprising a hydrophobic core, one or more lipids, and one or more surfactants; and (b) at least one nucleic acid sequence, which comprises a sequence which encodes a sequence capable of expressing an antigen, wherein the antigen is a SARS-CoV-2 spike protein.

### Exemplary Embodiments

Provided herein are compositions, wherein the compositions comprise: a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the at least one nucleic acid comprises a sequence at least 85% identical to SEQ ID NOS: 1-8. Further provided herein, the nucleic acid is at least 85% identical to the sequence SEQ ID NO: 4. Further provided herein, the nucleic acid is at least 85% identical to the sequence SEQ ID NO: 8. Further provided herein, the nucleic acid is identical to SEQ ID NO: 4. Further provided herein, the nucleic acid is identical to SEQ ID NO: 8. Further provided herein, the nucleic acid is in complex with the lipid carrier. Further provided herein, the nucleic acid further codes for an RNA polymerase. Further provided herein, the RNA polymerase is a Venezuelan equine encephalitis virus (VEEV) RNA polymerase. Further provided herein, the nucleic acid coding the RNA polymerase comprises the nucleic acid sequence of SEQ ID NO: 20. Further provided herein, the liquid oil is α-tocopherol, coconut oil, grapeseed oil, lauroyl polyoxylglyceride, mineral oil, monoacylglycerol, palmkemal oil, olive oil, paraffin oil, peanut oil, propolis, squalene, squalane, solanesol, soy lecithin, soybean oil, sunflower oil, a triglyceride, or vitamin E. Further provided herein, the triglyceride is capric triglyceride, caprylic triglyceride, a caprylic and capric triglyceride, a triglyceride ester, or myristic acid triglycerin. Further provided herein, the cationic lipid is 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[l-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 30-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9′″Z,12Z,12′,12″Z,12′″Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide.

Provided herein are compositions, wherein the compositions comprise: a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the nucleic acid comprises a sequence coding a SARS-CoV-2 omicron variant spike protein antigen or functional variant thereof. Further provided herein, the SARS-CoV-2 omicron variant spike protein antigen or functional variant thereof comprises the stem helix of the SARS-CoV-2 omicron variant spike protein antigen. Further provided herein, the stem helix of the SARS-CoV-2 omicron variant spike protein antigen comprises an amino acid sequence of SEQ ID NO: 19. Further provided herein, the nucleic acid coding the SARS-CoV-2 omicron variant spike protein antigen comprises a polynucleotide sequence of SEQ ID NO: 8. Further provided herein, the SARS-CoV-2 omicron variant spike protein comprises the amino acid sequence of SEQ ID NO: 16. Further provided herein, the nucleic acid is in complex with the lipid carrier. Further provided herein, the nucleic acid further codes for an RNA polymerase. Further provided herein, the RNA polymerase is a Venezuelan equine encephalitis virus (VEEV) RNA polymerase. Further provided herein, the nucleic acid encoding for the RNA polymerase comprises the nucleic acid sequence of SEQ ID NO: 20. Further provided herein, the liquid oil is α-tocopherol, coconut oil, grapeseed oil, lauroyl polyoxylglyceride, mineral oil, monoacylglycerol, palmkemal oil, olive oil, paraffin oil, peanut oil, propolis, squalene, squalane, solanesol, soy lecithin, soybean oil, sunflower oil, a triglyceride, or vitamin E. Further provided herein, the triglyceride is capric triglyceride, caprylic triglyceride, a caprylic and capric triglyceride, a triglyceride ester, or myristic acid triglycerin. Further provided herein, the cationic lipid is 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[l-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 30-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9″′Z,12Z,12′,12″Z,12″′Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide.

Provided herein are compositions, wherein the compositions comprise: a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the nucleic acid comprises a sequence coding a stem helix of SARS-CoV-2 spike protein antigen or functional variant thereof. Further provided herein, the SARS-CoV-2 spike protein antigen is derived from an alpha variant of SARS-CoV-2, a beta variant of SARS-CoV-2, a delta variant of SARS-CoV-2, a gamma variant of SARS-CoV-2, a mu variant of SARS-CoV-2, or an omicron variant of SARS-CoV-2. Further provided herein, the stem helix of the SARS-CoV-2 spike protein antigen comprises an amino acid sequence of SEQ ID NO: 18 or SEQ ID NO: 19. Further provided herein, the nucleic acid is in complex with the lipid carrier. Further provided herein, the nucleic acid further codes for an RNA polymerase. Further provided herein, the RNA polymerase is a Venezuelan equine encephalitis virus (VEEV) RNA polymerase. Further provided herein, the nucleic acid coding the RNA polymerase comprises the nucleic acid sequence of SEQ ID NO: 20. Further provided herein, the liquid oil is α-tocopherol, coconut oil, grapeseed oil, lauroyl polyoxylglyceride, mineral oil, monoacylglycerol, palmkernal oil, olive oil, paraffin oil, peanut oil, propolis, squalene, squalane, solanesol, soy lecithin, soybean oil, sunflower oil, a triglyceride, or vitamin E. Further provided herein, the triglyceride is capric triglyceride, caprylic triglyceride, a caprylic and capric triglyceride, a triglyceride ester, or myristic acid triglycerin. Further provided herein, the cationic lipid is 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[l-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3″′-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-TH-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-TH-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 30-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9″′Z,12Z,12′,12″Z,12″′Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide.

Provided herein are compositions, wherein the compositions comprise: a lipid carrier, wherein the lipid carrier comprises: liquid oil; an inorganic nanoparticle, wherein the inorganic nanoparticle comprises iron oxide present in an amount of about 0.2 mg/ml 12 nm iron oxide; and surfactants, wherein the surfactants comprise a cationic lipid; and at least one nucleic acid, wherein the nucleic acid comprises a sequence coding a SARS-CoV-2 spike protein antigen sequence or functional variant thereof. Further provided herein are compositions comprising: a nucleic acid present in an amount of up to about 200 ug; a cationic lipid present in a concentration of up to about 1.5 mg/ml; iron oxide present in a concentration of up to about 0.01 mg/ml; squalene present in a concentration of up to about 1.88 mg/ml; sorbitan monostearate present in a concentration of up to about 1.86 mg/ml; polysorbate 80 present in a concentration of up to about 1.86 mg/ml; sucrose present in a concentration of up to about 50 mg/ml; and optionally, citric acid monohydrate present in a concentration of up to about 2.1 mg/ml. Further provided herein are compositions wherein the nucleic acid is RNA or DNA. Further provided herein are compositions wherein the nucleic acid is RNA and present in an amount of up to about 50 ug. Further provided herein, the liquid oil is α-tocopherol, coconut oil, grapeseed oil, lauroyl polyoxylglyceride, mineral oil, monoacylglycerol, palmkemal oil, olive oil, paraffin oil, peanut oil, propolis, squalene, squalane, solanesol, soy lecithin, soybean oil, sunflower oil, a triglyceride, or vitamin E. Further provided herein, the triglyceride is capric triglyceride, caprylic triglyceride, a caprylic and capric triglyceride, a triglyceride ester, or myristic acid triglycerin. Further provided herein, the cationic lipid is 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[l-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3″′-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 30-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9″′Z,12Z,12′,12″Z,12″′Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein, the nucleic acid is in complex with the lipid carrier. Further provided herein, the SARS-CoV-2 spike protein antigen is derived from an alpha variant of SARS-CoV-2, a beta variant of SARS-CoV-2, a delta variant of SARS-CoV-2, a gamma variant of SARS-CoV-2, a mu variant of SARS-CoV-2, or an omicron variant of SARS-CoV-2. Further provided herein, the SARS-CoV-2 spike protein is derived from a variant of SARS-CoV-2 comprising an amino acid substitution of D614G. Further provided herein, the nucleic acid comprises a sequence as set forth in one of SEQ ID NOS: 1-8. Further provided herein, the SARS-CoV-2 spike protein antigen sequence or functional variant thereof has an amino acid sequence as set forth in one of SEQ ID NOS: 9-16. Further provided herein, the nucleic acid further codes for an RNA polymerase. Further provided herein, the RNA polymerase is a Venezuelan equine encephalitis virus (VEEV) RNA polymerase. Further provided herein, the nucleic acid coding the RNA polymerase comprises the nucleic acid sequence of SEQ ID NO: 20.

Provided herein are compositions, wherein the compositions comprise: (a) a lipid carrier, wherein the lipid carrier is a nanoemulsion comprising: about 30 mg/mL DOTAP chloride; about 37.5 mg/mL squalene; about 37 mg/ml sorbitan monostearate; about 37 mg/ml polysorbate 80; about 10 mM sodium citrate; and about 0.2 mg Fe/ml 12 nm oleic acid-coated iron oxide nanoparticles; and (b) at least one nucleic acid, wherein the at least one nucleic acid comprises a sequence coding a SARS-CoV-2 spike protein antigen sequence or functional variant thereof. Further provided herein are compositions comprising: a nucleic acid present in an amount of up to about 200 ug; a cationic lipid present in a concentration of up to about 1.5 mg/ml; iron oxide present in a concentration of up to about 0.01 mg/ml; squalene present in a concentration of up to about 1.88 mg/ml; sorbitan monostearate present in a concentration of up to about 1.86 mg/ml; polysorbate 80 present in a concentration of up to about 1.86 mg/ml; sucrose present in a concentration of up to about 50 mg/ml; and optionally, citric acid monohydrate present in a concentration of up to about 2.1 mg/ml. Further provided herein are compositions wherein the nucleic acid is RNA or DNA. Further provided herein are compositions wherein the nucleic acid is RNA and present in an amount of up to about 50 ug. Further provided herein, the composition further comprises sucrose. Further provided herein, the sucrose is present in an about of about 50 mg. Further provided herein, the nucleic acid is in complex with the lipid carrier. Further provided herein, the SARS-CoV-2 spike protein antigen is derived from an alpha variant of SARS-CoV-2, a beta variant of SARS-CoV-2, a delta variant of SARS-CoV-2, a gamma variant of SARS-CoV-2, a mu variant of SARS-CoV-2, or an omicron variant of SARS-CoV-2. Further provided herein, the SARS-CoV-2 spike protein is derived from a variant of SARS-CoV-2 comprising an amino acid substitution of D614G. Further provided herein, the nucleic acid comprises a sequence as set forth in one of SEQ ID NOS: 1-8. Further provided herein, the SARS-CoV-2 spike protein antigen sequence or functional variant thereof has an amino acid sequence as set forth in one of SEQ ID NOS: 9-17. Further provided herein, the nucleic acid further codes for an RNA polymerase. Further provided herein, the RNA polymerase is a Venezuelan equine encephalitis virus (VEEV) RNA polymerase. Further provided herein, the nucleic acid encoding the RNA polymerase comprises the nucleic acid sequence set forth in SEQ ID NO: 20.

Provided herein are compositions, wherein the compositions comprise: comprising: (a) a lipid carrier, wherein the lipid carrier is a nanoemulsion comprising: DOTAP chloride present in an amount of about 0.75 mg; squalene present in an amount of about 0.94 mg; sorbitan monostearate present in an amount of about 0.93 mg; polysorbate 80 present in an amount of about 0.93 mg; citric acid monohydrate present in an amount of about 1.05 mg; and oleic acid-coated iron oxide nanoparticles present in an amount of about 0.005 mg; and (b) at least one nucleic acid, wherein the at least one nucleic acid comprises a sequence coding a SARS-CoV-2 spike protein antigen sequence or functional variant thereof. Further provided herein, the at least one nucleic acid sequence is present in an amount of up to about 100 micrograms (μg). Further provided herein, the at least one nucleic acid sequence is present in an amount of up to about 25 μg. Further provided herein, the composition further comprises sucrose. Further provided herein the sucrose is present in an about of about 50 milligrams (mg). Further provided herein, the nucleic acid is in complex with the lipid carrier. Further provided herein, the SARS-CoV-2 spike protein antigen is derived from an alpha variant of SARS-CoV-2, a beta variant of SARS-CoV-2, a delta variant of SARS-CoV-2, a gamma variant of SARS-CoV-2, a mu variant of SARS-CoV-2, or an omicron variant of SARS-CoV-2. Further provided herein, the SARS-CoV-2 spike protein is derived from a variant of SARS-CoV-2 comprising an amino acid substitution of D614G. Further provided herein, the nucleic acid comprises a sequence as set forth in one of SEQ ID NOS: 1-8. Further provided herein, the SARS-CoV-2 spike protein antigen sequence or functional variant thereof has an amino acid sequence as set forth in one of SEQ ID NOS: 9-19. Further provided herein, the nucleic acid further codes for an RNA polymerase. Further provided herein, the RNA polymerase is a Venezuelan equine encephalitis virus (VEEV) RNA polymerase. Further provided herein, the nucleic acid coding the RNA polymerase comprises the nucleic acid sequence of SEQ ID NO: 20. Further provided herein, the composition is lyophilized.

Provided herein is a suspension comprising the composition provided herein. Further provided herein is a pharmaceutical composition comprising the composition provided herein and a pharmaceutically acceptable excipient.

Provided herein are compositions, wherein the compositions comprise a) a lipid carrier, wherein the lipid carrier is a nanoemulsion comprising a hydrophobic core, one or more inorganic nanoparticles and one or more lipids; b) at least one nucleic acid sequence, wherein the nucleic acid sequence comprises a sequence capable of expressing an antigen, wherein the antigen is a SARS-CoV-2 spike protein. Further provided herein, the nucleic acid is RNA. Further provided herein, the composition further comprises a nucleic acid polymerase or a further nucleic acid encoding a sequence capable of expressing a nucleic acid polymerase. Further provided herein, compositions further comprise an RNA polymerase or a further nucleic acid encoding a sequence capable of expressing an RNA polymerase. Further provided herein, the nucleic acid comprises a sequence as set forth in one of SEQ ID NOS: 1-8. Further provided herein, the SARS-CoV-2 spike protein has an amino acid sequence as set forth in one of SEQ ID NOS: 9-19. Further provided herein, the nucleic acid comprises a sequence as set forth in SEQ ID NO: 1. Further provided herein, the nucleic acid comprises a sequence as set forth in SEQ ID NO: 2. Further provided herein, the nucleic acid comprises a sequence as set forth in SEQ ID NO: 3. Further provided herein, the nucleic acid comprises a sequence as set forth in SEQ ID NO: 4. Further provided herein, the nucleic acid comprises a sequence as set forth in SEQ ID NO: 5. Further provided herein, the nucleic acid comprises a sequence as set forth in SEQ ID NO: 6. Further provided herein, the nucleic acid comprises a sequence as set forth in SEQ ID NO: 7. Further provided herein, the nucleic acid sequence comprises a sequence as set forth in SEQ ID NO: 8. Further provided herein, the SARS-CoV-2 spike protein is derived from the alpha variant of SARS-CoV-2. Further provided herein, the SARS-CoV-2 spike protein is derived from the beta variant of SARS-CoV-2. Further provided herein, the SARS-CoV-2 spike protein is derived from the delta variant of SARS-CoV-2. Further provided herein, the SARS-CoV-2 spike protein is derived from the gamma variant of SARS-CoV-2. Further provided herein, the SARS-CoV-2 spike protein is derived from the mu variant of SARS-CoV-2. Further provided herein, the SARS-CoV-2 spike protein is derived from a variant of SARS-CoV-2 comprising an amino acid substitution of D614G. Further provided herein, the hydrophobic core comprises an oil. Further provided herein, the oil comprises at least one of α-tocopherol, lauroyl polyoxylglyceride, monoacylglycerol, propolis, squalene, mineral oil, grapeseed oil, olive oil, paraffin oil, peanut oil, soybean oil, sunflower oil, soy lecithin, triglyceride, and vitamin E, and a medium chain triglyceride. Further provided herein, the one or more inorganic nanoparticles is selected from the group consisting of metal salts, metal oxides, metal hydroxides, metal phosphates, metalloids and any combinations thereof. Further provided herein, the one or more lipids is selected from the group consisting of cationic lipids, anionic lipids, neutral lipids, and any combinations thereof. Further provided herein, the one or more lipids is a cationic lipid. Further provided herein, the cationic lipid is selected from the group consisting of 1,2-dioleoyloxy-3-(trimethylammonium)propane (DOTAP); 3β-[N—(N′,N′-dimethylaminoethane)-carbamoyl]cholesterol (DC Cholesterol); dimethyldioctadecylammonium (DDA); 1,2-dimyristoyl-3-trimethylammoniumpropane (DMTAP), dipalmitoyl(C16:0)trimethyl ammonium propane (DPTAP); distearoyltrimethylammonium propane (DSTAP); N-[l-(2,3-dioleyloxy)propyl]-N,N,Ntrimethylammonium chloride (DOTMA); N,N-dioleoyl-N,N-dimethylammonium chloride (DODAC); 1,2-dioleoyl-sn-glycero-3-ethylphosphocholine (DOEPC); 1,2-dioleoyl-3-dimethylammonium-propane (DODAP); and 1,2-dilinoleyloxy-3-dimethylaminopropane (DLinDMA); 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), and any combinations thereof. Further provided herein, the lipid carrier optionally comprises one or more surfactants. Further provided herein, the one or more surfactants is selected from the group consisting of hydrophobic surfactant, hydrophilic surfactant, and any combinations thereof. Further provided herein, the hydrophobic surfactant comprises a sorbitan ester selected from the group consisting of sorbitan monostearate, sorbitan monooleate, and sorbitan trioleate; and the hydrophilic surfactant comprises a polysorbate. Further provided herein, the lipid carrier have a z-average hydrodynamic diameter ranging from about 40 nm to about 150 nm, with an average polydispersity index ranging from about 0.1 to about 0.4. Further provided herein are compositions, wherein the one or more nucleic acids is incorporated or complexed with the lipid carrier to form a lipid carrier-nucleic acid complex. Further provided herein, the complex is formed via non-covalent interactions or via reversible covalent interactions. Further provided herein, the molar ratio of the lipid carrier to the one or more nucleic acids, characterized by the nitrogen-to-phosphate (N:P) molar ratio, ranges from about 1:1 to about 150:1. Further provided herein, the composition is stable at 2 to 8 degrees Celsius.

Provided herein are vaccine compositions, wherein the vaccine comprises: (a) a lipid carrier, wherein the lipid carrier is a nanoemulsion comprising a hydrophobic core, one or more inorganic nanoparticles and one or more lipids; (b) at least one nucleic acid sequence, wherein the nucleic acid sequence comprises a sequence capable of expressing an antigen, wherein the antigen is a SARS-CoV-2 spike protein. Further provided herein, the nucleic acid is RNA. Further provided herein, the vaccine compositions further comprise a nucleic acid polymerase or a further nucleic acid encoding a sequence capable of expressing a nucleic acid polymerase. Further provided herein, the vaccine compositions further comprise an RNA polymerase or a further nucleic acid sequence encoding a sequence capable of expressing an RNA polymerase. Further provided herein, the nucleic acid comprises a sequence as set forth in one of SEQ ID NOS: 1-8. Further provided herein, the SARS-CoV-2 spike protein has an amino acid sequence as set forth in one of SEQ ID NOS: 9-17. Further provided herein, the nucleic acid comprises a sequence as set forth in SEQ ID NO: 1. Further provided herein, the nucleic acid comprises a sequence as set forth in SEQ ID NO: 2. Further provided herein, the nucleic acid comprises a sequence as set forth in SEQ ID NO: 3. Further provided herein, the nucleic acid comprises a sequence as set forth in SEQ ID NO: 4. Further provided herein, the nucleic acid comprises a sequence as set forth in SEQ ID NO: 5. Further provided herein, the nucleic acid comprises a sequence as set forth in SEQ ID NO: 6. Further provided herein, the nucleic acid comprises a sequence as set forth in SEQ ID NO: 7. Further provided herein, the nucleic acid sequence comprises a sequence as set forth in SEQ ID NO: 8. Further provided herein, the SARS-CoV-2 spike protein is the alpha variant of SARS-CoV-2. Further provided herein, the SARS-CoV-2 spike protein is the beta variant of SARS-CoV-2. Further provided herein, the SARS-CoV-2 spike protein is the delta variant of SARS-CoV-2. Further provided herein, the SARS-CoV-2 spike protein is the gamma variant of SARS-CoV-2. Further provided herein, the SARS-CoV-2 spike protein is the mu variant of SARS-CoV-2. Further provided herein, the SARS-CoV-2 spike protein is the D614G variant of SARS-CoV-2. Further provided herein, the hydrophobic core comprises an oil. Further provided herein, the oil comprises at least one of α-tocopherol, lauroyl polyoxylglyceride, monoacylglycerol, propolis, squalene, mineral oil, grapeseed oil, olive oil, paraffin oil, peanut oil, soybean oil, sunflower oil, soy lecithin, triglyceride, vitamin E, a medium chain triglyceride. Further provided herein, the one or more inorganic nanoparticles is selected from the group consisting of metal salts, metal oxides, metal hydroxides, metal phosphates, metalloids, and any combinations thereof. Further provided herein, the one or more lipids is selected from the group consisting of cationic lipids, anionic lipids, neutral lipids, and any combinations thereof. Further provided herein, the one or more lipids is a cationic lipid. Further provided herein, the cationic lipid is selected from the group consisting of 1,2-dioleoyloxy-3-(trimethylammonium)propane (DOTAP); 3β-[N—(N′,N′-dimethylaminoethane)-carbamoyl]cholesterol (DC Cholesterol); dimethyldioctadecylammonium (DDA); 1,2-dimyristoyl-3-trimethylammoniumpropane (DMTAP), dipalmitoyl(C16:0)trimethyl ammonium propane (DPTAP); distearoyltrimethylammonium propane (DSTAP); N-[l-(2,3-dioleyloxy)propyl]-N,N,Ntrimethylammonium chloride (DOTMA); N,N-dioleoyl-N,N-dimethylammonium chloride (DODAC); 1,2-dioleoyl-sn-glycero-3-ethylphosphocholine (DOEPC); 1,2-dioleoyl-3-dimethylammonium-propane (DODAP); and 1,2-dilinoleyloxy-3-dimethylaminopropane (DLinDMA); 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), and any combinations thereof. Further provided herein, the lipid carrier optionally comprises one or more surfactants. Further provided herein, the one or more surfactants is selected from the group consisting of hydrophobic surfactant, hydrophilic surfactant, and any combinations thereof. Further provided herein, the hydrophobic surfactant comprises a sorbitan ester selected from the group consisting of sorbitan monostearate, sorbitan monooleate, and sorbitan trioleate; and the hydrophilic surfactant comprises a polysorbate. Further provided herein, the lipid carrier have a z-average hydrodynamic diameter ranging from about 40 nm to about 150 nm, with an average polydispersity index ranging from about 0.1 to about 0.4. Further provided herein, the one or more nucleic acids is incorporated or complexed with the lipid carrier to form a lipid carrier-nucleic acid complex. Further provided herein, the complex is formed via non-covalent interactions or via reversible covalent interactions. Further provided herein, the molar ratio of the lipid carrier to the one or more nucleic acids, characterized by the nitrogen-to-phosphate (N:P) molar ratio, ranges from about 1:1 to about 150:1. Further provided herein, the composition is stable at 2 to 8 degrees Celsius.

Provided herein are methods of generating an immune response in a subject, wherein the methods comprise administering to said subject a composition comprising: a) a lipid carrier, wherein the lipid carrier is a nanoemulsion comprising a hydrophobic core, one or more inorganic nanoparticles and one or more lipids; b) at least one nucleic acid sequence, wherein the nucleic acid sequence comprises a sequence capable of expressing an antigen, wherein the antigen is a SARS-CoV-2 spike protein. Further provided herein, the composition is administered to the subject by two doses. Further provided herein, the second dose is administered at about 28 days after the first dose. Further provided herein, the methods further comprise administering a third dose of said composition to said subject. Further provided herein, 5 micrograms of said composition is administered to said subject. Further provided herein, 10 micrograms of said composition is administered to said subject. Further provided herein, 25 micrograms of said composition is administered to said subject. Further provided herein, the subject is a mammal. Further provided herein, the mammal is a human. Further provided herein, the composition is administered intramuscularly.

Provided herein are compositions for immunoprotecting a subject, the compositions comprising: a) a lipid carrier, wherein the lipid carrier is a nanoemulsion comprising a hydrophobic core, one or more inorganic nanoparticles and one or more lipids; b) at least one nucleic acid sequence, wherein the nucleic acid sequence comprises a sequence capable of expressing an antigen, wherein the antigen is a SARS-CoV-2 spike protein.

Provided herein are methods of reducing the severity of a SARS-CoV-2 infection, the methods comprising administering prior to infection a composition comprising: a) a lipid carrier, wherein the lipid carrier is a nanoemulsion comprising a hydrophobic core, one or more inorganic nanoparticles and one or more lipids; b) at least one nucleic acid sequence, wherein the nucleic acid sequence comprises a sequence capable of expressing an antigen, wherein the antigen is a SARS-CoV-2 spike protein. Further provided herein, the composition is administered to the subject by two doses. Further provided herein, the second dose is administered at about 28 days after the first dose. Further provided herein, the methods further comprise administering a third dose of said composition to said subject. Further provided herein, 5 micrograms of said composition is administered to said subject. Further provided herein, 10 micrograms of said composition is administered to said subject. Further provided herein, 25 micrograms of said composition is administered to said subject. Further provided herein, the subject is a mammal. Further provided herein, the mammal is a human. Further provided herein, the composition is administered intramuscularly.

Provided herein are methods of immunoprotecting a subject, the methods comprising: a) a lipid carrier, wherein the lipid carrier is a nanoemulsion comprising a hydrophobic core, one or more inorganic nanoparticles and one or more lipids; b) at least one nucleic acid sequence, wherein the nucleic acid sequence comprises a sequence capable of expressing an antigen, wherein the antigen is a SARS-CoV-2 spike protein. Further provided herein, the composition is administered to the subject by two doses. Further provided herein, the second dose is administered at about 28 days after the first dose. Further provided herein, the methods further comprise administering a third dose of said composition to said subject. Further provided herein, 5 micrograms of said composition is administered to said subject. Further provided herein, 10 micrograms of said composition is administered to said subject. Further provided herein, 25 micrograms of said composition is administered to said subject. Further provided herein, the subject is a mammal. Further provided herein, the mammal is a human. Further provided herein, the composition is administered intramuscularly.

Provided herein are kits, the kits comprising: (a) a lipid carrier, wherein the lipid carrier is a nanoemulsion comprising a hydrophobic core, one or more inorganic nanoparticles and one or more lipids; (b) at least one nucleic acid sequence, wherein the nucleic acid sequence comprises a sequence capable of expressing an antigen, wherein the antigen is a SARS-CoV-2 spike protein. Provided herein are kits, the kits comprising: (a) a lipid carrier, wherein the lipid carrier is a nanoemulsion comprising a hydrophobic core, one or more lipids, and one or more surfactants; (b) at least one nucleic acid sequence, which comprises a sequence which encodes a sequence capable of expressing an antigen, wherein the antigen is a SARS-CoV-2 spike protein. Further provided herein, the kit further comprises one or more surfactants. Further provided herein, the RNA comprises a sequence comprising a vector sequence as set forth in SEQ ID NO: 20. Further provided herein, the RNA comprises a sequence comprising an antigen sequence as set forth in one of SEQ ID NOS: 1-8. Further provided herein, the RNA comprises a sequence comprising a sequence as set forth in one of SEQ ID NOS: 24-31. Further provided herein, the RNA comprises a sequence comprising a vector sequence as set forth in one of SEQ ID NOS: 32-38.

Provided herein are dried compositions comprising: a) a lipid carrier, wherein the lipid carrier is a nanoemulsion comprising a hydrophobic core, one or more inorganic nanoparticles and one or more lipids; b) at least one nucleic acid sequence, which comprises a sequence which encodes a sequence capable of expressing an antigen, wherein the antigen is a SARS-CoV-2 spike protein; and c) at least one cryoprotectant. Further provided herein, the composition is lyophilized. Further provided herein, the composition is spray-dried. Further provided herein, the composition is thermally stable. Further provided herein, the composition is thermally stable at about 25 degrees C. Further provided herein, the composition is thermally stable at about 45 degrees C. Further provided herein, the composition is thermally stable at about −20 degrees C. Further provided herein, the composition is thermally stable at about 2 degrees C. to about 8 degrees C. Further provided herein, the composition is thermally stable for at least 1 week, at least 2 weeks, and/or at least 1 month. Further provided herein, the hydrophobic core comprises an oil. Further provided herein, the oil comprises at least one of α-tocopherol, lauroyl polyoxylglyceride, monoacylglycerol, propolis, squalene, mineral oil, grapeseed oil, olive oil, paraffin oil, peanut oil, soybean oil, sunflower oil, soy lecithin, triglyceride, vitamin E, a medium chain triglyceride, dihydroisosqualene (DHIS), famesene and squalane. Further provided herein, the one or more inorganic nanoparticles is selected from the group consisting of metal salts, metal oxides, metal hydroxides, metal phosphates, metalloids, and any combinations thereof. Further provided herein, the one or more lipids is selected from the group consisting of cationic lipids, anionic lipids, neutral lipids, and any combinations thereof. Further provided herein, the one or more lipids is a cationic lipid. Further provided herein, the cationic lipid is selected from the group consisting of 1,2-dioleoyloxy-3-(trimethylammonium)propane (DOTAP); 3β-[N—(N′,N′-dimethylaminoethane)-carbamoyl]cholesterol (DC Cholesterol); dimethyldioctadecylammonium (DDA); 1,2-dimyristoyl-3-trimethylammoniumpropane (DMTAP), dipalmitoyl(C16:0)trimethyl ammonium propane (DPTAP); distearoyltrimethylammonium propane (DSTAP); N-[l-(2,3-dioleyloxy)propyl]-N,N,Ntrimethylammonium chloride (DOTMA); N,N-dioleoyl-N,N-dimethylammonium chloride (DODAC); 1,2-dioleoyl-sn-glycero-3-ethylphosphocholine (DOEPC); 1,2-dioleoyl-3-dimethylammonium-propane (DODAP); and 1,2-dilinoleyloxy-3-dimethylaminopropane (DLin-DMA); 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), 1,2-dioleoyl-sn-glycero-3-phosphoethanolamine (DOPE); N-decyl-N,N-dimethyldecan-1-aminium bromide (DDAB); 2,3-dioleyloxy-N-[2-(sperminecarboxamido)ethyl]-N,N-dimethyl-1-propanaminium trifluoroacetate (DOSPA); ethylphosphatidylcholine (ePC); and any combinations thereof. Further provided herein, the lipid carrier optionally comprises at least one surfactant. Further provided herein, the at least one surfactant is selected from the group consisting of a hydrophobic surfactant, a hydrophilic surfactant, and any combinations thereof. Further provided herein, the hydrophobic surfactant comprises a sorbitan ester selected from the group consisting of Span 20, Span 40, Span 60, Span 65, Span 80 and Span 85; and the hydrophilic surfactant comprises a polysorbate. Further provided herein, the lipid carrier has a z-average hydrodynamic diameter ranging from about 40 nm to about 150 nm, with an average polydispersity index ranging from about 0.1 to about 0.4. Further provided herein, the one or more nucleic acid sequence is an RNA. Further provided herein, the RNA is a self-replicating RNA. Further provided herein, the one or more nucleic acid sequence comprises a sequence which encodes an antigen. Further provided herein, the antigen is derived from a virus. Further provided herein, the virus is selected from the group consisting of: a hepatitis virus, a coronavirus, a mosquito-borne virus, and an HIV virus. Further provided herein, the one or more nucleic acid sequence is incorporated or complexed with the lipid carrier to form a lipid carrier-nucleic acid complex. Further provided herein, the lipid carrier-nucleic acid complex is formed via non-covalent interactions or via reversible covalent interactions. Further provided herein, the molar ratio of the lipid carrier to the one or more nucleic acids, characterized by the nitrogen-to-phosphate (N:P) molar ratio, ranges from about 1:1 to about 150:1. Further provided herein, the at least one cryoprotectant is selected from the group consisting of sucrose, maltose, trehalose, mannitol, glucose, and any combinations thereof. Further provided herein, the at least one cryoprotectant is sucrose. Further provided herein, the at least one cryoprotectant is at about 1% w/v to at about 20% w/v. Further provided herein, the at least one cryoprotectant is at about 10% w/v to at about 20% w/v. Further provided herein, the at least one cryoprotectant is at about 10% w/v.

Provided herein are pharmaceutical compositions, wherein the pharmaceutical compositions comprise: a composition provided herein reconstituted in a suitable diluent and a pharmaceutically acceptable carrier. Further provided herein, the diluent is aqueous. Further provided herein, the diluent is water.

Provided herein are kits comprising the pharmaceutical composition provided herein and a delivery system for administration to a subject.

Provided herein are vaccine delivery systems comprising the pharmaceutical composition provided herein and optionally one or more vaccine adjuvants.

Provided herein are methods for generating an immune response in a subject, the methods comprise administering to a subject a composition or a pharmaceutical composition provided herein, thereby generating an immune response to an antigen. Further provided herein, the composition is administered to the subject by two doses. Further provided herein, the second dose is administered at about 28 days after the first dose. Further provided herein, the method further comprises administering a third dose of the composition to said subject. Further provided herein, 5 μg of the composition is administered to the subject. Further provided herein, 10 μg of the composition is administered to said subject. Further provided herein, 25 μg of the composition is administered to said subject. Further provided herein, the composition is administered intramuscularly or intranasally. Further provided herein, the subject is a human. Further provided herein, the subject has or is suspected of having a viral infection. Further provided herein, the viral infection is a coronavirus infection. Further provided herein the coronavirus infection is caused by a SARS-CoV-2 virus or a variant thereof. Further provided herein, the antigen is a SARS-CoV-2 spike protein or a fragment thereof. Further provided herein, the immune response comprises increasing the amount or titer of neutralizing antibodies to the antigen as compared to a subject that has not been administered the composition. Further provided herein, the immune response comprises increasing the amount of CD4+ and/or CD8+positive T cells as compared to a subject that has not been administered the composition. Further provided herein, the subject is immunocompromised or immunosuppressed.

Provided herein are methods of augmenting an immune response in a subject, the method comprising: administering to a subject the composition provided herein, thereby augmenting an immune response to an antigen.

Provided herein are methods of treating or preventing a disease in a subject, the methods comprise administering a therapeutically effective amount of the pharmaceutical provided herein to the subject.

Provided herein are methods of imaging and/or tracking delivery of one or more nucleic acids in a subject, the methods comprise administering a therapeutically effective amount of the pharmaceutical composition provided herein to the subject.

Provided herein are methods for preparing a lyophilized composition comprising: a) obtaining a lipid carrier, wherein the lipid carrier is a nanoemulsion comprising a hydrophobic core, one or more inorganic nanoparticles and one or more lipids; b) incorporating at least one nucleic acid into the lipid carrier to form a lipid carrier-nucleic acid complex, wherein the nucleic acid comprises a sequence comprising an antigen sequence which is at least 80% identical to a sequence as set forth in one SEQ ID NOS: 1-8, 24-31; c) adding at least one cryoprotectant to the lipid carrier-nucleic acid complex to form a formulation; and d) lyophilizing the formulation to form a lyophilized composition.

Provided herein are methods for preparing a spray-dried composition comprising: (a) obtaining a lipid carrier, wherein the lipid carrier is a nanoemulsion comprising a hydrophobic core, one or more inorganic nanoparticles and one or more lipids; (b) incorporating at least one nucleic acid into the lipid carrier to form a lipid carrier-nucleic acid complex, wherein the nucleic acid comprises a sequence comprising an antigen sequence which is at least 80% identical to a sequence as set forth in one of SEQ ID NOS: 1-8, 24-31; (c) adding at least one cryoprotectant to the lipid carrier-nucleic acid complex to form a formulation; and (d)
spray drying the formulation to form a spray-dried composition.

Provided herein are methods for reconstituting a lyophilized composition comprising: (a) obtaining a lipid carrier, wherein the lipid carrier is a nanoemulsion comprising a hydrophobic core, one or more inorganic nanoparticles, and one or more lipids; (b) incorporating at least one nucleic acid into the said lipid carrier to form a lipid carrier-nucleic acid complex, wherein the nucleic acid comprises a sequence comprising an antigen sequence which is at least 80% identical to a sequence as set forth in one of SEQ ID NOS: 1-8, 24-31; (c) adding at least one cryoprotectant to the lipid carrier-nucleic acid complex to form a formulation; (d) lyophilizing the formulation to form a lyophilized composition; and (e) reconstituting the lyophilized composition in a suitable diluent.

Provided herein are methods for reconstituting a spray-dried composition comprising: (a) obtaining a lipid carrier, wherein the lipid carrier is a nanoemulsion comprising a hydrophobic core, one or more inorganic nanoparticles, and one or more lipids; (b) incorporating at least one nucleic acid into the said lipid carrier to form a lipid carrier-nucleic acid complex, wherein the nucleic acid comprises a sequence comprising an antigen sequence which is at least 80% identical to a sequence as set forth in one of SEQ ID NOS: 1-8, 24-31; (c) adding at least one cryoprotectant to the lipid carrier-nucleic acid complex to form a formulation; (d) spray drying the formulation to form a spray-dried composition; and (e) reconstituting the spray-dried composition in a suitable diluent. Further provided herein, the diluent is aqueous. Further provided herein, the diluent is water. Further provided herein, the lyophilized composition is thermally stable. Further provided herein, the lyophilized composition is thermally stable at temperatures up to about 25° C. Further provided herein, the lyophilized composition is thermally stable at temperatures up to about 45° C. Further provided herein, the lyophilized composition is thermally stable at temperatures down to about −20° C. Further provided herein, the lyophilized composition is thermally stable at temperatures ranging from about 2° C. to about 8° C. Further provided herein, the lyophilized composition is thermally stable for at least 1 week, at least 2 weeks, and/or at least 1 month. Further provided herein, the hydrophobic core comprises an oil. Further provided herein, the oil comprises at least one of α-tocopherol, lauroyl polyoxylglyceride, monoacylglycerol, propolis, squalene, mineral oil, grapeseed oil, olive oil, paraffin oil, peanut oil, soybean oil, sunflower oil, soy lecithin, triglyceride, vitamin E, medium chain triglyceride, dihydroisosqualene (DHIS), farnesene and squalane. Further provided herein, the one or more inorganic nanoparticles is selected from the group consisting of metal salts, metal oxides, metal hydroxides, metal phosphates, metalloids and any combinations thereof. Further provided herein, the one or more lipids is selected from the group consisting of cationic lipids, anionic lipids, neutral lipids, and any combinations thereof. Further provided herein, the one or more lipids is a cationic lipid. Further provided herein, the cationic lipid is selected from the group consisting of 1,2-dioleoyloxy-3-(trimethylammonium)propane (DOTAP); 3β-[N—(N′,N′-dimethylaminoethane)-carbamoyl]cholesterol (DC Cholesterol); dimethyldioctadecylammonium (DDA); 1,2-dimyristoyl-3-trimethylammoniumpropane (DMTAP), dipalmitoyl(C16:0)trimethyl ammonium propane (DPTAP); distearoyltrimethylammonium propane (DSTAP); N-[l-(2,3-dioleyloxy)propyl]-N,N,Ntrimethylammonium chloride (DOTMA); N,N-dioleoyl-N,N-dimethylammonium chloride (DODAC); 1,2-dioleoyl-sn-glycero-3-ethylphosphocholine (DOEPC); 1,2-dioleoyl-3-dimethylammonium-propane (DODAP); and 1,2-dilinoleyloxy-3-dimethylaminopropane (DLin-DMA); 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), 1,2-dioleoyl-sn-glycero-3-phosphoethanolamine (DOPE); N-decyl-N,N-dimethyldecan-1-aminium bromide (DDAB); 2,3-dioleyloxy-N-[2-(sperminecarboxamido)ethyl]-N,N-dimethyl-1-propanaminium trifluoroacetate (DOSPA); ethylphosphatidylcholine (ePC); and any combinations thereof. Further provided herein, the lipid carrier optionally comprises one or more surfactant. Further provided herein, the one or more surfactant is selected from the group consisting of hydrophobic surfactant, hydrophilic surfactant, and any combinations thereof. Further provided herein, the hydrophobic surfactant comprises a sorbitan ester selected from the group consisting of Span 20, Span 40, Span 60, Span 65, Span 80 and Span 85; and the hydrophilic surfactant comprises a polysorbate. Further provided herein, the lipid carrier has a z-average hydrodynamic diameter ranging from about 40 nm to about 150 nm, with an average polydispersity index ranging from about 0.1 to about 0.4. Further provided herein, the one or more nucleic acid is an RNA. Further provided herein, the RNA is a self-replicating RNA. Further provided herein, the one or more nucleic acid encodes an antigen, wherein the antigen is derived from a bacterial infection, a bacterial disease, a viral infection, a viral disease, a protozoan infection, a protozoan disease, a non-communicable disease, one or more cancers, or an autoimmune disease. Further provided herein, the antigen is derived from a virus. Further provided herein, the virus is selected from the group consisting of a hepatitis virus, a corona virus, a mosquito-borne virus, and a HIV virus. Further provided herein, the one or more nucleic acid is incorporated or complexed with the lipid carrier to form a lipid carrier-nucleic acid complex. Further provided herein, the lipid carrier-nucleic acid complex is formed via non-covalent interactions or via reversible covalent interactions. Further provided herein, the at least one cryoprotectant is selected from the group consisting of sucrose, maltose, trehalose, mannitol, glucose, and any combinations thereof. Further provided herein, the at least one cryoprotectant is sucrose. Further provided herein, the at least one cryoprotectant is at about 1% w/v to at about 20% w/v. Further provided herein, the at least one cryoprotectant is at about 10% w/v to at about 20% w/v. Further provided herein, the at least one cryoprotectant is at about 10% w/v.

Provided herein are compositions for prophylaxis of SARS-CoV-2, the compositions comprising: a) a sorbitan fatty acid ester; b) an ethoxylated sorbitan ester; c) a cationic lipid; d) an immune stimulant; and e) at least one RNA encoding an antigen sequence or functional fragment thereof. Further provided herein, the sorbitan fatty acid ester is sorbitan monostearate. Further provided herein, the ethoxylated sorbitan ester is TWEEN-80. Further provided herein, the cationic lipid is DOTAP. Further provided herein, the immune stimulant is squalene. Further provided herein, the RNA encodes a SARS-CoV-2 spike protein. Further provided herein, the ratio of the esters yields a Hydrophilic-Lipophilic Balance between 8 and 11. Further provided herein, the ratio of esters and lipids yields a particle size between 30 and 200 nanometers. Further provided herein, the ratio of esters and lipids yields a particle size between 40 and 70 nanometers. Further provided herein, the immune stimulant decreases the total amount of protein produced, but increases the immune response to the vaccine. Further provided herein, the immune stimulant increases the total amount of protein, produced, but decreases the immune response to the vaccine. Further provided herein, the immune stimulant is Miglyol 810 or Miglyol 812. Further provided herein, the compositions further comprise sorbitan monostearate, polysorbate 80, DOTAP, and squalene and no solid particles.

Provided herein are compositions for prophylaxis of SARS-CoV-2. wherein the compositions comprise: (a) sorbitan monostearate; (b) polysorbate 80; (c) DOTAP; (d) an immune stimulant; and (e) at least one RNA encoding an antigen sequence or functional fragment thereof. Further provided herein, the immune stimulant decreases the total amount of protein produced, but increases the immune response to the vaccine. Further provided herein, the immune stimulant increases the total amount of protein, produced, but decreases the immune response to the vaccine. Further provided herein, the immune stimulant is Miglyol 810 or Miglyol 812. Further provided herein, the compositions further comprise squalene and no solid particles. Further provided herein, the ratio of the esters yields a Hydrophilic-Lipophilic Balance between 8 and 11. Further provided herein, the particle size is between 30 and 200 nanometers. Further provided herein, the N to P ratio is between 5 and 35. Further provided herein, the RNA encodes an amino acid sequence as set forth in one of SEQ ID NOS: 9-19. Further provided herein, the RNA comprises a sequence as set forth in one of SEQ ID NOS: 1-8. Further provided herein, the RNA comprises a sequence as set forth in one of SEQ ID NOS: 24-31. Further provided herein, the RNA comprises a sequence comprising a vector sequence as set forth in SEQ ID NO: 20. Further provided herein, the RNA comprises a sequence comprising an sequence as set forth in one of SEQ ID NOS: 1-8, 24-31. Further provided herein, the RNA comprises a sequence comprising a vector sequence as set forth in one of SEQ ID NOS: 32-38. Further provided herein, the compositions comprise a nucleic acid comprising an alphavirus replicon.

Provided here are dried compositions comprising: a lipid carrier, wherein the lipid carrier is a nanoemulsion comprising a hydrophobic core, and one or more lipids; at least one nucleic acid that comprises a sequence which encodes a sequence capable of expressing an antigen, optionally wherein the antigen is a SARS-CoV-2 spike protein or a functional variant thereof; and at least one sugar present in amount of (i) at least about 50% by weight of the dried composition, or (ii) present in an amount of least 50 mg. Further provided herein are compositions wherein the composition is lyophilized. Further provided herein are compositions wherein the composition is thermally stable at about 25 degrees Celsius. Further provided herein are compositions wherein the composition is thermally stable at about 45 degrees Celsius. Further provided herein are compositions wherein the composition is thermally stable at about −20 degrees Celsius. Further provided herein are compositions wherein the composition is thermally stable at about 2 degrees Celsius to about 8 degrees Celsius. Further provided herein are compositions wherein the composition is thermally stable for at least 1 week, at least 2 weeks, and/or at least 1 month. Further provided herein are compositions wherein the hydrophobic core comprises an oil. Further provided herein are compositions wherein the oil comprises at least one of α-tocopherol, lauroyl polyoxylglyceride, monoacylglycerol, propolis, squalene, mineral oil, grapeseed oil, olive oil, paraffin oil, peanut oil, soybean oil, sunflower oil, soy lecithin, triglyceride, vitamin E, a caprylic/capric triglyceride, a triglyceride ester of saturated coconut/palmkernel oil derived caprylic and capric fatty acids and plant derived glycerol, dihydroisosqualene (DHIS), famesene and squalane. Further provided herein are compositions wherein the one or more lipids is selected from the group consisting of cationic lipids, anionic lipids, neutral lipids, and any combinations thereof. Further provided herein are compositions wherein the one or more lipids comprises a cationic lipid. Further provided herein are compositions wherein the cationic lipid is selected from the group consisting of 1,2-dioleoyloxy-3-(trimethylammonium)propane (DOTAP); 3β-[N—(N′,N′-dimethylaminoethane)-carbamoyl]cholesterol (DC Cholesterol); dimethyldioctadecylammonium (DDA); 1,2-dimyristoyl-3-trimethylammoniumpropane (DMTAP), dipalmitoyl(C16:0)trimethyl ammonium propane (DPTAP); distearoyltrimethylammonium propane (DSTAP); N-[l-(2,3-dioleyloxy)propyl]-N,N,Ntrimethylammonium chloride (DOTMA); N,N-dioleoyl-N,N-dimethylammonium chloride (DODAC); 1,2-dioleoyl-sn-glycero-3-ethylphosphocholine (DOEPC); 1,2-dioleoyl-3-dimethylammonium-propane (DODAP); and 1,2-dilinoleyloxy-3-dimethylaminopropane (DLin-DMA); 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), 1,2-dioleoyl-sn-glycero-3-phosphoethanolamine (DOPE); N-decyl-N,N-dimethyldecan-1-aminium bromide (DDAB); 2,3-dioleyloxy-N-[2-(sperminecarboxamido)ethyl]-N,N-dimethyl-1-propanaminium trifluoroacetate (DOSPA); ethylphosphatidylcholine (ePC); and any combinations thereof. Further provided herein are compositions wherein the lipid carrier comprises at least one surfactant. Further provided herein are compositions wherein the at least one surfactant is selected from the group consisting of a hydrophobic surfactant, a hydrophilic surfactant, and any combinations thereof. Further provided herein are compositions wherein the hydrophobic surfactant comprises a sorbitan ester selected from the group consisting of sorbitan monolaurate, sorbitan monopalmitate, sorbitan monostearate, sorbitan tristearate, sorbitan monooleate, and sorbitan trioleate; and the hydrophilic surfactant comprises a polysorbate. Further provided herein are compositions wherein the lipid carrier has a z-average hydrodynamic diameter ranging from about 40 nm to about 150 nm, with an average polydispersity index ranging from about 0.1 to about 0.4. Further provided herein are compositions wherein the one or more nucleic acid is a DNA. Further provided herein are compositions wherein the one or more nucleic acid is a RNA. Further provided herein are compositions wherein the RNA is a self-replicating RNA. Further provided herein, are compositions wherein the hydrophobic core comprises one or more inorganic nanoparticles. Further provided herein are compositions, wherein the one or more inorganic nanoparticles is selected from the group consisting of a metal salt, metal oxide, metal hydroxide, metal phosphate, and any combinations thereof. Further provided herein are compositions wherein the one or more nucleic acid is incorporated or complexed with the lipid carrier to form a lipid carrier-nucleic acid complex. Further provided herein are compositions wherein the lipid carrier-nucleic acid complex is formed via non-covalent interactions or via reversible covalent interactions. Further provided herein are compositions wherein a molar ratio of the lipid carrier to the one or more nucleic acids, characterized by the nitrogen-to-phosphate (N:P) molar ratio, ranges from about 1:1 to about 150:1. Further provided herein are compositions wherein the at least one sugar is selected from the group consisting of sucrose, maltose, trehalose, mannitol, glucose, and any combinations thereof. Further provided herein are compositions wherein the at least one sugar is present in an amount of at least about 50, 60, 70, 80, 90, 100, 110, 120, 130, 140, 150, 160, 170, 180, 190, 200, 210, 220, 230, 240, 250, 260, 270, 280, 290, 300 or more mg. Further provided herein are compositions wherein the at least one sugar is present in an amount of 50 mg to 250 mg. Further provided herein are compositions wherein the at least one sugar is present in an amount of at least about 250 mg. Further provided herein are compositions wherein the sugar is present in amount of the composition by weight of at least about 50, 55, 60, 65, 70, 75, 80, 85, 90, 95% or more. Further provided herein are compositions wherein the sugar is present in amount of the composition by weight of 80 to 98%, optionally 94 to 96%. Further provided herein are compositions wherein the sugar is present in amount of the composition by weight of about 95%. Further provided herein are compositions wherein the at least one sugar comprises sucrose. Further provided herein are pharmaceutical compositions, comprising a dried composition disclosed herein reconstituted in a suitable diluent and a pharmaceutically acceptable carrier. Further provided herein are pharmaceutical compositions wherein the diluent is aqueous. Further provided herein are pharmaceutical compositions wherein the diluent is water. Further provided herein are kits comprising a pharmaceutical composition described herein and a delivery system for administration to a subject. Further provided are methods for generating an immune response in a subject, comprising administering a therapeutically effective amount of the pharmaceutical composition described herein. Further provided are methods of treating or preventing a disease in a subject, comprising administering a therapeutically effective amount of the pharmaceutical composition described herein. Further provided herein are methods of imaging and/or tracking delivery of one or more nucleic acids in a subject, comprising administering a therapeutically effective amount of the pharmaceutical composition described herein.

### Examples

### Example 1: Immune Response in Macrophages

Various formulations comprising lipid carrier and repRNA were prepared and analyzed in order to study the innate immune response of the lipid carrier in macrophages. Protein expression and stimulation of TNF production (e.g., TNF alpha production) in THP-1 macrophages were studied.

Initially, the THP-1 monocytes were differentiated into macrophages using phorbol 12-myristate 13-acetate (PMA). The cells were then transfected with various formulations with Nano Luciferase encoding replicon RNA. The cell culture media was then assessed for NanoLuc and TNF expression.

The formulations and their characteristics such as particle size and PDI that were used in this study are described in Table 4. The concentration of repRNA encoding NanoLuc was 909 ng/ul and maintained at −80° C. Miglyol 812 N (caprylic/capric triglyceride) was used in this study.

TABLE 4
Formulations for immune response study.

| | Particle | | | | | | | |
|---|---|---|---|---|---|---|---|---|
| | size | | Iron | | | | | |
| | [diameter, | | [mg | Aluminum | DOTAP | Squalene | Miglyol | Solanesol |
| Formulation | nm] | PDI | Fe/ml] | [mg Al/ml] | [mg/ml] | [mg/ml] | [mg/ml] | [mg/ml] |
| NP-1: Fe- | 59.3 | 0.23 | 0.19 | n/a | 27.9 | 39.4 | n/a | n/a |
| lipid carrier | | | | | | | | |
| NP-2: High | 57.5 | 0.24 | 0.85 | n/a | 29.1 | 40.5 | n/a | n/a |
| Fe-lipid | | | | | | | | |
| carrier | | | | | | | | |
| NP-3: Fe- | 48.7 | 0.2 | 0.18 | n/a | 28.3 | n/a | not | n/a |
| lipid carrier | | | | | | | measured | |
| miglyol | | | | | | | | |
| NP-4: High | 62.6 | 0.28 | 0.94 | n/a | 27.7 | n/a | not | n/a |
| Fe-lipid | | | | | | | measured | |
| carrier | | | | | | | | |
| miglyol | | | | | | | | |
| NP-5: Alum- | 64.5 | 0.25 | n/a | 0.88 | 27.4 | 41.2 | n/a | n/a |
| lipid carrier | | | | | | | | |
| NP-6: Fe- | 86.1 | 0.26 | 0.16 | n/a | 26.2 | n/a | n/a | 36 |
| lipid carrier | | | | | | | | |
| solanesol | | | | | | | | |
| (SLN) | | | | | | | | |
| NP-7: NLC | 50 | 0.26 | n/a | n/a | 26.7 | 34.1 | n/a | n/a |
| NP-8: CNE | 105.4 | 0.06 | n/a | n/a | 4.4 | 47.4 | n/a | n/a |
| NP-30: lipid | 54.2 | 0.22 | n/a | n/a | 19.3 | 32.6 | n/a | n/a |
| carrier (w/o | | | | | | | | |
| IO) | | | | | | | | |

Fe-lipid carrier formulation—NP-1 (prepared at 100 ml scale): Fe-lipid carrier formulation comprises 37.5 mg/ml squalene (SEPPIC), 37 mg/ml SPAN 60 (Millipore Sigma), 37 mg/ml TWEEN 80 (Fisher Chemical), 30 mg/ml DOTAP chloride (LIPOID), 0.2 mgFe/ml 12 nm oleic acid-coated iron oxide nanoparticles (ImagionBio) and 10 mM sodium citrate dihydrate (Fisher Chemical). 1 ml of 20 mgFe/ml 12 nm diameter oleic acid-coated iron oxide nanoparticles in chloroform (ImagionBio, lot #95-127) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams SPAN 60, and 3 grams DOTAP were added to produce the oil phase. The oil phase was sonicated for 45 minutes in a 65 degree C. water bath. Separately, the aqueous phase was prepared by dissolving 19.5 grams TWEEN 80 in 500 ml of 10 mM sodium citrate buffer prepared in nuclease free water. 92 ml of the aqueous phase was transferred to a separate glass bottle and heated to 65 degrees C. for 30 minutes. The oil phase was mixed with the 92 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.25 polydispersity index (PDI). The microfluidized formulation was terminally filtered with a 200 nm pore-size polyethersulfone (PES) filter and stored at 2 to 8 degrees C. Iron concentration was determined by ICP-OES. DOTAP and Squalene concentration were measured by RP-HPLC.

High Fe-lipid carrier formulation NP-2 (prepared at 100 ml scale): High Fe-lipid carrier formulation comprises 37.5 mg/ml squalene (SEPPIC), 37 mg/ml SPAN 60 (Millipore Sigma), 37 mg/ml TWEEN 80 (Fisher Chemical), 30 mg/ml DOTAP chloride (LIPOID), 1 mgFe/ml 15 nm oleic acid-coated iron oxide nanoparticles (ImagionBio) and 10 mM sodium citrate dihydrate (Fisher Chemical). 5 ml of 20 mgFe/ml 15 nm diameter oleic acid-coated iron oxide nanoparticles in chloroform (ImagionBio, Lot #95-133) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams SPAN 60, and 3 grams DOTAP were added to produce the oil phase. The oil phase was sonicated for 45 minutes in a 65 degree C. water bath. Separately, the aqueous phase was prepared by dissolving 19.5 grams TWEEN 80 in 500 ml of 10 mM sodium citrate buffer prepared in nuclease free water. 92 ml of the aqueous phase was transferred to a separate glass bottle and heated to 65 degrees C. for 30 minutes. The oil phase was mixed with the 92 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.3 polydispersity index (PDI). The microfluidized formulation was terminally filtered with a 200 nm pore-size polyethersulfone (PES) filter and stored at 2 to 8 degrees C. Iron concentration was determined by ICP-OES. DOTAP and Squalene concentration were measured by RP-HPLC.

### Example 2: Fe-Lipid Carrier Miglyol Formulation NP-3 (Prepared at 100 ml Scale)

Fe-lipid carrier Miglyol formulation comprises 37.5 mg/ml Miglyol 812 N (IOI Oleo GmbH), 37 mg/ml SPAN 60 (Millipore Sigma), 37 mg/ml TWEEN 80 (Fisher Chemical), 30 mg/ml DOTAP chloride (LIPOID), 0.2 mgFe/ml 15 nm oleic acid-coated iron oxide nanoparticles (ImagionBio) and 10 mM sodium citrate dihydrate (Fisher Chemical). 1 ml of 20 mgFe/ml 15 nm diameter oleic acid-coated iron oxide nanoparticles in chloroform (ImagionBio, Lot #95-127) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams SPAN 60, and 3 grams DOTAP were added to produce the oil phase. The oil phase was sonicated for 45 minutes in a 65 degree C. water bath. Separately, the aqueous phase was prepared by dissolving 19.5 grams TWEEN 80 in 500 ml of 10 mM sodium citrate buffer prepared in nuclease free water. 92 ml of the aqueous phase was transferred to a separate glass bottle and heated to 65 degree C. for 30 minutes. The oil phase was mixed with the 92 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.3 polydispersity index (PDI). The microfluidized formulation was terminally filtered with a 200 nm pore-size polyethersulfone (PES) filter and stored at 2 to 8 degrees C. Iron concentration was determined by ICP-OES. DOTAP concentration was measured by RP-HPLC.

### Example 3: High Fe-Lipid Carrier Miglyol Formulation NP-4 (Prepared at 100 ml Scale)

High Fe-lipid carrier Miglyol formulation comprises 37.5 mg/ml Miglyol 812 N (IOI Oleo GmbH), 37 mg/ml SPAN 60 (Millipore Sigma), 37 mg/ml TWEEN 80 (Fisher Chemical), 30 mg/ml DOTAP chloride (LIPOID), 1 mg/ml 15 nm oleic acid-coated iron oxide nanoparticles (ImagionBio) and 10 mM sodium citrate dihydrate (Fisher Chemical). 5 ml of 20 mgFe/ml 15 nm diameter oleic acid-coated iron oxide nanoparticles in chloroform (ImagionBio, Lot #95-127) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams SPAN 60, and 3 grams DOTAP were added to produce the oil phase. The oil phase was sonicated for 45 minutes in a 65 degree C. water bath. Separately, the aqueous phase was prepared by dissolving 19.5 grams TWEEN 80 in 500 ml of 10 mM sodium citrate buffer prepared in nuclease free water. 92 ml of the aqueous phase was transferred to a separate glass bottle and heated to 65 degrees C. for 30 minutes. The oil phase was mixed with the 92 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.3 polydispersity index (PDI). The microfluidized formulation was terminally filtered with a 200 nm pore-size polyethersulfone (PES) filter and stored at 2 to 8 degrees C. Iron concentration was determined by ICP-OES. DOTAP concentration was measured by RP-HPLC.

### Example 4: Alum-Lipid Carrier Formulation NP-5 (Prepared at 100 ml Scale)

Alum-lipid carrier formulation comprises 37.5 mg/ml squalene (SEPPIC), 37 mg/ml SPAN 60 (Millipore Sigma), 37 mg/ml TWEEN 80 (Fisher Chemical), 30 mg/ml DOTAP chloride (LIPOID), 1 mg Al/ml TOPO-coated Alhydrogel® (aluminum oxyhydroxide) particles (Croda) and 10 mM sodium citrate. 10 ml of Alhydrogel was washed three times in methanol by centrifuging at 1000 rpm for 20 minutes. After the third wash, Alhydrogel was dispersed in 10 ml methanol and to this dispersion was added 1 ml of 250 mg/ml trioctylphosphine oxide (TOPO) and incubated overnight in a 37° C. orbital shaker. Excess TOPO was removed by additional methanol washes and then dispersed in 11 ml methanol. Methanol was allowed to evaporate overnight in the fume hood leaving behind a dry layer of TOPO-Alhydrogel. To this dry TOPO-Alhydrogel layer, 3.75 grams squalene, 3.7 grams SPAN 60, and 3 grams DOTAP were added to produce the oil phase. The oil phase was sonicated for 45 minutes in a 65 degree C. water bath. Separately, the aqueous phase was prepared by dissolving 19.5 grams TWEEN 80 in 500 ml of 10 mM sodium citrate buffer prepared in nuclease free water. 92 ml of the aqueous phase was transferred to a separate glass bottle and heated to 65 degrees C. for 30 minutes. The oil phase was mixed with the 92 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.3 polydispersity index (PDI). The microfluidized formulation was terminally filtered with a 200 nm pore-size polyethersulfone (PES) filter and stored at 2 to 8 degrees C. Aluminum concentration was determined by ICP-OES. DOTAP and Squalene concentration were measured by RP-HPLC.

### Example 5: Fe-Lipid Carrier Solanesol Formulation NP-6 (Prepared at 100 ml Scale)

Fe-lipid carrier solanesol formulation (NP-6) comprises 37.5 mg/ml Solanesol (Cayman chemicals), 37 mg/ml SPAN 60 (Millipore Sigma), 37 mg/ml TWEEN 80 (Fisher Chemical), 30 mg/ml DOTAP chloride (LIPOID), 0.2 mgFe/ml oleic acid-coated iron oxide nanoparticles (ImagionBio) and 10 mM sodium citrate. 1 ml of 20 mgFe/ml 15 nm diameter oleic acid-coated iron oxide nanoparticles in chloroform (ImagionBio, Lot #95-133) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams solanesol, 3.7 grams SPAN 60, and 3 grams DOTAP were added to produce the oil phase. The oil phase was sonicated for 45 minutes in a 65 degree C. water bath. Separately, the aqueous phase was prepared by dissolving 19.5 grams TWEEN 80 in 500 ml of 10 mM sodium citrate buffer prepared in nuclease free water. 92 ml of the aqueous phase was transferred to a separate glass bottle and heated to 65 degrees C. for 30 minutes. The oil phase was mixed with the 92 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber. The microfluidized formulation was terminally filtered with a 200 nm pore-size polyethersulfone (PES) filter and stored at 2 to 8 degrees C. Iron concentration was determined by ICP-OES. DOTAP and solanesol concentration were measured by RP-HPLC.

### Example 6: NP-7 Formulation (Prepared at 100 ml Scale)

NP-7 formulation comprises 37.5 mg/ml squalene (SEPPIC), 37 mg/ml SPAN 60 (Millipore Sigma), 37 mg/ml TWEEN 80 (Fisher Chemical), 30 mg/ml DOTAP chloride (LIPOID), 2.4 mg/ml Dynasan 114 (IOI Oleo GmbH) and 10 mM sodium citrate. To a 200 ml beaker 3.75 grams squalene, 3.7 grams SPAN 60, 0.24 grams Dynasan 114 and 3 grams DOTAP were added to produce the oil phase. The oil phase was sonicated for 45 minutes in a 65 degree C. water bath. Separately, the aqueous phase was prepared by dissolving 19.5 grams TWEEN 80 in 500 ml of 10 mM sodium citrate buffer prepared in nuclease free water. 92 ml of the aqueous phase was transferred to a separate glass bottle and heated to 65 degrees C. for 30 minutes. The oil phase was mixed with the 92 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.3 polydispersity index (PDI). The microfluidized formulation was terminally filtered with a 200 nm pore-size polyethersulfone (PES) filter and stored at 2 to 8 degrees C. DOTAP and Squalene concentration were measured by RP-HPLC.

### Example 7: NP-8 Formulation (Prepared at 100 ml Scale)

NP-8 formulation comprises 43 mg/ml squalene (SEPPIC), 5 mg/ml SPAN 85 (Millipore Sigma), 5 mg/ml TWEEN 80 (Fisher Chemical), 4 mg/ml DOTAP chloride (LIPOID) and 10 mM sodium citrate. To a 200 ml beaker 4.3 grams squalene, 0.5 grams SPAN 85, and 0.4 grams DOTAP were added to produce the oil phase. The oil phase was sonicated for 45 minutes in a 65 degree C. water bath. Separately, the aqueous phase was prepared by dissolving 2.6 grams TWEEN 80 in 500 ml of 10 mM sodium citrate buffer prepared in nuclease free water. 95 ml of the aqueous phase was transferred to a separate glass bottle and heated to 65 degrees C. for 30 minutes. The oil phase was mixed with the 95 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 100±10 nm with a 0.05-0.1 polydispersity index (PDI). The microfluidized formulation was terminally filtered with a 200 nm pore-size polyethersulfone (PES) filter and stored at 2 to 8 degrees C. DOTAP and Squalene concentration were measured by RP-HPLC.

The treatment groups were prepared. Eight of those groups were NanoLuc repRNA groups, with 600 ng dose per well was prepared using the Fe-lipid carrier, High Fe-lipid carrier, Fe-lipid carrier miglyol, High Fe-lipid carrier miglyol, Alum-lipid carrier, Fe-lipid carrier solanesol (SLN), NLC, and CNE formulations. The untreated group did not have NanoLuc.

The various formulations were prepared by diluting NanoLuc repRNA to 8 ng/μL in 2.2 mL of RNAse-free water. The lipid carrier formulations and RNA master mix was complexed by adding 250 μL of each diluted formulation with 250 μL of diluted RNA, and mixed by pipetting up and down.

Cell transfections were carried out by seeding 7×10⁵ THP-1s per well in a 24 well plate. 80 μM of PMA per well was added and incubated at 37 degrees C. The next day, the PMA-containing media was removed and replaced with cRPMI for an hour before transfection. The samples were then serially diluted in Opti-MEM to make a 10-point 1.5-fold dilution series starting at 0.45 ng/μL. The culture media was then removed from the plates by pipetting. 450 μL of Opti-MEM and 150 μL of the complexed formulation was added to the plate in duplicate. The empty wells were given 450 μL of Opti-MEM only. After four hours, the samples were removed from the plate by pipetting and replaced with 500 μL of growth media. The plate was then incubated overnight at 37° C. The growth media was harvested the next day and stored at −80° C. Downstream assays were conducted.

The luciferase assay was performed by first diluting the Nano-Glo luciferase assay reagent 1:50 in buffer. 25 μL of supernatant was removed and mixed with 25 μL of Nano-Glo reagent in a 96-well plate. This was incubated at room temperature for 3 minutes. The luminescence was read.

ELISA assay was performed to evaluate the TNF-alpha protein level in the media using the Human TNF-alpha DuoSet ELISA by R&D Systems according to the manufacturer's protocol. The 96-well microplate was coated with anti-TNF capture antibody. The plate was blocked and then media samples were added directly without dilution. After addition of the biotinylated detection antibody, SA-HRP, and substrate, the absorbance was read at 450 nm on a Spectramax i3 plate reader. No other major differences between formulations were observed.

All studies in this example were done in duplicates. Results from the duplicates are presented as first experiment and second experiment respectively.

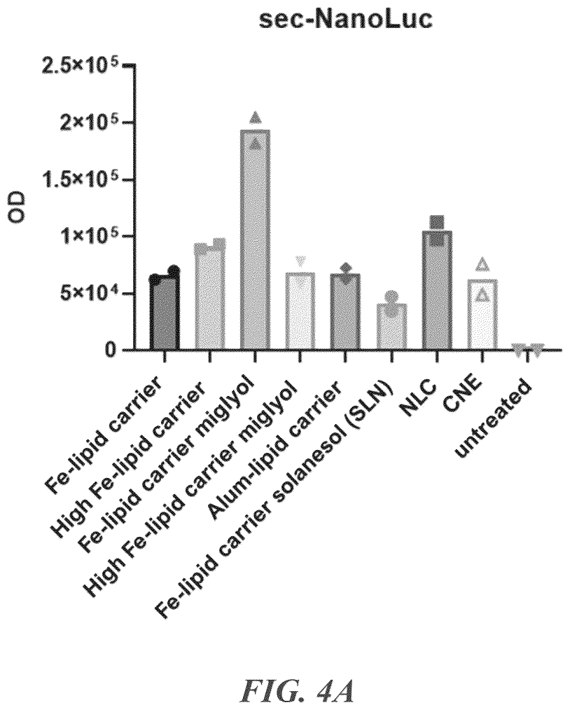

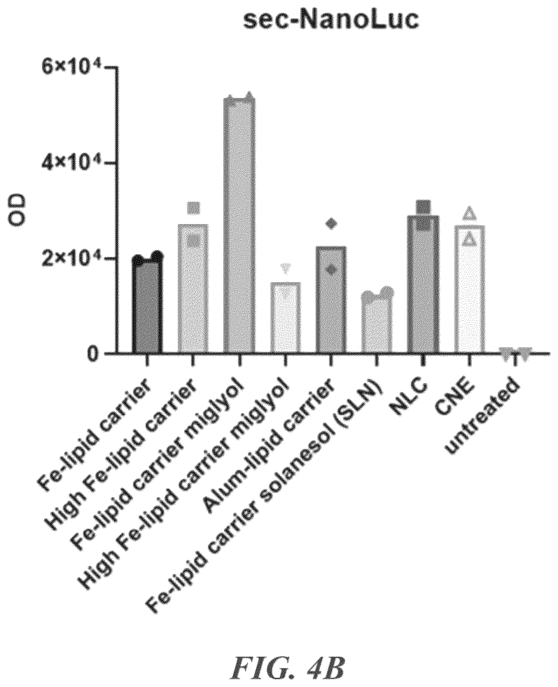

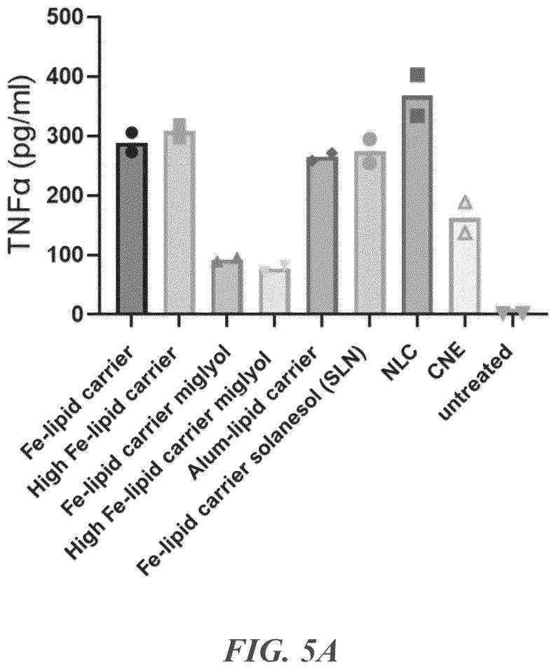

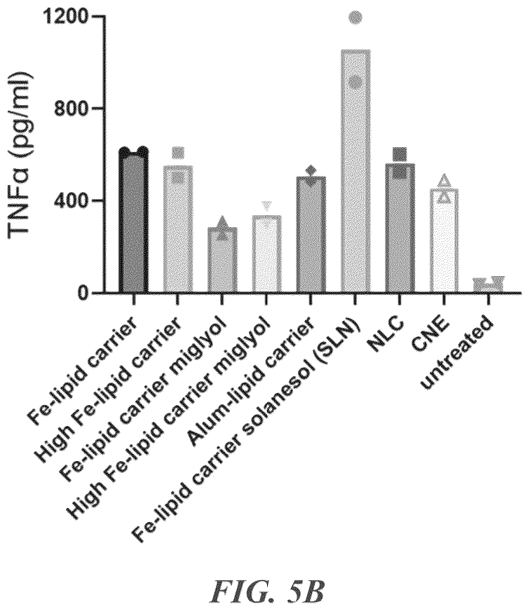

The formulation comprising a lipid carrier and Miglyol induced higher protein production off the replicon, as shown in the first experiment in FIG. 4A and in the second experiment in FIG. 4B. A reduced innate immune response was detected, as measured by TNF-alpha secretion and is shown in the first experiment in FIG. 5A and in the second experiment in FIG. 5B.

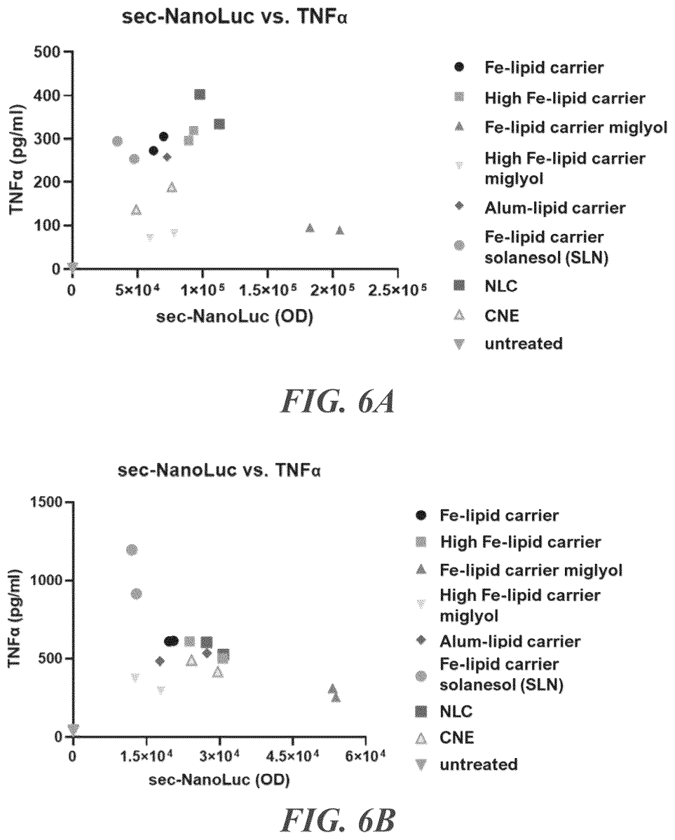

The correlation between enhanced protein production and low TNF-alpha stimulation was observed with Miglyol lipid carrier formulation, as shown in the first experiment in FIG. 6A and in the second experiment in FIG. 6B. The solanesol induced slightly lower protein production, but potentially higher TNF production, shown in the first experiment in FIG. 6A and in the second experiment in FIG. 6B.

### Example 8: Techniques and Materials for Production of Lipid Carriers

The following materials were used in the manufacturing of lipid-inorganic nanoparticles (i.e., lipid carriers). The compositions, kits and methods described herein are not limited to the techniques or materials described herein.

Iron oxide nanoparticles at 25 mgFe/ml in chloroform and of various average diameters (5, 10, 15, 20, 25 and 30 nm) were purchased from Ocean Nanotech (San Diego, CA). Squalene and Span® 60 (sorbitan monostearate) were purchased from Millipore Sigma. Tween® 80 (polyethylene glycol sorbitan monooleate) and sodium citrate dihydrate were purchased from Fisher Chemical. The chloride salt of the cationic lipid 1,2-dioleoyl-3-trimethylammoniumpropane (DOTAP chloride) was purchased from Corden Pharma. Ultrapure water (18.2 mega ohm centimeter (MOhm-cm) resistivity) was obtained from a Milli-Q water purification system (Millipore Sigma).

The lipid carrier comprises squalene, sorbitan monostearate (e.g., SPAN-60), polysorbate 80 (e.g., TWEEN-80), DOTAP chloride, iron oxide nanoparticles and sodium citrate dihydrate.

In general, to iron oxide nanoparticles with a number-weighted average diameter of 5 nm, chloroform was added. Chloroform was allowed to evaporate in a fume hood leaving behind a dry coating of iron oxide nanoparticles. To the iron oxide nanoparticles, Span® 60, squalene, and DOTAP chloride were added to prepare the “oil” phase.

The oil phase was sonicated 30 minutes in a water bath pre-heated to 60° C. Separately, in a 1 liter glass bottle, the “aqueous” phase was prepared by adding Tween® 80 to sodium citrate dihydrate solution prepared with Milli-Q water.

The aqueous phase was stirred for 30 minutes to allow complete dissolution of Tween® 80. After complete dissolution of Tween® 80, the aqueous phase was transferred to a beaker and incubated in a water bath pre-heated to 60° C. To the heated oil phase, the pre-heated aqueous phase was added.

The mixture was immediately emulsified using a VWR® 200 homogenizer (VWR International) until a homogenous colloid with a milk-like appearance was produced. The colloid was subsequently processed by passaging the fluid through a Y-type interaction chamber of a LM10 microfluidizer at 20,000 psi.

The fluid was passaged until the z-average hydrodynamic diameter, measured by dynamic light scattering (Malvern Zetasizer Nano S), was 59 nm with a 0.2 polydispersity index.

The microfluidized lipid carrier sample was terminally filtered with a 200 nm pore-size polyethersulfone (PES) syringe filter.

### Example 9: Exemplary Techniques and Materials for Producing Lipid Carriers

The following materials were used in the manufacturing of lipid-inorganic nanoparticles (i.e., lipid carriers). The compositions, kits and methods described herein are not limited to the techniques or materials describe herein.

Iron oxide nanoparticles at 25 mgFe/ml in chloroform and of various average diameters (5, 10, 15, 20, 25 and 30 nm) were purchased from Ocean Nanotech (San Diego, CA). Squalene and Span® 60 (sorbitan monostearate) were purchased from Millipore Sigma. Tween® 80 (polyethylene glycol sorbitan monooleate) and sodium citrate dihydrate were purchased from Fisher Chemical. The chloride salt of the cationic lipid 1,2-dioleoyl-3-trimethylammoniumpropane (DOTAP chloride) was purchased from Corden Pharma. Ultrapure water (18.2 MOhm-cm resistivity) was obtained from a Milli-Q water purification system (Millipore Sigma).

Lipid carriers were prepared which comprised 37.5 mg/ml squalene, 37 mg/ml Span® 60, 37 mg/ml Tween® 80, 30 mg/ml DOTAP chloride, 0.1 mg/ml 10 nm iron oxide nanoparticles and 10 mM sodium citrate dihydrate.

The lipid carriers were manufactured using the following procedures. In a 200 ml beaker, 0.4 ml of iron oxide nanoparticles at 25 mgFe/ml in chloroform, with a number-weighted average diameter of 10 nm, were added.

Chloroform was allowed to evaporate in a fume hood leaving behind a dry coating of iron oxide nanoparticles. To the iron oxide nanoparticles, 3.7 grams of Span® 60, 3.75 grams of squalene, and 3 grams of DOTAP chloride were added to prepare the “oil” phase.

The oil phase was sonicated 30 minutes in a water bath pre-heated to 60° C. Separately, in a 1 liter glass bottle, the “aqueous” phase was prepared by adding 39 grams of Tween® 80 to 1,000 ml 10 mM sodium citrate dihydrate solution prepared with Milli-Q water.

The aqueous phase was stirred for 30 minutes to allow complete dissolution of Tween® 80. After complete dissolution of Tween® 80, 96 ml of the aqueous phase was transferred to a 200 ml beaker and incubated in a water bath pre-heated to 60° C. To the heated oil phase, 96 ml of the pre-heated aqueous phase was added. The mixture was immediately emulsified using a VWR® 200 homogenizer (VWR International) until a homogenous colloid with a milk-like appearance was produced. The colloid was subsequently processed by passaging the fluid through a Y-type interaction chamber of a LM10 microfluidizer at 20,000 psi. The fluid was passaged until the z-average hydrodynamic diameter, measured by dynamic light scattering (Malvern Zetasizer Nano S), was 54 nm with a 0.2 polydispersity index. The microfluidized lipid carrier sample was terminally filtered with a 200 nm pore-size polyethersulfone (PES) syringe filter.

### Example 10: Exemplary Techniques and Materials for Producing Nanoparticles Described Herein

Lipid carriers were prepared which comprised 37.5 mg/ml squalene, 37 mg/ml Span® 60, 37 mg/ml Tween® 80, 30 mg/ml DOTAP chloride, 0.2 mg/ml 15 nm iron oxide nanoparticles, and 10 M sodium citrate dihydrate.

The lipid carriers of Example 4 were manufactured using the following procedures.

In a 200 ml beaker, 0.8 ml of iron oxide nanoparticles at 25 mgFe/ml in chloroform, with a number-weighted average diameter of 15 nm, was added. Chloroform was allowed to evaporate in a fume hood leaving behind a dry coating of iron oxide nanoparticles. To the iron oxide nanoparticles, 3.7 grams of Span® 60, 3.75 grams of squalene, and 3 grams of DOTAP chloride were added to prepare the “oil” phase.

The oil phase was sonicated 30 minutes in a water bath pre-heated to 60° C. Separately, in a 1 liter glass bottle, the “aqueous” phase was prepared by adding 39 grams of Tween® 80 to 1,000 ml of 10 mM sodium citrate dihydrate solution prepared with Milli-Q water.

The aqueous phase was stirred for 30 minutes to allow complete dissolution of Tween® 80.

After complete dissolution of Tween® 80, 96 ml of the aqueous phase was transferred to a 200 ml beaker and incubated in a water bath pre-heated to 60° C. To the heated oil phase, 96 ml of the pre-heated aqueous phase was added.

The mixture was immediately emulsified using a VWR® 200 homogenizer (VWR International) until a homogenous colloid with a milk-like appearance was produced. The colloid was subsequently processed by passaging the fluid through a Y-type interaction chamber of a LM10 microfluidizer at 20,000 psi.

The fluid was passaged until the z-average hydrodynamic diameter, measured by dynamic light scattering (Malvern Zetasizer Nano S), was 52 nm with a 0.2 polydispersity index.

The microfluidized lipid carrier sample was terminally filtered with a 200 nm pore-size polyethersulfone (PES) syringe filter.

### Example 11: Lipid Carrier without Inorganic Core Formulation (Prepared at 100 mL Scale)

Described herein is a lipid carrier formulation without inorganic core particles comprising: 37.5 mg/ml squalene (SEPPIC), 37 mg/ml Span® 60 (Millipore Sigma), 37 mg/ml Tween® 80 (Fisher Chemical), 30 mg/ml DOTAP chloride (LIPOID) and 10 mM sodium citrate.

This composition was prepared as follows. To a 200 ml beaker 3.75 grams squalene, 3.7 grams span 60, and 3.0 grams DOTAP were added to produce the oil phase. The oil phase was sonicated for 45 minutes in a 65 degree C. water bath. Separately, the aqueous phase was prepared by dissolving 19.5 grams Tween 80 in 500 ml of 10 mM sodium citrate buffer prepared in nuclease free water. 96 ml of the aqueous phase was transferred to a separate glass bottle and heated to 65 degrees C. for 30 minutes. The oil phase was mixed with the 96 ml of aqueous phase by adding the warm oil phase to the warm aqueous phase.

The mixture was then emulsified using a VWR 200® homogenizer (VWR International) and the resulting crude emulsion was processed by passing through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1 to 0.3 polydispersity index (PDI). The microfluidized lipid carrier composition (without inorganic core) formulation was then terminally filtered with a 200 nm pore-size polyethersulfone (PES) filter and stored at 2 to 8 degrees (° C.). DOTAP and squalene concentration were then measured by RP-HPLC.

### Example 12: Exemplary Techniques and Materials for Producing Nanoparticles Described Herein

In a further murine study, C57BL/6 mice were inoculated as described in Table 6 below, after which secreted embryonic alkaline phosphatase (SEAP) levels were measured in serum. A summary of the materials used in the study is provided in Table 5.

TABLE 5
Materials.

| Description | Concentration | | Temperature/Location |
|---|---|---|---|
| DNA encoding SEAP | 5 | mg/mL | −20° C. |
| (DNA sequence as set forth in | | | |
| SEQ ID NO: 39) | | | |
| repRNA encoding SEAP | 2217 | μg/mL | −80° C. |
| Lipid carrier formulation | 30 | mg | 4° C. |

DOTAP/mL

TABLE 6
Treatment Groups.

| | | | | RNA | DNA | | Injection |
|---|---|---|---|---|---|---|---|
| | | | DNA/RNA- | dose | dose | | Volume |
| Group | n | Formulation | SEAP | [μg] | [μg] | N:P | [μL] |
| 1 | 5 | Naked | DNA-SEAP | | 20 | n/a | 50 |
| 2 | 5 | Lipid carrier | DNA-SEAP | | 10 | 15 | 50 |
| 3 | 5 | Lipid carrier | DNA-SEAP | | 10 | 7.5 | |
| 4 | 5 | Lipid carrier | DNA-SEAP | | 20 | 15 | |
| 5 | 5 | Lipid carrier | DNA-SEAP | | 20 | 7.5 | |
| 6 | 5 | Lipid carrier | RNA-SEAP | 1 | | 15 | 50 |
| 7 | 5 | Miglyol + lipid | RNA-SEAP | 1 | | 15 | 50 |
| | | carrier | | | | | |

Seven different formulations were prepared and administered intramuscularly across the seven treatment groups (Groups 1-7). DNA-SEAP or RNA-SEAP was diluted according to the volumes set forth in Table 7 to prepare the formulations for Groups 1-7.

TABLE 7
Formulation Preparation; Dilution of RNA/DNA

| | | DNA or | 40% | | |
|---|---|---|---|---|---|
| | DNA-or | RNA | sucrose | water | Total |
| Group | RNA-SEAP | [μL] | [μL] | [μL] | [μL] |
| 1 | DNA-SEAP | 40.0 | 125.0 | 85.0 | 250.0 |
| 2 | DNA-SEAP | 20.0 | 0.0 | 230.0 | 250.0 |
| 3 | DNA-SEAP | 20.0 | 0.0 | 230.0 | 250.0 |
| 4 | DNA-SEAP | 40.0 | 125.0 | 85.0 | 250.0 |
| 5 | DNA-SEAP | 40.0 | 0.0 | 210.0 | 250.0 |
| 6 | RNA-SEAP | 4.5 | 0.0 | 245.5 | 250.0 |
| 7 | RNA-SEAP | 4.5 | 0.0 | 245.5 | 250.0 |

The concentrations of diluted DNA or RNA prior to complexing with the lipid carrier was as follows (measured by NanoDrop spec): Groups 1, 4 and 5 contains about 820 μg/ml DNA; Groups 2 and 3 contained about 480 μg/ml DNA; and Groups 6 and 7 contained about 43 μg/ml RNA.

Formulations for Groups 1-7 were diluted with 100 mM citrate as set forth in Table 8 below.

TABLE 8
Dilution of lipid carrier formulations.

| | | Lipid | 40% | 100 mM | | |
|---|---|---|---|---|---|---|
| | | Carrier | sucrose | citrate | Water | Total |
| Group | Formulation | [μl] | [μl] | [μl] | [μl] | [μl] |
| 1 | Naked | 0 | 0 | 30 | 270 | 300 |
| 2 | Lipid carrier | 120 | 150 | 30 | 0 | 300 |
| 3 | Lipid carrier | 60 | 150 | 30 | 60 | 300 |
| 4 | Lipid carrier | 240 | 0 | 30 | 30 | 300 |
| 5 | Lipid carrier | 120 | 150 | 30 | 0 | 300 |
| 6 | Lipid carrier | 12 | 150 | 30 | 108 | 300 |
| 7 | Miglyol + | 12 | 150 | 30 | 108 | 300 |
| | lipid carrier | | | | | |

The above formulations were complexed by adding 250 μl diluted lipid carrier to 250 μl diluted DNA or RNA. The resulting complexed formulations were incubated on ice for at least 30 minutes. Table 9 sets forth the experiment schedule for this study.

TABLE 9
Experiment schedule.

| Day | Procedure | Notes on Mice |
|---|---|---|
| 0 | All inoculations | None |
| 4 | Bleed | Group 4 had ruffled fur, one mouse |
| | | emaciated (died during collection). |
| | | Hydropaque placed in cage. |
| 6 | Bleed | None |
| 8 | Bleed | None |
| 11 | Bleed | None |
| 14 | Bleed | None |

Mice were bled at regular intervals and serum was prepared immediately and stored at −80 degrees C. until analyses for SEAP activity.

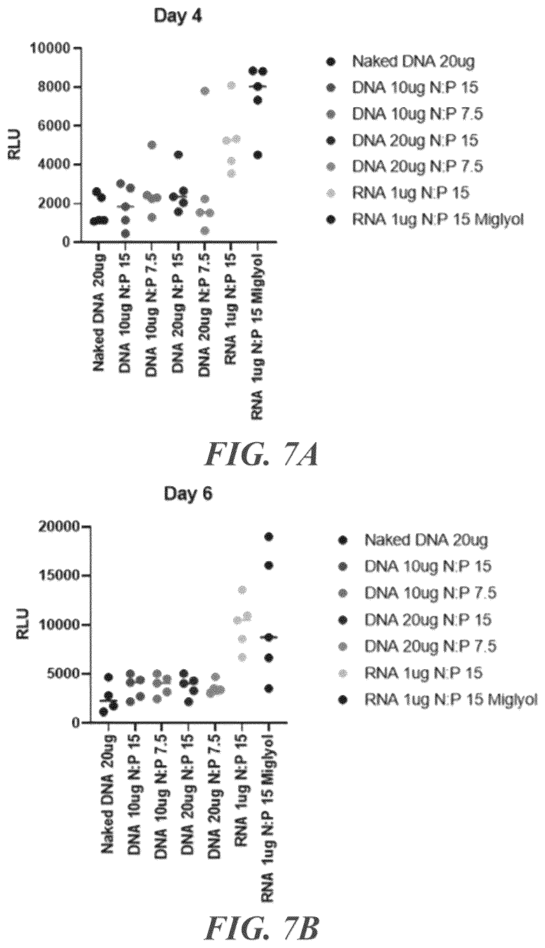

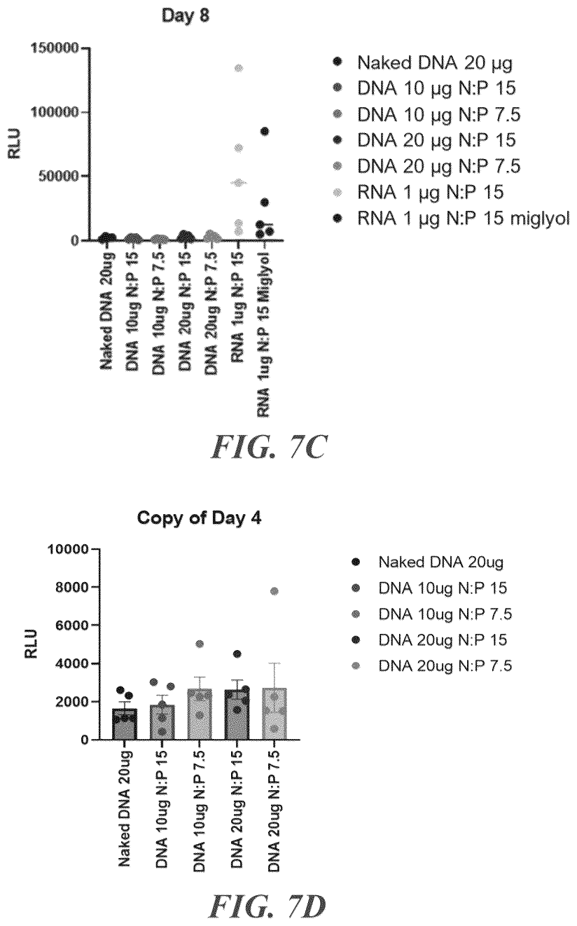

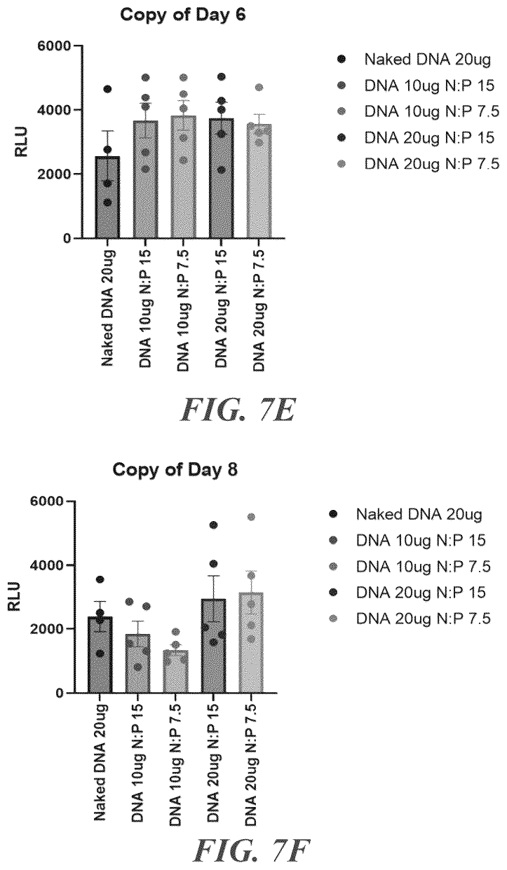

To evaluate SEAP levels in serum, all serum samples were thawed at the same time and SEAP detection was conducted. FIGS. 7A-7F illustrate the SEAP levels in BALB/c mice injected intramuscularly with varying iterations of lipid carrier-formulated DNA SEAP. Mice were bled at regular intervals, serum prepared and stored until analysis by SEAP assay. Data are displayed as mean and SE (n=5 per group).

As can be seen from FIGS. 7A-7F, lipid carrier formulations aid target protein production over delivery of DNA alone, particularly after day 6 following injection. Additionally, the data shows that inclusion of Miglyol enhances protein production from an RNA replicon over lipid carrier formulations lacking Miglyol.

### Example 13: Additional Nanoparticle Formulations

Additional nanoparticle formulations are produced according to the following tables (Table 10 and Table 11). The mRNA comprises a sequence encoding the SARS-CoV-2 omicron variant spike protein with a VEEV replicon mRNA backbone (SEQ ID NO: 8).

TABLE 10
mRNA Vaccine Formulation.

| Dosage form: | Solution for Injection for IM route of administration | | |
|---|---|---|---|
| Com- | Each 0.5 ml | | Concentration |
| position: | Vial Contains: | Quantity | (mg/ml) |
| | mRNA | 25 mcg | 0.05 |
| | DOTAP | 0.75 mg | 1.5 |
| | Iron Oxide Nanoparticles | 0.005 mg | 0.01 |
| | Squalene | 0.94 mg | 1.88 |
| | Sorbitan Monostearate | 0.93 mg | 1.86 |
| | Polysorbate 80 | 0.93 mg | 1.86 |
| | Sucrose IP | 50 mg | 100 |
| | Citric Acid Monohydrate | 1.05 mg | 2.1 |

| | |
|---|---|
| Water for Injection | q.s. to 0.5 ml |

TABLE 11
Lyophilized mRNA Vaccine Formulation.

| Dosage form: | Lyophilized powder | | | |
|---|---|---|---|---|
| | Each 5 | | Con- | Approximate |
| Com- | dose vial | | centration | dry |
| position: | contains: | Quantity | (mg/ml) | weight % |
| | mRNA | 50 mcg | 0.02 | 0.02 |
| | DOTAP | 1.5 mg | 0.6 | 0.57 |
| | Squalene | 1.88 mg | 0.752 | 0.72 |
| | Sorbitan | 1.86 mg | 0.744 | 0.71 |
| | Monostearate | | | |
| | Polysorbate 80 | 1.86 mg | 0.744 | 0.71 |
| | Sucrose IP | 250 mg | 100 | 95.3 |
| | Citric Acid | 5.25 mg | 2.1 | 2 |
| | Monohydrate | | | |
| | Water for | 2.5 ml | | |
| | Injection (for | | | |
| | reconstitution) | | | |

### Example 14: Self-Replicating mRNA Construct

A plasmid encoding a T7 promoter followed by the 5′ and 3′ UTRs and nonstructural genes of Venezuelan equine encephalitis virus (VEEV) strain TC-83 was generated using standard DNA synthesis and cloning methods. The VEEV replicon mRNA backbone is set forth in SEQ ID NO: 20.

### Example 15: An Alphavirus-Derived Replicon RNA Vaccine for SARS-CoV-2 Neutralizing Antibody and T Cell Responses in Nonhuman Primates

The immunogenicity and safety of NP-1 to NP-31 in combination with repRNA-CoV2 S protein vaccines are assessed in pigtail macaques. To protect the RNA replicons from degradation, NP-1 which consists of inorganic superparamagnetic iron oxide (SPIO) nanoparticles within a hydrophobic squalene core is used to enhance formulation stability. Replicon RNA (SEQ ID NOS: 1 to 8) are complexed with NP formulations. Pigtail macaques are used to test the response to the vaccines. Blood is collected at baseline (week −2 or −1), and at days 10, 14, 28, and 42 post-prime vaccination. Blood is also collected 10 days post-boost (38 days post-prime) in 50 μg vaccinated animals. Serum and plasma are collected and PBMCs are isolated from whole blood. Animals are sedated with an intramuscular injection (10 mg/kg) of ketamine (Ketaset®; Henry Schein) prior to blood collection or vaccination. The 50 μg vaccine is delivered intramuscularly into the quadriceps muscle with one 250 μl injection on weeks 0 and 4. To maintain consistency in the vaccine formulation and concentration, the 250 μg vaccine is delivered intramuscularly by inoculating 250 μl injections into 5 intramuscular injections sites, 2 in the right quadriceps, 1 in the left quadricep, and 1 each in the left and right deltoids on week 0. All injection sites are monitored post-injection for any signs of local reactogenicity. Serum chemistries and complete blood counts are assessed along with antigen-specific antibody responses.

Blood is collected from venipuncture of anesthetized macaques. Antigen-specific IgG, IgG1, IgG2a, and IgG2c responses are detected by enzyme linked immuno-sorbent assay (ELISA) using recombinant SARS-CoV-2 S as the capture antigen. SARS-CoV-2 neutralization assays are also performed to determine whether vaccine compositions can induce antibody responses to SARS-CoV-2 infection. Immune responses in macaques are monitored by IFN-γ ELISpot assay and intracellular cytokine staining. Multiparameter flow cytometry is used to determine T-cell immune responses using peptide stimulated PBMCs. S-specific CD4+ or CD8+T cells are screened before and after immunization.

### Example 16: Evaluation of Lyophilized COVID Vaccines in Mice

The following was performed to assay activity of lyophilized NP-1 with replicon RNA encoded SARS-CoV-2 spike antigen sequence, physicochemical properties of reconstituted vaccines, potency, and immunogenicity. Briefly, materials in Table 12 were used.

TABLE 12

| Name | Stock concentration |
|---|---|
| NP-1 | 30 mg/ml (measuring DOTAP conc.) |
| NP-7 | 30 mg/ml (measuring DOTAP conc.) |
| repRNA-CoV2-spike (wild type) | 1687 ug/ml |
| VEE-S-v5 Delta (“WT-S”) | |
| repRNA-CoV2-spike (delta) | 783 ug/ml |
| VEE-nCoV19-S-Delta.AY1- | |
| S2P-wtFur (“Delta-S”) | |
| Sucrose (EMD, Millipore) | |
| Na-citrate (Teknova) | 1M |

Preparation of formulation complexes. Compositions of lipid nanoparticle/RNA complexes were prepared in this study as shown below in Table 13. NP-1 or NP-7 and repRNAs were complexed at a N-to-P ratio of 15 and complexed to obtain a final repRNA concentration of 50 mg/ml or 100 mg/ml (“2X” material), and 10% or 20% w/v sucrose content, respectively. Complexed material with 10% sucrose (50 mg/ml repRNA) contained 5 mM sodium citrate while that with 20% sucrose (100 mg/ml repRNA) contained 10 mM citrate. Complexes were filled in 2 ml sterile, depyrogenated and baked vials. Complexes with 10% sucrose were filled at 0.7 ml per vial and 20% sucrose at 0.35 ml per vial. Vials were then either lyophilized and stored or stored as is in liquid form. Storage temperature was 25 degrees C. or 42 degrees C. for 1 week. Quantity of lyophilized and liquid vials per composition is summarized in Table 13.

TABLE 13

| | | DOTAP | RNA | Volume per | Lyo | Liquid |
|---|---|---|---|---|---|---|
| Description | N:P | [μg/ml] | [μg/ml] | vial [ml] | vials | vials |
| NP-1 + WT-S in 10% | 15 | 1500 | 50 | 0.7 | 8 | 6 |
| sucrose | | | | | | |
| NP-1 + Delta-S in 10% | 15 | 1500 | 50 | 0.7 | 2 | 0 |
| sucrose | | | | | | |
| NP-1 + WT-S in 20% | 15 | 3000 | 100 | 0.35 | 8 | 0 |
| sucrose | | | | | | |
| NP-1 + Delta-S in 20% | 15 | 3000 | 100 | 0.35 | 2 | 0 |
| sucrose | | | | | | |
| NP-7 + WT-S in 10% | 15 | 1500 | 50 | 0.7 | 8 | 6 |
| sucrose | | | | | | |

Lyophilization cycle. An SP VirTis Advantage Pro tray and batch lyophilizer with inert gas fill and stoppering capability was used. Summary of the lyophilization cycle is shown in Table 14 below. After end of cycle, vials were backfilled with nitrogen at 48 torr and stoppered, before equilibrating to room pressure.

TABLE 14

| Time | Temp | Pressure | |
|---|---|---|---|
| [hours] | [° C.] | [mT] | Notes |
| 0 | 5 | 760 | Shelf pre-cooled to 5 degrees C. |
| 0.5 | 5 | 760 | Freezing |
| 2 | −50 | 760 | |
| 2.5 | −50 | 50 | Evacuation |
| 3 | −30 | 50 | Primary drying |
| 20.5 | −30 | 50 | |
| 22.5 | 25 | 50 | Secondary drying |
| 24 | 25 | 50 | |

Condition groups. A summary of 14 groups analyzed in this assay is provided in Table 15 below. Groups 1 and 4, as indicated in the storage column, were prepared fresh to serve as positive controls for comparison with standard protocol for vaccine preparation.

TABLE 15

| | | | Sucrose | | Storage |
|---|---|---|---|---|---|
| Group | Formulation | RNA | [% w/v] | Form | [temp/time] |
| 1 | NP-1 | WT-S | 10 | Liquid | Fresh |
| 2 | NP-1 | WT-S | 10 | Liquid | 25 C./1 wk |
| 3 | NP-1 | WT-S | 10 | Liquid | 42 C./1 wk |
| 4 | NP-7 | WT-S | 10 | Liquid | Fresh |
| 5 | NP-7 | WT-S | 10 | Liquid | 25 C./1 wk |
| 6 | NP-7 | WT-S | 10 | Liquid | 42 C./1 wk |
| 7 | NP-1 | WT-S | 10 | Lyo | 25 C./1 wk |
| 8 | NP-1 | WT-S | 10 | Lyo | 42 C./1 wk |
| 9 | NP-1 | WT-S | 20 | Lyo | 25 C./1 wk |
| 10 | NP-1 | WT-S | 20 | Lyo | 42 C./1 wk |
| 11 | NP-7 | WT-S | 10 | Lyo | 25 C./1 wk |
| 12 | NP-7 | WT-S | 10 | Lyo | 42 C./1 wk |
| 13 | NP-1 | Delta-S | 10 | Lyo | 25 C./1 wk |
| 14 | NP-1 | Delta-S | 20 | Lyo | 25 C./1 wk |

Immunogenicity study. Induction of anti-spike IgG responses were evaluated in 6 to 8 weeks old female C571B1/6 mice. A group size of 5 mice was used. Study schedule is shown in Table 16.

TABLE 16
Immunogenicity study schedule.

| Date | Study Day | Procedure |
|---|---|---|
| Aug. 23, 2021 | −7 | Lyophilization |
| Sep. 1, 2021 | 0 | Immunization by IM route |
| Sep. 15, 2021 | 14 | Bleed |
| Sep. 29, 2021 | 28 | Bleed |
| Oct. 8, 2021 | 37 | Mice sacrificed |

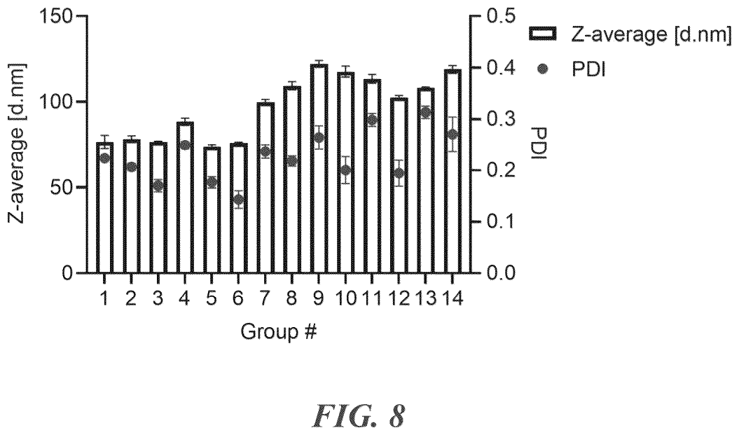

After 1 week of storage in 25 degrees C. or 42 degrees C. stability chamber, lyophilized nanoparticle/RNA complexes were reconstituted in 0.7 ml sterile milliQ water and gently swirled until no particles were visible to the naked eye. Particle size (z-average) and size distribution (PDI) of the complexes was measured and is summarized in FIG. 8, with group designations shown in Table 15. Particle size and PDI of freshly prepared NP-1/WT-S complex (group 1) was 76.8 nm and 0.223, respectively. After reconstitution, lyophilized samples (groups 7-14) grew by an average of 45% (+/−11%). Summary of % change in z-average relative to group 1 is included in Table 17.

TABLE 17

| Group # | 2 | 3 | 4 | 5 | 6 | 7 | 8 | 9 | 10 | 11 | 12 | 13 | 14 |
|---|---|---|---|---|---|---|---|---|---|---|---|---|---|
| % change | 2% | 0% | 15% | −4% | −1% | 30% | 42% | 59% | 53% | 48% | 33% | 41% | 55% |
| z-average | | | | | | | | | | | | | |
| vs. group | | | | | | | | | | | | | |
| 1 | | | | | | | | | | | | | |

Agarose gel electrophoresis of phenol-chloroform extracted repRNA. Liquid formulations of NP-1/repRNA and NP-7+repRNA in 10% sucrose or 20% sucrose, stored for 1 week at NP-1/repRNA and NP-7+repRNA, resulted in partial or full degradation of repRNA product, respectively. (Data not shown.) Lyophilization of NP-1/repRNA and NP-7+repRNA in 10% sucrose or 20% sucrose preserved repRNA integrity after 1 week storage at NP-1/repRNA and NP-7+repRNA. (Data not shown.)

Potency Assay. Lyophilized NP-1/WT-S in 10% sucrose stored for 1 week at 25 degrees C. produced a dose-dependent expression of spike protein in transfected BHK cells. The expression profile was similar to freshly complexed NP-1/WT-S. 1 week storage at 42 degrees C. of lyophilized NP-1/WT-S in 10% sucrose significantly reduced in vitro protein expression. Liquid NP-1/WT-S in 10% sucrose stored for 1 week at 25 degrees C. or 42 degrees C. did not produce spike protein in BHK cells. (Data not shown.)

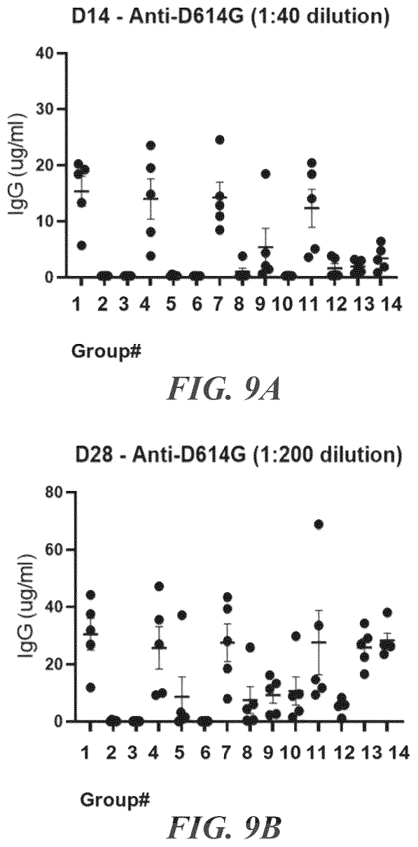

Anti-D614G spike IgG responses by ELISA. Serum anti-D614G spike IgG levels was assessed on days 14 and 28 post-prime shown below in FIGS. 9A and 9B, respectively. Mouse sera were assayed in an anti-D614G spike ELISA at 1:40 (day 14) or 1:200 (day 28) dilution. Serum IgG level in μg/ml was interpolated from a 4PL standard curve generated by a known concentration of mouse IgG standard.

Day 28 post-prime anti-D614G IgG response. After 1 week at 25 degrees C., liquid NP-1/WT-S in 10% sucrose resulted in a statistically significant reduction in anti-spike IgG compared to the freshly prepared NP-1/WT-S positive control. There was no significant difference in mean IgG levels between freshly prepared NP-1/WT-S and lyophilized NP-1/WT-S in 10% sucrose stored for 1 week at 25 degrees C.

After 1 week at 42 degrees C., lyophilized NP-1/WT-S in 10% sucrose induced 100% seroconversion but mean IgG level was significantly reduced compared to freshly prepared NP-1/WT-S. Summary mean +/−standard deviation IgG concentration data from day 28 post-immunization, including p-values determined by ordinary one-way ANOVA comparing against the freshly prepared NP-1/WT-S positive control, shown in Table 18. P<0.05 are considered statistically significant differences.

TABLE 18

| | | | | | | D 28 mean IgG | | |
|---|---|---|---|---|---|---|---|---|
| | | | | | Storage | at 1:200 serum | | P-value |
| | | | Sucrose | | temp. | dilution | SD | vs. |
| Group | Formulation | RNA | [% w/v] | State | and time | [μg/ml] | [ug/ml] | group 1 |
| 1 | NP-1 | WT-S | 10 | Liquid | Fresh | 30.38 | 12.29 | n/a |
| 2 | NP-1 | WT-S | 10 | Liquid | 25 C./1 wk | 0.17 | 0.23 | 0.0014 |
| 3 | NP-1 | WT-S | 10 | Liquid | 42 C./1 wk | 0.02 | 0.02 | 0.0013 |
| 4 | NP-7 | WT-S | 10 | Liquid | Fresh | 25.69 | 16.45 | 0.9990 |
| 5 | NP-7 | WT-S | 10 | Liquid | 25 C./1 wk | 8.47 | 15.99 | 0.0385 |
| 6 | NP-7 | WT-S | 10 | Liquid | 42 C./1 wk | 0.00 | 0.00 | 0.0013 |
| 7 | NP-1 | WT-S | 10 | Lyo | 25 C./1 wk | 27.44 | 14.68 | 0.9994 |
| 8 | NP-1 | WT-S | 10 | Lyo | 42 C./1 wk | 7.34 | 10.63 | 0.0257 |
| 9 | NP-1 | WT-S | 20 | Lyo | 25 C./1 wk | 9.07 | 6.41 | 0.0474 |
| 10 | NP-1 | WT-S | 20 | Lyo | 42 C./1 wk | 10.56 | 11.25 | 0.0777 |
| 11 | NP-7 | WT-S | 10 | Lyo | 25 C./1 wk | 27.53 | 24.96 | 0.9994 |
| 12 | NP-7 | WT-S | 10 | Lyo | 42 C./1 wk | 5.33 | 2.68 | 0.0121 |
| 13 | NP-1 | Delta- | 10 | Lyo | 25 C./1 wk | 25.83 | 6.66 | 0.9990 |
| | | S | | | | | | |
| 14 | NP-1 | Delta- | 20 | Lyo | 25 C./1 wk | 28.25 | 5.60 | 0.9996 |
| | | S | | | | | | |

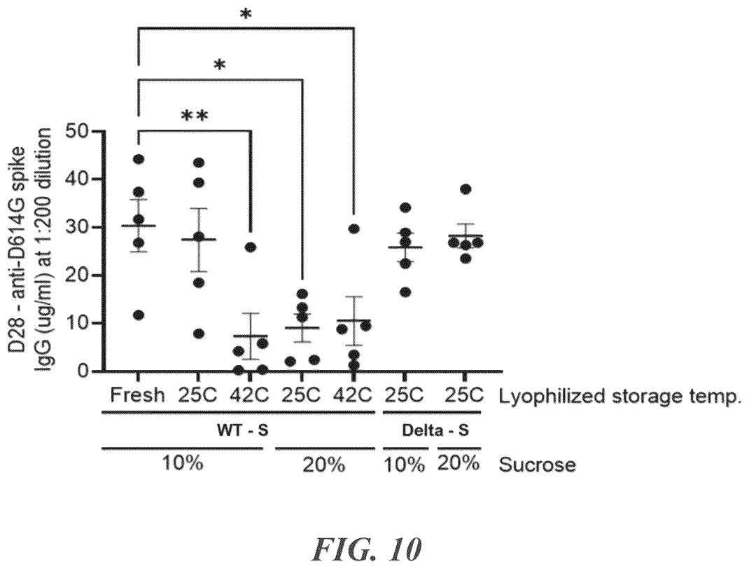

Comparison of fresh versus lyophilized formulations. Day 28 post-prime anti-D614G spike IgG concentration in serum is shown in FIG. 10. Statistical differences between mean IgG values were determined by ordinary one-way ANOVA with Dunnett's multiple comparisons test. All groups compared to freshly prepared NP-1/RNA in 10% sucrose. No significant difference was shown between freshly prepared NP-1/RNA and lyophilized NP-1/RNA in 10% sucrose stored for 7 days at 25 degrees C. At 42 degrees C., lyophilized NP-1/RNA in 10% or 20% sucrose induced significantly lower anti-spike IgG compared to freshly prepared NP-1/RNA. Lyophilized NP-1/RNA in 20% sucrose, and stored at 25 degrees C. or 42 degrees C., induced significantly lower IgG than freshly prepared NP-1/WT-S. Lyophilized NP-1/Delta-S in 10% or 20% sucrose, and stored at 25 degrees C., induced similar mean IgG (statistically not significant) than freshly prepared NP-1/WT-S.

SEQ ID NO: 1
(Delta V5 Antigen Encoding RNA Sequence)
auguuucugcucacaaccaaacgcacuauguuuguuuuccucgugcugcucccuuugguaaguucucaguguguaa
accugacaacacgaacccaguugccuccagcuuauaccaacucauuuacucgcggaguauauuaucccgauaaggu
cuuuagaaguagcguguugcacucuacacaggaucuguucuugcccuucuuuaguaacguuaccugguuucaugca
auacaugugagcggaacaaauggaacaaaaagauuugacaauccagugcuuccauuuaaugaugggguuuacuuug
ccaguaccgaaaagucaaacauaauccggggguggaucuuuggaaccacuuuggacucuaagacacagucucuccu
cauaguaaacaacgccaccaauguugucauaaaaguaugcgaauuucaguuuugcaacgaucccuuucucggggug
uauuaccauaagaauaauaaauccuggauggagucugaguuccggguuuauaguagugcuaauaauugcacuuucg
aauacgugucccaaccauuccucauggaccuugagggcaaacaggggaauuuuaaaaacuugcgcgaauuugucuu
uaagaauaucgacggauacuuuaagaucuauaguaaacacacuccuaucaaccucguucgggaucuuccccaaggc
uuuucugcucucgaaccccucguagacuugccaauugggauaaauaucacucgcuuucaaacuuugcuugcccucc
acaggagcuaccugacacccggcgacucuucuucugguuggaccgccggcgccgcugccuauuauguugguuaccu
ucagccacgaacauucuugcucaaguauaacgagaauggcaccauuaccgacgccgucgauugugcauuggauccc
uugucugaaacaaaauguaccuugaaguccuuuaccguagagaaaggcauauaccagacuuccaacuuccgaguuc
agccuacagaauccauugugagauuucccaacaucacaaaccucugcccuuucggugaaguauuuaaugcuacacg
cuucgcuucagucuaugccuggaauaggaagcgcauaucaaauugcguggccgauuauucaguccucuauaauagc
gcauccuucaguacuuucaagugcuacggcguuucccccaccaaacucaaugaucuuugcuucaccaacgucuaug
cugacaguuuugucauacgaggcgacgaaguacgccagauugcccccgggcagacagguaaaauugcugauuauaa
uuauaaacucccagaugacuuuacuggaugcgucauagccuggaauuccaacaaucuugauuccaagguugguggg
aauuauaauuaccuuuaucgacuguucagaaagaguaacuugaaaccauuugagagagacauauccaccgagauuu
accaggcaggcaguacuccuuguaacggcguugagggauuuaacugcuauuuuccuuugcaauccuauggcuuuca
accaacaaacgggguuggcuaucaacccuaucgagugguuguccugagcuuugaacuuuugcacgcucccgccaca
gucugcggaccaaaaaagaguacaaaucuugucaagaauaagugcguaaauuucaauuucaauggccuuacaggaa
caggcgugcugacugagucaaacaagaaguuccugccauuucagcaguuuggaggggauauagcagacacaacuga
cgcuguacgcgauccucagacuuuggagaucuuggacaucacucccuguucuuucggagggguaucugucaucacc
cccggaacuaauacaucaaaucaggucgcuguguuguaccaagaugucaacugcacagaaguccccguugcuauac
acgcagaccagcucacccccacauggcggguguacucaacuggcucaaacguauuccagaccagagcugggugcuu
gaucggugcugaacacguaaacaauagcuaugaaugcgauauucccaucggugccgggaucugcgcuagcuaucag
acacagaccaauuccccccggcgagcacgaucuguagcaucccagucuauuauugccuacacuaugucauugggcg
ccgagaauagcgucgcauauucaaauaauucuauugcaauacccaccaacuucacaaucuccguaacuacagaaau
acuuccaguuuccaugacaaagacaucaguggauuguacaauguauauaugcggagauuccacagaauguucaaau
uugcucuugcaguacggcuccuucugcacccagcucaacagggcccuuacagguauugcugucgaacaggacaaga
acacacaagaagucuucgcccaagucaaacagauauacaaaacuccucccauaaaggauuuuggcggcuucaacuu
uagucagauccucccagacccuucaaaaccaucuaaacgaucauuuauugaagaucugcuguucaacaaggucacu
cuugccgaugcuggauucauuaagcaauacggugacugccuuggugauauugcugcccgagaucugaucugugccc
agaaauucaacgggcucacuguacucccuccacugcucacagacgaaaugauugcacaguacacaagugcccuguu
ggcaggcacaaucacuagcggcuggaccuuuggcgcaggugcagcacuccaaauaccuuuugccaugcagauggcc
uaucgguuuaaugggauaggcgugacucaaaauguccucuacgaaaaccaaaaguugauagcuaaccaauucaauu
cagcaaucgggaagauacaggauucacugucuaguacugcuagugcccuugguaagcugcaggacguugucaacca
gaaugcucaagcucugaauacauugguuaagcagcucucuaguaauuuuggggccaucucuucaguacuuaaugau
auuuugagccgauuggacaaaguggaagcugaaguacagaucgacaggcugauaacaggccggcuccaaucccucc
aaacauacgugacacaacaacucauacgcgcagccgaaauccgagccagcgcuaaccuggcagcuaccaagauguc
agaaugcguucugggccagaguaaacgcguagauuucugcgggaaaggguaccaccugauguccuuuccacaaucu
gcaccucacggggucgucuuuuugcauguaacauauguacccgcacaagagaagaauuuuacuaccgcuccugcca
ucugucaugacgggaaagcucauuuuccucgcgaagguguguuuguaucuaaugguacacauugguuugucacaca
gcggaauuucuaugaaccccagaucauuacaacugacaacacuuuuguuuccgggaauugugacguggucauagga
aucguaaauaacacuguauaugauccccuccaaccagagcuggacucuuuuaaagaagaacuggauaaauauuuca
agaaccacacaagucccgacguggaccuuggggacauaagugguauuaacgcaucugugguuaacauucaaaagga
aaucgacagacucaacgagguggccaaaaaccugaacgaaagcuugauagaucuccaggaguugggcaaguaugaa
caguacauuaaauggccaugguacauauggcuuggcuuuaucgcuggccuuaucgccaucguaaugguuacaauca
ugcugugcugcaugaccuccugcuguucuuguuugaaaggguguuguucuugugguaguuguugcaaguuugacga
agaugauuccgaaccuguucuuaaagggguaaagcuucacuauaca
SEQ ID NO: 2
(K995P-V996P Antigen Encoding RNA Sequence)
auguuucugcucacaaccaaacgcacuauguuuguuuuccucgugcugcucccuuugguaaguucucaguguguaa
accugacaacacgaacccaguugccuccagcuuauaccaacucauuuacucgcggaguauauuaucccgauaaggu
cuuuagaaguagcguguugcacucuacacaggaucuguucuugcccuucuuuaguaacguuaccugguuucaugca
auacaugugagcggaacaaauggaacaaaaagauuugacaauccagugcuuccauuuaaugaugggguuuacuuug
ccaguaccgaaaagucaaacauaauccggggguggaucuuuggaaccacuuuggacucuaagacacagucucuccu
cauaguaaacaacgccaccaauguugucauaaaaguaugcgaauuucaguuuugcaacgaucccuuucucggggug
uauuaccauaagaauaauaaauccuggauggagucugaguuccggguuuauaguagugcuaauaauugcacuuucg
aauacgugucccaaccauuccucauggaccuugagggcaaacaggggaauuuuaaaaacuugcgcgaauuugucuu
uaagaauaucgacggauacuuuaagaucuauaguaaacacacuccuaucaaccucguucgggaucuuccccaaggc
uuuucugcucucgaaccccucguagacuugccaauugggauaaauaucacucgcuuucaaacuuugcuugcccucc
acaggagcuaccugacacccggcgacucuucuucugguuggaccgccggcgccgcugccuauuauguugguuaccu
ucagccacgaacauucuugcucaaguauaacgagaauggcaccauuaccgacgccgucgauugugcauuggauccc
uugucugaaacaaaauguaccuugaaguccuuuaccguagagaaaggcauauaccagacuuccaacuuccgaguuc
agccuacagaauccauugugagauuucccaacaucacaaaccucugcccuuucggugaaguauuuaaugcuacacg
cuucgcuucagucuaugccuggaauaggaagcgcauaucaaauugcguggccgauuauucaguccucuauaauagc
gcauccuucaguacuuucaagugcuacggcguuucccccaccaaacucaaugaucuuugcuucaccaacgucuaug
cugacaguuuugucauacgaggcgacgaaguacgccagauugcccccgggcagacagguaaaauugcugauuauaa
uuauaaacucccagaugacuuuacuggaugcgucauagccuggaauuccaacaaucuugauuccaagguugguggg
aauuauaauuaccuuuaucgacuguucagaaagaguaacuugaaaccauuugagagagacauauccaccgagauuu
accaggcaggcaguacuccuuguaacggcguugagggauuuaacugcuauuuuccuuugcaauccuauggcuuuca
accaacaaacgggguuggcuaucaacccuaucgagugguuguccugagcuuugaacuuuugcacgcucccgccaca
gucugcggaccaaaaaagaguacaaaucuugucaagaauaagugcguaaauuucaauuucaauggccuuacaggaa
caggcgugcugacugagucaaacaagaaguuccugccauuucagcaguuugggcgggauauagcagacacaacuga
cgcuguacgcgauccucagacuuuggagaucuuggacaucacucccuguucuuucggagggguaucugucaucacc
cccggaacuaauacaucaaaucaggucgcuguguuguaccaagaugucaacugcacagaaguccccguugcuauac
acgcagaccagcucacccccacauggcggguguacucaacuggcucaaacguauuccagaccagagcugggugcuu
gaucggugcugaacacguaaacaauagcuaugaaugcgauauucccaucggugccgggaucugcgcuagcuaucag
acacagaccaauuccccccggcgagcacgaucuguagcaucccagucuauuauugccuacacuaugucauugggcg
ccgagaauagcgucgcauauucaaauaauucuauugcaauacccaccaacuucacaaucuccguaacuacagaaau
acuuccaguuuccaugacaaagacaucaguggauuguacaauguauauaugcggagauuccacagaauguucaaau
uugcucuugcaguacggcuccuucugcacccagcucaacagggcccuuacagguauugcugucgaacaggacaaga
acacacaagaagucuucgcccaagucaaacagauauacaaaacuccucccauaaaggauuuuggcggcuucaacuu
uagucagauccucccagacccuucaaaaccaucuaaacgaucauuuauugaagaucugcuguucaacaaggucacu
cuugccgaugcuggauucauuaagcaauacggugacugccuuggugauauugcugcccgagaucugaucugugccc
agaaauucaacgggcucacuguacucccuccacugcucacagacgaaaugauugcacaguacacaagugcccuguu
ggcaggcacaaucacuagcggcuggaccuuuggcgcaggugcagcacuccaaauaccuuuugccaugcagauggcc
uaucgguuuaaugggauaggcgugacucaaaauguccucuacgaaaaccaaaaguugauagcuaaccaauucaauu
cagcaaucgggaagauacaggauucacugucuaguacugcuagugcccuugguaagcugcaggacguugucaacca
gaaugcucaagcucugaauacauugguuaagcagcucucuaguaauuuuggggccaucucuucaguacuuaaugau
auuuugagccgauuggacccacccgaagcugaaguacagaucgacaggcugauaacaggccggcuccaaucccucc
aaacauacgugacacaacaacucauacgcgcagccgaaauccgagccagcgcuaaccuggcagcuaccaagauguc
agaaugcguucugggccagaguaaacgcguagauuucugcgggaaaggguaccaccugauguccuuuccacaaucu
gcaccucacggggucgucuuuuugcauguaacauauguacccgcacaagagaagaauuuuacuaccgcuccugcca
ucugucaugacgggaaagcucauuuuccucgcgaagguguguuuguaucuaaugguacacauugguuugucacaca
gcggaauuucuaugaaccccagaucauuacaacugacaacacuuuuguuuccgggaauugugacguggucauagga
aucguaaauaacacuguauaugauccccuccaaccagagcuggacucuuuuaaagaagaacuggauaaauauuuca
agaaccacacaagucccgacguggaccuuggggacauaagugguauuaacgcaucugugguuaacauucaaaagga
aaucgacagacucaacgagguggccaaaaaccugaacgaaagcuugauagaucuccaggaguugggcaaguaugaa
caguacauuaaauggccaugguacauauggcuuggcuuuaucgcuggccuuaucgccaucguaaugguuacaauca
ugcugugcugcaugaccuccugcuguucuuguuugaaaggguguuguucuugugguaguuguugcaaguuugacga
agaugauuccgaaccuguucuuaaagggguaaagcuucacuauaca
SEQ ID NO: 3
(D614G Antigen Encoding RNA Sequence)
auguuucugcucacaaccaaacgcacuauguuuguuuuccucgugcugcucccuuugguaaguucucaguguguaa
accugacaacacgaacccaguugccuccagcuuauaccaacucauuuacucgcggaguauauuaucccgauaaggu
cuuuagaaguagcguguugcacucuacacaggaucuguucuugcccuucuuuaguaacguuaccugguuucaugca
auacaugugagcggaacaaauggaacaaaaagauuugacaauccagugcuuccauuuaaugaugggguuuacuuug
ccaguaccgaaaagucaaacauaauccggggguggaucuuuggaaccacuuuggacucuaagacacagucucuccu
cauaguaaacaacgccaccaauguugucauaaaaguaugcgaauuucaguuuugcaacgaucccuuucucggggug
uauuaccauaagaauaauaaauccuggauggagucugaguuccggguuuauaguagugcuaauaauugcacuuucg
aauacgugucccaaccauuccucauggaccuugagggcaaacaggggaauuuuaaaaacuugcgcgaauuugucuu
uaagaauaucgacggauacuuuaagaucuauaguaaacacacuccuaucaaccucguucgggaucuuccccaaggc
uuuucugcucucgaaccccucguagacuugccaauugggauaaauaucacucgcuuucaaacuuugcuugcccucc
acaggagcuaccugacacccggcgacucuucuucugguuggaccgccggcgccgcugccuauuauguugguuaccu
ucagccacgaacauucuugcucaaguauaacgagaauggcaccauuaccgacgccgucgauugugcauuggauccc
uugucugaaacaaaauguaccuugaaguccuuuaccguagagaaaggcauauaccagacuuccaacuuccgaguuc
agccuacagaauccauugugagauuucccaacaucacaaaccucugcccuuucggugaaguauuuaaugcuacacg
cuucgcuucagucuaugccuggaauaggaagcgcauaucaaauugcguggccgauuauucaguccucuauaauagc
gcauccuucaguacuuucaagugcuacggcguuucccccaccaaacucaaugaucuuugcuucaccaacgucuaug
cugacaguuuugucauacgaggcgacgaaguacgccagauugcccccgggcagacagguaaaauugcugauuauaa
uuauaaacucccagaugacuuuacuggaugcgucauagccuggaauuccaacaaucuugauuccaagguugguggg
aauuauaauuaccuuuaucgacuguucagaaagaguaacuugaaaccauuugagagagacauauccaccgagauuu
accaggcaggcaguacuccuuguaacggcguugagggauuuaacugcuauuuuccuuugcaauccuauggcuuuca
accaacaaacgggguuggcuaucaacccuaucgagugguuguccugagcuuugaacuuuugcacgcucccgccaca
gucugcggaccaaaaaagaguacaaaucuugucaagaauaagugcguaaauuucaauuucaauggccuuacaggaa
caggcgugcugacugagucaaacaagaaguuccugccauuucagcaguuugggcgggauauagcagacacaacuga
cgcuguacgcgauccucagacuuuggagaucuuggacaucacucccuguucuuucggagggguaucugucaucacc
cccggaacuaauacaucaaaucaggucgcuguguuguaccaagaugucaacugcacagaaguccccguugcuauac
acgcaggccagcucacccccacauggcggguguacucaacuggcucaaacguauuccagaccagagcugggugcuu
gaucggugcugaacacguaaacaauagcuaugaaugcgauauucccaucggugccgggaucugcgcuagcuaucag
acacagaccaauuccccccggcgagcacgaucuguagcaucccagucuauuauugccuacacuaugucauugggcg
ccgagaauagcgucgcauauucaaauaauucuauugcaauacccaccaacuucacaaucuccguaacuacagaaau
acuuccaguuuccaugacaaagacaucaguggauuguacaauguauauaugcggagauuccacagaauguucaaau
uugcucuugcaguacggcuccuucugcacccagcucaacagggcccuuacagguauugcugucgaacaggacaaga
acacacaagaagucuucgcccaagucaaacagauauacaaaacuccucccauaaaggauuuuggcggcuucaacuu
uagucagauccucccagacccuucaaaaccaucuaaacgaucauuuauugaagaucugcuguucaacaaggucacu
cuugccgaugcuggauucauuaagcaauacggugacugccuuggugauauugcugcccgagaucugaucugugccc
agaaauucaacgggcucacuguacucccuccacugcucacagacgaaaugauugcacaguacacaagugcccuguu
ggcaggcacaaucacuagcggcuggaccuuuggcgcaggugcagcacuccaaauaccuuuugccaugcagauggcc
uaucgguuuaaugggauaggcgugacucaaaauguccucuacgaaaaccaaaaguugauagcuaaccaauucaauu
cagcaaucgggaagauacaggauucacugucuaguacugcuagugcccuugguaagcugcaggacguugucaacca
gaaugcucaagcucugaauacauugguuaagcagcucucuaguaauuuuggggccaucucuucaguacuuaaugau
auuuugagccgauuggacaaaguggaagcugaaguacagaucgacaggcugauaacaggccggcuccaaucccucc
aaacauacgugacacaacaacucauacgcgcagccgaaauccgagccagcgcuaaccuggcagcuaccaagauguc
agaaugcguucugggccagaguaaacgcguagauuucugcgggaaaggguaccaccugauguccuuuccacaaucu
gcaccucacggggucgucuuuuugcauguaacauauguacccgcacaagagaagaauuuuacuaccgcuccugcca
ucugucaugacgggaaagcucauuuuccucgcgaagguguguuuguaucuaaugguacacauugguuugucacaca
gcggaauuucuaugaaccccagaucauuacaacugacaacacuuuuguuuccgggaauugugacguggucauagga
aucguaaauaacacuguauaugauccccuccaaccagagcuggacucuuuuaaagaagaacuggauaaauauuuca
agaaccacacaagucccgacguggaccuuggggacauaagugguauuaacgcaucugugguuaacauucaaaagga
aaucgacagacucaacgagguggccaaaaaccugaacgaaagcuugauagaucuccaggaguugggcaaguaugaa
caguacauuaaauggccaugguacauauggcuuggcuuuaucgcuggccuuaucgccaucguaaugguuacaauca
ugcugugcugcaugaccuccugcuguucuuguuugaaaggguguuguucuugugguaguuguugcaaguuugaga
agaugauuccgaaccuguucuuaaagggguaaagcuucacuauaca
SEQ ID NO: 4
(B.1.351-PP-D614G Antigen Encoding RNA Sequence)
auguuucugcucacaaccaaacgcacuauguuuguuuuccucgugcugcucccuuugguaaguucucaguguguaa
acuucacaacacgaacccaguugccuccagcuuauaccaacucauuuacucgcggaguauauuaucccgauaaggu
cuuuagaaguagcguguugcacucuacacaggaucuguucuugcccuucuuuaguaacguuaccugguuucaugca
auacaugugagcggaacaaauggaacaaaaagauuugccaauccagugcuuccauuuaaugaugggguuuacuuug
ccaguaccgaaaagucaaacauaauccggggguggaucuuuggaaccacuuuggacucuaagacacagucucuccu
cauaguaaacaacgccaccaauguugucauaaaaguaugcgaauuucaguuuugcaacgaucccuuucucggggug
uauuaccauaagaauaauaaauccuggauggagucugaguuccggguuuauaguagugcuaauaauugcacuuucg
aauacgugucccaaccauuccucauggaccuugagggcaaacaggggaauuuuaaaaacuugcgcgaauuugucuu
uaagaauaucgacggauacuuuaagaucuauaguaaacacacuccuaucaaccucguucggggccuuccccaaggc
uuuucugcucucgaaccccucguagacuugccaauugggauaaauaucacucgcuuucaaacuuugcacaucagcu
accugacacccggcgacucuucuucugguuggaccgccggcgccgcugccuauuauguugguuaccuucagccacg
aacauucuugcucaaguauaacgagaauggcaccauuaccgacgccgucgauugugcauuggaucccuugucugaa
acaaaauguaccuugaaguccuuuaccguagagaaaggcauauaccagacuuccaacuuccgaguucagccuacag
aauccauugugagauuucccaacaucacaaaccucugcccuuucggugaaguauuuaaugcuacacgcuucgcuuc
agucuaugccuggaauaggaagcgcauaucaaauugcguggccgauuauucaguccucuauaauagcgcauccuuc
aguacuuucaagugcuacggcguuucccccaccaaacucaaugaucuuugcuucaccaacgucuaugcugacaguu
uugucauacgaggcgacgaaguacgccagauugcccccgggcagacagguaacauugcugauuauaauuauaaacu
cccagaugacuuuacuggaugcgucauagccuggaauuccaacaaucuugauuccaagguuggugggaauuauaau
uaccuuuaucgacuguucagaaagaguaacuugaaaccauuugagagagacauauccaccgagauuuaccaggcag
gcaguacuccuuguaacggcguuaagggauuuaacugcuauuuuccuuugcaauccuauggcuuucaaccaacaua
cgggguuggcuaucaacccuaucgagugguuguccugagcuuugaacuuuugcacgcucccgccacagucugcgga
ccaaaaaagaguacaaaucuugucaagaauaagugcguaaauuucaauuucaauggccuuacaggaacaggcgugc
ugacugagucaaacaagaaguuccugccauuucagcaguuugggcgggauauagcagacacaacugacgcuguacg
cgauccucagacuuuggagaucuuggacaucacucccuguucuuucggagggguaucugucaucacccccggaacu
aauacaucaaaucaggucgcuguguuguaccaaggcgucaacugcacagaaguccccguugcuauacacgcagacc
agcucacccccacauggcggguguacucaacuggcucaaacguauuccagaccagagcugggugcuugaucggugc
ugaacacguaaacaauagcuaugaaugcgauauucccaucggugccgggaucugcgcuagcuaucagacacagacc
aauuccccccggcgagcacgaucuguagcaucccagucuauuauugccuacacuaugucauugggcguggagaaua
gcgucgcauauucaaauaauucuauugcaauacccaccaacuucacaaucuccguaacuacagaaauacuuccagu
uuccaugacaaagacaucaguggauuguacaauguauauaugcggagauuccacagaauguucaaauuugcucuug
caguacggcuccuucugcacccagcucaacagggcccuuacagguauugcugucgaacaggacaagaacacacaag
aagucuucgcccaagucaaacagauauacaaaacuccucccauaaaggauuuuggcggcuucaacuuuagucagau
ccucccagacccuucaaaaccaucuaaacgaucauuuauugaagaucugcuguucaacaaggucacucuugccgau
gcuggauucauuaagcaauacggugacugccuuggugauauugcugcccgagaucugaucugugcccagaaauuca
acgggcucacuguacucccuccacugcucacagacgaaaugauugcacaguacacaagugcccuguuggcaggcac
aaucacuagcggcuggaccuuuggcgcaggugcagcacuccaaauaccuuuugccaugcagauggccuaucgguuu
aaugggauaggcgugacucaaaauguccucuacgaaaaccaaaaguugauagcuaaccaauucaauucagcaaucg
ggaagauacaggauucacugucuaguacugcuagugcccuugguaagcugcaggacguugucaaccagaaugcuca
agcucugaauacauugguuaagcagcucucuaguaauuuuggggccaucucuucaguacuuaaugauauuuugagc
cgauuggacccacccgaagcugaaguacagaucgacaggcugauaacaggccggcuccaaucccuccaaacauacg
ugacacaacaacucauacgcgcagccgaaauccgagccagcgcuaaccuggcagcuaccaagaugucagaaugcgu
ucugggccagaguaaacgcguagauuucugcgggaaaggguaccaccugauguccuuuccacaaucugcaccucac
ggggucgucuuuuugcauguaacauauguacccgcacaagagaagaauuuuacuaccgcuccugccaucugucaug
acgggaaagcucauuuuccucgcgaagguguguuuguaucuaaugguacacauugguuugucacacagcggaauuu
cuaugaaccccagaucauuacaacugacaacacuuuuguuuccgggaauugugacguggucauaggaaucguaaau
aacacuguauaugauccccuccaaccagagcuggacucuuuuaaagaagaacuggauaaauauuucaagaaccaca
caagucccgacguggaccuuggggacauaagugguauuaacgcaucugugguuaacauucaaaaggaaaucgacag
acucaacgagguggccaaaaaccugaacgaaagcuugauagaucuccaggaguugggcaaguaugaacaguacauu
aaauggccaugguacauauggcuuggcuuuaucgcuggccuuaucgccaucguaaugguuacaaucaugcugugcu
gcaugaccuccugcuguucuuguuugaaaggguguuguucuugugguaguuguugcaaguuugacgaagaugauuc
cgaaccuguucuuaaagggguaaagcuucacuauaca
SEQ ID NO: 5
(B.1.1.7-PP-D614G Antigen Encoding RNA Sequence)
auguuucugcucacaaccaaacgcacuauguuuguuuuccucgugcugcucccuuugguaaguucucaguguguaa
accugacaacacgaacccaguugccuccagcuuauaccaacucauuuacucgcggaguauauuaucccgauaaggu
cuuuagaaguagcguguugcacucuacacaggaucuguucuugcccuucuuuaguaacguuaccugguuucaugca
auaagcggaacaaauggaacaaaaagauuugacaauccagugcuuccauuuaaugaugggguuuacuuugccagua
ccgaaaagucaaacauaauccggggguggaucuuuggaaccacuuuggacucuaagacacagucucuccucauagu
aaacaacgccaccaauguugucauaaaaguaugcgaauuucaguuuugcaacgaucccuuucucgggguguaccau
aagaauaauaaauccuggauggagucugaguuccggguuuauaguagugcuaauaauugcacuuucgaauacgugu
cccaaccauuccucauggaccuugagggcaaacaggggaauuuuaaaaacuugcgcgaauuugucuuuaagaauau
cgacggauacuuuaagaucuauaguaaacacacuccuaucaaccucguucgggaucuuccccaaggcuuuucugcu
cucgaaccccucguagacuugccaauugggauaaauaucacucgcuuucaaacuuugcuugcccuccacaggagcu
accugacacccggcgacucuucuucugguuggaccgccggcgccgcugccuauuauguugguuaccuucagccacg
aacauucuugcucaaguauaacgagaauggcaccauuaccgacgccgucgauugugcauuggaucccuugucugaa
acaaaauguaccuugaaguccuuuaccguagagaaaggcauauaccagacuuccaacuuccgaguucagccuacag
aauccauugugagauuucccaacaucacaaaccucugcccuuucggugaaguauuuaaugcuacacgcuucgcuuc
agucuaugccuggaauaggaagcgcauaucaaauugcguggccgauuauucaguccucuauaauagcgcauccuuc
aguacuuucaagugcuacggcguuucccccaccaaacucaaugaucuuugcuucaccaacgucuaugcugacaguu
uugucauacgaggcgacgaaguacgccagauugcccccgggcagacagguaaaauugcugauuauaauuauaaacu
cccagaugacuuuacuggaugcgucauagccuggaauuccaacaaucuugauuccaagguuggugggaauuauaau
uaccuuuaucgacuguucagaaagaguaacuugaaaccauuugagagagacauauccaccgagauuuaccaggcag
gcaguacuccuuguaacggcguugagggauuuaacugcuauuuuccuuugcaauccuauggcuuucaaccaacaua
cgggguuggcuaucaacccuaucgagugguuguccugagcuuugaacuuuugcacgcucccgccacagucugcgga
ccaaaaaagaguacaaaucuugucaagaauaagugcguaaauuucaauuucaauggccuuacaggaacaggcgugc
ugacugagucaaacaagaaguuccugccauuucagcaguuugggcgggauauagacgacacaacugacgcuguacg
cgauccucagacuuuggagaucuuggacaucacucccuguucuuucggagggguaucugucaucacccccggaacu
aauacaucaaaucaggucgcuguguuguaccaaggcgucaacugcacagaaguccccguugcuauacacgcagauc
agcucacccccacauggcggguguacucaacuggcucaaacguauuccagaccagagcugggugcuugaucggugc
ugaacacguaaacaauagcuaugaaugcgauauucccaucggugccgggaucugcgcuagcuaucagacacagacc
aauucccaucggcgagcacgaucuguagcaucccagucuauuauugccuacacuaugucauugggcgccgagaaua
gcgucgcauauucaaauaauucuauugcaauacccaucaacuucacaaucuccguaacuacagaaauacuuccagu
uuccaugacaaagacaucaguggauuguacaauguauauaugcggagauuccacagaauguucaaauuugcucuug
caguacggcuccuucugcacccagcucaacagggcccuuacagguauugcugucgaacaggacaagaacacacaag
aagucuucgcccaagucaaacagauauacaaaacuccucccauaaaggauuuugggugcuucaacuuuagucagau
ccucccagacccuucaaaaccaucuaaacgaucauuuauugaagaucugcuguucaacaaggucacucuugccgau
gcuggauucauuaagcaauacggugacugccuuggugauauugcugcccgagaucugaucugugcccagaaauuca
acgggcucacuguacucccuccacugcucacagacgaaaugauugcacaguacacaagugcccuguuggcaggcac
aaucacuagcggcuggaccuuuggcgcaggugcagcacuccaaauaccuuuugccaugcagauggccuaucgguuu
aaugggauaggcgugacucaaaauguccucuacgaaaaccaaaaguugauagcuaaccaauucaauucagcaaucg
ggaagauacaggauucacugucuaguacugcuagugcccuugguaagcugcaggacguugucaaccagaaugcuca
agcucugaauacauugguuaagcagcucucuaguaauuuuggggccaucucuucaguacuuaaugauauuuuggcc
cgauuggacccacccgaagcugaaguacagaucgacaggcugauaacaggccggcuccaaucccuccaaacauacg
ugacacaacaacucauacgcgcagccgaaauccgagccagcgcuaaccuggcagcuaccaagaugucagaaugcgu
ucugggccagaguaaacgcguagauuucugcgggaaaggguaccaccugauguccuuuccacaaucugcaccucac
ggggucgucuuuuugcauguaacauauguacccgcacaagagaagaauuuuacuaccgcuccugccaucugucaug
acgggaaagcucauuuuccucgcgaagguguguuuguaucuaaugguacacauugguuugucacacagcggaauuu
cuaugaaccccagaucauuacaacucacaacacuuuuguuuccgggaauugugacguggucauaggaaucguaaau
aacacuguauaugauccccuccaaccagagcuggacucuuuuaaagaagaacuggauaaauauuucaagaaccaca
caagucccgacguggaccuuggggacauaagugguauuaacgcaucugugguuaacauucaaaaggaaaucgacag
acucaacgagguggccaaaaaccugaacgaaagcuugauagaucuccaggaguugggcaaguaugaacaguacauu
aaauggccaugguacauauggcuuggcuuuaucgcuggccuuaucgccaucguaaugguuacaaucaugcugugcu
gcaugaccuccugcuguucuuguuugaaaggguguuguucuugugguaguuguugaaguuugagaagaugauuc
cgaaccuguucuuaaagggguaaagcuucacuauaca
SEQ ID NO: 6
(Delta.AY1-S2P-wtFur Antigen Encoding RNA Sequence)
auguuucugcucacaaccaaacgcacuauguuuguuuuccucgugcugcucccuuugguaaguucucaguguguaa
accugagaacacgaacccaguugccuccagcuuauaccaacucauuuacucgcggaguauauuaucccgauaaggu
cuuuagaaguagcguguugcacucuacacaggaucuguuuugcccuuuuuaguaacguuaccuguuucaucaugca
auacaugugagcggaacaaauggaacaaaaagauuugacaauccagugcuuccauuuaaugaugggguuuacuuug
ccaguaucgaaaagucaaacauaauccggggguggaucuuuggaaccacuuuggacucuaagacacagucucuccu
cauaguaaacaacgccaccaauguugucauaaaaguaugcgaauuucaguuuugcaacgaucccuuucucgacgug
uauuaccauaagaauaauaaauccuggauggagucugggguuuauaguagugcuaauaauugcacuuucgaauacg
ugucccaaccauuccucauggaccuugagggcaaacaggggaauuuuaaaaacuugcgcgaauuugucuuuaagaa
uaucgacggauacuuuaagaucuauaguaaacacacuccuaucaaccucguucgggaucuuccccaaggcuuuucu
gcucucgaaccccucguagacuugccaauugggauaaauaucacucgcuuucaaacuuugcuugcccuccacagga
gcuaccugacacccggcgacucuucuucugguuugaccgccggcgccgcugccuauuauguugguuaccuucagcc
acgaacauucuugcucaaguauaacgagaauggcaccauuaccgacgccgucgauugugcauuggaucccuugucu
gaaacaaaauguaccuugaaguccuuuaccguagagaaaggcauauaccagacuuccaacuuccgaguucagccua
cagaauccaucguacgauuucccaacaucacaaaccucugcccuuucggugaaguauuuaaugcuacacgcuucgc
uucagucuaugccuggaauaggaagcgcauaucaaauugcguggccgauuauucaguccucuauaauagcgcaucc
uucaguacuuucaagugcuacggcguuucccccaccaaacucaaugaucuuugCuucaccaacgucuaugcugaca
guuuugucauacgaggcgacgaaguacgccagauugcccccgggcagacagguaauauugcugauuauaauuauaa
acucccagaugacuuuacuggaugcgucauagccuggaauuccaacaaucuagauuccaagguuggugggaauuau
aauuaccguuaucgacuguucagaaagaguaacuugaaaccauuugagagagacauauccaccgagauuuaccagg
caggcaguaagccuuguaacggcguugagggauuuaacugcuauuuuccuuugcaauccuauggcuuucaaccaac
aaacgggguuggcuaucaacccuaucgagugguuguccucagcuuugaacuuuugcacgcucccgccacagucugc
ggaccaaaaaagaguacaaaucuugucaagaauaagugcguaaauuucaauuucaauggccuuacaggaacaggcg
ugcugacugagucaaacaagaauuuccugccauuucagcaguuugggCgggauauagcagacacaacugacgcugu
acgcgauccucagacuuuggagaucuuggacaucacucccuguuCuuucggagggguaucugucaucacccccgga
acuaauacaucaaaucaggucgcuguguuguaccaaggugucaacugcacagaaguccccguugcuauacacgcag
accagcucacccccacauggCggguguacucaacuggcucaaacguauuccagaccagagcugggugcuugaucgg
ugcugaacacgugaacaauagcuaugaaugcgauauucccaucggugccgggaucugcgcuagcuaucagacacag
accaauucccgcaggCgggcucgcucuguagcaucccagucuauuauugccuacacuaugucauugggcgccgaga
auagcgucgcauauucaaauaauucuauugcaauacccaccaacuucacaaucuccguaacuacagaaauacuucc
aguuuccaugacaaagacaucaguggauuguacaauguauauaugcggagauuccacagaauguucaaauuugcuc
uugcaguacggcuccuucugcacccagcucaacagggcacuuacagguauugcugucgaacaggacaagaacacac
aagaagucuucgcccaagucaaacagauauacaaaacuccucccauaaaggauuuuggcggcuucaacuuuaguca
gauccucccagacccuucaaaaccaucuaaacgaucauuuauugaagaucugcuguucaacaaggucacucuugcc
gaugcuggauucauuaagcaauacggugacugccuuggugauauugcugcccgagaucugaucugugcccagaaau
ucaacgggcucacuguacucccuccacugcucacagacgaaaugauugcacaguacacaagugcccuguuggcagg
cacaaucacuagcggcuggaccuuuggcgcaggugcagcacuccaaauaccuuuugccaugcagauggccuaucgg
uuuaaugggauaggcgugacucaaaauguccucuacgaaaaccaaaaguugauagcuaaccaauucaauucagcaa
ucgggaagauacaggauucacugucuaguacugcuagugcccuugguaagcugcagaacguugucaaccagaaugc
agccgauuggacccaccugaagcugaaguacagaucgacaggcugauaacaggccggcuccaaucccuccaaacau
acgugacacaacaacucauacgcgcagccgaaauccgagccagcgcuaaccuggcagcuaccaagaugucagaaug
cguucugggccagaguaaacgcguagauuucugcgggaaaggguaccaccugauguccuuuccacaaucugcaccu
cacggggucgucuuuuugcauguaacauacguacccgcacaagagaagaauuuuacuaccgcuccugccaucuguc
augacgggaaagcucauuuuccucgcgaagguguguuuguaucuaaugguacacauugguuugucacacagcggaa
uuucuaugaaccccagaucauuacaacugacaacacuuuuguuuccgggaauugugacguggucauaggaaucgua
aauaacacuguauaugauccccuccaaccagagcuggacucuuuuaaagaagaacuggauaaauauuucaagaacc
acacaagucccgacguggaccuuggggacauaagugguauuaacgcaucugugguuaacauucaaaaggaaaucga
cagacucaacgagguggccaaaaaccugaacgaaagcuugauagaucuccaggaguugggcaaguaugaacaguac
auuaaauggccaugguacauauggcuuggcuuuaucgcuggccuuaucgccaucguaaugguuacaaucaugcugu
gcugcaugaccuccugcuguucuuguuugaaaggguguuguucuugugguaguuguugcaaguuugacgaagauga
uuccgaaccuguucuuaaggggguaaagcuucacuauacauga
SEQ ID NO: 7
(Delta.AY1-S2P-wtFur-newKozak Antigen Encoding RNA Sequence)
auguuucugcucacaaccaaacgcacuauguuuguuuuccucgugcugcucccuuugguaaguucucaguguguaa
accugagaacacgaacccaguugccuccagcuuauaccaacucauuuacucgcggaguauauuaucccgauaaggu
auacaugugagcggaacaaauggaacaaaaagauuugacaauccagugcuuccauuuaaugaugggguuuacuuug
ccaguaucgaaaagucaaacauaauccggggguggaucuuuggaaccacuuuggacucuaagacacagucucuccu
ccaguaucgaaaagucaaacauaauccggggguggaucuuuggaaccacuuuggacucuaagacacagucucuccu
cauaguaaacaacgccaccaauguugucauaaaaguaugcgaauuucaguuuugcaacgaucccuuucucgacgug
uauuaccauaagaauaauaaauccuggauggagucugggguuuauaguagugcuaauaauugcacuuucgaauacg
ugucccaaccauuccucauggaccuugagggcaaacaggggaauuuuaaaaacuugcgcgaauuugucuuuaagaa
uaucgacggauacuuuaagaucuauaguaaacacacuccuaucaaccucguucgggaucuuccccaaggcuuuucu
gcucucgaaccccucguagacuugccaauugggauaaauaucacucgcuuucaaacuuugcuugcccuccacagga
gcuaccugacacccggcgacucuucuucugguuugaccgccggcgccgcugccuauuauguugguuaccuucagcc
acgaacauucuugcucaaguauaacgagaauggcaccauuaccgacgccgucgauugugcauuggaucccuugucu
gaaacaaaauguaccuugaaguccuuuaccguagagaaaggcauauaccagacuuccaacuuccgaguucagccua
cagaauccaucguacgauuucccaacaucacaaaccucugcccuuucggugaaguauuuaaugcuacacgcuucgc
uucagucuaugccuggaauaggaagcgcauaucaaauugcguggccgauuauucaguccucuauaauagcgcaucc
uucaguacuuucaagugcuacggcguuucccccaccaaacucaaugaucucuuguucaccaacgucuaugcugaca
guuuugucauacgaggcgacgaaguacgccagauugcccccgggcagacagguaauauugcugauuauaauuauaa
acucccagaugacuuuacuggaugcgucauagccuggaauuccaacaaucuagauuccaagguuggugggaauuau
aauuaccguuaucgacuguucagaaagaguaacuugaaaccauuugagagagacauauccaccgagauuuaccagg
caggcaguaagccuuguaacggcguugagggauuuaacugcuauuuuccuuugcaauccuauggcuuucaaccaac
aaacgggguuggcuaucaacccuaucgagugguuguccucagcuuugaacuuuugcacgcucccgccacagucugc
ggaccaaaaaagaguacaaaucuugucaagaauaagugcguaaauuucaauuucaauggccuuacaggaacaggcg
ugcugacugagucaaacaagaauuuccugccauuucagcaguuugggcgggauauagcagacacaacugacgcugu
acgcgauccucagacuuuggagaucuuggacaucacucccuguucuuucggagggguaucugucaucacccccgga
acuaauacaucaaaucaggucgcuguguuguaccaaggugucaacugcacagaaguccccguugcuauacacgcag
accagcucacccccacauggcggguguacucaacuggcucaaacguauuccagaccagagcugggugcuugaucgg
ugcugaacacgugaacaauagcuaugaaugcgauauucccaucggugccgggaucugcgcuagcuaucagacacag
accaauucccgcaggcgggcucgcucuguagcaucccagucuauuauugccuacacuaugucauugggcgccgaga
auagcgucgcauauucaaauaauucuauugcaauacccaccaacuucacaaucuccguaacuacagaaauacuucc
aguuuccaugacaaagacaucaguggauuguacaauguauauaugcggagauuccacagaauguucaaauuugcuc
uugcaguacggcuccuucugcacccagcucaacagggcacuuacagguauugcugucgaacaggacaagaacacac
aagaagucuucgcccaagucaaacagauauacaaaacuccucccauaaaggauuuuggcggcuucaacuuuaguca
gauccucccagacccuucaaaaccaucuaaacgaucauuuauugaagaucugcuguucaacaaggucacucuugcc
gaugcuggauucauuaagcaauacggugacugccuuggugauauugcugcccgagaucugaucugugcccagaaau
ucaacgggcucacuguacucccuccacugcucacagacgaaaugauugcacaguacacaagugcccuguuggcagg
cacaaucacuagcggcuggaccuuuggcgcaggugcagcacuccaaauaccuuuugccaugcagauggccuaucgg
uuuaaugggauaggcgugacucaaaauguccucuacgaaaaccaaaaguugauagcuaaccaauucaauucagcaa
ucgggaagauacaggauucacugucuaguacugcuagugcccuugguaagcugcagaacguugucaaccagaaugc
ucaagcucugaauacauugguuaagcagcucucuaguaauuuuggggccaucucuucaguacuuaaugauauuuug
agccgauuggacccaccugaagcugaaguacagaucgacaggcugauaacaggccggcuccaaucccuccaaacau
acgugacacaacaacucauacgcgcagccgaaauccgagccagcgcuaaccuggcagcuaccaagaugucagaaug
cguucugggccagaguaaacgcguagauuucugcgggaaaggguaccaccugauguccuuuccacaaucugcaccu
cacggggucgucuuuuugcauguaacauacguacccgcacaagagaagaauuuuacuaccgcuccugccaucuguc
augacgggaaagcucauuuuccucgcgaagguguguuuguaucuaaugguacacauugguuugucacacagcggaa
uuucuaugaaccccagaucauuacaacugacaacacuuuuguuuccgggaauugugacguggucauaggaaucgua
aauaacacuguauaugauccccuccaaccagagcuggacucuuuuaaagaagaacuggauaaauauuucaagaacc
acacaagucccgacguggaccuuggggacauaagugguauuaacgcaucugugguuaacauucaaaaggaaaucga
cagacucaacgagguggccaaaaaccugaacgaaagcuugauagaucuccaggaguugggcaaguaugaacaguac
auuaaauggccaugguacauauggcuuggcuuuaucgcuggccuuaucgccaucguaaugguuacaaucaugcugu
gcugcaugaccuccugcuguucuuguuugaaaggguguuguucuugugguaguuguugcaaguuugacgaagauga
uuccgaaccuguucuuaaggggguaaagcuucacuauacauga
SEQ ID NO: 8
(Omicron-B.1.1.529 Antigen Encoding RNA Sequence)
cgccaccauguuucuguugacgaccaagcgaacgauguucguuuucuuggugcuuuugccacuugucaguucccag
ugcgucaaucugacgacacgaacacagcugccuccugcguacacuaacaguuuuacgcgaggaguguauuacccug
acaaaguuuuccgcucuaguguccuccauagcacacaggacuuguuucuccccuucuuuuccaacguuacgugguu
ccaugugauuaguggaacuaacgguacuaaaagauucgacaauccaguauugccuuucaacgauggggucuauuuc
gcguccaucgagaaaucaaauaucauucgcgguuggauuuuuggaacgacacucgauucaaagacgcaaucccucc
uuauugucaacaacgccacuaacguagucauuaagguuugugaguuccaguuuuguaaugaucccuuuuuugacca
caagaauaacaagagcuggauggaaagcgaguucagaguguauagcucugcaaacaacuguacuuuugaauacgug
agucaaccuuuccuuauggaccuugaagguaaacaggguaacuuuaagaauuugcgcgaauuuguuuucaaaaaca
uugaugguuacuuuaaaaucuauaguaagcacacuccuaucauuguaagagagccggaggaccuuccacaggguuu
uagugcgcucgagccccucguugaccugcccauugggaucaacauaacucgauuccaaacauugcucgcccuucau
cgguccuaucugacucccggugacuccucuagcggauggacggcaggugccgccgcauacuacgugggguaccuuc
aaccucggacauuuuuguugaaauacaaugagaauggcacuauaacugacgcgguugauugcgcgcucgacccauu
guccgaaacuaaguguacuuugaagucauuuacaguggagaaaggaauauaucagacuagcaauuuucggguacag
cccacggagucuaucguacgguuuccuaacaucacgaaucugugcccuuuugaugaggucuuuaaugcaacacggu
ucgccuccgucuaugcguggaauagaaagcgcaucucaaauuguguagcugauuauuccguacuuuacaacuuggc
cccguucuucacauuuaagugcuacgguguaagcccuacuaaacugaacgauuuguguuucaccaacgucuaugca
gauagcuuuguuauucgaggcgaugagguacgccagauugcgccuggucaaacggguaauaucgccgacuacaauu
auaaauugccagacgauuuuacugguugugucaucgcuuggaauaguaauaaguuggacaguaagguauccggcaa
uuacaacuaucucuaccgauuguuccggaagucuaaccucaagccguuugaaagagacauauccacugagauauac
caagcaggcaauaaaccaugcaacggaguugcugguuucaacugcuauuucccguugcggucuuauuccuucagac
cuacuuacggagucggacaccaacccuacagggucgucguuuugaguuuugaauuguugcaugcuccagcaaccgu
guguggaccuaaaaaguccacgaaucucgugaagaauaagugcguaaacuucaauuucaacggucugaaagggacu
gguguauugacagaaagcaacaagaaguuucugccauuccagcaauuugguagggauauagcggauacaacugaug
ccguucgggauccucaaacauuggagaucuuggacaucacaccguguucuuuugggggugucuccguuaucacacc
ggguacaaauacgagcaaucagguugcgguccuuuaccaaggcguuaauuguaccgagguuccaguagcaauacac
gcggaucaacucacgcccacauggaggguuuacaguacaggcaguaauguuuuccaaacgagagcgggaugccuca
ucggggcagaauacguaaauaauucuuacgaaugcgacaucccuauuggcgcaggaauuugcgcaaguuaccaaac
ccagaccaagucucauaggcgggcgcggucuguugcaagccaaucuauaauagcguacacuaugucccucggcgcg
gagaacagugucgcauauuccaacaacucuauugcgauaccuacuaauuucacuauuagcgucacaacugagaucc
uucccgucaguaugaccaaaacgucugucgacuguacuauguauauuugcggcgacaguaccgaaugcucuaaucu
uuuguugcaguaugguucuuuuugcacgcaacuuaagagagcuuugacggggauagcuguggaacaagauaaaaac
acacaggagguauuugcacaagugaaacagaucuauaaaacuccaccgaucaaguacuuuggcggcuuuaacuucu
cccagaucuugcccgacccgucuaaaccaaguaaacggaguuuuauagaggaccuucucuucaauaagguaacauu
ggcagacgccggcuucauuaaacaauacggagauugccuuggagacaucgcugcgcgcgacuugaucugcgcacaa
aaauuuaaaggcuugacgguccucccuccuuugcucacagacgagaugauagcacaauacacuuccgcacugcuug
cuggaaccaucaccucugguuggacauucggugcgggagcggcuuugcagauuccguuugcgaugcaaauggcuua
ucgguuuaacggcauuggaguaacacagaaugugcucuacgagaaucaaaagcuuauugcgaaucaauucaacucu
gcgauuggcaaaauucaagauucauugaguagcaccgccagugcucuuggcaagcuucaggaugucguaaaccaca
augcacaagcucugaauacacugguuaaacaauuguccaguaaauuuggggcaaucucuucagugcugaacgacau
uuucucaagauuggauccacccgaagcggagguacagauugaccgccugauaaccgggagguugcaaagccuucag
acuuauguuacacaacaguugauucgggcagcagagauaagagccucagcaaaccucgcagcuacgaagaugucag
aguguguccuugggcaaucuaagcggguagauuucugcggcaaaggauaucauuugaugagcuuuccccaaucagc
cccagaucuugcccgacccgucuaaaccaaguaaacggaguuuuauagaggaccuucucuucaauaagguaacauu
ggcagacgccggcuucauuaaacaauacggagauugccuuggagacaucgcugcgcgcgacuugaucugcgcacaa
ggaauuucuacgaaccacagaucaucacuaccgacaacacguuugucucuggaaauugugacguugucauagggau
agugaacaauacaguauaugauccacuucagccugaacuugacucuuuuaaggaggagcuggacaaauauuucaaa
aaucauacaagcccggacgucgaucuuggagauauuucagguaucaacgcaagugucguaaauauucagaaggaga
ucgaucgauugaacgagguugcaaaaaaccuuaaugagagccuuauagaucuucaagagcuggggaaguaugaaca
auauaucaaguggccuugguacauuuggcucggguucauugcggacuuaugcgauguaaugguacaaucaug
augauucugaaccagugcuuaaaggcgugaagcuccacuauaccuga
SEQ ID NO: 9
(A.1 Delta V5 Amino Acid Sequence)
MFLLTTKRTMFVFLVLLPLVSSQCVNLRTRTQLPPAYTNSFTRGVYYPDKVFRSSVLHSTQDLFLPFFSNVTWFHA
IHVSGTNGTKRFDNPVLPFNDGVYFASIEKSNIIRGWIFGTTLDSKTQSLLIVNNATNVVIKVCEFQFCNDPFLDV
YYHKNNKSWMESGVYSSANNCTFEYVSQPFLMDLEGKQGNFKNLREFVFKNIDGYFKIYSKHTPINLVRDLPQGFS
ALEPLVDLPIGINITRFQTLLALHRSYLTPGDSSSGLTAGAAAYYVGYLQPRTFLLKYNENGTITDAVDCALDPLS
ETKCTLKSFTVEKGIYQTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGNIADYNYKLPDDFTGCVIAWNSNNLDSKVGGNY
NYRYRLFRKSNLKPFERDISTEIYQAGSKPCNGVEGENCYFPLQSYGFQPTNGVGYQPYRVVVLSFELLHAPATVC
GPKKSTNLVKNKCVNFNFNGLTGTGVLTESNKNFLPFQQFGRDIADTTDAVRDPQTLEILDITPCSFGGVSVITPG
TNTSNQVAVLYQGVNCTEVPVAIHADQLTPTWRVYSTGSNVFQTRAGCLIGAEHVNNSYECDIPIGAGICASYQTQ
TNSRRRARSVASQSIIAYTMSLGAENSVAYSNNSIAIPTNFTISVTTEILPVSMTKTSVDCTMYICGDSTECSNLL
LQYGSFCTQLNRALTGIAVEQDKNTQEVFAQVKQIYKTPPIKDFGGFNFSQILPDPSKPSKRSFIEDLLFNKVTLA
DAGFIKQYGDCLGDIAARDLICAQKFNGLTVLPPLLTDEMIAQYTSALLAGTITSGWTFGAGAALQIPFAMQMAYR
FNGIGVTQNVLYENQKLIANQFNSAIGKIQDSLSSTASALGKLQNVVNQNAQALNTLVKQLSSNFGAISSVLNDIL
SRLDPPEAEVQIDRLITGRLQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQIITTDNTFVSGNCDVVIGIV
NNTVYDPLQPELDSFKEELDKYFKNHTSPDVDLGDISGINASVVNIQKEIDRLNEVAKNLNESLIDLQELGKYEQY
IKWPWYIWLGFIAGLIAIVMVTIMLCCMTSCCSCLKGCCSCGSCCKFDEDDSEPVLKGVKLHYT
SEQ ID NO: 10
(A.1-preF K995P-V996P Amino Acid Sequence)
MFLLTTKRTMFVFLVLLPLVSSQCVNLRTRTQLPPAYTNSFTRGVYYPDKVFRSSVLHSTQDLFLPFFSNVTWFHA
IHVSGTNGTKRFDNPVLPFNDGVYFASIEKSNIIRGWIFGTTLDSKTQSLLIVNNATNVVIKVCEFQFCNDPFLDV
YYHKNNKSWMESGVYSSANNCTFEYVSQPFLMDLEGKQGNFKNLREFVFKNIDGYFKIYSKHTPINLVRDLPQGFS
ALEPLVDLPIGINITRFQTLLALHRSYLTPGDSSSGLTAGAAAYYVGYLQPRTFLLKYNENGTITDAVDCALDPLS
ETKCTLKSFTVEKGIYQTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGNIADYNYKLPDDFTGCVIAWNSNNLDSKVGGNY
NYRYRLFRKSNLKPFERDISTEIYQAGSKPCNGVEGFNCYFPLQSYGFQPTNGVGYQPYRVVVLSFELLHAPATVC
GPKKSTNLVKNKCVNFNFNGLTGTGVLTESNKNFLPFQQFGRDIADTTDAVRDPQTLEILDITPCSFGGVSVITPG
TNTSNQVAVLYQGVNCTEVPVAIHADQLTPTWRVYSTGSNVFQTRAGCLIGAEHVNNSYECDIPIGAGICASYQTQ
TNSRRRARSVASQSIIAYTMSLGAENSVAYSNNSIAIPTNFTISVTTEILPVSMTKTSVDCTMYICGDSTECSNLL
LQYGSFCTQLNRALTGIAVEQDKNTQEVFAQVKQIYKTPPIKDFGGFNFSQILPDPSKPSKRSFIEDLLFNKVTLA
DAGFIKQYGDCLGDIAARDLICAQKFNGLTVLPPLLTDEMIAQYTSALLAGTITSGWTFGAGAALQIPFAMQMAYR
FNGIGVTQNVLYENQKLIANQFNSAIGKIQDSLSSTASALGKLQNVVNQNAQALNTLVKQLSSNFGAISSVLNDIL
SRLDPPEAEVQIDRLITGRLQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQIITTDNTFVSGNCDVVIGIV
NNTVYDPLQPELDSFKEELDKYFKNHTSPDVDLGDISGINASVVNIQKEIDRLNEVAKNLNESLIDLQELGKYEQY
IKWPWYIWLGFIAGLIAIVMVTIMLCCMTSCCSCLKGCCSCGSCCKFDEDDSEPVLKGVKLHYT
SEQ ID NO: 11
(B.1 D614G Amino Acid Sequence)
MFLLTTKRTMFVFLVLLPLVSSQCVNLRTRTQLPPAYTNSFTRGVYYPDKVFRSSVLHSTQDLFLPFFSNVTWFHA
IHVSGTNGTKRFDNPVLPFNDGVYFASIEKSNIIRGWIFGTTLDSKTQSLLIVNNATNVVIKVCEFQFCNDPFLDV
YYHKNNKSWMESGVYSSANNCTFEYVSQPFLMDLEGKQGNFKNLREFVFKNIDGYFKIYSKHTPINLVRDLPQGFS
ALEPLVDLPIGINITRFQTLLALHRSYLTPGDSSSGLTAGAAAYYVGYLQPRTFLLKYNENGTITDAVDCALDPLS
ETKCTLKSFTVEKGIYQTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGNIADYNYKLPDDFTGCVIAWNSNNLDSKVGGNY
NYRYRLFRKSNLKPFERDISTEIYQAGSKPCNGVEGFNCYFPLQSYGFQPTNGVGYQPYRVVVLSFELLHAPATVC
GPKKSTNLVKNKCVNFNFNGLTGTGVLTESNKNFLPFQQFGRDIADTTDAVRDPQTLEILDITPCSFGGVSVITPG
TNTSNQVAVLYQGVNCTEVPVAIHADQLTPTWRVYSTGSNVFQTRAGCLIGAEHVNNSYECDIPIGAGICASYQTQ
TNSRRRARSVASQSIIAYTMSLGAENSVAYSNNSIAIPTNFTISVTTEILPVSMTKTSVDCTMYICGDSTECSNLL
LQYGSFCTQLNRALTGIAVEQDKNTQEVFAQVKQIYKTPPIKDFGGFNFSQILPDPSKPSKRSFIEDLLFNKVTLA
DAGFIKQYGDCLGDIAARDLICAQKFNGLTVLPPLLTDEMIAQYTSALLAGTITSGWTFGAGAALQIPFAMQMAYR
FNGIGVTQNVLYENQKLIANQFNSAIGKIQDSLSSTASALGKLQNVVNQNAQALNTLVKQLSSNFGAISSVLNDIL
SRLDPPEAEVQIDRLITGRLQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQIITTDNTFVSGNCDVVIGIV
NNTVYDPLQPELDSFKEELDKYFKNHTSPDVDLGDISGINASVVNIQKEIDRLNEVAKNLNESLIDLQELGKYEQY
IKWPWYIWLGFIAGLIAIVMVTIMLCCMTSCCSCLKGCCSCGSCCKFDEDDSEPVLKGVKLHYT
SEQ ID NO: 12
(Beta-preF B.1.351-PP-D614G Amino Acid Sequence)
MFLLTTKRTMFVFLVLLPLVSSQCVNFTTRTQLPPAYTNSFTRGVYYPDKVFRSSVLHSTQDLFLPFFSNVTWFHA
IHVSGTNGTKRFANPVLPFNDGVYFASTEKSNIIRGWIFGTTLDSKTQSLLIVNNATNVVIKVCEFQFCNDPFLGV
YYHKNNKSWMESEFRVYSSANNCTFEYVSQPFLMDLEGKQGNFKNLREFVFKNIDGYFKIYSKHTPINLVRGLPQG
FSALEPLVDLPIGINITRFQTLHISYLTPGDSSSGWTAGAAAYYVGYLQPRTFLLKYNENGTITDAVDCALDPLSE
TKCTLKSFTVEKGIYQTSNFRVQPTESIVRFPNITNLCPFGEVFNATRFASVYAWNRKRISNCVADYSVLYNSASF
STFKCYGVSPTKLNDLCFTNVYADSFVIRGDEVRQIAPGQTGNIADYNYKLPDDFTGCVIAWNSNNLDSKVGGNYN
YLYRLFRKSNLKPFERDISTEIYQAGSTPCNGVKGFNCYFPLQSYGFQPTYGVGYQPYRVVVLSFELLHAPATVCG
PKKSTNLVKNKCVNFNFNGLTGTGVLTESNKKFLPFQQFGRDIADTTDAVRDPQTLEILDITPCSFGGVSVITPGT
NTSNQVAVLYQGVNCTEVPVAIHADQLTPTWRVYSTGSNVFQTRAGCLIGAEHVNNSYECDIPIGAGICASYQTQT
NSPRRARSVASQSIIAYTMSLGVENSVAYSNNSIAIPTNFTISVTTEILPVSMTKTSVDCTMYICGDSTECSNLLL
QYGSFCTQLNRALTGIAVEQDKNTQEVFAQVKQIYKTPPIKDFGGFNFSQILPDPSKPSKRSFIEDLLFNKVTLAD
AGFIKQYGDCLGDIAARDLICAQKFNGLTVLPPLLTDEMIAQYTSALLAGTITSGWTFGAGAALQIPFAMQMAYRF
NGIGVTQNVLYENQKLIANQFNSAIGKIQDSLSSTASALGKLQDVVNQNAQALNTLVKQLSSNFGAISSVLNDILS
RLDPPEAEVQIDRLITGRLQSLQTYVTQQLIRAAEIRASANLAATKMSECVLGQSKRVDFCGKGYHLMSFPQSAPH
GVVFLHVTYVPAQEKNFTTAPAICHDGKAHFPREGVFVSNGTHWFVTQRNFYEPQIITTDNTFVSGNCDVVIGIVN
NTVYDPLQPELDSFKEELDKYFKNHTSPDVDLGDISGINASVVNIQKEIDRLNEVAKNLNESLIDLQELGKYEQYI
KWPWYIWLGFIAGLIAIVMVTIMLCCMTSCCSCLKGCCSCGSCCKFDEDDSEPVLKGVKLHYT
SEQ ID NO: 13
(Alpha-preF B.1.1.7-PP-D614G Amino Acid Sequence)
MFLLTTKRTMFVFLVLLPLVSSQCVNLTTRTQLPPAYTNSFTRGVYYPDKVFRSSVLHSTQDLFLPFFSNVTWFHA
IHVSGTNGTKRFDNPVLPFNDGVYFASTEKSNIIRGWIFGTTLDSKTQSLLIVNNATNVVIKVCEFQFCNDPFLGV
YYHKNNKSWMESEFRVYSSANNCTFEYVSQPFLMDLEGKQGNFKNLREFVFKNIDGYFKIYSKHTPINLVRDLPQG
FSALEPLVDLPIGINITRFQTLLALHRSYLTPGDSSSGWTAGAAAYYVGYLQPRTFLLKYNENGTITDAVDCALDP
LSETKCTLKSFTVEKGIYQTSNFRVQPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYNYKLPDDFTGCVIAWNSNNLDSKVGG
NYNYLYRLFRKSNLKPFERDISTEIYQAGSTPCNGVEGFNCYFPLQSYGFQPTNGVGYQPYRVVVLSFELLHAPAT
VCGPKKSTNLVKNKCVNFNFNGLTGTGVLTESNKKFLPFQQFGRDIADTTDAVRDPQTLEILDITPCSFGGVSVIT
PGTNTSNQVAVLYQDVNCTEVPVAIHAGQLTPTWRVYSTGSNVFQTRAGCLIGAEHVNNSYECDIPIGAGICASYQ
TQTNSPRRARSVASQSIIAYTMSLGAENSVAYSNNSIAIPTNFTISVTTEILPVSMTKTSVDCTMYICGDSTECSN
LLLQYGSFCTQLNRALTGIAVEQDKNTQEVFAQVKQIYKTPPIKDFGGFNFSQILPDPSKPSKRSFIEDLLFNKVT
LADAGFIKQYGDCLGDIAARDLICAQKFNGLTVLPPLLTDEMIAQYTSALLAGTITSGWTFGAGAALQIPFAMQMA
YRFNGIGVTQNVLYENQKLIANQFNSAIGKIQDSLSSTASALGKLQDVVNQNAQALNTLVKQLSSNFGAISSVLND
ILSRLDKVEAEVQIDRLITGRLQSLQTYVTQQLIRAAEIRASANLAATKMSECVLGQSKRVDFCGKGYHLMSFPQS
APHGVVFLHVTYVPAQEKNFTTAPAICHDGKAHFPREGVFVSNGTHWFVTQRNFYEPQIITTDNTFVSGNCDVVIG
IVNNTVYDPLQPELDSFKEELDKYFKNHTSPDVDLGDISGINASVVNIQKEIDRLNEVAKNLNESLIDLQELGKYE
QYIKWPWYIWLGFIAGLIAIVMVTIMLCCMTSCCSCLKGCCSCGSCCKFDEDDSEPVLKGVKLHYT
SEQ ID NO: 14
(Delta-preF AY1-S2P-wtFur Amino Acid Sequence)
MFLLTTKRTMFVFLVLLPLVSSQCVNLTTRTQLPPAYTNSFTRGVYYPDKVFRSSVLHSTQDLFLPFFSNVTWFHA
IHVSGTNGTKRFDNPVLPFNDGVYFASTEKSNIIRGWIFGTTLDSKTQSLLIVNNATNVVIKVCEFQFCNDPFLGV
YYHKNNKSWMESEFRVYSSANNCTFEYVSQPFLMDLEGKQGNFKNLREFVFKNIDGYFKIYSKHTPINLVRDLPQG
FSALEPLVDLPIGINITRFQTLLALHRSYLTPGDSSSGWTAGAAAYYVGYLQPRTFLLKYNENGTITDAVDCALDP
LSETKCTLKSFTVEKGIYQTSNFRVQPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYNYKLPDDFTGCVIAWNSNNLDSKVGG
NYNYLYRLFRKSNLKPFERDISTEIYQAGSTPCNGVEGFNCYFPLQSYGFQPTNGVGYQPYRVVVLSFELLHAPAT
VCGPKKSTNLVKNKCVNFNFNGLTGTGVLTESNKKFLPFQQFGRDIADTTDAVRDPQTLEILDITPCSFGGVSVIT
PGTNTSNQVAVLYQDVNCTEVPVAIHADQLTPTWRVYSTGSNVFQTRAGCLIGAEHVNNSYECDIPIGAGICASYQ
TQTNSPRRARSVASQSIIAYTMSLGAENSVAYSNNSIAIPTNFTISVTTEILPVSMTKTSVDCTMYICGDSTECSN
LLLQYGSFCTQLNRALTGIAVEQDKNTQEVFAQVKQIYKTPPIKDFGGFNFSQILPDPSKPSKRSFIEDLLFNKVT
LADAGFIKQYGDCLGDIAARDLICAQKFNGLTVLPPLLTDEMIAQYTSALLAGTITSGWTFGAGAALQIPFAMQMA
YRFNGIGVTQNVLYENQKLIANQFNSAIGKIQDSLSSTASALGKLQDVVNQNAQALNTLVKQLSSNFGAISSVLND
ILSRLDPPEAEVQIDRLITGRLQSLQTYVTQQLIRAAEIRASANLAATKMSECVLGQSKRVDFCGKGYHLMSFPQS
APHGVVFLHVTYVPAQEKNFTTAPAICHDGKAHFPREGVFVSNGTHWFVTQRNFYEPQIITTDNTFVSGNCDVVIG
IVNNTVYDPLQPELDSFKEELDKYFKNHTSPDVDLGDISGINASVVNIQKEIDRLNEVAKNLNESLIDLQELGKYE
QYIKWPWYIWLGFIAGLIAIVMVTIMLCCMTSCCSCLKGCCSCGSCCKFDEDDSEPVLKGVKLHYT
SEQ ID NO: 15
(Delta-preF-AY1-S2P-wtFur-newKozak Amino Acid Sequence)
MFLLTTKRTMFVFLVLLPLVSSQCVNLTTRTQLPPAYTNSFTRGVYYPDKVFRSSVLHSTQDLFLPFFSNVTWFHA
IHVSGTNGTKRFDNPVLPFNDGVYFASTEKSNIIRGWIFGTTLDSKTQSLLIVNNATNVVIKVCEFQFCNDPFLGV
YYHKNNKSWMESEFRVYSSANNCTFEYVSQPFLMDLEGKQGNFKNLREFVFKNIDGYFKIYSKHTPINLVRDLPQG
FSALEPLVDLPIGINITRFQTLLALHRSYLTPGDSSSGWTAGAAAYYVGYLQPRTFLLKYNENGTITDAVDCALDP
LSETKCTLKSFTVEKGIYQTSNFRVQPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYNYKLPDDFTGCVIAWNSNNLDSKVGG
NYNYLYRLFRKSNLKPFERDISTEIYQAGSTPCNGVEGFNCYFPLQSYGFQPTNGVGYQPYRVVVLSFELLHAPAT
VCGPKKSTNLVKNKCVNFNFNGLTGTGVLTESNKKFLPFQQFGRDIADTTDAVRDPQTLEILDITPCSFGGVSVIT
PGTNTSNQVAVLYQDVNCTEVPVAIHADQLTPTWRVYSTGSNVFQTRAGCLIGAEHVNNSYECDIPIGAGICASYQ
TQTNSPRRARSVASQSIIAYTMSLGAENSVAYSNNSIAIPTNFTISVTTEILPVSMTKTSVDCTMYICGDSTECSN
LLLQYGSFCTQLNRALTGIAVEQDKNTQEVFAQVKQIYKTPPIKDFGGFNFSQILPDPSKPSKRSFIEDLLFNKVT
LADAGFIKQYGDCLGDIAARDLICAQKFNGLTVLPPLLTDEMIAQYTSALLAGTITSGWTFGAGAALQIPFAMQMA
YRFNGIGVTQNVLYENQKLIANQFNSAIGKIQDSLSSTASALGKLQDVVNQNAQALNTLVKQLSSNFGAISSVLND
ILSRLDKVEAEVQIDRLITGRLQSLQTYVTQQLIRAAEIRASANLAATKMSECVLGQSKRVDFCGKGYHLMSFPQS
APHGVVFLHVTYVPAQEKNFTTAPAICHDGKAHFPREGVFVSNGTHWFVTQRNFYEPQIITTDNTFVSGNCDVVIG
IVNNTVYDPLQPELDSFKEELDKYFKNHTSPDVDLGDISGINASVVNIQKEIDRLNEVAKNLNESLIDLQELGKYE
QYIKWPWYIWLGFIAGLIAIVMVTIMLCCMTSCCSCLKGCCSCG
SEQ ID NO: 16
(SARS-COV-2-Omicron B.1.1.529 Amino Acid Sequence)
MFLLTTKRTMFVFLVLLPLVSSQCVNLTTRTQLPPAYTNSFTRGVYYPDKVFRSSVLHSTQDLFLPFFSNVTWFHV
ISGTNGTKRFDNPVLPFNDGVYFASIEKSNIIRGWIFGTTLDSKTQSLLIVNNATNVVIKVCEFQFCNDPFFDHKN
NKSWMESEFRVYSSANNCTFEYVSQPFLMDLEGKQGNFKNLREFVFKNIDGYFKIYSKHTPIIVREPEDLPQGFSA
LEPLVDLPIGINITRFQTLLALHRSYLTPGDSSSGWTAGAAAYYVGYLQPRTFLLKYNENGTITDAVDCALDPLSE
TKCTLKSFTVEKGIYQTSNFRVQPTESIVRFPNITNLCPFDEVFNATRFASVYAWNRKRISNCVADYSVLYNLAPF
FTFKCYGVSPTKLNDLCFTNVYADSFVIRGDEVRQIAPGQTGNIADYNYKLPDDFTGCVIAWNSNKLDSKVSGNYN
YLYRLFRKSNLKPFERDISTEIYQAGNKPCNGVAGFNCYFPLRSYSFRPTYGVGHQPYRVVVLSFELLHAPATVCG
PKKSTNLVKNKCVNFNFNGLKGTGVLTESNKKFLPFQQFGRDIADTTDAVRDPQTLEILDITPCSFGGVSVITPGT
NTSNQVAVLYQGVNCTEVPVAIHADQLTPTWRVYSTGSNVFQTRAGCLIGAEYVNNSYECDIPIGAGICASYQTQT
KSHRRARSVASQSIIAYTMSLGAENSVAYSNNSIAIPTNFTISVTTEILPVSMTKTSVDCTMYICGDSTECSNLLL
QYGSFCTQLKRALTGIAVEQDKNTQEVFAQVKQIYKTPPIKYFGGFNFSQILPDPSKPSKRSFIEDLLFNKVTLAD
AGFIKQYGDCLGDIAARDLICAQKFKGLTVLPPLLTDEMIAQYTSALLAGTITSGWTFGAGAALQIPFAMQMAYRF
NGIGVTQNVLYENQKLIANQFNSAIGKIQDSLSSTASALGKLQDVVNHNAQALNTLVKQLSSKFGAISSVLNDIFS
RLDPPEAEVQIDRLITGRLQSLQTYVTQQLIRAAEIRASANLAATKMSECVLGQSKRVDFCGKGYHLMSFPQSAPH
GVVFLHVTYVPAQEKNFTTAPAICHDGKAHFPREGVFVSNGTHWFVTQRNFYEPQIITTDNTFVSGNCDVVIGIVN
NTVYDPLQPELDSFKEELDKYFKNHTSPDVDLGDISGINASVVNIQKEIDRLNEVAKNLNESLIDLQELGKYEQYI
KWPWYIWLGFIAGLIAIVMVTIMLCCMTSCCSCLKGCCSCGSCCKFDEDDSEPVLKGVKLHYT
SEQ ID NO: 17
(Wuhan-Hu-1-Full-Length Wild-Type Spike
Protein Amino Acid Sequence-NCBI Reference ID: PODTC2
>YP_009724390.1 surface glycoprotein
[Severe acute respiratory syndrome coronavirus 2]
MFVFLVLLPLVSSQCVNLTTRTQLPPAYTNSFTRGVYYPDKVFRSSVLHSTQDLFLPFFSNVTWFHAIHVSGTNGT
KRFDNPVLPFNDGVYFASTEKSNIIRGWIFGTTLDSKTQSLLIVNNATNVVIKVCEFQFCNDPFLGVYYHKNNKSW
MESEFRVYSSANNCTFEYVSQPFLMDLEGKQGNFKNLREFVFKNIDGYFKIYSKHTPINLVRDLPQGFSALEPLVD
LPIGINITRFQTLLALHRSYLTPGDSSSGWTAGAAAYYVGYLQPRTFLLKYNENGTITDAVDCALDPLSETKCTLK
SFTVEKGIYQTSNFRVQPTESIVRFPNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNLDSKVGGNYNYLYRLF
RKSNLKPFERDISTEIYQAGSTPCNGVEGFNCYFPLQSYGFQPTNGVGYQPYRVVVLSFELLHAPATVCGPKKSTN
LVKNKCVNFNFNGLTGTGVLTESNKKFLPFQQFGRDIADTTDAVRDPQTLEILDITPCSFGGVSVITPGTNTSNQV
AVLYQDVNCTEVPVAIHADQLTPTWRVYSTGSNVFQTRAGCLIGAEHVNNSYECDIPIGAGICASYQTQTNSPRRA
RSVASQSIIAYTMSLGAENSVAYSNNSIAIPTNFTISVTTEILPVSMTKTSVDCTMYICGDSTECSNLLLQYGSFC
TQLNRALTGIAVEQDKNTQEVFAQVKQIYKTPPIKDFGGFNFSQILPDPSKPSKRSFIEDLLFNKVTLADAGFIKQ
YGDCLGDIAARDLICAQKFNGLTVLPPLLTDEMIAQYTSALLAGTITSGWTFGAGAALQIPFAMQMAYRFNGIGVT
QNVLYENQKLIANQFNSAIGKIQDSLSSTASALGKLQDVVNQNAQALNTLVKQLSSNFGAISSVLNDILSRLDKVE
AEVQIDRLITGRLQSLQTYVTQQLIRAAEIRASANLAATKMSECVLGQSKRVDFCGKGYHLMSFPQSAPHGVVFLH
VTYVPAQEKNFTTAPAICHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDLQELGKYEQYIKWPWYI
WLGFIAGLIAIVMVTIMLCCMTSCCSCLKGCCSCGSCCKFDEDDSEPVLKGVKLHYT
SEQ ID NO: 18
(SARS-COV-2 Wuhan-Hu-1-Wild-Type Spike Protein-
Stem Helix Amino Acid Sequence
[residues 913 to 1213])
QNVLYENQKLIANQFNSAIGKIQDSLSSTASALGKLQDVVNQNAQALNTLVKQLSSNFGAISSVLNDILSRLDKVE
AEVQIDRLITGRLQSLQTYVTQQLIRAAEIRASANLAATKMSECVLGQSKRVDFCGKGYHLMSFPQSAPHGVVFLH
VTYVPAQEKNFTTAPAICHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDLQELGKYEQYIKWPW
SEQ ID NO: 19
(SARS-COV-2-Omicron Spike Protein Stem Helix Amino Acid Sequence)-
bold indicate stem mutations associated with the omicron variant
QNVLYENQKLIANQFNSAIGKIQDSLSSTASALGKLQDVVNHNAQALNTLVKQLSSKFGAISSVLNDIFSRLDPPE
AEVQIDRLITGRLQSLQTYVTQQLIRAAEIRASANLAATKMSECVLGQSKRVDFCGKGYHLMSFPQSAPHGVVFLH
VTYVPAQEKNFTTAPAICHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDLQELGKYEQYIKWPW
SEQ ID NO: 20:
VEEV RNA Sequence
AUAGGCGGCGCAUGAGAGAAGCCCAGACCAAUUACCUACCCAAAAUGGAGAAAGUUCACGUUGACAUCGAGGAAGA
CAGCCCAUUCCUCAGAGCUUUGCAGCGGAGCUUCCCGCAGUUUGAGGUAGAAGCCAAGCAGGUCACUGAUAAUGAC
CAUGCUAAUGCCAGAGCGUUUUCGCAUCUGGCUUCAAAACUGAUCGAAACGGAGGUGGACCCAUCCGACACGAUCC
UUGACAUUGGAAGUGCGCCCGCCCGCAGAAUGUAUUCUAAGCACAAGUAUCAUUGUAUCUGUCCGAUGAGAUGUGC
GGAAGAUCCGGACAGAUUGUAUAAGUAUGCAACUAAGCUGAAGAAAAACUGUAAGGAAAUAACUGAUAAGGAAUUG
GACAAGAAAAUGAAGGAGCUGGCCGCCGUCAUGAGCGACCCUGACCUGGAAACUGAGACUAUGUGCCUCCACGACG
ACGAGUCGUGUCGCUACGAAGGGCAAGUCGCUGUUUACCAGGAUGUAUACGCGGUUGACGGACCGACAAGUCUCUA
UCACCAAGCCAAUAAGGGAGUUAGAGUCGCCUACUGGAUAGGCUUUGACACCACCCCUUUUAUGUUUAAGAACUUG
GCUGGAGCAUAUCCAUCAUACUCUACCAACUGGGCCGACGAAACCGUGUUAACGGCUCGUAACAUAGGCCUAUGCA
GCUCUGACGUUAUGGAGCGGUCACGUAGAGGGAUGUCCAUUCUUAGAAAGAAGUAUUUGAAACCAUCCAACAAUGU
UCUAUUCUCUGUUGGCUCGACCAUCUACCACGAGAAGAGGGACUUACUGAGGAGCUGGCACCUGCCGUCUGUAUUU
CACUUACGUGGCAAGCAAAAUUACACAUGUCGGUGUGAGACUAUAGUUAGUUGCGACGGGUACGUCGUUAAAAGAA
UAGCUAUCAGUCCAGGCCUGUAUGGGAAGCCUUCAGGCUAUGCUGCUACGAUGCACCGCGAGGGAUUCUUGUGCUG
CAAAGUGACAGACACAUUGAACGGGGAGAGGGUCUCUUUUCCCGUGUGCACGUAUGUGCCAGCUACAUUGUGUGAC
CAAAUGACUGGCAUACUGGCAACAGAUGUCAGUGCGGACGACGCGCAAAAACUGCUGGUUGGGCUCAACCAGCGUA
UAGUCGUCAACGGUCGCACCCAGAGAAACACCAAUACCAUGAAAAAUUACCUUUUGCCCGUAGUGGCCCAGGCAUU
UGCUAGGUGGGCAAAGGAAUAUAAGGAAGAUCAAGAAGAUGAAAGGCCACUAGGACUACGAGAUAGACAGUUAGUC
AUGGGGUGUUGUUGGGCUUUUAGAAGGCACAAGAUAACAUCUAUUUAUAAGCGCCCGGAUACCCAAACCAUCAUCA
AAGUGAACAGCGAUUUCCACUCAUUCGUGCUGCCCAGGAUAGGCAGUAACACAUUGGAGAUCGGGCUGAGAACAAG
AAUCAGGAAAAUGUUAGAGGAGCACAAGGAGCCGUCACCUCUCAUUACCGCCGAGGACGUACAAGAAGCUAAGUGC
GCAGCCGAUGAGGCUAAGGAGGUGCGUGAAGCCGAGGAGUUGCGCGCAGCUCUACCACCUUUGGCAGCUGAUGUUG
AGGAGCCCACUCUGGAGGCAGACGUCGACUUGAUGUUACAAGAGGCUGGGGCCGGCUCAGUGGAGACACCUCGUGG
CUUGAUAAAGGUUACCAGCUACGAUGGCGAGGACAAGAUCGGCUCUUACGCUGUGCUUUCUCCGCAGGCUGUACUC
AAGAGUGAAAAAUUAUCUUGCAUCCACCCUCUCGCUGAACAAGUCAUAGUGAUAACACACUCUGGCCGAAAAGGGC
GUUAUGCCGUGGAACCAUACCAUGGUAAAGUAGUGGUGCCAGAGGGACAUGCAAUACCCGUCCAGGACUUUCAAGC
UCUGAGUGAAAGUGCCACCAUUGUGUACAACGAACGUGAGUUCGUAAACAGGUACCUGCACCAUAUUGCCACACAU
GGAGGAGCGCUGAACACUGAUGAAGAAUAUUACAAAACUGUCAAGCCCAGCGAGCACGACGGCGAAUACCUGUACG
ACAUCGACAGGAAACAGUGCGUCAAGAAAGAACUAGUCACUGGGCUAGGGCUCACAGGCGAGCUGGUGGAUCCUCC
CUUCCAUGAAUUCGCCUACGAGAGUCUGAGAACACGACCAGCCGCUCCUUACCAAGUACCAACCAUAGGGGUGUAU
GGCGUGCCAGGAUCAGGCAAGUCUGGCAUCAUUAAAAGCGCAGUCACCAAAAAAGAUCUAGUGGUGAGCGCCAAGA
AAGAAAACUGUGCAGAAAUUAUAAGGGACGUCAAGAAAAUGAAAGGGCUGGACGUCAAUGCCAGAACUGUGGACUC
AGUGCUCUUGAAUGGAUGCAAACACCCCGUAGAGACCCUGUAUAUUGACGAAGCUUUUGCUUGUCAUGCAGGUACU
CUCAGAGCGCUCAUAGCCAUUAUAAGACCUAAAAAGGCAGUGCUCUGCGGGGAUCCCAAACAGUGCGGUUUUUUUA
ACAUGAUGUGCCUGAAAGUGCAUUUUAACCACGAGAUUUGCACACAAGUCUUCCACAAAAGCAUCUCUCGCCGUUG
CACUAAAUCUGUGACUUCGGUCGUCUCAACCUUGUUUUACGACAAAAAAAUGAGAACGACGAAUCCGAAAGAGACU
AAGAUUGUGAUUGACACUACCGGCAGUACCAAACCUAAGCAGGACGAUCUCAUUCUCACUUGUUUCAGAGGGUGGG
UGAAGCAGUUGCAAAUAGAUUACAAAGGCAACGAAAUAAUGACGGCAGCUGCCUCUCAAGGGCUGACCCGUAAAGG
UGUGUAUGCCGUUCGGUACAAGGUGAAUGAAAAUCCUCUGUACGCACCCACCUCAGAACAUGUGAACGUCCUACUG
ACCCGCACGGAGGACCGCAUCGUGUGGAAAACACUAGCCGGCGACCCAUGGAUAAAAACACUGACUGCCAAGUACC
CUGGGAAUUUCACUGCCACGAUAGAGGAGUGGCAAGCAGAGCAUGAUGCCAUCAUGAGGCACAUCUUGGAGAGACC
GGACCCUACCGACGUCUUCCAGAAUAAGGCAAACGUGUGUUGGGCCAAGGCUUUAGUGCCGGUGCUGAAGACCGCU
GGCAUAGACAUGACCACUGAACAAUGGAACACUGUGGAUUAUUUUGAAACGGACAAAGCUCACUCAGCAGAGAUAG
UAUUGAACCAACUAUGCGUGAGGUUCUUUGGACUCGAUCUGGACUCCGGUCUAUUUUCUGCACCCACUGUUCCGUU
AUCCAUUAGGAAUAAUCACUGGGAUAACUCCCCGUCGCCUAACAUGUACGGGCUGAAUAAAGAAGUGGUCCGUCAG
CUCUCUCGCAGGUACCCACAACUGCCUCGGGCAGUUGCCACUGGAAGAGUCUAUGACAUGAACACUGGUACACUGC
GCAAUUAUGAUCCGCGCAUAAACCUAGUACCUGUAAACAGAAGACUGCCUCAUGCUUUAGUCCUCCACCAUAAUGA
ACACCCACAGAGUGACUUUUCUUCAUUCGUCAGCAAAUUGAAGGGCAGAACUGUCCUGGUGGUCGGGGAAAAGUUG
UCCGUCCCAGGCAAAAUGGUUGACUGGUUGUCAGACCGGCCUGAGGCUACCUUCAGAGCUCGGCUGGAUUUAGGCA
UCCCAGGUGAUGUGCCCAAAUAUGACAUAAUAUUUGUUAAUGUGAGGACCCCAUAUAAAUACCAUCACUAUCAGCA
GUGUGAAGACCAUGCCAUUAAGCUUAGCAUGUUGACCAAGAAAGCUUGUCUGCAUCUGAAUCCCGGCGGAACCUGU
GUCAGCAUAGGUUAUGGUUACGCUGACAGGGCCAGCGAAAGCAUCAUUGGUGCUAUAGCGCGGCAGUUCAAGUUUU
CCCGGGUAUGCAAACCGAAAUCCUCACUUGAAGAGACGGAAGUUCUGUUUGUAUUCAUUGGGUACGAUCGCAAGGC
CCGUACGCACAAUCCUUACAAGCUUUCAUCAACCUUGACCAACAUUUAUACAGGUUCCAGACUCCACGAAGCCGGA
UGUGCACCCUCAUAUCAUGUGGUGCGAGGGGAUAUUGCCACGGCCACCGAAGGAGUGAUUAUAAAUGCUGCUAACA
GCAAAGGACAACCUGGCGGAGGGGUGUGCGGAGCGCUGUAUAAGAAAUUCCCGGAAAGCUUCGAUUUACAGCCGAU
CGAAGUAGGAAAAGCGCGACUGGUCAAAGGUGCAGCUAAACAUAUCAUUCAUGCCGUAGGACCAAACUUCAACAAA
GUUUCGGAGGUUGAAGGUGACAAACAGUUGGCAGAGGCUUAUGAGUCCAUCGCUAAGAUUGUCAACGAUAACAAUU
ACAAGUCAGUAGCGAUUCCACUGUUGUCCACCGGCAUCUUUUCCGGGAACAAAGAUCGACUAACCCAAUCAUUGAA
CCAUUUGCUGACAGCUUUAGACACCACUGAUGCAGAUGUAGCCAUAUACUGCAGGGACAAGAAAUGGGAAAUGACU
CUCAAGGAAGCAGUGGCUAGGAGAGAAGCAGUGGAGGAGAUAUGCAUAUCCGACGACUCUUCAGUGACAGAACCUG
AUGCAGAGCUGGUGAGGGUGCAUCCGAAGAGUUCUUUGGCUGGAAGGAAGGGCUACAGCACAAGCGAUGGCAAAAC
UUUCUCAUAUUUGGAAGGGACCAAGUUUCACCAGGCGGCCAAGGAUAUAGCAGAAAUUAAUGCCAUGUGGCCCGUU
GCAACGGAGGCCAAUGAGCAGGUAUGCAUGUAUAUCCUCGGAGAAAGCAUGAGCAGUAUUAGGUCGAAAUGCCCCG
UCGAAGAGUCGGAAGCCUCCACACCACCUAGCACGCUGCCUUGCUUGUGCAUCCAUGCCAUGACUCCAGAAAGAGU
ACAGCGCCUAAAAGCCUCACGUCCAGAACAAAUUACUGUGUGCUCAUCCUUUCCAUUGCCGAAGUAUAGAAUCACU
GGUGUGCAGAAGAUCCAAUGCUCCCAGCCUAUAUUGUUCUCACCGAAAGUGCCUGCGUAUAUUCAUCCAAGGAAGU
AUCUCGUGGAAACACCACCGGUAGACGAGACUCCGGAGCCAUCGGCAGAGAACCAAUCCACAGAGGGGACACCUGA
ACAACCACCACUUAUAACCGAGGAUGAGACCAGGACUAGAACGCCUGAGCCGAUCAUCAUCGAAGAGGAAGAAGAG
GAUAGCAUAAGUUUGCUGUCAGAUGGCCCGACCCACCAGGUGCUGCAAGUCGAGGCAGACAUUCACGGGCCGCCCU
CUGUAUCUAGCUCAUCCUGGUCCAUUCCUCAUGCAUCCGACUUUGAUGUGGACAGUUUAUCCAUACUUGACACCCU
GGAGGGAGCUAGCGUGACCAGCGGGGCAACGUCAGCCGAGACUAACUCUUACUUCGCAAAGAGUAUGGAGUUUCUG
GCGCGACCGGUGCCUGCGCCUCGAACAGUAUUCAGGAACCCUCCACAUCCCGCUCCGCGCACAAGAACACCGUCAC
UUGCACCCAGCAGGGCCUGCUCGAGAACCAGCCUAGUUUCCACCCCGCCAGGCGUGAAUAGGGUGAUCACUAGAGA
GGAGCUCGAGGCGCUUACCCCGUCACGCACUCCUAGCAGGUCGGUCUCGAGAACCAGCCUGGUCUCCAACCCGCCA
GGCGUAAAUAGGGUGAUUACAAGAGAGGAGUUUGAGGCGUUCGUAGCACAACAACAAUGACGGUUUGAUGCGGGUG
CAUACAUCUUUUCCUCCGACACCGGUCAAGGGCAUUUACAACAAAAAUCAGUAAGGCAAACGGUGCUAUCCGAAGU
GGUGUUGGAGAGGACCGAAUUGGAGAUUUCGUAUGCCCCGCGCCUCGACCAAGAAAAAGAAGAAUUACUACGCAAG
AAAUUACAGUUAAAUCCCACACCUGCUAACAGAAGCAGAUACCAGUCCAGGAAGGUGGAGAACAUGAAAGCCAUAA
CAGCUAGACGUAUUCUGCAAGGCCUAGGGCAUUAUUUGAAGGCAGAAGGAAAAGUGGAGUGCUACCGAACCCUGCA
UCCUGUUCCUUUGUAUUCAUCUAGUGUGAACCGUGCCUUUUCAAGCCCCAAGGUCGCAGUGGAAGCCUGUAACGCC
AUGUUGAAAGAGAACUUUCCGACUGUGGCUUCUUACUGUAUUAUUCCAGAGUACGAUGCCUAUUUGGACAUGGUUG
ACGGAGCUUCAUGCUGCUUAGACACUGCCAGUUUUUGCCCUGCAAAGCUGCGCAGCUUUCCAAAGAAACACUCCUA
UUUGGAACCCACAAUACGAUCGGCAGUGCCUUCAGCGAUCCAGAACACGCUCCAGAACGUCCUGGCAGCUGCCACA
AAAAGAAAUUGCAAUGUCACGCAAAUGAGAGAAUUGCCCGUAUUGGAUUCGGCGGCCUUUAAUGUGGAAUGCUUCA
AGAAAUAUGCGUGUAAUAAUGAAUAUUGGGAAACGUUUAAAGAAAACCCCAUCAGGCUUACUGAAGAAAACGUGGU
AAAUUACAUUACCAAAUUAAAAGGACCAAAAGCUGCUGCUCUUUUUGCGAAGACACAUAAUUUGAAUAUGUUGCAG
GACAUACCAAUGGACAGGUUUGUAAUGGACUUAAAGAGAGACGUGAAAGUGACUCCAGGAACAAAACAUACUGAAG
AACGGCCCAAGGUACAGGUGAUCCAGGCUGCCGAUCCGCUAGCAACAGCGUAUCUGUGCGGAAUCCACCGAGAGCU
GGUUAGGAGAUUAAAUGCGGUCCUGCUUCCGAACAUUCAUACACUGUUUGAUAUGUCGGCUGAAGACUUUGACGCU
AUUAUAGCCGAGCACUUCCAGCCUGGGGAUUGUGUUCUGGAAACUGACAUCGCGUCGUUUGAUAAAAGUGAGGACG
ACGCCAUGGCUCUGACCGCGUUAAUGAUUCUGGAAGACUUAGGUGUGGACGCAGAGCUGUUGACGCUGAUUGAGGC
GGCUUUCGGCGAAAUUUCAUCAAUACAUUUGCCCACUAAAACUAAAUUUAAAUUCGGAGCCAUGAUGAAAUCUGGA
AUGUUCCUCACACUGUUUGUGAACACAGUCAUUAACAUUGUAAUCGCAAGCAGAGUGUUGAGAGAACGGCUAACCG
GAUCACCAUGUGCAGCAUUCAUUGGAGAUGACAAUAUCGUGAAAGGAGUCAAAUCGGACAAAUUAAUGGCAGACAG
GUGCGCCACCUGGUUGAAUAUGGAAGUCAAGAUUAUAGAUGCUGUGGUGGGCGAGAAAGCGCCUUAUUUCUGUGGA
GGGUUUAUUUUGUGUGACUCCGUGACCGGCACAGCGUGCCGUGUGGCAGACCCCCUAAAAAGGCUGUUUAAGCUUG
GCAAACCUCUGGCAGCAGACGAUGAACAUGAUGAUGACAGGAGAAGGGCAUUGCAUGAAGAGUCAACACGCUGGAA
CCGAGUGGGUAUUCUUUCAGAGCUGUGCAAGGCAGUAGAAUCAAGGUAUGAAACCGUAGGAACUUCCAUCAUAGUU
AUGGCCAUGACUACUCUAGCUAGCAGUGUUAAAUCAUUCAGCUACCUGAGAGGGGCCCCUAUAACUCUCUACGGCU
AACCUGAAUGGACUACGACAUAGUCUAGUCCGCCAAG
SEQ ID NO: 21-VEEV RNA polymerase Amino Acid Sequence
(NCBI Accession: AXP98866.1)
RELPVLDSAAFNVECFKKYACNNEYWETFKENPIRLTEENVVNYITKLKGP
SEQ ID NO: 22-VEEV RNA polymerase Amino Acid Sequence
(NCBI Accession: AXP98867.1)
TQMRELPVLDSAAFNVECFKKYACNNEYWETFKENPIRLTE
SEQ ID NO: 23:
Polyprotein Amino Acid Sequence [Venezuelan equine encephalitis virus]
(GenBank: ALE15116.1)
MKAITARRILQGLGHYLKAEGKVECYRTLHPVPLYSSSVNRAFSSPKVAVEACNAMLKENFPTVASYCIIPEYDAY
LDMIDGASCCLDTASFCPAKLRSFPKKHSYLEPTIRSAVPSAIQNTLQNVLAAATKRNCNVTQMRELPVLDSAAFN
VECFKKYACNNEYWKTFKENPIRLTEENVINYITKLKGPKAAALYAKTHNLNMLQDIPMDRFVMDLKRDVKVTPGT
KHTEERPKVQVIQAADPLATAYLCGIHRELVRRLNAVLLPNIHTLFDMSAEDFDAIIAEHFQPGDCVLETDIASFD
KSEDDAMALTAMMILEDLGVDAELLTLIEAAFGEISSIHLPTKTKFKFGAMMKSGMFLTLFVNTVINIVIASRVLR
ERLTGSPCAAFIGDDNIVKGVKSDKLMADRCATWLNMEVKIIDAVVGEKAPYFCGGFILCDSVTGTACRVADPLKR
LFKLGKPLAADDEHDDDRRRALHEESTRWNRVGILPELCKAVESRYETVGTSVIVMAMATLASSVKSFSYLRGAPI
TLYG

The following sequences (SEQ ID NOS: 24-31) are formatted to signify vector backbone and antigen open reading frames as follows: lower case letter signify the VEEV replicon backbone sequence; UPPER CASE letters signify spike open reading frame; bold signifies start codons; and lowercase-italicized signifies mutated codons relative to the parental Wuhan spike sequence.

Full-length VEEV+ Delta V5 Antigen Encoding RNA Sequence
SEQ ID NO: 24
auaggcggcgcaugagagaagcccagaccaauuaccuacccaaaauggagaaaguucacguugacaucgaggaagacagc
ccauuccucagagcuuugcagcggagcuucccgcaguuugagguagaagccaagcaggucacugauaaugaccaugcuaa
ugccagagcguuuucgcaucuggcuucaaaacugaucgaaacggagguggacccauccgacacgauccuugacauuggaa
gugcgcccgcccgcagaauguauucuaagcacaaguaucauuguaucuguccgaugagaugugcggaagauccggacaga
uuguauaaguaugcaacuaagcugaagaaaaacuguaaggaaauaacugauaaggaauuggacaagaaaaugaaggagcu
ggccgccgucaugagcgacccugaccuggaaacugagacuaugugccuccacgacgacgagucgugucgcuacgaagggc
aagucgcuguuuaccaggauguauacgcgguugacggaccgacaagucucuaucaccaagccaauaagggaguuagaguc
gccuacuggauaggcuuugacaccaccccuuuuauguuuaagaacuuggcuggagcauauccaucauacucuaccaacug
ggccgacgaaaccguguuaacggcucguaacauaggccuaugcagcucugacguuauggagcggucacguagagggaugu
ccauucuuagaaagaaguauuugaaaccauccaacaauguucuauucucuguuggcucgaccaucuaccacgagaagagg
gacuuacugaggagcuggcaccugccgucuguauuucacuuacguggcaagcaaaauuacacaugucggugugagacuau
aguuaguugcgacggguacgucguuaaaagaauagcuaucaguccaggccuguaugggaagccuucaggcuaugcugcua
cgaugcaccgcgagggauucuugugcugcaaagugacagacacauugaacggggagagggucucuuuucccgugugcacg
uaugugccagcuacauugugugaccaaaugacuggcauacuggcaacagaugucagugcggacgacgcgcaaaaacugcu
gguugggcucaaccagcguauagucgucaacggucgcacccagagaaacaccaauaccaugaaaaauuaccuuuugcccg
uaguggcccaggcauuugcuaggugggcaaaggaauauaaggaagaucaagaagaugaaaggccacuaggacuacgagau
agacaguuagucaugggguguuguugggcuuuuagaaggcacaagauaacaucuauuuauaagcgcccggauacccaaac
caucaucaaagugaacagcgauuuccacucauucgugcugcccaggauaggcaguaacacauuggagaucgggcugagaa
caagaaucaggaaaauguuagaggagcacaaggagccgucaccucucauuaccgccgaggacguacaagaagcuaagugc
gcagccgaugaggcuaaggaggugcgugaagccgaggaguugcgcgcagcucuaccaccuuuggcagcugauguugagga
gcccacucuggaggcagacgucgacuugauguuacaagaggcuggggccggcucaguggagacaccucguggcuugauaa
agguuaccagcuacgauggcgaggacaagaucggcucuuacgcugugcuuucuccgcaggcuguacucaagagugaaaaa
uuaucuugcauccacccucucgcugaacaagucauagugauaacacacucuggccgaaaagggcguuaugccguggaacc
auaccaugguaaaguaguggugccagagggacaugcaauacccguccaggacuuucaagcucugagugaaagugccacca
uuguguacaacgaacgugaguucguaaacagguaccugcaccauauugccacacauggaggagcgcugaacacugaugaa
gaauauuacaaaacugucaagcccagcgagcacgacggcgaauaccuguacgacaucgacaggaaacagugcgucaagaa
agaacuagucacugggcuagggcucacaggcgagcugguggauccucccuuccaugaauucgccuacgagagucugagaa
cacgaccagccgcuccuuaccaaguaccaaccauagggguguauggcgugccaggaucaggcaagucuggcaucauuaaa
agcgcagucaccaaaaaagaucuaguggugagcgccaagaaagaaaacugugcagaaauuauaagggacgucaagaaaau
gaaagggcuggacgucaaugccagaacuguggacucagugcucuugaauggaugcaaacaccccguagagacccuguaua
uugacgaagcuuuugcuugucaugcagguacucucagagcgcucauagccauuauaagaccuaaaaaggcagugcucugc
ggggaucccaaacagugcgguuuuuuuaacaugaugugccugaaagugcauuuuaaccacgagauuugcacacaagucuu
ccacaaaagcaucucucgccguugcacuaaaucugugacuucggucgucucaaccuuguuuuacgacaaaaaaaugagaa
cgacgaauccgaaagagacuaagauugugauugacacuaccggcaguaccaaaccuaagcaggacgaucucauucucacu
uguuucagagggugggugaagcaguugcaaauagauuacaaaggcaacgaaauaaugacggcagcugccucucaagggcu
gacccguaaagguguguaugccguucgguacaaggugaaugaaaauccucuguacgcacccaccucagaacaugugaacg
uccuacugacccgcacggaggaccgcaucguguggaaaacacuagccggcgacccauggauaaaaacacugacugccaag
uacccugggaauuucacugccacgauagaggaguggcaagcagagcaugaugccaucaugaggcacaucuuggagagacc
ggacccuaccgacgucuuccagaauaaggcaaacguguguugggccaaggcuuuagugccggugcugaagaccgcuggca
uagacaugaccacugaacaauggaacacuguggauuauuuugaaacggacaaagcucacucagcagagauaguauugaac
caacuaugcgugagguucuuuggacucgaucuggacuccggucuauuuucugcacccacuguuccguuauccauuaggaa
uaaucacugggauaacuccccgucgccuaacauguacgggcugaauaaagaagugguccgucagcucucucgcagguacc
cacaacugccucgggcaguugccacuggaagagucuaugacaugaacacugguacacugcgcaauuaugauccgcgcaua
aaccuaguaccuguaaacagaagacugccucaugcuuuaguccuccaccauaaugaacacccacagagugacuuuucuuc
auucgucagcaaauugaagggcagaacuguccugguggucggggaaaaguuguccgucccaggcaaaaugguugacuggu
ugucagaccggccugaggcuaccuucagagcucggcuggauuuaggcaucccaggugaugugcccaaauaugacauaaua
uuuguuaaugugaggaccccauauaaauaccaucacuaucagcagugugaagaccaugccauuaagcuuagcauguugac
caagaaagcuugucugcaucugaaucccggcggaaccugugucagcauagguuaugguuacgcugacagggccagcgaaa
gcaucauuggugcuauagcgcggcaguucaaguuuucccggguaugcaaaccgaaauccucacuugaagagacggaaguu
cuguuuguauucauuggguacgaucgcaaggcccguacgcacaauccuuacaagcuuucaucaaccuugaccaacauuua
uacagguuccagacuccacgaagccggaugugcacccucauaucauguggugcgaggggauauugccacggccaccgaag
gagugauuauaaaugcugcuaacagcaaaggacaaccuggcggaggggugugcggagcgcuguauaagaaauucccggaa
agcuucgauuuacagccgaucgaaguaggaaaagcgcgacuggucaaaggugcagcuaaacauaucauucaugccguagg
accaaacuucaacaaaguuucggagguugaaggugacaaacaguuggcagaggcuuaugaguccaucgcuaagauuguca
acgauaacaauuacaagucaguagcgauuccacuguuguccaccggcaucuuuuccgggaacaaagaucgacuaacccaa
ucauugaaccauuugcugacagcuuuagacaccacugaugcagauguagccauauacugcagggacaagaaaugggaaau
gacucucaaggaagcaguggcuaggagagaagcaguggaggagauaugcauauccgacgacucuucagugacagaaccug
augcagagcuggugagggugcauccgaagaguucuuuggcuggaaggaagggcuacagcacaagcgauggcaaaacuuuc
ucauauuuggaagggaccaaguuucaccaggcggccaaggauauagcagaaauuaaugccauguggcccguugcaacgga
ggccaaugagcagguaugcauguauauccucggagaaagcaugagcaguauuaggucgaaaugccccgucgaagagucgg
aagccuccacaccaccuagcacgcugccuugcuugugcauccaugccaugacuccagaaagaguacagcgccuaaaagcc
ucacguccagaacaaauuacugugugcucauccuuuccauugccgaaguauagaaucacuggugugcagaagauccaaug
cucccagccuauauuguucucaccgaaagugccugcguauauucauccaaggaaguaucucguggaaacaccaccgguag
acgagacuccggagccaucggcagagaaccaauccacagaggggacaccugaacaaccaccacuuauaaccgaggaugag
accaggacuagaacgccugagccgaucaucaucgaagaggaagaagaggauagcauaaguuugcugucagauggcccgac
ccaccaggugcugcaagucgaggcagacauucacgggccgcccucuguaucuagcucauccugguccauuccucaugcau
ccgacuuugauguggacaguuuauccauacuugacacccuggagggagcuagcgugaccagcggggcaacgucagccgag
acuaacucuuacuucgcaaagaguauggaguuucuggcgcgaccggugccugcgccucgaacaguauucaggaacccucc
acaucccgcuccgcgcacaagaacaccgucacuugcacccagcagggccugcucgagaaccagccuaguuuccaccccgc
caggcgugaauagggugaucacuagagaggagcucgaggcgcuuaccccgucacgcacuccuagcaggucggucucgaga
accagccuggucuccaacccgccaggcguaaauagggugauuacaagagaggaguuugaggcguucguagcacaacaaca
augacgguuugaugcgggugcauacaucuuuuccuccgacaccggucaagggcauuuacaacaaaaaucaguaaggcaaa
cggugcuauccgaagugguguuggagaggaccgaauuggagauuucguaugccccgcgccucgaccaagaaaaagaagaa
uuacuacgcaagaaauuacaguuaaaucccacaccugcuaacagaagcagauaccaguccaggaagguggagaacaugaa
agccauaacagcuagacguauucugcaaggccuagggcauuauuugaaggcagaaggaaaaguggagugcuaccgaaccc
ugcauccuguuccuuuguauucaucuagugugaaccgugccuuuucaagccccaaggucgcaguggaagccuguaacgcc
augacgguuugaugcgggugcauacaucuuuuccuccgacaccggucaagggcauuuacaacaaaaaucaguaaggcaaa
agcuucaugcugcuuagacacugccaguuuuugcccugcaaagcugcgcagcuuuccaaagaaacacuccuauuuggaac
ccacaauacgaucggcagugccuucagcgauccagaacacgcuccagaacguccuggcagcugccacaaaaagaaauugc
aaugucacgcaaaugagagaauugcccguauuggauucggcggccuuuaauguggaaugcuucaagaaauaugcguguaa
uaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuugua
auggacuuaaagagagacgugaaagugacuccaggaacaaaacauacugaagaacggcccaagguacaggugauccaggc
ugccgauccgcuagcaacagcguaucugugcggaauccaccgagagcugguuaggagauuaaaugcgguccugcuuccga
acauucauacacuguuugauaugucggcugaagacuuugacgcuauuauagccgagcacuuccagccuggggauuguguu
cuggaaacugacaucgcgucguuugauaaaagugaggacgacgccauggcucugaccgcguuaaugauucuggaagacuu
agguguggacgcagagcuguugacgcugauugaggcggcuuucggcgaaauuucaucaauacauuugcccacuaaaacua
aauuuaaauucggagccaugaugaaaucuggaauguuccucacacuguuugugaacacagucauuaacauuguaaucgca
agcagaguguugagagaacggcuaaccggaucaccaugugcagcauucauuggagaugacaauaucgugaaaggagucaa
aucggacaaauuaauggcagacaggugcgccaccugguugaauauggaagucaagauuauagaugcuguggugggcgaga
aggcuguuuaagcuuggcaaaccucuggcagcagacgaugaacaugaugaugacaggagaagggcauugcaugaagaguc
aacacgcuggaaccgaguggguauucuuucagagcugugcaaggcaguagaaucaagguaugaaaccguaggaacuucca
ucauaguuauggccaugacuacucuagcuagcaguguuaaaucauucagcuaccugagaggggccccuauaacucucuac
ggcuaaccugaauggacuacgacauagucuaguccgccaagAUGUUUCUGCUCACAACCAAACGCACUAUGUUUGUUUUC
CUCGUGCUGCUCCCUUUGGUAAGUUCUCAGUGUGUAAACCUGACAACACGAACCCAGUUGCCUCCAGCUUAUACCAACUC
AUUUACUCGCGGAGUAUAUUAUCCCGAUAAGGUCUUUAGAAGUAGCGUGUUGCACUCUACACAGGAUCUGUUCUUGCCCU
UCUUUAGUAACGUUACCUGGUUUCAUGCAAUACAUGUGAGCGGAACAAAUGGAACAAAAAGAUUUGACAAUCCAGUGCUU
CCAUUUAAUGAUGGGGUUUACUUUGCCAGUACCGAAAAGUCAAACAUAAUCCGGGGGUGGAUCUUUGGAACCACUUUGGA
CUCUAAGACACAGUCUCUCCUCAUAGUAAACAACGCCACCAAUGUUGUCAUAAAAGUAUGCGAAUUUCAGUUUUGCAACG
AUCCCUUUCUCGGGGUGUAUUACCAUAAGAAUAAUAAAUCCUGGAUGGAGUCUGAGUUCCGGGUUUAUAGUAGUGCUAAU
AAUUGCACUUUCGAAUACGUGUCCCAACCAUUCCUCAUGGACCUUGAGGGCAAACAGGGGAAUUUUAAAAACUUGCGCGA
AUUUGUCUUUAAGAAUAUCGACGGAUACUUUAAGAUCUAUAGUAAACACACUCCUAUCAACCUCGUUCGGGAUCUUCCCC
AAGGCUUUUCUGCUCUCGAACCCCUCGUAGACUUGCCAAUUGGGAUAAAUAUCACUCGCUUUCAAACUUUGCUUGCCCUC
CACAGGAGCUACCUGACACCCGGCGACUCUUCUUCUGGUUGGACCGCCGGCGCCGCUGCCUAUUAUGUUGGUUACCUUCA
GCCACGAACAUUCUUGCUCAAGUAUAACGAGAAUGGCACCAUUACCGACGCCGUCGAUUGUGCAUUGGAUCCCUUGUCUG
AAACAAAAUGUACCUUGAAGUCCUUUACCGUAGAGAAAGGCAUAUACCAGACUUCCAACUUCCGAGUUCAGCCUACAGAA
UCCAUUGUGAGAUUUCCCAACAUCACAAACCUCUGCCCUUUCGGUGAAGUAUUUAAUGCUACACGCUUCGCUUCAGUCUA
UGCCUGGAAUAGGAAGCGCAUAUCAAAUUGCGUGGCCGAUUAUUCAGUCCUCUAUAAUAGCGCAUCCUUCAGUACUUUCA
AGUGCUACGGCGUUUCCCCCACCAAACUCAAUGAUCUUUGCUUCACCAACGUCUAUGCUGACAGUUUUGUCAUACGAGGC
GACGAAGUACGCCAGAUUGCCCCCGGGCAGACAGGUAAAAUUGCUGAUUAUAAUUAUAAACUCCCAGAUGACUUUACUGG
AUGCGUCAUAGCCUGGAAUUCCAACAAUCUUGAUUCCAAGGUUGGUGGGAAUUAUAAUUACCUUUAUCGACUGUUCAGAA
AGAGUAACUUGAAACCAUUUGAGAGAGACAUAUCCACCGAGAUUUACCAGGCAGGCAGUACUCCUUGUAACGGCGUUGAG
GGAUUUAACUGCUAUUUUCCUUUGCAAUCCUAUGGCUUUCAACCAACAAACGGGGUUGGCUAUCAACCCUAUCGAGUGGU
UGUCCUGAGCUUUGAACUUUUGCACGCUCCCGCCACAGUCUGCGGACCAAAAAAGAGUACAAAUCUUGUCAAGAAUAAGU
GCGUAAAUUUCAAUUUCAAUGGCCUUACAGGAACAGGCGUGCUGACUGAGUCAAACAAGAAGUUCCUGCCAUUUCAGCAG
UUUGGGCGGGAUAUAGCAGACACAACUGACGCUGUACGCGAUCCUCAGACUUUGGAGAUCUUGGACAUCACUCCCUGUUC
UUUCGGAGGGGUAUCUGUCAUCACCCCCGGAACUAAUACAUCAAAUCAGGUCGCUGUGUUGUACCAAGAUGUCAACUGCA
CAGAAGUCCCCGUUGCUAUACACGCAGACCAGCUCACCCCCACAUGGCGGGUGUACUCAACUGGCUCAAACGUAUUCCAG
ACCAGAGCUGGGUGCUUGAUCGGUGCUGAACACGUAAACAAUAGCUAUGAAUGCGAUAUUCCCAUCGGUGCCGGGAUCUG
CGCUAGCUAUCAGACACAGACCAAUUCCCCCCGGCGAGCACGAUCUGUAGCAUCCCAGUCUAUUAUUGCCUACACUAUGU
CAUUGGGCGCCGAGAAUAGCGUCGCAUAUUCAAAUAAUUCUAUUGCAAUACCCACCAACUUCACAAUCUCCGUAACUACA
GAAAUACUUCCAGUUUCCAUGACAAAGACAUCAGUGGAUUGUACAAUGUAUAUAUGCGGAGAUUCCACAGAAUGUUCAAA
UUUGCUCUUGCAGUACGGCUCCUUCUGCACCCAGCUCAACAGGGCCCUUACAGGUAUUGCUGUCGAACAGGACAAGAACA
CACAAGAAGUCUUCGCCCAAGUCAAACAGAUAUACAAAACUCCUCCCAUAAAGGAUUUUGGCGGCUUCAACUUUAGUCAG
AUCCUCCCAGACCCUUCAAAACCAUCUAAACGAUCAUUUAUUGAAGAUCUGCUGUUCAACAAGGUCACUCUUGCCGAUGC
UGGAUUCAUUAAGCAAUACGGUGACUGCCUUGGUGAUAUUGCUGCCCGAGAUCUGAUCUGUGCCCAGAAAUUCAACGGGC
UCACUGUACUCCCUCCACUGCUCACAGACGAAAUGAUUGCACAGUACACAAGUGCCCUGUUGGCAGGCACAAUCACUAGC
GGCUGGACCUUUGGCGCAGGUGCAGCACUCCAAAUACCUUUUGCCAUGCAGAUGGCCUAUCGGUUUAAUGGGAUAGGCGU
GACUCAAAAUGUCCUCUACGAAAACCAAAAGUUGAUAGCUAACCAAUUCAAUUCAGCAAUCGGGAAGAUACAGGAUUCAC
UGUCUAGUACUGCUAGUGCCCUUGGUAAGCUGCAGGACGUUGUCAACCAGAAUGCUCAAGCUCUGAAUACAUUGGUUAAG
CAGCUCUCUAGUAAUUUUGGGGCCAUCUCUUCAGUACUUAAUGAUAUUUUGAGCCGAUUGGACAAAGUGGAAGCUGAAGU
ACAGAUCGACAGGCUGAUAACAGGCCGGCUCCAAUCCCUCCAAACAUACGUGACACAACAACUCAUACGCGCAGCCGAAA
UCCGAGCCAGCGCUAACCUGGCAGCUACCAAGAUGUCAGAAUGCGUUCUGGGCCAGAGUAAACGCGUAGAUUUCUGCGGG
AAAGGGUACCACCUGAUGUCCUUUCCACAAUCUGCACCUCACGGGGUCGUCUUUUUGCAUGUAACAUAUGUACCCGCACA
AGAGAAGAAUUUUACUACCGCUCCUGCCAUCUGUCAUGACGGGAAAGCUCAUUUUCCUCGCGAAGGUGUGUUUGUAUCUA
AUGGUACACAUUGGUUUGUCACACAGCGGAAUUUCUAUGAACCCCAGAUCAUUACAACUGACAACACUUUUGUUUCCGGG
AAUUGUGACGUGGUCAUAGGAAUCGUAAAUAACACUGUAUAUGAUCCCCUCCAACCAGAGCUGGACUCUUUUAAAGAAGA
ACUGGAUAAAUAUUUCAAGAACCACACAAGUCCCGACGUGGACCUUGGGGACAUAAGUGGUAUUAACGCAUCUGUGGUUA
ACAUUCAAAAGGAAAUCGACAGACUCAACGAGGUGGCCAAAAACCUGAACGAAAGCUUGAUAGAUCUCCAGGAGUUGGGC
AAGUAUGAACAGUACAUUAAAUGGCCAUGGUACAUAUGGCUUGGCUUUAUCGCUGGCCUUAUCGCCAUCGUAAUGGUUAC
AAUCAUGCUGUGCUGCAUGACCUCCUGCUGUUCUUGUUUGAAAGGGUGUUGUUCUUGUGGUAGUUGUUGCAAGUUUGACG
AAGAUGAUUCCGAACCUGUUCUUAAAGGGGUAAAGCUUCACUAUACAugauaaccgcggugucaaaaaccgcguggacgu
cugaccaaccagaaacauaauugaauacagcagcaauuggcaagcugcuuacauagaacucgcggcgauuggcaugccgc
cuuaaaauuuuuauuuuauuuuuuucuuucuuuccgaaucggauuuuguuuuuaauauuucaaaaaaaaaaaaaaaaaaa
aaaaaaaaaaaaaaaaaaaaaaaaaaa
Full-length VEEV+ K995P-V996P RNA Sequence Antigen Encoding RNA Sequence
SEQ ID NO: 25
auaggcggcgcaugagagaagcccagaccaauuaccuacccaaaauggagaaaguucacguugacaucgaggaagacagc
ccauuccucagagcuuugcagcggagcuucccgcaguuugagguagaagccaagcaggucacugauaaugaccaugcuaa
ugccagagcguuuucgcaucuggcuucaaaacugaucgaaacggagguggacccauccgacacgauccuugacauuggaa
gugcgcccgcccgcagaauguauucuaagcacaaguaucauuguaucuguccgaugagaugugcggaagauccggacaga
uuguauaaguaugcaacuaagcugaagaaaaacuguaaggaaauaacugauaaggaauuggacaagaaaaugaaggagcu
ggccgccgucaugagcgacccugaccuggaaacugagacuaugugccuccacgacgacgagucgugucgcuacgaagggc
aagucgcuguuuaccaggauguauacgcgguugacggaccgacaagucucuaucaccaagccaauaagggaguuagaguc
gccuacuggauaggcuuugacaccaccccuuuuauguuuaagaacuuggcuggagcauauccaucauacucuaccaacug
ggccgacgaaaccguguuaacggcucguaacauaggccuaugcagcucugacguuauggagcggucacguagagggaugu
ccauucuuagaaagaaguauuugaaaccauccaacaauguucuauucucuguuggcucgaccaucuaccacgagaagagg
gacuuacugaggagcuggcaccugccgucuguauuucacuuacguggcaagcaaaauuacacaugucggugugagacuau
aguuaguugcgacggguacgucguuaaaagaauagcuaucaguccaggccuguaugggaagccuucaggcuaugcugcua
cgaugcaccgcgagggauucuugugcugcaaagugacagacacauugaacggggagagggucucuuuucccgugugcacg
uaugugccagcuacauugugugaccaaaugacuggcauacuggcaacagaugucagugcggacgacgcgcaaaaacugcu
gguugggcucaaccagcguauagucgucaacggucgcacccagagaaacaccaauaccaugaaaaauuaccuuuugcccg
uaguggcccaggcauuugcuaggugggcaaaggaauauaaggaagaucaagaagaugaaaggccacuaggacuacgagau
agacaguuagucaugggguguuguugggcuuuuagaaggcacaagauaacaucuauuuauaagcgcccggauacccaaac
caucaucaaagugaacagcgauuuccacucauucgugcugcccaggauaggcaguaacacauuggagaucgggcugagaa
caagaaucaggaaaauguuagaggagcacaaggagccgucaccucucauuaccgccgaggacguacaagaagcuaagugc
gcagccgaugaggcuaaggaggugcgugaagccgaggaguugcgcgcagcucuaccaccuuuggcagcugauguugagga
gcccacucuggaggcagacgucgacuugauguuacaagaggcuggggccggcucaguggagacaccucguggcuugauaa
agguuaccagcuacgauggcgaggacaagaucggcucuuacgcugugcuuucuccgcaggcuguacucaagagugaaaaa
uuaucuugcauccacccucucgcugaacaagucauagugauaacacacucuggccgaaaagggcguuaugccguggaacc
auaccaugguaaaguaguggugccagagggacaugcaauacccguccaggacuuucaagcucugagugaaagugccacca
uuguguacaacgaacgugaguucguaaacagguaccugcaccauauugccacacauggaggagcgcugaacacugaugaa
gaauauuacaaaacugucaagcccagcgagcacgacggcgaauaccuguacgacaucgacaggaaacagugcgucaagaa
agaacuagucacugggcuagggcucacaggcgagcugguggauccucccuuccaugaauucgccuacgagagucugagaa
cacgaccagccgcuccuuaccaaguaccaaccauagggguguauggcgugccaggaucaggcaagucuggcaucauuaaa
agcgcagucaccaaaaaagaucuaguggugagcgccaagaaagaaaacugugcagaaauuauaagggacgucaagaaaau
gaaagggcuggacgucaaugccagaacuguggacucagugcucuugaauggaugcaaacaccccguagagacccuguaua
uugacgaagcuuuugcuugucaugcagguacucucagagcgcucauagccauuauaagaccuaaaaaggcagugcucugc
ggggaucccaaacagugcgguuuuuuuaacaugaugugccugaaagugcauuuuaaccacgagauuugcacacaagucuu
ccacaaaagcaucucucgccguugcacuaaaucugugacuucggucgucucaaccuuguuuuacgacaaaaaaaugagaa
cgacgaauccgaaagagacuaagauugugauugacacuaccggcaguaccaaaccuaagcaggacgaucucauucucacu
uguuucagagggugggugaagcaguugcaaauagauuacaaaggcaacgaaauaaugacggcagcugccucucaagggcu
gacccguaaagguguguaugccguucgguacaaggugaaugaaaauccucuguacgcacccaccucagaacaugugaacg
uccuacugacccgcacggaggaccgcaucguguggaaaacacuagccggcgacccauggauaaaaacacugacugccaag
uacccugggaauuucacugccacgauagaggaguggcaagcagagcaugaugccaucaugaggcacaucuuggagagacc
ggacccuaccgacgucuuccagaauaaggcaaacguguguugggccaaggcuuuagugccggugcugaagaccgcuggca
uagacaugaccacugaacaauggaacacuguggauuauuuugaaacggacaaagcucacucagcagagauaguauugaac
cacaacugccucgggcaguugccacuggaagagucuaugacaugaacacugguacacugcgcaauuaugauccgcgcaua
uaaucacugggauaacuccccgucgccuaacauguacgggcugaauaaagaagugguccgucagcucucucgcagguacc
cacaacugccucgggcaguugccacuggaagagucuaugacaugaacacugguacacugcgcaauuaugauccgcgcaua
aaccuaguaccuguaaacagaagacugccucaugcuuuaguccuccaccauaaugaacacccacagagugacuuuucuuc
auucgucagcaaauugaagggcagaacuguccugguggucggggaaaaguuguccgucccaggcaaaaugguugacuggu
ugucagaccggccugaggcuaccuucagagcucggcuggauuuaggcaucccaggugaugugcccaaauaugacauaaua
uuuguuaaugugaggaccccauauaaauaccaucacuaucagcagugugaagaccaugccauuaagcuuagcauguugac
caagaaagcuugucugcaucugaaucccggcggaaccugugucagcauagguuaugguuacgcugacagggccagcgaaa
gcaucauuggugcuauagcgcggcaguucaaguuuucccggguaugcaaaccgaaauccucacuugaagagacggaaguu
cuguuuguauucauuggguacgaucgcaaggcccguacgcacaauccuuacaagcuuucaucaaccuugaccaacauuua
uacagguuccagacuccacgaagccggaugugcacccucauaucauguggugcgaggggauauugccacggccaccgaag
gagugauuauaaaugcugcuaacagcaaaggacaaccuggcggaggggugugcggagcgcuguauaagaaauucccggaa
agcuucgauuuacagccgaucgaaguaggaaaagcgcgacuggucaaaggugcagcuaaacauaucauucaugccguagg
accaaacuucaacaaaguuucggagguugaaggugacaaacaguuggcagaggcuuaugaguccaucgcuaagauuguca
acgauaacaauuacaagucaguagcgauuccacuguuguccaccggcaucuuuuccgggaacaaagaucgacuaacccaa
ucauugaaccauuugcugacagcuuuagacaccacugaugcagauguagccauauacugcagggacaagaaaugggaaau
gacucucaaggaagcaguggcuaggagagaagcaguggaggagauaugcauauccgacgacucuucagugacagaaccug
augcagagcuggugagggugcauccgaagaguucuuuggcuggaaggaagggcuacagcacaagcgauggcaaaacuuuc
ucauauuuggaagggaccaaguuucaccaggcggccaaggauauagcagaaauuaaugccauguggcccguugcaacgga
ggccaaugagcagguaugcauguauauccucggagaaagcaugagcaguauuaggucgaaaugccccgucgaagagucgg
aagccuccacaccaccuagcacgcugccuugcuugugcauccaugccaugacuccagaaagaguacagcgccuaaaagcc
ucacguccagaacaaauuacugugugcucauccuuuccauugccgaaguauagaaucacuggugugcagaagauccaaug
cucccagccuauauuguucucaccgaaagugccugcguauauucauccaaggaaguaucucguggaaacaccaccgguag
acgagacuccggagccaucggcagagaaccaauccacagaggggacaccugaacaaccaccacuuauaaccgaggaugag
accaggacuagaacgccugagccgaucaucaucgaagaggaagaagaggauagcauaaguuugcugucagauggcccgac
ccaccaggugcugcaagucgaggcagacauucacgggccgcccucuguaucuagcucauccugguccauuccucaugcau
ccgacuuugauguggacaguuuauccauacuugacacccuggagggagcuagcgugaccagcggggcaacgucagccgag
acuaacucuuacuucgcaaagaguauggaguuucuggcgcgaccggugccugcgccucgaacaguauucaggaacccucc
acaucccgcuccgcgcacaagaacaccgucacuugcacccagcagggccugcucgagaaccagccuaguuuccaccccgc
caggcgugaauagggugaucacuagagaggagcucgaggcgcuuaccccgucacgcacuccuagcaggucggucucgaga
accagccuggucuccaacccgccaggcguaaauagggugauuacaagagaggaguuugaggcguucguagcacaacaaca
augacgguuugaugcgggugcauacaucuuuuccuccgacaccggucaagggcauuuacaacaaaaaucaguaaggcaaa
cggugcuauccgaagugguguuggagaggaccgaauuggagauuucguaugccccgcgccucgaccaagaaaaagaagaa
uuacuacgcaagaaauuacaguuaaaucccacaccugcuaacagaagcagauaccaguccaggaagguggagaacaugaa
agccauaacagcuagacguauucugcaaggccuagggcauuauuugaaggcagaaggaaaaguggagugcuaccgaaccc
ugcauccuguuccuuuguauucaucuagugugaaccgugccuuuucaagccccaaggucgcaguggaagccuguaacgcc
auguugaaagagaacuuuccgacuguggcuucuuacuguauuauuccagaguacgaugccuauuuggacaugguugacgg
auguugaaagagaacuuuccgacuguggcuucuuacuguauuauuccagaguacgaugccuauuuggacaugguugacgg
ccacaauacgaucggcagugccuucagcgauccagaacacgcuccagaacguccuggcagcugccacaaaaagaaauugc
aaugucacgcaaaugagagaauugcccguauuggauucggcggccuuuaauguggaaugcuucaagaaauaugcguguaa
uaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuugua
auggacuuaaagagagacgugaaagugacuccaggaacaaaacauacugaagaacggcccaagguacaggugauccaggc
ugccgauccgcuagcaacagcguaucugugcggaauccaccgagagcugguuaggagauuaaaugcgguccugcuuccga
acauucauacacuguuugauaugucggcugaagacuuugacgcuauuauagccgagcacuuccaccugcgggauuguguu
cuggaaacugacaucgcgucguuugauaaaagugaggacgacgccauggcucugaccgcguuaaugauucuggaagacuu
agguguggacgcagagcuguugacgcugauugaggcggcuuucggcgaaauuucaucaauacauuugcccacuaaaacua
aauuuaaauucggagccaugaugaaaucuggaauguuccucacacuguuugugaacacagucauuaacauuguaaucgca
agcagaguguugagagaacggcuaaccggaucaccaugugcagcauucauuggagaugacaauaucgugaaaggagucaa
aucggacaaauuaauggcagacaggugcgccaccugguugaauauggaagucaagauuauagaugcuguggugggcgaga
aggcuguuuaagcuuggcaaaccucuggcagcagacgaugaacaugaugaugacaggagaagggcauugcaugaagaguc
aacacgcuggaaccgaguggguauucuuucagagcugugcaaggcaguagaaucaagguaugaaaccguaggaacuucca
ucauaguuauggccaugacuacucuagcuagcaguguuaaaucauucagcuaccugagaggggccccuauaacucucuac
ggcuaaccugaauggacuacgacauagucuaguccgccaagAUGUUUCUGCUCACAACCAAACGCACUAUGUUUGUUUUC
CUCGUGCUGCUCCCUUUGGUAAGUUCUCAGUGUGUAAACCUGACAACACGAACCCAGUUGCCUCCAGCUUAUACCAACUC
AUUUACUCGCGGAGUAUAUUAUCCCGAUAAGGUCUUUAGAAGUAGCGUGUUGCACUCUACACAGGAUCUGUUCUUGCCCU
UCUUUAGUAACGUUACCUGGUUUCAUGCAAUACAUGUGAGCGGAACAAAUGGAACAAAAAGAUUUGACAAUCCAGUGCUU
CCAUUUAAUGAUGGGGUUUACUUUGCCAGUACCGAAAAGUCAAACAUAAUCCGGGGGUGGAUCUUUGGAACCACUUUGGA
CUCUAAGACACAGUCUCUCCUCAUAGUAAACAACGCCACCAAUGUUGUCAUAAAAGUAUGCGAAUUUCAGUUUUGCAACG
AUCCCUUUCUCGGGGUGUAUUACCAUAAGAAUAAUAAAUCCUGGAUGGAGUCUGAGUUCCGGGUUUAUAGUAGUGCUAAU
AAUUGCACUUUCGAAUACGUGUCCCAACCAUUCCUCAUGGACCUUGAGGGCAAACAGGGGAAUUUUAAAAACUUGCGCGA
AUUUGUCUUUAAGAAUAUCGACGGAUACUUUAAGAUCUAUAGUAAACACACUCCUAUCAACCUCGUUCGGGAUCUUCCCC
AAGGCUUUUCUGCUCUCGAACCCCUCGUAGACUUGCCAAUUGGGAUAAAUAUCACUCGCUUUCAAACUUUGCUUGCCCUC
CACAGGAGCUACCUGACACCCGGCGACUCUUCUUCUGGUUGGACCGCCGGCGCCGCUGCCUAUUAUGUUGGUUACCUUCA
GCCACGAACAUUCUUGCUCAAGUAUAACGAGAAUGGCACCAUUACCGACGCCGUCGAUUGUGCAUUGGAUCCCUUGUCUG
AAACAAAAUGUACCUUGAAGUCCUUUACCGUAGAGAAAGGCAUAUACCAGACUUCCAACUUCCGAGUUCAGCCUACAGAA
UCCAUUGUGAGAUUUCCCAACAUCACAAACCUCUGCCCUUUCGGUGAAGUAUUUAAUGCUACACGCUUCGCUUCAGUCUA
UGCCUGGAAUAGGAAGCGCAUAUCAAAUUGCGUGGCCGAUUAUUCAGUCCUCUAUAAUAGCGCAUCCUUCAGUACUUUCA
AGUGCUACGGCGUUUCCCCCACCAAACUCAAUGAUCUUUGCUUCACCAACGUCUAUGCUGACAGUUUUGUCAUACGAGGC
GACGAAGUACGCCAGAUUGCCCCCGGGCAGACAGGUAAAAUUGCUGAUUAUAAUUAUAAACUCCCAGAUGACUUUACUGG
AUGCGUCAUAGCCUGGAAUUCCAACAAUCUUGAUUCCAAGGUUGGUGGGAAUUAUAAUUACCUUUAUCGACUGUUCAGAA
AGAGUAACUUGAAACCAUUUGAGAGAGACAUAUCCACCGAGAUUUACCAGGCAGGCAGUACUCCUUGUAACGGCGUUGAG
GGAUUUAACUGCUAUUUUCCUUUGCAAUCCUAUGGCUUUCAACCAACAAACGGGGUUGGCUAUCAACCCUAUCGAGUGGU
UGUCCUGAGCUUUGAACUUUUGCACGCUCCCGCCACAGUCUGCGGACCAAAAAAGAGUACAAAUCUUGUCAAGAAUAAGU
GCGUAAAUUUCAAUUUCAAUGGCCUUACAGGAACAGGCGUGCUGACUGAGUCAAACAAGAAGUUCCUGCCAUUUCAGCAG
UUUGGGCGGGAUAUAGCAGACACAACUGACGCUGUACGCGAUCCUCAGACUUUGGAGAUCUUGGACAUCACUCCCUGUUC
UUUCGGAGGGGUAUCUGUCAUCACCCCCGGAACUAAUACAUCAAAUCAGGUCGCUGUGUUGUACCAAGAUGUCAACUGCA
CAGAAGUCCCCGUUGCUAUACACGCAGACCAGCUCACCCCCACAUGGCGGGUGUACUCAACUGGCUCAAACGUAUUCCAG
ACCAGAGCUGGGUGCUUGAUCGGUGCUGAACACGUAAACAAUAGCUAUGAAUGCGAUAUUCCCAUCGGUGCCGGGAUCUG
CGCUAGCUAUCAGACACAGACCAAUUCCCCCCGGCGAGCACGAUCUGUAGCAUCCCAGUCUAUUAUUGCCUACACUAUGU
CAUUGGGCGCCGAGAAUAGCGUCGCAUAUUCAAAUAAUUCUAUUGCAAUACCCACCAACUUCACAAUCUCCGUAACUACA
GAAAUACUUCCAGUUUCCAUGACAAAGACAUCAGUGGAUUGUACAAUGUAUAUAUGCGGAGAUUCCACAGAAUGUUCAAA
UUUGCUCUUGCAGUACGGCUCCUUCUGCACCCAGCUCAACAGGGCCCUUACAGGUAUUGCUGUCGAACAGGACAAGAACA
CACAAGAAGUCUUCGCCCAAGUCAAACAGAUAUACAAAACUCCUCCCAUAAAGGAUUUUGGCGGCUUCAACUUUAGUCAG
AUCCUCCCAGACCCUUCAAAACCAUCUAAACGAUCAUUUAUUGAAGAUCUGCUGUUCAACAAGGUCACUCUUGCCGAUGC
UGGAUUCAUUAAGCAAUACGGUGACUGCCUUGGUGAUAUUGCUGCCCGAGAUCUGAUCUGUGCCCAGAAAUUCAACGGGC
UCACUGUACUCCCUCCACUGCUCACAGACGAAAUGAUUGCACAGUACACAAGUGCCCUGUUGGCAGGCACAAUCACUAGC
GGCUGGACCUUUGGCGCAGGUGCAGCACUCCAAAUACCUUUUGCCAUGCAGAUGGCCUAUCGGUUUAAUGGGAUAGGCGU
GACUCAAAAUGUCCUCUACGAAAACCAAAAGUUGAUAGCUAACCAAUUCAAUUCAGCAAUCGGGAAGAUACAGGAUUCAC
UGUCUAGUACUGCUAGUGCCCUUGGUAAGCUGCAGGACGUUGUCAACCAGAAUGCUCAAGCUCUGAAUACAUUGGUUAAG
CAGCUCUCUAGUAAUUUUGGGGCCAUCUCUUCAGUACUUAAUGAUAUUUUGAGCCGAUUGGACccacccGAAGCUGAAGU
ACAGAUCGACAGGCUGAUAACAGGCCGGCUCCAAUCCCUCCAAACAUACGUGACACAACAACUCAUACGCGCAGCCGAAA
UCCGAGCCAGCGCUAACCUGGCAGCUACCAAGAUGUCAGAAUGCGUUCUGGGCCAGAGUAAACGCGUAGAUUUCUGCGGG
AAAGGGUACCACCUGAUGUCCUUUCCACAAUCUGCACCUCACGGGGUCGUCUUUUUGCAUGUAACAUAUGUACCCGCACA
AGAGAAGAAUUUUACUACCGCUCCUGCCAUCUGUCAUGACGGGAAAGCUCAUUUUCCUCGCGAAGGUGUGUUUGUAUCUA
AUGGUACACAUUGGUUUGUCACACAGCGGAAUUUCUAUGAACCCCAGAUCAUUACAACUGACAACACUUUUGUUUCCGGG
AAUUGUGACGUGGUCAUAGGAAUCGUAAAUAACACUGUAUAUGAUCCCCUCCAACCAGAGCUGGACUCUUUUAAAGAAGA
ACUGGAUAAAUAUUUCAAGAACCACACAAGUCCCGACGUGGACCUUGGGGACAUAAGUGGUAUUAACGCAUCUGUGGUUA
ACAUUCAAAAGGAAAUCGACAGACUCAACGAGGUGGCCAAAAACCUGAACGAAAGCUUGAUAGAUCUCCAGGAGUUGGGC
AAGUAUGAACAGUACAUUAAAUGGCCAUGGUACAUAUGGCUUGGCUUUAUCGCUGGCCUUAUCGCCAUCGUAAUGGUUAC
AAUCAUGCUGUGCUGCAUGACCUCCUGCUGUUCUUGUUUGAAAGGGUGUUGUUCUUGUGGUAGUUGUUGCAAGUUUGACG
AAGAUGAUUCCGAACCUGUUCUUAAAGGGGUAAAGCUUCACUAUACAugauaaccgcggugucaaaaaccgcguggacgu
cugaccaaccagaaacauaauugaauacagcagcaauuggcaagcugcuuacauagaacucgcggcgauuggcaugccgc
cuuaaaauuuuuauuuuauuuuuucuuuucuuuuccgaaucggauuuuguuuuuaauauuucaaaaaaaaaaaaaaaaaa
aaaaaaaaaaaaaaaaaaaaaaaaaaa
Full-length VEEV+ D614G Antigen Encoding RNA Sequence
SEQ ID NO: 26
auaggcggcgcaugagagaagcccagaccaauuaccuacccaaaauggagaaaguucacguugacaucgaggaagacagc
ccauuccucagagcuuugcagcggagcuucccgcaguuugagguagaagccaagcaggucacugauaaugaccaugcuaa
ugccagagcguuuucgcaucuggcuucaaaacugaucgaaacggagguggacccauccgacacgauccuugacauuggaa
gugcgcccgcccgcagaauguauucuaagcacaaguaucauuguaucuguccgaugagaugugcggaagauccggacaga
uuguauaaguaugcaacuaagcugaagaaaaacuguaaggaaauaacugauaaggaauuggacaagaaaaugaaggagcu
ggccgccgucaugagcgacccugaccuggaaacugagacuaugugccuccacgacgacgagucgugucgcuacgaagggc
aagucgcuguuuaccaggauguauacgcgguugacggaccgacaagucucuaucaccaagccaauaagggaguuagaguc
gccuacuggauaggcuuugacaccaccccuuuuauguuuaagaacuuggcuggagcauauccaucauacucuaccaacug
ggccgacgaaaccguguuaacggcucguaacauaggccuaugcagcucugacguuauggagcggucacguagagggaugu
ccauucuuagaaagaaguauuugaaaccauccaacaauguucuauucucuguuggcucgaccaucuaccacgagaagagg
gacuuacugaggagcuggcaccugccgucuguauuucacuuacguggcaagcaaaauuacacaugucggugugagacuau
aguuaguugcgacggguacgucguuaaaagaauagcuaucaguccaggccuguaugggaagccuucaggcuaugcugcua
cgaugcaccgcgagggauucuugugcugcaaagugacagacacauugaacggggagagggucucuuuucccgugugcacg
uaugugccagcuacauugugugaccaaaugacuggcauacuggcaacagaugucagugcggacgacgcgcaaaaacugcu
gguugggcucaaccagcguauagucgucaacggucgcacccagagaaacaccaauaccaugaaaaauuaccuuuugcccg
uaguggcccaggcauuugcuaggugggcaaaggaauauaaggaagaucaagaagaugaaaggccacuaggacuacgagau
agacaguuagucaugggguguuguugggcuuuuagaaggcacaagauaacaucuauuuauaagcgcccggauacccaaac
caucaucaaagugaacagcgauuuccacucauucgugcugcccaggauaggcaguaacacauuggagaucgggcugagaa
caagaaucaggaaaauguuagaggagcacaaggagccgucaccucucauuaccgccgaggacguacaagaagcuaagugc
gcagccgaugaggcuaaggaggugcgugaagccgaggaguugcgcgcagcucuaccaccuuuggcagcugauguugagga
gcccacucuggaggcagacgucgacuugauguuacaagaggcuggggccggcucaguggagacaccucguggcuugauaa
agguuaccagcuacgauggcgaggacaagaucggcucuuacgcugugcuuucuccgcaggcuguacucaagagugaaaaa
uuaucuugcauccacccucucgcugaacaagucauagugauaacacacucuggccgaaaagggcguuaugccguggaacc
auaccaugguaaaguaguggugccagagggacaugcaauacccguccaggacuuucaagcucugagugaaagugccacca
uuguguacaacgaacgugaguucguaaacagguaccugcaccauauugccacacauggaggagcgcugaacacugaugaa
gaauauuacaaaacugucaagcccagcgagcacgacggcgaauaccuguacgacaucgacaggaaacagugcgucaagaa
agaacuagucacugggcuagggcucacaggcgagcugguggauccucccuuccaugaauucgccuacgagagucugagaa
cacgaccagccgcuccuuaccaaguaccaaccauagggguguauggcgugccaggaucaggcaagucuggcaucauuaaa
agcgcagucaccaaaaaagaucuaguggugagcgccaagaaagaaaacugugcagaaauuauaagggacgucaagaaaau
gaaagggcuggacgucaaugccagaacuguggacucagugcucuugaauggaugcaaacaccccguagagacccuguaua
uugacgaagcuuuugcuugucaugcagguacucucagagcgcucauagccauuauaagaccuaaaaaggcagugcucugc
ggggaucccaaacagugcgguuuuuuuaacaugaugugccugaaagugcauuuuaaccacgagauuugcacacaagucuu
ccacaaaagcaucucucgccguugcacuaaaucugugacuucggucgucucaaccuuguuuuacgacaaaaaaaugagaa
cgacgaauccgaaagagacuaagauugugauugacacuacoggcaguaccaaaccuaagcaggacgaucucauucucacu
uguuucagagggugggugaagcaguugcaaauagauuacaaaggcaacgaaauaaugacggcagcugccucucaagggcu
gacccguaaagguguguaugccguucgguacaaggugaaugaaaauccucuguacgcacccaccucagaacaugugaacg
uccuacugacccgcacggaggaccgcaucguguggaaaacacuagccggcgacccauggauaaaaacacugacugccaag
uacccugggaauuucacugccacgauagaggaguggcaagcagagcaugaugccaucaugaggcacaucuuggagagacc
ggacccuaccgacgucuuccagaauaaggcaaacguguguugggccaaggcuuuagugccggugcugaagaccgcuggca
uagacaugaccacugaacaauggaacacuguggauuauuuugaaacggacaaagcucacucagcagagauaguauugaac
uaaucacugggauaacuccccgucgccuaacauguacgggcugaauaaagaagugguccgucagcucucucgcagguacc
cacaacugccucgggcaguugccacuggaagagucuaugacaugaacacugguacacugcgcaauuaugauccgcgcaua
aaccuaguaccuguaaacagaagacugccucaugcuuuaguccuccaccauaaugaacacccacagagugacuuuucuuc
auucgucagcaaauugaagggcagaacuguccugguggucggggaaaaguuguccgucccaggcaaaaugguugacuggu
ugucagaccggccugaggcuaccuucagagcucggcuggauuuaggcaucccaggugaugugcccaaauaugacauaaua
uuuguuaaugugaggaccccauauaaauaccaucacuaucagcagugugaagaccaugccauuaagcuuagcauguugac
caagaaagcuugucugcaucugaaucccggcggaaccugugucagcauagguuaugguuacgcugacagggccagcgaaa
gcaucauuggugcuauagcgcggcaguucaaguuuucccggguaugcaaaccgaaauccucacuugaagagacggaaguu
cuguuuguauucauuggguacgaucgcaaggcccguacgcacaauccuuacaagcuuucaucaaccuugaccaacauuua
uacagguuccagacuccacgaagccggaugugcacccucauaucauguggugcgaggggauauugccacggccaccgaag
gagugauuauaaaugcugcuaacagcaaaggacaaccuggcggaggggugugcggagcgcuguauaagaaauucccggaa
agcuucgauuuacagccgaucgaaguaggaaaagcgcgacuggucaaaggugcagcuaaacauaucauucaugccguagg
accaaacuucaacaaaguuucggagguugaaggugacaaacaguuggcagaggcuuaugaguccaucgcuaagauuguca
acgauaacaauuacaagucaguagcgauuccacuguuguccaccggcaucuuuuccgggaacaaagaucgacuaacccaa
ucauugaaccauuugcugacagcuuuagacaccacugaugcagauguagccauauacugcagggacaagaaaugggaaau
gacucucaaggaagcaguggcuaggagagaagcaguggaggagauaugcauauccgacgacucuucagugacagaaccug
augcagagcuggugagggugcauccgaagaguucuuuggcuggaaggaagggcuacagcacaagcgauggcaaaacuuuc
ucauauuuggaagggaccaaguuucaccaggcggccaaggauauagcagaaauuaaugccauguggcccguugcaacgga
ggccaaugagcagguaugcauguauauccucggagaaagcaugagcaguauuaggucgaaaugccccgucgaagagucgg
aagccuccacaccaccuagcacgcugccuugcuugugcauccaugccaugacuccagaaagaguacagcgccuaaaagcc
ucacguccagaacaaauuacugugugcucauccuuuccauugccgaaguauagaaucacuggugugcagaagauccaaug
cucccagccuauauuguucucaccgaaagugccugcguauauucauccaaggaaguaucucguggaaacaccaccgguag
acgagacuccggagccaucggcagagaaccaauccacagaggggacaccugaacaaccaccacuuauaaccgaggaugag
accaggacuagaacgccugagccgaucaucaucgaagaggaagaagaggauagcauaaguuugcugucagauggcccgac
ccaccaggugcugcaagucgaggcagacauucacgggccgcccucuguaucuagcucauccugguccauuccucaugcau
ccgacuuugauguggacaguuuauccauacuugacacccuggagggagcuagcgugaccagcggggcaacgucagccgag
acuaacucuuacuucgcaaagaguauggaguuucuggcgcgaccggugccugcgccucgaacaguauucaggaacccucc
acaucccgcuccgcgcacaagaacaccgucacuugcacccagcagggccugcucgagaaccagccuaguuuccaccccgc
caggcgugaauagggugaucacuagagaggagcucgaggcgcuuaccccgucacgcacuccuagcaggucggucucgaga
accagccuggucuccaacccgccaggcguaaauagggugauuacaagagaggaguuugaggcguucguagcacaacaaca
augacgguuugaugcgggugcauacaucuuuuccuccgacaccggucaagggcauuuacaacaaaaaucaguaaggcaaa
cggugcuauccgaagugguguuggagaggaccgaauuggagauuucguaugccccgcgccucgaccaagaaaaagaagaa
uuacuacgcaagaaauuacaguuaaaucccacaccugcuaacagaagcagauaccaguccaggaagguggagaacaugaa
agccauaacagcuagacguauucugcaaggccuagggcauuauuugaaggcagaaggaaaaguggagugcuaccgaaccc
ugcauccuguuccuuuguauucaucuagugugaaccgugccuuuucaagccccaaggucgcaguggaagccuguaacgcc
auguugaaagagaacuuuccgacuguggcuucuuacuguauuauuccagaguacgauguauuuggacaugguuguugagg
agcuucaugcugcuuagacacugccaguuuuugcccugcaaagcugcgcagcuuuccaaagaaacacuccuauuuggaac
ccacaauacgaucggcagugccuucagcgauccagaacacgcuccagaacguccuggcagcugccacaaaaagaaauugc
aaugucacgcaaaugagagaauugcccguauuggauucggcggccuuuaauguggaaugcuucaagaaauaugcguguaa
uaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuugua
auggacuuaaagagagacgugaaagugacuccaggaacaaaacauacugaagaacggcccaagguacaggugauccaggc
ugccgauccgcuagcaacagcguaucugugcggaauccaccgagagcugguuaggagauuaaaugcgguccugcuuccga
acauucauacacuguuugauaugucggcugaagacuuugacgcuauuauagccgagcacuuccagccuggggauuguguu
cuggaaacugacaucgcgucguuugauaaaagugaggacgacgccauggcucugaccgcguuaaugauucuggaagacuu
agguguggacgcagagcuguugacgcugauugaggcggcuuucggcgaaauuucaucaauacauuugcccacuaaaacua
aauuuaaauucggagccaugaugaaaucuggaauguuccucacacuguuugugaacacagucauuaacauuguaaucgca
agcagaguguugagagaacggcuaaccggaucaccaugugcagcauucauuggagaugacaauaucgugaaaggagucaa
aucggacaaauuaauggcagacaggugcgccaccugguugaauauggaagucaagauuauagaugcuguggugggcgaga
aggcuguuuaagcuuggcaaaccucuggcagcagacgaugaacaugaugaugacaggagaagggcauugcaugaagaguc
aacacgcuggaaccgaguggguauucuuucagagcugugcaaggcaguagaaucaagguaugaaaccguaggaacuucca
ucauaguuauggccaugacuacucuagcuagcaguguuaaaucauucagcuaccugagaggggccccuauaacucucuac
ggcuaaccugaauggacuacgacauagucuaguccgccaagAUGUUUCUGCUCACAACCAAACGCACUAUGUUUGUUUUC
CUCGUGCUGCUCCCUUUGGUAAGUUCUCAGUGUGUAAACCUGACAACACGAACCCAGUUGCCUCCAGCUUAUACCAACUC
AUUUACUCGCGGAGUAUAUUAUCCCGAUAAGGUCUUUAGAAGUAGCGUGUUGCACUCUACACAGGAUCUGUUCUUGCCCU
UCUUUAGUAACGUUACCUGGUUUCAUGCAAUACAUGUGAGCGGAACAAAUGGAACAAAAAGAUUUGACAAUCCAGUGCUU
CCAUUUAAUGAUGGGGUUUACUUUGCCAGUACCGAAAAGUCAAACAUAAUCCGGGGGUGGAUCUUUGGAACCACUUUGGA
CUCUAAGACACAGUCUCUCCUCAUAGUAAACAACGCCACCAAUGUUGUCAUAAAAGUAUGCGAAUUUCAGUUUUGCAACG
AUCCCUUUCUCGGGGUGUAUUACCAUAAGAAUAAUAAAUCCUGGAUGGAGUCUGAGUUCCGGGUUUAUAGUAGUGCUAAU
AAUUGCACUUUCGAAUACGUGUCCCAACCAUUCCUCAUGGACCUUGAGGGCAAACAGGGGAAUUUUAAAAACUUGCGCGA
AUUUGUCUUUAAGAAUAUCGACGGAUACUUUAAGAUCUAUAGUAAACACACUCCUAUCAACCUCGUUCGGGAUCUUCCCC
AAGGCUUUUCUGCUCUCGAACCCCUCGUAGACUUGCCAAUUGGGAUAAAUAUCACUCGCUUUCAAACUUUGCUUGCCCUC
CACAGGAGCUACCUGACACCCGGCGACUCUUCUUCUGGUUGGACCGCCGGCGCCGCUGCCUAUUAUGUUGGUUACCUUCA
GCCACGAACAUUCUUGCUCAAGUAUAACGAGAAUGGCACCAUUACCGACGCCGUCGAUUGUGCAUUGGAUCCCUUGUCUG
AAACAAAAUGUACCUUGAAGUCCUUUACCGUAGAGAAAGGCAUAUACCAGACUUCCAACUUCCGAGUUCAGCCUACAGAA
UCCAUUGUGAGAUUUCCCAACAUCACAAACCUCUGCCCUUUCGGUGAAGUAUUUAAUGCUACACGCUUCGCUUCAGUCUA
UGCCUGGAAUAGGAAGCGCAUAUCAAAUUGCGUGGCCGAUUAUUCAGUCCUCUAUAAUAGCGCAUCCUUCAGUACUUUCA
AGUGCUACGGCGUUUCCCCCACCAAACUCAAUGAUCUUUGCUUCACCAACGUCUAUGCUGACAGUUUUGUCAUACGAGGC
GACGAAGUACGCCAGAUUGCCCCCGGGCAGACAGGUAAAAUUGCUGAUUAUAAUUAUAAACUCCCAGAUGACUUUACUGG
AUGCGUCAUAGCCUGGAAUUCCAACAAUCUUGAUUCCAAGGUUGGUGGGAAUUAUAAUUACCUUUAUCGACUGUUCAGAA
AGAGUAACUUGAAACCAUUUGAGAGAGACAUAUCCACCGAGAUUUACCAGGCAGGCAGUACUCCUUGUAACGGCGUUGAG
GGAUUUAACUGCUAUUUUCCUUUGCAAUCCUAUGGCUUUCAACCAACAAACGGGGUUGGCUAUCAACCCUAUCGAGUGGU
UGUCCUGAGCUUUGAACUUUUGCACGCUCCCGCCACAGUCUGCGGACCAAAAAAGAGUACAAAUCUUGUCAAGAAUAAGU
GCGUAAAUUUCAAUUUCAAUGGCCUUACAGGAACAGGCGUGCUGACUGAGUCAAACAAGAAGUUCCUGCCAUUUCAGCAG
UUUGGGCGGGAUAUAGCAGACACAACUGACGCUGUACGCGAUCCUCAGACUUUGGAGAUCUUGGACAUCACUCCCUGUUC
UUUCGGAGGGGUAUCUGUCAUCACCCCCGGAACUAAUACAUCAAAUCAGGUCGCUGUGUUGUACCAAGAUGUCAACUGCA
CAGAAGUCCCCGUUGCUAUACACGCAGGCCAGCUCACCCCCACAUGGCGGGUGUACUCAACUGGCUCAAACGUAUUCCAG
ACCAGAGCUGGGUGCUUGAUCGGUGCUGAACACGUAAACAAUAGCUAUGAAUGCGAUAUUCCCAUCGGUGCCGGGAUCUG
CGCUAGCUAUCAGACACAGACCAAUUCCCCCCGGCGAGCACGAUCUGUAGCAUCCCAGUCUAUUAUUGCCUACACUAUGU
CAUUGGGCGCCGAGAAUAGCGUCGCAUAUUCAAAUAAUUCUAUUGCAAUACCCACCAACUUCACAAUCUCCGUAACUACA
GAAAUACUUCCAGUUUCCAUGACAAAGACAUCAGUGGAUUGUACAAUGUAUAUAUGCGGAGAUUCCACAGAAUGUUCAAA
UUUGCUCUUGCAGUACGGCUCCUUCUGCACCCAGCUCAACAGGGCCCUUACAGGUAUUGCUGUCGAACAGGACAAGAACA
CACAAGAAGUCUUCGCCCAAGUCAAACAGAUAUACAAAACUCCUCCCAUAAAGGAUUUUGGCGGCUUCAACUUUAGUCAG
AUCCUCCCAGACCCUUCAAAACCAUCUAAACGAUCAUUUAUUGAAGAUCUGCUGUUCAACAAGGUCACUCUUGCCGAUGC
UGGAUUCAUUAAGCAAUACGGUGACUGCCUUGGUGAUAUUGCUGCCCGAGAUCUGAUCUGUGCCCAGAAAUUCAACGGGC
UCACUGUACUCCCUCCACUGCUCACAGACGAAAUGAUUGCACAGUACACAAGUGCCCUGUUGGCAGGCACAAUCACUAGC
GGCUGGACCUUUGGCGCAGGUGCAGCACUCCAAAUACCUUUUGCCAUGCAGAUGGCCUAUCGGUUUAAUGGGAUAGGCGU
GACUCAAAAUGUCCUCUACGAAAACCAAAAGUUGAUAGCUAACCAAUUCAAUUCAGCAAUCGGGAAGAUACAGGAUUCAC
UGUCUAGUACUGCUAGUGCCCUUGGUAAGCUGCAGGACGUUGUCAACCAGAAUGCUCAAGCUCUGAAUACAUUGGUUAAG
CAGCUCUCUAGUAAUUUUGGGGCCAUCUCUUCAGUACUUAAUGAUAUUUUGAGCCGAUUGGACAAAGUGGAAGCUGAAGU
ACAGAUCGACAGGCUGAUAACAGGCCGGCUCCAAUCCCUCCAAACAUACGUGACACAACAACUCAUACGCGCAGCCGAAA
UCCGAGCCAGCGCUAACCUGGCAGCUACCAAGAUGUCAGAAUGCGUUCUGGGCCAGAGUAAACGCGUAGAUUUCUGCGGG
AAAGGGUACCACCUGAUGUCCUUUCCACAAUCUGCACCUCACGGGGUCGUCUUUUUGCAUGUAACAUAUGUACCCGCACA
AGAGAAGAAUUUUACUACCGCUCCUGCCAUCUGUCAUGACGGGAAAGCUCAUUUUCCUCGCGAAGGUGUGUUUGUAUCUA
AUGGUACACAUUGGUUUGUCACACAGCGGAAUUUCUAUGAACCCCAGAUCAUUACAACUGACAACACUUUUGUUUCCGGG
AAUUGUGACGUGGUCAUAGGAAUCGUAAAUAACACUGUAUAUGAUCCCCUCCAACCAGAGCUGGACUCUUUUAAAGAAGA
ACUGGAUAAAUAUUUCAAGAACCACACAAGUCCCGACGUGGACCUUGGGGACAUAAGUGGUAUUAACGCAUCUGUGGUUA
ACAUUCAAAAGGAAAUCGACAGACUCAACGAGGUGGCCAAAAACCUGAACGAAAGCUUGAUAGAUCUCCAGGAGUUGGGC
AAGUAUGAACAGUACAUUAAAUGGCCAUGGUACAUAUGGCUUGGCUUUAUCGCUGGCCUUAUCGCCAUCGUAAUGGUUAC
AAUCAUGCUGUGCUGCAUGACCUCCUGCUGUUCUUGUUUGAAAGGGUGUUGUUCUUGUGGUAGUUGUUGCAAGUUUGACG
AAGAUGAUUCCGAACCUGUUCUUAAAGGGGUAAAGCUUCACUAUACAuaguaaccgcggugucaaaaaccgcguggacgu
cugaccaaccagaaacauaauugaauacagcagcaauuggcaagcugcuuacauagaacucgcggcgauuggcaugccgc
cuuaaaauuuuauuuuauuuuuucuuuucuuuuccgaaucggauuuuguuuuuaauauuucaaaaaaaaaaaaaaaaaaa
aaaaaaaaaaaaaaaaaaaaaaaaaaa
Full-length VEEV+ B.1.351-PP-D614G Antigen Encoding RNA Sequence
SEQ ID NO: 27
auaggcggcgcaugagagaagcccagaccaauuaccuacccaaaauggagaaaguucacguugacaucgaggaagacagc
ccauuccucagagcuuugcagcggagcuucccgcaguuugagguagaagccaagcaggucacugauaaugaccaugcuaa
ugccagagcguuuucgcaucuggcuucaaaacugaucgaaacggagguggacccauccgacacgauccuugacauuggaa
gugcgcccgcccgcagaauguauucuaagcacaaguaucauuguaucuguccgaugagaugugcggaagauccggacaga
uuguauaaguaugcaacuaagcugaagaaaaacuguaaggaaauaacugauaaggaauuggacaagaaaaugaaggagcu
ggccgccgucaugagcgacccugaccuggaaacugagacuaugugccuccacgacgacgagucgugucgcuacgaagggc
aagucgcuguuuaccaggauguauacgcgguugacggaccgacaagucucuaucaccaagccaauaagggaguuagaguc
gccuacuggauaggcuuugacaccaccccuuuuauguuuaagaacuuggcuggagcauauccaucauacucuaccaacug
ggccgacgaaaccguguuaacggcucguaacauaggccuaugcagcucugacguuauggagcggucacguagagggaugu
ccauucuuagaaagaaguauuugaaaccauccaacaauguucuauucucuguuggcucgaccaucuaccacgagaagagg
gacuuacugaggagcuggcaccugccgucuguauuucacuuacguggcaagcaaaauuacacaugucggugugagacuau
aguuaguugcgacggguacgucguuaaaagaauagcuaucaguccaggccuguaugggaagccuucaggcuaugcugcua
cgaugcaccgcgagggauucuugugcugcaaagugacagacacauugaacggggagagggucucuuuucccgugugcacg
uaugugccagcuacauugugugaccaaaugacuggcauacuggcaacagaugucagugcggacgacgcgcaaaaacugcu
gguugggcucaaccagcguauagucgucaacggucgcacccagagaaacaccaauaccaugaaaaauuaccuuuugcccg
uaguggcccaggcauuugcuaggugggcaaaggaauauaaggaagaucaagaagaugaaaggccacuaggacuacgagau
agacaguuagucaugggguguuguugggcuuuuagaaggcacaagauaacaucuauuuauaagcgcccggauacccaaac
caucaucaaagugaacagcgauuuccacucauucgugcugcccaggauaggcaguaacacauuggagaucgggcugagaa
caagaaucaggaaaauguuagaggagcacaaggagccgucaccucucauuaccgccgaggacguacaagaagcuaagugc
gcagccgaugaggcuaaggaggugcgugaagccgaggaguugcgcgcagcucuaccaccuuuggcagcugauguugagga
gcccacucuggaggcagacgucgacuugauguuacaagaggcuggggccggcucaguggagacaccucguggcuugauaa
agguuaccagcuacgauggcgaggacaagaucggcucuuacgcugugcuuucuccgcaggcuguacucaagagugaaaaa
uuaucuugcauccacccucucgcugaacaagucauagugauaacacacucuggccgaaaagggcguuaugccguggaacc
auaccaugguaaaguaguggugccagagggacaugcaauacccguccaggacuuucaagcucugagugaaagugccacca
uuguguacaacgaacgugaguucguaaacagguaccugcaccauauugccacacauggaggagcgcugaacacugaugaa
gaauauuacaaaacugucaagcccagcgagcacgacggcgaauaccuguacgacaucgacaggaaacagugcgucaagaa
agaacuagucacugggcuagggcucacaggcgagcugguggauccucccuuccaugaauucgccuacgagagucugagaa
cacgaccagccgcuccuuaccaaguaccaaccauagggguguauggcgugccaggaucaggcaagucuggcaucauuaaa
agcgcagucaccaaaaaagaucuaguggugagcgccaagaaagaaaacugugcagaaauuauaagggacgucaagaaaau
gaaagggcuggacgucaaugccagaacuguggacucagugcucuugaauggaugcaaacaccccguagagacccuguaua
uugacgaagcuuuugcuugucaugcagguacucucagagcgcucauagccauuauaagaccuaaaaaggcagugcucugc
ggggaucccaaacagugcgguuuuuuuaacaugaugugccugaaagugcauuuuaaccacgagauuugcacacaagucuu
ccacaaaagcaucucucgccguugcacuaaaucugugacuucggucgucucaaccuuguuuuacgacaaaaaaaugagaa
cgacgaauccgaaagagacuaagauugugauugacacuacoggcaguaccaaaccuaagcaggacgaucucauucucacu
uguuucagagggugggugaagcaguugcaaauagauuacaaaggcaacgaaauaaugacggcagcugccucucaagggcu
gacccguaaagguguguaugccguucgguacaaggugaaugaaaauccucuguacgcacccaccucagaacaugugaacg
uccuacugacccgcacggaggaccgcaucguguggaaaacacuagccggcgacccauggauaaaaacacugacugccaag
uacccugggaauuucacugccacgauagaggaguggcaagcagagcaugaugccaucaugaggcacaucuuggagagacc
ggacccuaccgacgucuuccagaauaaggcaaacguguguugggccaaggcuuuagugccggugcugaagaccgcuggca
uagacaugaccacugaacaauggaacacuguggauuauuuugaaacggacaaagcucacucagcagagauaguauugaac
caacuaugcgugagguucuuuggacucgaucuggacuccggucuauuuucugcacccacuguuccguuauccauuaggaa
uaaucacugggauaacuccccgucgccuaacauguacgggcugaauaaagaagugguccgucagcucucucgcagguacc
cacaacugccucgggcaguugccacuggaagagucuaugacaugaacacugguacacugcgcaauuaugauccgcgcaua
aaccuaguaccuguaaacagaagacugccucaugcuuuaguccuccaccauaaugaacacccacagagugacuuuucuuc
auucgucagcaaauugaagggcagaacuguccugguggucggggaaaaguuguccgucccaggcaaaaugguugacuggu
ugucagaccggccugaggcuaccuucagagcucggcuggauuuaggcaucccaggugaugugcccaaauaugacauaaua
uuuguuaaugugaggaccccauauaaauaccaucacuaucagcagugugaagaccaugccauuaagcuuagcauguugac
caagaaagcuugucugcaucugaaucccggcggaaccugugucagcauagguuaugguuacgcugacagggccagcgaaa
gcaucauuggugcuauagcgcggcaguucaaguuuucccggguaugcaaaccgaaauccucacuugaagagacggaaguu
cuguuuguauucauuggguacgaucgcaaggcccguacgcacaauccuuacaagcuuucaucaaccuugaccaacauuua
uacagguuccagacuccacgaagccggaugugcacccucauaucauguggugcgaggggauauugccacggccaccgaag
gagugauuauaaaugcugcuaacagcaaaggacaaccuggcggaggggugugcggagcgcuguauaagaaauucccggaa
agcuucgauuuacagccgaucgaaguaggaaaagcgcgacuggucaaaggugcagcuaaacauaucauucaugccguagg
accaaacuucaacaaaguuucggagguugaaggugacaaacaguuggcagaggcuuaugaguccaucgcuaagauuguca
acgauaacaauuacaagucaguagcgauuccacuguuguccaccggcaucuuuuccgggaacaaagaucgacuaacccaa
ucauugaaccauuugcugacagcuuuagacaccacugaugcagauguagccauauacugcagggacaagaaaugggaaau
gacucucaaggaagcaguggcuaggagagaagcaguggaggagauaugcauauccgacgacucuucagugacagaaccug
augcagagcuggugagggugcauccgaagaguucuuuggcuggaaggaagggcuacagcacaagcgauggcaaaacuuuc
ucauauuuggaagggaccaaguuucaccaggcggccaaggauauagcagaaauuaaugccauguggcccguugcaacgga
ggccaaugagcagguaugcauguauauccucggagaaagcaugagcaguauuaggucgaaaugccccgucgaagagucgg
aagccuccacaccaccuagcacgcugccuugcuugugcauccaugccaugacuccagaaagaguacagcgccuaaaagcc
ucacguccagaacaaauuacugugugcucauccuuuccauugccgaaguauagaaucacuggugugcagaagauccaaug
cucccagccuauauuguucucaccgaaagugccugcguauauucauccaaggaaguaucucguggaaacaccaccgguag
acgagacuccggagccaucggcagagaaccaauccacagaggggacaccugaacaaccaccacuuauaaccgaggaugag
accaggacuagaacgccugagccgaucaucaucgaagaggaagaagaggauagcauaaguuugcugucagauggcccgac
ccaccaggugcugcaagucgaggcagacauucacgggccgcccucuguaucuagcucauccugguccauuccucaugcau
ccgacuuugauguggacaguuuauccauacuugacacccuggagggagcuagcgugaccagcggggcaacgucagccgag
acuaacucuuacuucgcaaagaguauggaguuucuggcgcgaccggugccugcgccucgaacaguauucaggaacccucc
acaucccgcuccgcgcacaagaacaccgucacuugcacccagcagggccugcucgagaaccagccuaguuuccaccccgc
caggcgugaauagggugaucacuagagaggagcucgaggcgcuuaccccgucacgcacuccuagcaggucggucucgaga
accagccuggucuccaacccgccaggcguaaauagggugauuacaagagaggaguuugaggcguucguagcacaacaaca
augacgguuugaugcgggugcauacaucuuuuccuccgacaccggucaagggcauuuacaacaaaaaucaguaaggcaaa
cggugcuauccgaagugguguuggagaggaccgaauuggagauuucguaugccccgcgccucgaccaagaaaaagaagaa
uuacuacgcaagaaauuacaguuaaaucccacaccugcuaacagaagcagauaccaguccaggaagguggagaacaugaa
agccauaacagcuagacguauucugcaaggccuagggcauuauuugaaggcagaaggaaaaguggagugcuaccgaaccc
ugcauccuguuccuuuguauucaucuagugugaaccgugccuuuucaagccccaaggucgcaguggaagccuguaacgcc
auguugaaagagaacuuuccgacuguggcuucuuacuguauuauuccagaguacgaugccuauuuggacaugguugacgg
agcuucaugcugcuuagacacugccaguuuuugcccugcaaagcugcgcagcuuuccaaagaaacacuccuauuuggaac
ccacaauacgaucggcagugccuucagcgauccagaacacgcuccagaacguccuggcagcugccacaaaaagaaauugc
aaugucacgcaaaugagagaauugcccguauuggauucggcggccuuuaauguggaaugcuucaagaaauaugcguguaa
uaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuugua
auggacuuaaagagagacgugaaagugacuccaggaacaaaacauacugaagaacggcccaagguacaggugauccaggc
ugccgauccgcuagcaacagcguaucugugcggaauccaccgagagcugguuaggagauuaaaugcgguccugcuuccga
acauucauacacuguuugauaugucggcugaagacuuugacgcuauuauagccgagcacuuccagccuggggauuguguu
cuggaaacugacaucgcgucguuugauaaaagugaggacgacgccauggcucugaccgcguuaaugauucuggaagacuu
agguguggacgcagagcuguugacgcugauugaggcggcuuucggcgaaauuucaucaauacauuugcccacuaaaacua
aauuuaaauucggagccaugaugaaaucuggaauguuccucacacuguuugugaacacagucauuaacauuguaaucgca
agcagaguguugagagaacggcuaaccggaucaccaugugcagcauucauuggagaugacaauaucgugaaaggagucaa
aucggacaaauuaauggcagacaggugcgccaccugguugaauauggaagucaagauuauagaugcuguggugggcgaga
aagcgccuuauuucuguggaggguuuauuuugugugacuccgugaccggcacagcgugccguguggcagacccccuaaaa
aggcuguuuaagcuuggcaaaccucuggcagcagacgaugaacaugaugaugacaggagaagggcauugcaugaagaguc
aacacgcuggaaccgaguggguauucuuucagagcugugcaaggcaguagaaucaagguaugaaaccguaggaacuucca
ucauaguuauggccaugacuacucuagcuagcaguguuaaaucauucagcuaccugagaggggccccuauaacucucuac
ggcuaaccugaauggacuacgacauagucuaguccgccaagAUGUUUCUGCUCACAACCAAACGCACUAUGUUUGUUUUC
CUCGUGCUGCUCCCUUUGGUAAGUUCUCAGUGUGUAAACuucACAACACGAACCCAGUUGCCUCCAGCUUAUACCAACUC
AUUUACUCGCGGAGUAUAUUAUCCCGAUAAGGUCUUUAGAAGUAGCGUGUUGCACUCUACACAGGAUCUGUUCUUGCCCU
UCUUUAGUAACGUUACCUGGUUUCAUGCAAUACAUGUGAGCGGAACAAAUGGAACAAAAAGAUUUgccAAUCCAGUGCUU
CCAUUUAAUGAUGGGGUUUACUUUGCCAGUACCGAAAAGUCAAACAUAAUCCGGGGGUGGAUCUUUGGAACCACUUUGGA
CUCUAAGACACAGUCUCUCCUCAUAGUAAACAACGCCACCAAUGUUGUCAUAAAAGUAUGCGAAUUUCAGUUUUGCAACG
AUCCCUUUCUCGGGGUGUAUUACCAUAAGAAUAAUAAAUCCUGGAUGGAGUCUGAGUUCCGGGUUUAUAGUAGUGCUAAU
AAUUGCACUUUCGAAUACGUGUCCCAACCAUUCCUCAUGGACCUUGAGGGCAAACAGGGGAAUUUUAAAAACUUGCGCGA
AUUUGUCUUUAAGAAUAUCGACGGAUACUUUAAGAUCUAUAGUAAACACACUCCUAUCAACCUCGUUCGGggcCUUCCCC
AAGGCUUUUCUGCUCUCGAACCCCUCGUAGACUUGCCAAUUGGGAUAAAUAUCACUCGCUUUCAAACUUUGCACAUCAGC
UACCUGACACCCGGCGACUCUUCUUCUGGUUGGACCGCCGGCGCCGCUGCCUAUUAUGUUGGUUACCUUCAGCCACGAAC
AUUCUUGCUCAAGUAUAACGAGAAUGGCACCAUUACCGACGCCGUCGAUUGUGCAUUGGAUCCCUUGUCUGAAACAAAAU
GUACCUUGAAGUCCUUUACCGUAGAGAAAGGCAUAUACCAGACUUCCAACUUCCGAGUUCAGCCUACAGAAUCCAUUGUG
AGAUUUCCCAACAUCACAAACCUCUGCCCUUUCGGUGAAGUAUUUAAUGCUACACGCUUCGCUUCAGUCUAUGCCUGGAA
UAGGAAGCGCAUAUCAAAUUGCGUGGCCGAUUAUUCAGUCCUCUAUAAUAGCGCAUCCUUCAGUACUUUCAAGUGCUACG
GCGUUUCCCCCACCAAACUCAAUGAUCUUUGCUUCACCAACGUCUAUGCUGACAGUUUUGUCAUACGAGGCGACGAAGUA
CGCCAGAUUGCCCCCGGGCAGACAGGUAACAUUGCUGAUUAUAAUUAUAAACUCCCAGAUGACUUUACUGGAUGCGUCAU
AGCCUGGAAUUCCAACAAUCUUGAUUCCAAGGUUGGUGGGAAUUAUAAUUACCUUUAUCGACUGUUCAGAAAGAGUAACU
UGAAACCAUUUGAGAGAGACAUAUCCACCGAGAUUUACCAGGCAGGCAGUACUCCUUGUAACGGCGUUaagGGAUUUAAC
UGCUAUUUUCCUUUGCAAUCCUAUGGCUUUCAACCAACAuacGGGGUUGGCUAUCAACCCUAUCGAGUGGUUGUCCUGAG
CUUUGAACUUUUGCACGCUCCCGCCACAGUCUGCGGACCAAAAAAGAGUACAAAUCUUGUCAAGAAUAAGUGCGUAAAUU
UCAAUUUCAAUGGCCUUACAGGAACAGGCGUGCUGACUGAGUCAAACAAGAAGUUCCUGCCAUUUCAGCAGUUUGGGCGG
GAUAUAGCAGACACAACUGACGCUGUACGCGAUCCUCAGACUUUGGAGAUCUUGGACAUCACUCCCUGUUCUUUCGGAGG
GGUAUCUGUCAUCACCCCCGGAACUAAUACAUCAAAUCAGGUCGCUGUGUUGUACCAAggcGUCAACUGCACAGAAGUCC
CCGUUGCUAUACACGCAGACCAGCUCACCCCCACAUGGCGGGUGUACUCAACUGGCUCAAACGUAUUCCAGACCAGAGCU
GGGUGCUUGAUCGGUGCUGAACACGUAAACAAUAGCUAUGAAUGCGAUAUUCCCAUCGGUGCCGGGAUCUGCGCUAGCUA
UCAGACACAGACCAAUUCCCCCCGGCGAGCACGAUCUGUAGCAUCCCAGUCUAUUAUUGCCUACACUAUGUCAUUGGGCG
UGGAGAAUAGCGUCGCAUAUUCAAAUAAUUCUAUUGCAAUACCCACCAACUUCACAAUCUCCGUAACUACAGAAAUACUU
CCAGUUUCCAUGACAAAGACAUCAGUGGAUUGUACAAUGUAUAUAUGCGGAGAUUCCACAGAAUGUUCAAAUUUGCUCUU
GCAGUACGGCUCCUUCUGCACCCAGCUCAACAGGGCCCUUACAGGUAUUGCUGUCGAACAGGACAAGAACACACAAGAAG
UCUUCGCCCAAGUCAAACAGAUAUACAAAACUCCUCCCAUAAAGGAUUUUGGCGGCUUCAACUUUAGUCAGAUCCUCCCA
GACCCUUCAAAACCAUCUAAACGAUCAUUUAUUGAAGAUCUGCUGUUCAACAAGGUCACUCUUGCCGAUGCUGGAUUCAU
UAAGCAAUACGGUGACUGCCUUGGUGAUAUUGCUGCCCGAGAUCUGAUCUGUGCCCAGAAAUUCAACGGGCUCACUGUAC
UCCCUCCACUGCUCACAGACGAAAUGAUUGCACAGUACACAAGUGCCCUGUUGGCAGGCACAAUCACUAGCGGCUGGACC
UUUGGCGCAGGUGCAGCACUCCAAAUACCUUUUGCCAUGCAGAUGGCCUAUCGGUUUAAUGGGAUAGGCGUGACUCAAAA
UGUCCUCUACGAAAACCAAAAGUUGAUAGCUAACCAAUUCAAUUCAGCAAUCGGGAAGAUACAGGAUUCACUGUCUAGUA
CUGCUAGUGCCCUUGGUAAGCUGCAGGACGUUGUCAACCAGAAUGCUCAAGCUCUGAAUACAUUGGUUAAGCAGCUCUCU
AGUAAUUUUGGGGCCAUCUCUUCAGUACUUAAUGAUAUUUUGAGCCGAUUGGACccacccGAAGCUGAAGUACAGAUCGA
CAGGCUGAUAACAGGCCGGCUCCAAUCCCUCCAAACAUACGUGACACAACAACUCAUACGCGCAGCCGAAAUCCGAGCCA
GCGCUAACCUGGCAGCUACCAAGAUGUCAGAAUGCGUUCUGGGCCAGAGUAAACGCGUAGAUUUCUGCGGGAAAGGGUAC
CACCUGAUGUCCUUUCCACAAUCUGCACCUCACGGGGUCGUCUUUUUGCAUGUAACAUAUGUACCCGCACAAGAGAAGAA
UUUUACUACCGCUCCUGCCAUCUGUCAUGACGGGAAAGCUCAUUUUCCUCGCGAAGGUGUGUUUGUAUCUAAUGGUACAC
AUUGGUUUGUCACACAGCGGAAUUUCUAUGAACCCCAGAUCAUUACAACUGACAACACUUUUGUUUCCGGGAAUUGUGAC
GUGGUCAUAGGAAUCGUAAAUAACACUGUAUAUGAUCCCCUCCAACCAGAGCUGGACUCUUUUAAAGAAGAACUGGAUAA
AUAUUUCAAGAACCACACAAGUCCCGACGUGGACCUUGGGGACAUAAGUGGUAUUAACGCAUCUGUGGUUAACAUUCAAA
AGGAAAUCGACAGACUCAACGAGGUGGCCAAAAACCUGAACGAAAGCUUGAUAGAUCUCCAGGAGUUGGGCAAGUAUGAA
CAGUACAUUAAAUGGCCAUGGUACAUAUGGCUUGGCUUUAUCGCUGGCCUUAUCGCCAUCGUAAUGGUUACAAUCAUGCU
GUGCUGCAUGACCUCCUGCUGUUCUUGUUUGAAAGGGUGUUGUUCUUGUGGUAGUUGUUGCAAGUUUGACGAAGAUGAUU
CCGAACCUGUUCUUAAAGGGGUAAAGCUUCACUAUACAugauaaccgcggugucaaaaaccgcguggacgugguuaacau
cccugcugggaggaucagccguaauuauuauaauuggcuuggugcuggcuacuauuguggccauguacgugcugaccaac
cagaaacauaauugaauacagcagcaauuggcaagcugcuuacauagaacucgcggcgauuggcaugccgccuuaaaauu
uuuauuuuauuuuuucuuuucuuuccgaaucggauuuuguuuuuaauauuucaaaaaaaaaaaaaaaaaaaaaaaaaaaa
aaaaaaaaaaaaaaaaaaaaa
Full-length VEEV+ B.1.1.7-PP-D614G Antigen Encoding RNA Sequence
SEQ ID NO: 28
auaggcggcgcaugagagaagcccagaccaauuaccuacccaaaauggagaaaguucacguugacaucgaggaagac
agcccauuccucagagcuuugcagcggagcuucccgcaguuugagguagaagccaagcaggucacugauaaugacca
ugcuaaugccagagcguuuucgcaucuggcuucaaaacugaucgaaacggagguggacccauccgacacgauccuug
acauuggaagugcgcccgcccgcagaauguauucuaagcacaaguaucauuguaucuguccgaugagaugugcggaa
gauccggacagauuguauaaguaugcaacuaagcugaagaaaaacuguaaggaaauaacugauaaggaauuggacaa
gaaaaugaaggagcuggccgccgucaugagcgacccugaccuggaaacugagacuaugugccuccacgacgacgagu
cgugucgcuacgaagggcaagucgcuguuuaccaggauguauacgcgguugacggaccgacaagucucuaucaccaa
gccaauaagggaguuagagucgccuacuggauaggcuuugacaccaccccuuuuauguuuaagaacuuggcuggagc
auauccaucauacucuaccaacugggccgacgaaaccguguuaacggcucguaacauaggccuaugcagcucugacg
uuauggagcggucacguagagggauguccauucuuagaaagaaguauuugaaaccauccaacaauguucuauucucu
guuggcucgaccaucuaccacgagaagagggacuuacugaggagcuggcaccugccgucuguauuucacuuacgugg
caagcaaaauuacacaugucggugugagacuauaguuaguugcgacggguacgucguuaaaagaauagcuaucaguc
caggccuguaugggaagccuucaggcuaugcugcuacgaugcaccgcgagggauucuugugcugcaaagugacagac
acauugaacggggagagggucucuuuucccgugugcacguaugugccagcuacauugugugaccaaaugacuggcau
acuggcaacagaugucagugcggacgacgcgcaaaaacugcugguugggcucaaccagcguauagucgucaacgguc
gcacccagagaaacaccaauaccaugaaaaauuaccuuuugcccguaguggcccaggcauuugcuaggugggcaaag
gaauauaaggaagaucaagaagaugaaaggccacuaggacuacgagauagacaguuagucaugggguguuguugggc
uuuuagaaggcacaagauaacaucuauuuauaagcgcccggauacccaaaccaucaucaaagugaacagcgauuucc
acucauucgugcugcccaggauaggcaguaacacauuggagaucgggcugagaacaagaaucaggaaaauguuagag
gagcacaaggagccgucaccucucauuaccgccgaggacguacaagaagcuaagugcgcagccgaugaggcuaagga
ggugcgugaagccgaggaguugcgcgcagcucuaccaccuuuggcagcugauguugaggagcccacucuggaggcag
acgucgacuugauguuacaagaggcuggggccggcucaguggagacaccucguggcuugauaaagguuaccagcuac
gauggcgaggacaagaucggcucuuacgcugugcuuucuccgcaggcuguacucaagagugaaaaauuaucuugcau
ccacccucucgcugaacaagucauagugauaacacacucuggccgaaaagggcguuaugccguggaaccauaccaug
guaaaguaguggugccagagggacaugcaauacccguccaggacuuucaagcucugagugaaagugccaccauugug
uacaacgaacgugaguucguaaacagguaccugcaccauauugccacacauggaggagcgcugaacacugaugaaga
auauuacaaaacugucaagcccagcgagcacgacggcgaauaccuguacgacaucgacaggaaacagugcgucaaga
aagaacuagucacugggcuagggcucacaggcgagcugguggauccucccuuccaugaauucgccuacgagagucug
agaacacgaccagccgcuccuuaccaaguaccaaccauagggguguauggcgugccaggaucaggcaagucuggcau
cauuaaaagcgcagucaccaaaaaagaucuaguggugagcgccaagaaagaaaacugugcagaaauuauaagggacg
ucaagaaaaugaaagggcuggacgucaaugccagaacuguggacucagugcucuugaauggaugcaaacaccccgua
gagacccuguauauugacgaagcuuuugcuugucaugcagguacucucagagcgcucauagccauuauaagaccuaa
aaaggcagugcucugcggggaucccaaacagugcgguuuuuuuaacaugaugugccugaaagugcauuuuaaccacg
agauuugcacacaagucuuccacaaaagcaucucucgccguugcacuaaaucugugacuucggucgucucaaccuug
uuuuacgacaaaaaaaugagaacgacgaauccgaaagagacuaagauugugauugacacuaccggcaguaccaaacc
uaagcaggacgaucucauucucacuuguuucagagggugggugaagcaguugcaaauagauuacaaaggcaacgaaa
uaaugacggcagcugccucucaagggcugacccguaaagguguguaugccguucgguacaaggugaaugaaaauccu
cuguacgcacccaccucagaacaugugaacguccuacugacccgcacggaggaccgcaucguguggaaaacacuagc
cggcgacccauggauaaaaacacugacugccaaguacccugggaauuucacugccacgauagaggaguggcaagcag
agcaugaugccaucaugaggcacaucuuggagagaccggacccuaccgacgucuuccagaauaaggcaaacgugugu
ugggccaaggcuuuagugccggugcugaagaccgcuggcauagacaugaccacugaacaauggaacacuguggauua
uuuugaaacggacaaagcucacucagcagagauaguauugaaccaacuaugcgugagguucuuuggacucgaucugg
acuccggucuauuuucugcacccacuguuccguuauccauuaggaauaaucacugggauaacuccccgucgccuaac
auguacgggcugaauaaagaagugguccgucagcucucucgcagguacccacaacugccucgggcaguugccacugg
aagagucuaugacaugaacacugguacacugcgcaauuaugauccgcgcauaaaccuaguaccuguaaacagaagac
ugccucaugcuuuaguccuccaccauaaugaacacccacagagugacuuuucuucauucgucagcaaauugaagggc
agaacuguccugguggucggggaaaaguuguccgucccaggcaaaaugguugacugguugucagaccggccugaggc
uaccuucagagcucggcuggauuuaggcaucccaggugaugugcccaaauaugacauaauauuuguuaaugugagga
ccccauauaaauaccaucacuaucagcagugugaagaccaugccauuaagcuuagcauguugaccaagaaagcuugu
cugcaucugaaucccggcggaaccugugucagcauagguuaugguuacgcugacagggccagcgaaagcaucauugg
ugcuauagcgcggcaguucaaguuuucccggguaugcaaaccgaaauccucacuugaagagacggaaguucuguuug
uauucauuggguacgaucgcaaggcccguacgcacaauccuuacaagcuuucaucaaccuugaccaacauuuauaca
gguuccagacuccacgaagccggaugugcacccucauaucauguggugcgaggggauauugccacggccaccgaagg
agugauuauaaaugcugcuaacagcaaaggacaaccuggcggaggggugugcggagcgcuguauaagaaauucccgg
aaagcuucgauuuacagccgaucgaaguaggaaaagcgcgacuggucaaaggugcagcuaaacauaucauucaugcc
guaggaccaaacuucaacaaaguuucggagguugaaggugacaaacaguuggcagaggcuuaugaguccaucgcuaa
gauugucaacgauaacaauuacaagucaguagcgauuccacuguuguccaccggcaucuuuuccgggaacaaagauc
gacuaacccaaucauugaaccauuugcugacagcuuuagacaccacugaugcagauguagccauauacugcagggac
aagaaaugggaaaugacucucaaggaagcaguggcuaggagagaagcaguggaggagauaugcauauccgacgacuc
uucagugacagaaccugaugcagagcuggugagggugcauccgaagaguucuuuggcuggaaggaagggcuacagca
caagcgauggcaaaacuuucucauauuuggaagggaccaaguuucaccaggcggccaaggauauagcagaaauuaau
gccauguggcccguugcaacggaggccaaugagcagguaugcauguauauccucggagaaagcaugagcaguauuag
gucgaaaugccccgucgaagagucggaagccuccacaccaccuagcacgcugccuugcuugugcauccaugccauga
cuccagaaagaguacagcgccuaaaagccucacguccagaacaaauuacugugugcucauccuuuccauugccgaag
uauagaaucacuggugugcagaagauccaaugcucccagccuauauuguucucaccgaaagugccugcguauauuca
uccaaggaaguaucucguggaaacaccaccgguagacgagacuccggagccaucggcagagaaccaauccacagagg
ggacaccugaacaaccaccacuuauaaccgaggaugagaccaggacuagaacgccugagccgaucaucaucgaagag
gaagaagaggauagcauaaguuugcugucagauggcccgacccaccaggugcugcaagucgaggcagacauucacgg
gccgcccucuguaucuagcucauccugguccauuccucaugcauccgacuuugauguggacaguuuauccauacuug
acacccuggagggagcuagcgugaccagcggggcaacgucagccgagacuaacucuuacuucgcaaagaguauggag
uuucuggcgcgaccggugccugcgccucgaacaguauucaggaacccuccacaucccgcuccgcgcacaagaacacc
gucacuugcacccagcagggccugcucgagaaccagccuaguuuccaccccgccaggcgugaauagggugaucacua
gagaggagcucgaggcgcuuaccccgucacgcacuccuagcaggucggucucgagaaccagccuggucuccaacccg
ccaggcguaaauagggugauuacaagagaggaguuugaggcguucguagcacaacaacaaugacgguuugaugcggg
ugcauacaucuuuuccuccgacaccggucaagggcauuuacaacaaaaaucaguaaggcaaacggugcuauccgaag
ugguguuggagaggaccgaauuggagauuucguaugccccgcgccucgaccaagaaaaagaagaauuacuacgcaag
aaauuacaguuaaaucccacaccugcuaacagaagcagauaccaguccaggaagguggagaacaugaaagccauaac
agcuagacguauucugcaaggccuagggcauuauuugaaggcagaaggaaaaguggagugcuaccgaacccugcauc
cuguuccuuuguauucaucuagugugaaccgugccuuuucaagccccaaggucgcaguggaagccuguaacgccaug
uugaaagagaacuuuccgacuguggcuucuuacuguauuauuccagaguacgaugccuauuuggacaugguugacgg
agcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaaga
aauugcaaugucacgcaaaugagagaauugcccguauuggauucggcggccuuuaauguggaaugcuucaagaaaua
ugcguguaauaaugaauauugggaaacguuuaaagaaaaccccaucaggcuuacugaagaaaacgugguaaauuaca
uuaccaaauuaaaaggaccaaaagcugcugcucuuuuugcgaagacacauaauuugaauauguugcaggacauacca
auggacagguuuguaauggacuuaaagagagacgugaaagugacuccaggaacaaaacauacugaagaacggcccaa
gguacaggugauccaggcugccgauccgcuagcaacagcguaucugugcggaauccaccgagagcugguuaggagau
uaaaugcgguccugcuuccgaacauucauacacuguuugauaugucggcugaagacuuugacgcuauuauagccgag
cacuuccagccuggggauuguguucuggaaacugacaucgcgucguuugauaaaagugaggacgacgccauggcucu
gaccgcguuaaugauucuggaagacuuagguguggacgcagagcuguugacgcugauugaggcggcuuucggcgaaa
uuucaucaauacauuugcccacuaaaacuaaauuuaaauucggagccaugaugaaaucuggaauguuccucacacug
uuugugaacacagucauuaacauuguaaucgcaagcagaguguugagagaacggcuaaccggaucaccaugugcagc
auucauuggagaugacaauaucgugaaaggagucaaaucggacaaauuaauggcagacaggugcgccaccugguuga
auauggaagucaagauuauagaugcuguggugggcgagaaagcgccuuauuucuguggaggguuuauuuugugugac
uccgugaccggcacagcgugccguguggcagacccccuaaaaaggcuguuuaagcuuggcaaaccucuggcagcaga
cgaugaacaugaugaugacaggagaagggcauugcaugaagagucaacacgcuggaaccgaguggguauucuuucag
agcugugcaaggcaguagaaucaagguaugaaaccguaggaacuuccaucauaguuauggccaugacuacucuagcu
agcaguguuaaaucauucagcuaccugagaggggccccuauaacucucuacggcuaaccugaauggacuacgacaua
gucuaguccgccaagAUGUUUCUGCUCACAACCAAACGCACUAUGUUUGUUUUCCUCGUGCUGCUCCCUUUGGUAAG
UUCUCAGUGUGUAAACCUGACAACACGAACCCAGUUGCCUCCAGCUUAUACCAACUCAUUUACUCGCGGAGUAUAUU
AUCCCGAUAAGGUCUUUAGAAGUAGCGUGUUGCACUCUACACAGGAUCUGUUCUUGCCCUUCUUUAGUAACGUUACC
UGGUUUCAUGCAAUAAGCGGAACAAAUGGAACAAAAAGAUUUGACAAUCCAGUGCUUCCAUUUAAUGAUGGGGUUUA
CUUUGCCAGUACCGAAAAGUCAAACAUAAUCCGGGGGUGGAUCUUUGGAACCACUUUGGACUCUAAGACACAGUCUC
UCCUCAUAGUAAACAACGCCACCAAUGUUGUCAUAAAAGUAUGCGAAUUUCAGUUUUGCAACGAUCCCUUUCUCGGG
GUGUACCAUAAGAAUAAUAAAUCCUGGAUGGAGUCUGAGUUCCGGGUUUAUAGUAGUGCUAAUAAUUGCACUUUCGA
AUACGUGUCCCAACCAUUCCUCAUGGACCUUGAGGGCAAACAGGGGAAUUUUAAAAACUUGCGCGAAUUUGUCUUUA
AGAAUAUCGACGGAUACUUUAAGAUCUAUAGUAAACACACUCCUAUCAACCUCGUUCGGGAUCUUCCCCAAGGCUUU
UCUGCUCUCGAACCCCUCGUAGACUUGCCAAUUGGGAUAAAUAUCACUCGCUUUCAAACUUUGCUUGCCCUCCACAG
GAGCUACCUGACACCCGGCGACUCUUCUUCUGGUUGGACCGCCGGCGCCGCUGCCUAUUAUGUUGGUUACCUUCAGC
CACGAACAUUCUUGCUCAAGUAUAACGAGAAUGGCACCAUUACCGACGCCGUCGAUUGUGCAUUGGAUCCCUUGUCU
GAAACAAAAUGUACCUUGAAGUCCUUUACCGUAGAGAAAGGCAUAUACCAGACUUCCAACUUCCGAGUUCAGCCUAC
AGAAUCCAUUGUGAGAUUUCCCAACAUCACAAACCUCUGCCCUUUCGGUGAAGUAUUUAAUGCUACACGCUUCGCUU
CAGUCUAUGCCUGGAAUAGGAAGCGCAUAUCAAAUUGCGUGGCCGAUUAUUCAGUCCUCUAUAAUAGCGCAUCCUUC
AGUACUUUCAAGUGCUACGGCGUUUCCCCCACCAAACUCAAUGAUCUUUGCUUCACCAACGUCUAUGCUGACAGUUU
UGUCAUACGAGGCGACGAAGUACGCCAGAUUGCCCCCGGGCAGACAGGUAAAAUUGCUGAUUAUAAUUAUAAACUCC
CAGAUGACUUUACUGGAUGCGUCAUAGCCUGGAAUUCCAACAAUCUUGAUUCCAAGGUUGGUGGGAAUUAUAAUUAC
CUUUAUCGACUGUUCAGAAAGAGUAACUUGAAACCAUUUGAGAGAGACAUAUCCACCGAGAUUUACCAGGCAGGCAG
UACUCCUUGUAACGGCGUUGAGGGAUUUAACUGCUAUUUUCCUUUGCAAUCCUAUGGCUUUCAACCAACAuacGGGG
UUGGCUAUCAACCCUAUCGAGUGGUUGUCCUGAGCUUUGAACUUUUGCACGCUCCCGCCACAGUCUGCGGACCAAAA
AAGAGUACAAAUCUUGUCAAGAAUAAGUGCGUAAAUUUCAAUUUCAAUGGCCUUACAGGAACAGGCGUGCUGACUGA
GUCAAACAAGAAGUUCCUGCCAUUUCAGCAGUUUGGGCGGGAUAUAgacGACACAACUGACGCUGUACGCGAUCCUC
AGACUUUGGAGAUCUUGGACAUCACUCCCUGUUCUUUCGGAGGGGUAUCUGUCAUCACCCCCGGAACUAAUACAUCA
AAUCAGGUCGCUGUGUUGUACCAAggcGUCAACUGCACAGAAGUCCCCGUUGCUAUACACGCAGAUCAGCUCACCCC
CACAUGGCGGGUGUACUCAACUGGCUCAAACGUAUUCCAGACCAGAGCUGGGUGCUUGAUCGGUGCUGAACACGUAA
ACAAUAGCUAUGAAUGCGAUAUUCCCAUCGGUGCCGGGAUCUGCGCUAGCUAUCAGACACAGACCAAUUCCcauCGG
CGAGCACGAUCUGUAGCAUCCCAGUCUAUUAUUGCCUACACUAUGUCAUUGGGCGCCGAGAAUAGCGUCGCAUAUUC
AAAUAAUUCUAUUGCAAUACCCaucAACUUCACAAUCUCCGUAACUACAGAAAUACUUCCAGUUUCCAUGACAAAGA
CAUCAGUGGAUUGUACAAUGUAUAUAUGCGGAGAUUCCACAGAAUGUUCAAAUUUGCUCUUGCAGUACGGCUCCUUC
UGCACCCAGCUCAACAGGGCCCUUACAGGUAUUGCUGUCGAACAGGACAAGAACACACAAGAAGUCUUCGCCCAAGU
CAAACAGAUAUACAAAACUCCUCCCAUAAAGGAUUUUGGCGGCUUCAACUUUAGUCAGAUCCUCCCAGACCCUUCAA
AACCAUCUAAACGAUCAUUUAUUGAAGAUCUGCUGUUCAACAAGGUCACUCUUGCCGAUGCUGGAUUCAUUAAGCAA
UACGGUGACUGCCUUGGUGAUAUUGCUGCCCGAGAUCUGAUCUGUGCCCAGAAAUUCAACGGGCUCACUGUACUCCC
UCCACUGCUCACAGACGAAAUGAUUGCACAGUACACAAGUGCCCUGUUGGCAGGCACAAUCACUAGCGGCUGGACCU
UUGGCGCAGGUGCAGCACUCCAAAUACCUUUUGCCAUGCAGAUGGCCUAUCGGUUUAAUGGGAUAGGCGUGACUCAA
AAUGUCCUCUACGAAAACCAAAAGUUGAUAGCUAACCAAUUCAAUUCAGCAAUCGGGAAGAUACAGGAUUCACUGUC
UAGUACUGCUAGUGCCCUUGGUAAGCUGCAGGACGUUGUCAACCAGAAUGCUCAAGCUCUGAAUACAUUGGUUAAGC
AGCUCUCUAGUAAUUUUGGGGCCAUCUCUUCAGUACUUAAUGAUAUUUUGgccCGAUUGGACccacccGAAGCUGAA
GUACAGAUCGACAGGCUGAUAACAGGCCGGCUCCAAUCCCUCCAAACAUACGUGACACAACAACUCAUACGCGCAGC
CGAAAUCCGAGCCAGCGCUAACCUGGCAGCUACCAAGAUGUCAGAAUGCGUUCUGGGCCAGAGUAAACGCGUAGAUU
UCUGCGGGAAAGGGUACCACCUGAUGUCCUUUCCACAAUCUGCACCUCACGGGGUCGUCUUUUUGCAUGUAACAUAU
GUACCCGCACAAGAGAAGAAUUUUACUACCGCUCCUGCCAUCUGUCAUGACGGGAAAGCUCAUUUUCCUCGCGAAGG
UGUGUUUGUAUCUAAUGGUACACAUUGGUUUGUCACACAGCGGAAUUUCUAUGAACCCCAGAUCAUUACAACUcacA
ACACUUUUGUUUCCGGGAAUUGUGACGUGGUCAUAGGAAUCGUAAAUAACACUGUAUAUGAUCCCCUCCAACCAGAG
CUGGACUCUUUUAAAGAAGAACUGGAUAAAUAUUUCAAGAACCACACAAGUCCCGACGUGGACCUUGGGGACAUAAG
UGGUAUUAACGCAUCUGUGGUUAACAUUCAAAAGGAAAUCGACAGACUCAACGAGGUGGCCAAAAACCUGAACGAAA
GCUUGAUAGAUCUCCAGGAGUUGGGCAAGUAUGAACAGUACAUUAAAUGGCCAUGGUACAUAUGGCUUGGCUUUAUC
GCUGGCCUUAUCGCCAUCGUAAUGGUUACAAUCAUGCUGUGCUGCAUGACCUCCUGCUGUUCUUGUUUGAAAGGGUG
UUGUUCUUGUGGUAGUUGUUGCAAGUUUGACGAAGAUGAUUCCGAACCUGUUCUUAAAGGGGUAAAGCUUCACUAUA
CAugauaaccgcggugucaaaaaccgcguggacgugguuaacaucccugcugggaggaucagccguaauuauuauaa
uuggcuuggugcuggcuacuauuguggccauguacgugcugaccaaccagaaacauaauugaauacagcagcaauug
gaaucggauuuuguuuuuaauauuucaaaaaaaaaaaaaaaaaaaaaaaaaaaaaaaaaaaaaaaaaaaaa
Full-length VEEV+ Delta.AY1-S2P-wtFur Antigen Encoding RNA Sequence
SEQ ID NO: 29
auaggcggcgcaugagagaagcccagaccaauuaccuacccaaaauggagaaaguucacguugacaucgaggaagac
agcccauuccucagagcuuugcagcggagcuucccgcaguuugagguagaagccaagcaggucacugauaaugacca
ugcuaaugccagagcguuuucgcaucuggcuucaaaacugaucgaaacggagguggacccauccgacacgauccuug
acauuggaagugcgcccgcccgcagaauguauucuaagcacaaguaucauuguaucuguccgaugagaugugcggaa
gauccggacagauuguauaaguaugcaacuaagcugaagaaaaacuguaaggaaauaacugauaaggaauuggacaa
gaaaaugaaggagcuggccgccgucaugagcgacccugaccuggaaacugagacuaugugccuccacgacgacgagu
cgugucgcuacgaagggcaagucgcuguuuaccaggauguauacgcgguugacggaccgacaagucucuaucaccaa
gccaauaagggaguuagagucgccuacuggauaggcuuugacaccaccccuuuuauguuuaagaacuuggcuggagc
auauccaucauacucuaccaacugggccgacgaaaccguguuaacggcucguaacauaggccuaugcagcucugacg
uuauggagcggucacguagagggauguccauucuuagaaagaaguauuugaaaccauccaacaauguucuauucucu
guuggcucgaccaucuaccacgagaagagggacuuacugaggagcuggcaccugccgucuguauuucacuuacgugg
caagcaaaauuacacaugucggugugagacuauaguuaguugcgacggguacgucguuaaaagaauagcuaucaguc
caggccuguaugggaagccuucaggcuaugcugcuacgaugcaccgcgagggauucuugugcugcaaagugacagac
acauugaacggggagagggucucuuuucccgugugcacguaugugccagcuacauugugugaccaaaugacuggcau
acuggcaacagaugucagugcggacgacgcgcaaaaacugcugguugggcucaaccagcguauagucgucaacgguc
gcacccagagaaacaccaauaccaugaaaaauuaccuuuugcccguaguggcccaggcauuugcuaggugggcaaag
gaauauaaggaagaucaagaagaugaaaggccacuaggacuacgagauagacaguuagucaugggguguuguugggc
uuuuagaaggcacaagauaacaucuauuuauaagcgcccggauacccaaaccaucaucaaagugaacagcgauuucc
acucauucgugcugcccaggauaggcaguaacacauuggagaucgggcugagaacaagaaucaggaaaauguuagag
gagcacaaggagccgucaccucucauuaccgccgaggacguacaagaagcuaagugcgcagccgaugaggcuaagga
ggugcgugaagccgaggaguugcgcgcagcucuaccaccuuuggcagcugauguugaggagcccacucuggaggcag
acgucgacuugauguuacaagaggcuggggccggcucaguggagacaccucguggcuugauaaagguuaccagcuac
gauggcgaggacaagaucggcucuuacgcugugcuuucuccgcaggcuguacucaagagugaaaaauuaucuugcau
ccacccucucgcugaacaagucauagugauaacacacucuggccgaaaagggcguuaugccguggaaccauaccaug
guaaaguaguggugccagagggacaugcaauacccguccaggacuuucaagcucugagugaaagugccaccauugug
uacaacgaacgugaguucguaaacagguaccugcaccauauugccacacauggaggagcgcugaacacugaugaaga
auauuacaaaacugucaagcccagcgagcacgacggcgaauaccuguacgacaucgacaggaaacagugcgucaaga
aagaacuagucacugggcuagggcucacaggcgagcugguggauccucccuuccaugaauucgccuacgagagucug
agaacacgaccagccgcuccuuaccaaguaccaaccauagggguguauggcgugccaggaucaggcaagucuggcau
cauuaaaagcgcagucaccaaaaaagaucuaguggugagcgccaagaaagaaaacugugcagaaauuauaagggacg
ucaagaaaaugaaagggcuggacgucaaugccagaacuguggacucagugcucuugaauggaugcaaacaccccgua
gagacccuguauauugacgaagcuuuugcuugucaugcagguacucucagagcgcucauagccauuauaagaccuaa
aaaggcagugcucugcggggaucccaaacagugcgguuuuuuuaacaugaugugccugaaagugcauuuuaaccacg
agauuugcacacaagucuuccacaaaagcaucucucgccguugcacuaaaucugugacuucggucgucucaaccuug
uuuuacgacaaaaaaaugagaacgacgaauccgaaagagacuaagauugugauugacacuacoggcaguaccaaacc
uaagcaggacgaucucauucucacuuguuucagagggugggugaagcaguugcaaauagauuacaaaggcaacgaaa
uaaugacggcagcugccucucaagggcugacccguaaagguguguaugccguucgguacaaggugaaugaaaauccu
cuguacgcacccaccucagaacaugugaacguccuacugacccgcacggaggaccgcaucguguggaaaacacuagc
cggcgacccauggauaaaaacacugacugccaaguacccugggaauuucacugccacgauagaggaguggcaagcag
agcaugaugccaucaugaggcacaucuuggagagaccggacccuaccgacgucuuccagaauaaggcaaacgugugu
ugggccaaggcuuuagugccggugcugaagaccgcuggcauagacaugaccacugaacaauggaacacuguggauua
uuuugaaacggacaaagcucacucagcagagauaguauugaaccaacuaugcgugagguucuuuggacucgaucugg
acuccggucuauuuucugcacccacuguuccguuauccauuaggaauaaucacugggauaacuccccgucgccuaac
auguacgggcugaauaaagaagugguccgucagcucucucgcagguacccacaacugccucgggcaguugccacugg
aagagucuaugacaugaacacugguacacugcgcaauuaugauccgcgcauaaaccuaguaccuguaaacagaagac
ugccucaugcuuuaguccuccaccauaaugaacacccacagagugacuuuucuucauucgucagcaaauugaagggc
agaacuguccugguggucggggaaaaguuguccgucccaggcaaaaugguugacugguugucagaccggccugaggc
uaccuucagagcucggcuggauuuaggcaucccaggugaugugcccaaauaugacauaauauuuguuaaugugagga
ccccauauaaauaccaucacuaucagcagugugaagaccaugccauuaagcuuagcauguugaccaagaaagcuugu
cugcaucugaaucccggcggaaccugugucagcauagguuaugguuacgcugacagggccagcgaaagcaucauugg
ugcuauagcgcggcaguucaaguuuucccggguaugcaaaccgaaauccucacuugaagagacggaaguucuguuug
uauucauuggguacgaucgcaaggcccguacgcacaauccuuacaagcuuucaucaaccuugaccaacauuuauaca
gguuccagacuccacgaagccggaugugcacccucauaucauguggugcgaggggauauugccacggccaccgaagg
agugauuauaaaugcugcuaacagcaaaggacaaccuggcggaggggugugcggagcgcuguauaagaaauucccgg
aaagcuucgauuuacagccgaucgaaguaggaaaagcgcgacuggucaaaggugcagcuaaacauaucauucaugcc
guaggaccaaacuucaacaaaguuucggagguugaaggugacaaacaguuggcagaggcuuaugaguccaucgcuaa
gauugucaacgauaacaauuacaagucaguagcgauuccacuguuguccaccggcaucuuuuccgggaacaaagauc
gacuaacccaaucauugaaccauuugcugacagcuuuagacaccacugaugcagauguagccauauacugcagggac
aagaaaugggaaaugacucucaaggaagcaguggcuaggagagaagcaguggaggagauaugcauauccgacgacuc
uucagugacagaaccugaugcagagcuggugagggugcauccgaagaguucuuuggcuggaaggaagggcuacagca
caagcgauggcaaaacuuucucauauuuggaagggaccaaguuucaccaggcggccaaggauauagcagaaauuaau
gccauguggcccguugcaacggaggccaaugagcagguaugcauguauauccucggagaaagcaugagcaguauuag
gucgaaaugccccgucgaagagucggaagccuccacaccaccuagcacgcugccuugcuugugcauccaugccauga
cuccagaaagaguacagcgccuaaaagccucacguccagaacaaauuacugugugcucauccuuuccauugccgaag
uauagaaucacuggugugcagaagauccaaugcucccagccuauauuguucucaccgaaagugccugcguauauuca
uccaaggaaguaucucguggaaacaccaccgguagacgagacuccggagccaucggcagagaaccaauccacagagg
ggacaccugaacaaccaccacuuauaaccgaggaugagaccaggacuagaacgccugagccgaucaucaucgaagag
gaagaagaggauagcauaaguuugcugucagauggcccgacccaccaggugcugcaagucgaggcagacauucacgg
gccgcccucuguaucuagcucauccugguccauuccucaugcauccgacuuugauguggacaguuuauccauacuug
acacccuggagggagcuagcgugaccagcggggcaacgucagccgagacuaacucuuacuucgcaaagaguauggag
uuucuggcgcgaccggugccugcgccucgaacaguauucaggaacccuccacaucccgcuccgcgcacaagaacacc
gucacuugcacccagcagggccugcucgagaaccagccuaguuuccaccccgccaggcgugaauagggugaucacua
gagaggagcucgaggcgcuuaccccgucacgcacuccuagcaggucggucucgagaaccagccuggucuccaacccg
ccaggcguaaauagggugauuacaagagaggaguuugaggcguucguagcacaacaacaaugacgguuugaugcggg
ugcauacaucuuuuccuccgacaccggucaagggcauuuacaacaaaaaucaguaaggcaaacggugcuauccgaag
ugguguuggagaggaccgaauuggagauuucguaugccccgcgccucgaccaagaaaaagaagaauuacuacgcaag
aaauuacaguuaaaucccacaccugcuaacagaagcagauaccaguccaggaagguggagaacaugaaagccauaac
agcuagacguauucugcaaggccuagggcauuauuugaaggcagaaggaaaaguggagugcuaccgaacccugcauc
cuguuccuuuguauucaucuagugugaaccgugccuuuucaagccccaaggucgcaguggaagccuguaacgccaug
uugaaagagaacuuuccgacuguggcuucuuacuguauuauuccagaguacgaugccuauuuggacaugguugacgg
agcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaaga
aauugcaaugucacgcaaaugagagaauugcccguauuggauucggcggccuuuaauguggaaugcuucaagaaaua
ugcguguaauaaugaauauugggaaacguuuaaagaaaaccccaucaggcuuacugaagaaaacgugguaaauuaca
uuaccaaauuaaaaggaccaaaagcugcugcucuuuuugcgaagacacauaauuugaauauguugcaggacauacca
auggacagguuuguaauggacuuaaagagagacgugaaagugacuccaggaacaaaacauacugaagaacggcccaa
gguacaggugauccaggcugccgauccgcuagcaacagcguaucugugcggaauccaccgagagcugguuaggagau
uaaaugcgguccugcuuccgaacauucauacacuguuugauaugucggcugaagacuuugacgcuauuauagccgag
cacuuccagccuggggauuguguucuggaaacugacaucgcgucguuugauaaaagugaggacgacgccauggcucu
gaccgcguuaaugauucuggaagacuuagguguggacgcagagcuguugacgcugauugaggcggcuuucggcgaaa
uuucaucaauacauuugcccacuaaaacuaaauuuaaauucggagccaugaugaaaucuggaauguuccucacacug
uuugugaacacagucauuaacauuguaaucgcaagcagaguguugagagaacggcuaaccggaucaccaugugcagc
auucauuggagaugacaauaucgugaaaggagucaaaucggacaaauuaauggcagacaggugcgccaccugguuga
uccgugaccggcacagcgugccguguggcagacccccuaaaaaggcuguuuaagcuuggcaaaccucuggcagcaga
cgaugaacaugaugaugacaggagaagggcauugcaugaagagucaacacgcuggaaccgaguggguauucuuucag
agcugugcaaggcaguagaaucaagguaugaaaccguaggaacuuccaucauaguuauggccaugacuacucuagcu
agcaguguuaaaucauucagcuaccugagaggggccccuauaacucucuacggcuaaccugaauggacuacgacaua
gucuaguccgccaagAUGUUUCUGCUCACAACCAAACGCACUAUGUUUGUUUUCCUCGUGCUGCUCCCUUUGGUAAG
UUCUCAGUGUGUAAACCUGagaACACGAACCCAGUUGCCUCCAGCUUAUACCAACUCAUUUACUCGCGGAGUAUAUU
AUCCCGAUAAGGUCUUUAGAAGUAGCGUGUUGCACUCUACACAGGAUCUGUUCUUGCCCUUCUUUAGUAACGUUACC
UGGUUUCAUGCAAUACAUGUGAGCGGAACAAAUGGAACAAAAAGAUUUGACAAUCCAGUGCUUCCAUUUAAUGAUGG
GGUUUACUUUGCCAGUaucGAAAAGUCAAACAUAAUCCGGGGGUGGAUCUUUGGAACCACUUUGGACUCUAAGACAC
AGUCUCUCCUCAUAGUAAACAACGCCACCAAUGUUGUCAUAAAAGUAUGCGAAUUUCAGUUUUGCAACGAUCCCUUU
CUCgacGUGUAUUACCAUAAGAAUAAUAAAUCCUGGAUGGAGUCUgggGUUUAUAGUAGUGCUAAUAAUUGCACUUU
CGAAUACGUGUCCCAACCAUUCCUCAUGGACCUUGAGGGCAAACAGGGGAAUUUUAAAAACUUGCGCGAAUUUGUCU
UUAAGAAUAUCGACGGAUACUUUAAGAUCUAUAGUAAACACACUCCUAUCAACCUCGUUCGGGAUCUUCCCCAAGGC
UUUUCUGCUCUCGAACCCCUCGUAGACUUGCCAAUUGGGAUAAAUAUCACUCGCUUUCAAACUUUGCUUGCCCUCCA
CAGGAGCUACCUGACACCCGGCGACUCUUCUUCUGGUuugACCGCCGGCGCCGCUGCCUAUUAUGUUGGUUACCUUC
AGCCACGAACAUUCUUGCUCAAGUAUAACGAGAAUGGCACCAUUACCGACGCCGUCGAUUGUGCAUUGGAUCCCUUG
UCUGAAACAAAAUGUACCUUGAAGUCCUUUACCGUAGAGAAAGGCAUAUACCAGACUUCCAACUUCCGAGUUCAGCC
UACAGAAUCCAUCGUACGAUUUCCCAACAUCACAAACCUCUGCCCUUUCGGUGAAGUAUUUAAUGCUACACGCUUCG
CUUCAGUCUAUGCCUGGAAUAGGAAGCGCAUAUCAAAUUGCGUGGCCGAUUAUUCAGUCCUCUAUAAUAGCGCAUCC
UUCAGUACUUUCAAGUGCUACGGCGUUUCCCCCACCAAACUCAAUGAUCUUUGCUUCACCAACGUCUAUGCUGACAG
UUUUGUCAUACGAGGCGACGAAGUACGCCAGAUUGCCCCCGGGCAGACAGGUaauAUUGCUGAUUAUAAUUAUAAAC
UCCCAGAUGACUUUACUGGAUGCGUCAUAGCCUGGAAUUCCAACAAUCUAGAUUCCAAGGUUGGUGGGAAUUAUAAU
UACcguUAUCGACUGUUCAGAAAGAGUAACUUGAAACCAUUUGAGAGAGACAUAUCCACCGAGAUUUACCAGGCAGG
CAGUaagCCUUGUAACGGCGUUGAGGGAUUUAACUGCUAUUUUCCUUUGCAAUCCUAUGGCUUUCAACCAACAAACG
GGGUUGGCUAUCAACCCUAUCGAGUGGUUGUCCUCAGCUUUGAACUUUUGCACGCUCCCGCCACAGUCUGCGGACCA
AAAAAGAGUACAAAUCUUGUCAAGAAUAAGUGCGUAAAUUUCAAUUUCAAUGGCCUUACAGGAACAGGCGUGCUGAC
UGAGUCAAACAAGaauUUCCUGCCAUUUCAGCAGUUUGGGCGGGAUAUAGCAGACACAACUGACGCUGUACGCGAUC
CUCAGACUUUGGAGAUCUUGGACAUCACUCCCUGUUCUUUCGGAGGGGUAUCUGUCAUCACCCCCGGAACUAAUACA
UCAAAUCAGGUCGCUGUGUUGUACCAAgguGUCAACUGCACAGAAGUCCCCGUUGCUAUACACGCAGACCAGCUCAC
CCCCACAUGGCGGGUGUACUCAACUGGCUCAAACGUAUUCCAGACCAGAGCUGGGUGCUUGAUCGGUGCUGAACACG
UGAACAAUAGCUAUGAAUGCGAUAUUCCCAUCGGUGCCGGGAUCUGCGCUAGCUAUCAGACACAGACCAAUUCCcgc
AGGCGGGCUCGCUCUGUAGCAUCCCAGUCUAUUAUUGCCUACACUAUGUCAUUGGGCGCCGAGAAUAGCGUCGCAUA
UUCAAAUAAUUCUAUUGCAAUACCCACCAACUUCACAAUCUCCGUAACUACAGAAAUACUUCCAGUUUCCAUGACAA
AGACAUCAGUGGAUUGUACAAUGUAUAUAUGCGGAGAUUCCACAGAAUGUUCAAAUUUGCUCUUGCAGUACGGCUCC
UUCUGCACCCAGCUCAACAGGGCACUUACAGGUAUUGCUGUCGAACAGGACAAGAACACACAAGAAGUCUUCGCCCA
AGUCAAACAGAUAUACAAAACUCCUCCCAUAAAGGAUUUUGGCGGCUUCAACUUUAGUCAGAUCCUCCCAGACCCUU
CAAAACCAUCUAAACGAUCAUUUAUUGAAGAUCUGCUGUUCAACAAGGUCACUCUUGCCGAUGCUGGAUUCAUUAAG
CAAUACGGUGACUGCCUUGGUGAUAUUGCUGCCCGAGAUCUGAUCUGUGCCCAGAAAUUCAACGGGCUCACUGUACU
CCCUCCACUGCUCACAGACGAAAUGAUUGCACAGUACACAAGUGCCCUGUUGGCAGGCACAAUCACUAGCGGCUGGA
CCUUUGGCGCAGGUGCAGCACUCCAAAUACCUUUUGCCAUGCAGAUGGCCUAUCGGUUUAAUGGGAUAGGCGUGACU
CAAAAUGUCCUCUACGAAAACCAAAAGUUGAUAGCUAACCAAUUCAAUUCAGCAAUCGGGAAGAUACAGGAUUCACU
GUCUAGUACUGCUAGUGCCCUUGGUAAGCUGCAGaacGUUGUCAACCAGAAUGCUCAAGCUCUGAAUACAUUGGUUA
AGCAGCUCUCUAGUAAUUUUGGGGCCAUCUCUUCAGUACUUAAUGAUAUUUUGAGCCGAUUGGACccaccuGAAGCU
GAAGUACAGAUCGACAGGCUGAUAACAGGCCGGCUCCAAUCCCUCCAAACAUACGUGACACAACAACUCAUACGCGC
AGCCGAAAUCCGAGCCAGCGCUAACCUGGCAGCUACCAAGAUGUCAGAAUGCGUUCUGGGCCAGAGUAAACGCGUAG
AUUUCUGCGGGAAAGGGUACCACCUGAUGUCCUUUCCACAAUCUGCACCUCACGGGGUCGUCUUUUUGCAUGUAACA
UACGUACCCGCACAAGAGAAGAAUUUUACUACCGCUCCUGCCAUCUGUCAUGACGGGAAAGCUCAUUUUCCUCGCGA
AGGUGUGUUUGUAUCUAAUGGUACACAUUGGUUUGUCACACAGCGGAAUUUCUAUGAACCCCAGAUCAUUACAACUG
ACAACACUUUUGUUUCCGGGAAUUGUGACGUGGUCAUAGGAAUCGUAAAUAACACUGUAUAUGAUCCCCUCCAACCA
GAGCUGGACUCUUUUAAAGAAGAACUGGAUAAAUAUUUCAAGAACCACACAAGUCCCGACGUGGACCUUGGGGACAU
AAGUGGUAUUAACGCAUCUGUGGUUAACAUUCAAAAGGAAAUCGACAGACUCAACGAGGUGGCCAAAAACCUGAACG
AAAGCUUGAUAGAUCUCCAGGAGUUGGGCAAGUAUGAACAGUACAUUAAAUGGCCAUGGUACAUAUGGCUUGGCUUU
AUCGCUGGCCUUAUCGCCAUCGUAAUGGUUACAAUCAUGCUGUGCUGCAUGACCUCCUGCUGUUCUUGUUUGAAAGG
GUGUUGUUCUUGUGGUAGUUGUUGCAAGUUUGACGAAGAUGAUUCCGAACCUGUUCUUAAGGGGGUAAAGCUUCACU
AUACAUGAuaaccgcggugucaaaaaccgcguggacgugguuaacaucccugcugggaggaucagccguaauuauua
uaauuggcuuggugcuggcuacuauuguggccauguacgugcugaccaaccagaaacauaauugaauacagcagcaa
uuggcaagcugcuuacauagaacucgcggcgauuggcaugccgccuuaaaauuuuuauuuuauuuuuucuuuucuuu
uccgaaucggauuuuguuuuuaauauuucaaaaaaaaaaaaaaaaaaaaaaaaaaaaaaaaaaaaaaaaaaaaa
Full-length VEEV+ AY1-S2P-wtFur-newKozak Antigen Encoding RNA Sequence
SEQ ID NO: 30
auaggcggcgcaugagagaagcccagaccaauuaccuacccaaaauggagaaaguucacguugacaucgaggaagacagc
ccauuccucagagcuuugcagcggagcuucccgcaguuugagguagaagccaagcaggucacugauaaugaccaugcuaa
ugccagagcguuuucgcaucuggcuucaaaacugaucgaaacggagguggacccauccgacacgauccuugacauuggaa
gugcgcccgcccgcagaauguauucuaagcacaaguaucauuguaucuguccgaugagaugugcggaagauccggacaga
uuguauaaguaugcaacuaagcugaagaaaaacuguaaggaaauaacugauaaggaauuggacaagaaaaugaaggagcu
ggccgccgucaugagcgacccugaccuggaaacugagacuaugugccuccacgacgacgagucgugucgcuacgaagggc
aagucgcuguuuaccaggauguauacgcgguugacggaccgacaagucucuaucaccaagccaauaagggaguuagaguc
gccuacuggauaggcuuugacaccaccccuuuuauguuuaagaacuuggcuggagcauauccaucauacucuaccaacug
ggccgacgaaaccguguuaacggcucguaacauaggccuaugcagcucugacguuauggagcggucacguagagggaugu
ccauucuuagaaagaaguauuugaaaccauccaacaauguucuauucucuguuggcucgaccaucuaccacgagaagagg
gacuuacugaggagcuggcaccugccgucuguauuucacuuacguggcaagcaaaauuacacaugucggugugagacuau
aguuaguugcgacggguacgucguuaaaagaauagcuaucaguccaggccuguaugggaagccuucaggcuaugcugcua
cgaugcaccgcgagggauucuugugcugcaaagugacagacacauugaacggggagagggucucuuuucccgugugcacg
uaugugccagcuacauugugugaccaaaugacuggcauacuggcaacagaugucagugcggacgacgcgcaaaaacugcu
gguugggcucaaccagcguauagucgucaacggucgcacccagagaaacaccaauaccaugaaaaauuaccuuuugcccg
uaguggcccaggcauuugcuaggugggcaaaggaauauaaggaagaucaagaagaugaaaggccacuaggacuacgagau
agacaguuagucaugggguguuguugggcuuuuagaaggcacaagauaacaucuauuuauaagcgcccggauacccaaac
caucaucaaagugaacagcgauuuccacucauucgugcugcccaggauaggcaguaacacauuggagaucgggcugagaa
caagaaucaggaaaauguuagaggagcacaaggagccgucaccucucauuaccgccgaggacguacaagaagcuaagugc
gcagccgaugaggcuaaggaggugcgugaagccgaggaguugcgcgcagcucuaccaccuuuggcagcugauguugagga
gcccacucuggaggcagacgucgacuugauguuacaagaggcuggggccggcucaguggagacaccucguggcuugauaa
agguuaccagcuacgauggcgaggacaagaucggcucuuacgcugugcuuucuccgcaggcuguacucaagagugaaaaa
uuaucuugcauccacccucucgcugaacaagucauagugauaacacacucuggccgaaaagggcguuaugccguggaacc
auaccaugguaaaguaguggugccagagggacaugcaauacccguccaggacuuucaagcucugagugaaagugccacca
uuguguacaacgaacgugaguucguaaacagguaccugcaccauauugccacacauggaggagcgcugaacacugaugaa
gaauauuacaaaacugucaagcccagcgagcacgacggcgaauaccuguacgacaucgacaggaaacagugcgucaagaa
agaacuagucacugggcuagggcucacaggcgagcugguggauccucccuuccaugaauucgccuacgagagucugagaa
cacgaccagccgcuccuuaccaaguaccaaccauagggguguauggcgugccaggaucaggcaagucuggcaucauuaaa
agcgcagucaccaaaaaagaucuaguggugagcgccaagaaagaaaacugugcagaaauuauaagggacgucaagaaaau
gaaagggcuggacgucaaugccagaacuguggacucagugcucuugaauggaugcaaacaccccguagagacccuguaua
uugacgaagcuuuugcuugucaugcagguacucucagagcgcucauagccauuauaagaccuaaaaaggcagugcucugc
ggggaucccaaacagugcgguuuuuuuaacaugaugugccugaaagugcauuuuaaccacgagauuugcacacaagucuu
ccacaaaagcaucucucgccguugcacuaaaucugugacuucggucgucucaaccuuguuuuacgacaaaaaaaugagaa
cgacgaauccgaaagagacuaagauugugauugacacuacoggcaguaccaaaccuaagcaggacgaucucauucucacu
uguuucagagggugggugaagcaguugcaaauagauuacaaaggcaacgaaauaaugacggcagcugccucucaagggcu
gacccguaaagguguguaugccguucgguacaaggugaaugaaaauccucuguacgcacccaccucagaacaugugaacg
uccuacugacccgcacggaggaccgcaucguguggaaaacacuagccggcgacccauggauaaaaacacugacugccaag
uacccugggaauuucacugccacgauagaggaguggcaagcagagcaugaugccaucaugaggcacaucuuggagagacc
ggacccuaccgacgucuuccagaauaaggcaaacguguguugggccaaggcuuuagugccggugcugaagaccgcuggca
uagacaugaccacugaacaauggaacacuguggauuauuuugaaacggacaaagcucacucagcagagauaguauugaac
caacuaugcgugagguucuuuggacucgaucuggacuccggucuauuuucugcacccacuguuccguuauccauuaggaa
uaaucacugggauaacuccccgucgccuaacauguacgggcugaauaaagaagugguccgucagcucucucgcagguacc
cacaacugccucgggcaguugccacuggaagagucuaugacaugaacacugguacacugcgcaauuaugauccgcgcaua
aaccuaguaccuguaaacagaagacugccucaugcuuuaguccuccaccauaaugaacacccacagagugacuuuucuuc
auucgucagcaaauugaagggcagaacuguccugguggucggggaaaaguuguccgucccaggcaaaaugguugacuggu
ugucagaccggccugaggcuaccuucagagcucggcuggauuuaggcaucccaggugaugugcccaaauaugacauaaua
uuuguuaaugugaggaccccauauaaauaccaucacuaucagcagugugaagaccaugccauuaagcuuagcauguugac
caagaaagcuugucugcaucugaaucccggcggaaccugugucagcauagguuaugguuacgcugacagggccagcgaaa
gcaucauuggugcuauagcgcggcaguucaaguuuucccggguaugcaaaccgaaauccucacuugaagagacggaaguu
cuguuuguauucauuggguacgaucgcaaggcccguacgcacaauccuuacaagcuuucaucaaccuugaccaacauuua
uacagguuccagacuccacgaagccggaugugcacccucauaucauguggugcgaggggauauugccacggccaccgaag
gagugauuauaaaugcugcuaacagcaaaggacaaccuggcggaggggugugcggagcgcuguauaagaaauucccggaa
agcuucgauuuacagccgaucgaaguaggaaaagcgcgacuggucaaaggugcagcuaaacauaucauucaugccguagg
accaaacuucaacaaaguuucggagguugaaggugacaaacaguuggcagaggcuuaugaguccaucgcuaagauuguca
acgauaacaauuacaagucaguagcgauuccacuguuguccaccggcaucuuuuccgggaacaaagaucgacuaacccaa
ucauugaaccauuugcugacagcuuuagacaccacugaugcagauguagccauauacugcagggacaagaaaugggaaau
gacucucaaggaagcaguggcuaggagagaagcaguggaggagauaugcauauccgacgacucuucagugacagaaccug
augcagagcuggugagggugcauccgaagaguucuuuggcuggaaggaagggcuacagcacaagcgauggcaaaacuuuc
ucauauuuggaagggaccaaguuucaccaggcggccaaggauauagcagaaauuaaugccauguggcccguugcaacgga
ggccaaugagcagguaugcauguauauccucggagaaagcaugagcaguauuaggucgaaaugccccgucgaagagucgg
aagccuccacaccaccuagcacgcugccuugcuugugcauccaugccaugacuccagaaagaguacagcgccuaaaagcc
ucacguccagaacaaauuacugugugcucauccuuuccauugccgaaguauagaaucacuggugugcagaagauccaaug
cucccagccuauauuguucucaccgaaagugccugcguauauucauccaaggaaguaucucguggaaacaccaccgguag
acgagacuccggagccaucggcagagaaccaauccacagaggggacaccugaacaaccaccacuuauaaccgaggaugag
accaggacuagaacgccugagccgaucaucaucgaagaggaagaagaggauagcauaaguuugcugucagauggcccgac
ccaccaggugcugcaagucgaggcagacauucacgggccgcccucuguaucuagcucauccugguccauuccucaugcau
ccgacuuugauguggacaguuuauccauacuugacacccuggagggagcuagcgugaccagcggggcaacgucagccgag
acuaacucuuacuucgcaaagaguauggaguuucuggcgcgaccggugccugcgccucgaacaguauucaggaacccucc
acaucccgcuccgcgcacaagaacaccgucacuugcacccagcagggccugcucgagaaccagccuaguuuccaccccgc
caggcgugaauagggugaucacuagagaggagcucgaggcgcuuaccccgucacgcacuccuagcaggucggucucgaga
accagccuggucuccaacccgccaggcguaaauagggugauuacaagagaggaguuugaggcguucguagcacaacaaca
augacgguuugaugcgggugcauacaucuuuuccuccgacaccggucaagggcauuuacaacaaaaaucaguaaggcaaa
cggugcuauccgaagugguguuggagaggaccgaauuggagauuucguaugccccgcgccucgaccaagaaaaagaagaa
uuacuacgcaagaaauuacaguuaaaucccacaccugcuaacagaagcagauaccaguccaggaagguggagaacaugaa
agccauaacagcuagacguauucugcaaggccuagggcauuauuugaaggcagaaggaaaaguggagugcuaccgaaccc
ugcaucuguuccuuuguauucaucuagugugaaccgugccuuuucaagccccaaggucgcaguggaagccuguaacgcca
uguugaaagagaacuuuccgacuguggcuucuuacuguauuauuccagaguacgaugccuauuuggacaugguugacgga
gcuucaugcugcuuagacacugccaguuuuugcccugcaaagcugcgcagcuuuccaaagaaacacuccuauuuggaacc
cacaauacgaucggcagugccuucagcgauccagaacacgcuccagaacguccuggcagcugccacaaaaagaaauugca
augucacgcaaaugagagaauugcccguauuggauucggcggccuuuaauguggaaugcuucaagaaauaugcguguaau
aaugaauauugggaaacguuuaaagaaaaccccaucaggcuuacugaagaaaacgugguaaauuacauuaccaaauuaaa
aggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcu
gccgauccgcuagcaacagcguaucugugcggaauccaccgagagcugguuaggagauuaaaugcgguccugcuuccgaa
cauucauacacuguuugauaugucggcugaagacuuugacgcuauuauagccgagcacuuccagccuggggauuguguuc
uggaaacugacaucgcgucguuugauaaaagugaggacgacgccauggcucugaccgcguuaaugauucuggaagacuua
gguguggacgcagagcuguugacgcugauugaggcggcuuucggcgaaauuucaucaauacauuugcccacuaaaacuaa
auuuaaauucggagccaugaugaaaucuggaauguuccucacacuguuugugaacacagucauuaacauuguaaucgcaa
gcagaguguugagagaacggcuaaccggaucaccaugugcagcauucauuggagaugacaauaucgugaaaggagucaaa
ucggacaaauuaauggcagacaggugcgccaccugguugaauauggaagucaagauuauagaugcuguggugggcgagaa
agcgccuuauuucuguggaggguuuauuuugugugacuccgugaccggcacagcgugccguguggcagacccccuaaaaa
ggcuguuuaagcuuggcaaaccucuggcagcagacgaugaacaugaugaugacaggagaagggcauugcaugaagaguca
acacgcuggaaccgaguggguauucuuucagagcugugcaaggcaguagaaucaagguaugaaaccguaggaacuuccau
cauaguuauggccaugacuacucuagcuagcaguguuaaaucauucagcuaccugagaggggccccuauaacucucuacg
gcuaaccugaauggacuacgacauagucuaguccgccgccaccAUGUUUCUGCUCACAACCAAACGCACUAUGUUUGUUU
UCCUCGUGCUGCUCCCUUUGGUAAGUUCUCAGUGUGUAAACCUGagaACACGAACCCAGUUGCCUCCAGCUUAUACCAAC
UCAUUUACUCGCGGAGUAUAUUAUCCCGAUAAGGUCUUUAGAAGUAGCGUGUUGCACUCUACACAGGAUCUGUUCUUGCC
CUUCUUUAGUAACGUUACCUGGUUUCAUGCAAUACAUGUGAGCGGAACAAAUGGAACAAAAAGAUUUGACAAUCCAGUGC
UUCCAUUUAAUGAUGGGGUUUACUUUGCCAGUaucGAAAAGUCAAACAUAAUCCGGGGGUGGAUCUUUGGAACCACUUUG
GACUCUAAGACACAGUCUCUCCUCAUAGUAAACAACGCCACCAAUGUUGUCAUAAAAGUAUGCGAAUUUCAGUUUUGCAA
CGAUCCCUUUCUCgacGUGUAUUACCAUAAGAAUAAUAAAUCCUGGAUGGAGUCUgggGUUUAUAGUAGUGCUAAUAAUU
GCACUUUCGAAUACGUGUCCCAACCAUUCCUCAUGGACCUUGAGGGCAAACAGGGGAAUUUUAAAAACUUGCGCGAAUUU
GUCUUUAAGAAUAUCGACGGAUACUUUAAGAUCUAUAGUAAACACACUCCUAUCAACCUCGUUCGGGAUCUUCCCCAAGG
CUUUUCUGCUCUCGAACCCCUCGUAGACUUGCCAAUUGGGAUAAAUAUCACUCGCUUUCAAACUUUGCUUGCCCUCCACA
GGAGCUACCUGACACCCGGCGACUCUUCUUCUGGUuugACCGCCGGCGCCGCUGCCUAUUAUGUUGGUUACCUUCAGCCA
CGAACAUUCUUGCUCAAGUAUAACGAGAAUGGCACCAUUACCGACGCCGUCGAUUGUGCAUUGGAUCCCUUGUCUGAAAC
AAAAUGUACCUUGAAGUCCUUUACCGUAGAGAAAGGCAUAUACCAGACUUCCAACUUCCGAGUUCAGCCUACAGAAUCCA
UCGUACGAUUUCCCAACAUCACAAACCUCUGCCCUUUCGGUGAAGUAUUUAAUGCUACACGCUUCGCUUCAGUCUAUGCC
UGGAAUAGGAAGCGCAUAUCAAAUUGCGUGGCCGAUUAUUCAGUCCUCUAUAAUAGCGCAUCCUUCAGUACUUUCAAGUG
CUACGGCGUUUCCCCCACCAAACUCAAUGAUCUUUGCUUCACCAACGUCUAUGCUGACAGUUUUGUCAUACGAGGCGACG
AAGUACGCCAGAUUGCCCCCGGGCAGACAGGUaauAUUGCUGAUUAUAAUUAUAAACUCCCAGAUGACUUUACUGGAUGC
GUCAUAGCCUGGAAUUCCAACAAUCUAGAUUCCAAGGUUGGUGGGAAUUAUAAUUACcguUAUCGACUGUUCAGAAAGAG
UAACUUGAAACCAUUUGAGAGAGACAUAUCCACCGAGAUUUACCAGGCAGGCAGUaagCCUUGUAACGGCGUUGAGGGAU
UUAACUGCUAUUUUCCUUUGCAAUCCUAUGGCUUUCAACCAACAAACGGGGUUGGCUAUCAACCCUAUCGAGUGGUUGUC
CUCAGCUUUGAACUUUUGCACGCUCCCGCCACAGUCUGCGGACCAAAAAAGAGUACAAAUCUUGUCAAGAAUAAGUGCGU
AAAUUUCAAUUUCAAUGGCCUUACAGGAACAGGCGUGCUGACUGAGUCAAACAAGaauUUCCUGCCAUUUCAGCAGUUUG
GGCGGGAUAUAGCAGACACAACUGACGCUGUACGCGAUCCUCAGACUUUGGAGAUCUUGGACAUCACUCCCUGUUCUUUC
GGAGGGGUAUCUGUCAUCACCCCCGGAACUAAUACAUCAAAUCAGGUCGCUGUGUUGUACCAAgguGUCAACUGCACAGA
AGUCCCCGUUGCUAUACACGCAGACCAGCUCACCCCCACAUGGCGGGUGUACUCAACUGGCUCAAACGUAUUCCAGACCA
GAGCUGGGUGCUUGAUCGGUGCUGAACACGUGAACAAUAGCUAUGAAUGCGAUAUUCCCAUCGGUGCCGGGAUCUGCGCU
AGCUAUCAGACACAGACCAAUUCCcgcAGGCGGGCUCGCUCUGUAGCAUCCCAGUCUAUUAUUGCCUACACUAUGUCAUU
GGGCGCCGAGAAUAGCGUCGCAUAUUCAAAUAAUUCUAUUGCAAUACCCACCAACUUCACAAUCUCCGUAACUACAGAAA
UACUUCCAGUUUCCAUGACAAAGACAUCAGUGGAUUGUACAAUGUAUAUAUGCGGAGAUUCCACAGAAUGUUCAAAUUUG
CUCUUGCAGUACGGCUCCUUCUGCACCCAGCUCAACAGGGCACUUACAGGUAUUGCUGUCGAACAGGACAAGAACACACA
AGAAGUCUUCGCCCAAGUCAAACAGAUAUACAAAACUCCUCCCAUAAAGGAUUUUGGCGGCUUCAACUUUAGUCAGAUCC
UCCCAGACCCUUCAAAACCAUCUAAACGAUCAUUUAUUGAAGAUCUGCUGUUCAACAAGGUCACUCUUGCCGAUGCUGGA
UUCAUUAAGCAAUACGGUGACUGCCUUGGUGAUAUUGCUGCCCGAGAUCUGAUCUGUGCCCAGAAAUUCAACGGGCUCAC
UGUACUCCCUCCACUGCUCACAGACGAAAUGAUUGCACAGUACACAAGUGCCCUGUUGGCAGGCACAAUCACUAGCGGCU
GGACCUUUGGCGCAGGUGCAGCACUCCAAAUACCUUUUGCCAUGCAGAUGGCCUAUCGGUUUAAUGGGAUAGGCGUGACU
CAAAAUGUCCUCUACGAAAACCAAAAGUUGAUAGCUAACCAAUUCAAUUCAGCAAUCGGGAAGAUACAGGAUUCACUGUC
UAGUACUGCUAGUGCCCUUGGUAAGCUGCAGaacGUUGUCAACCAGAAUGCUCAAGCUCUGAAUACAUUGGUUAAGCAGC
UCUCUAGUAAUUUUGGGGCCAUCUCUUCAGUACUUAAUGAUAUUUUGAGCCGAUUGGACccaccuGAAGCUGAAGUACAG
AUCGACAGGCUGAUAACAGGCCGGCUCCAAUCCCUCCAAACAUACGUGACACAACAACUCAUACGCGCAGCCGAAAUCCG
AGCCAGCGCUAACCUGGCAGCUACCAAGAUGUCAGAAUGCGUUCUGGGCCAGAGUAAACGCGUAGAUUUCUGCGGGAAAG
GGUACCACCUGAUGUCCUUUCCACAAUCUGCACCUCACGGGGUCGUCUUUUUGCAUGUAACAUACGUACCCGCACAAGAG
AAGAAUUUUACUACCGCUCCUGCCAUCUGUCAUGACGGGAAAGCUCAUUUUCCUCGCGAAGGUGUGUUUGUAUCUAAUGG
UACACAUUGGUUUGUCACACAGCGGAAUUUCUAUGAACCCCAGAUCAUUACAACUGACAACACUUUUGUUUCCGGGAAUU
GUGACGUGGUCAUAGGAAUCGUAAAUAACACUGUAUAUGAUCCCCUCCAACCAGAGCUGGACUCUUUUAAAGAAGAACUG
GAUAAAUAUUUCAAGAACCACACAAGUCCCGACGUGGACCUUGGGGACAUAAGUGGUAUUAACGCAUCUGUGGUUAACAU
UCAAAAGGAAAUCGACAGACUCAACGAGGUGGCCAAAAACCUGAACGAAAGCUUGAUAGAUCUCCAGGAGUUGGGCAAGU
AUGAACAGUACAUUAAAUGGCCAUGGUACAUAUGGCUUGGCUUUAUCGCUGGCCUUAUCGCCAUCGUAAUGGUUACAAUC
AUGCUGUGCUGCAUGACCUCCUGCUGUUCUUGUUUGAAAGGGUGUUGUUCUUGUGGUAGUUGUUGCAAGUUUGACGAAGA
UGAUUCCGAACCUGUUCUUAAGGGGGUAAAGCUUCACUAUACAUGAuaaccgcggugucaaaaaccgcguggacgugguu
ccaaccagaaacauaauugaauacagcagcaauuggcaagcugcuuacauagaacucgcggcgauuggcaugccgccuua
aaauuuuuauuuuauuuuuucuuuucuuuuccgaaucggauuuuguuuuuaauauuucaaaaaaaaaaaaaaaaaaaaaa
aaaaaaaaaaaaaaaaaaaaaaa
Full-length VEEV+ Omicron-B.1.1.529 Antigen Encoding RNA Sequence
SEQ ID NO: 31
auaggcggcgcaugagagaagcccagaccaauuaccuacccaaaauggagaaaguucacguugacaucgaggaaga
cagcccauuccucagagcuuugcagcggagcuucccgcaguuugagguagaagccaagcaggucacugauaaugac
caugcuaaugccagagcguuuucgcaucuggcuucaaaacugaucgaaacggagguggacccauccgacacgaucc
uugacauuggaagugcgcccgcccgcagaauguauucuaagcacaaguaucauuguaucuguccgaugagaugugc
ggaagauccggacagauuguauaaguaugcaacuaagcugaagaaaaacuguaaggaaauaacugauaaggaauug
gacaagaaaaugaaggagcuggccgccgucaugagcgacccugaccuggaaacugagacuaugugccuccacgacg
acgagucgugucgcuacgaagggcaagucgcuguuuaccaggauguauacgcgguugacggaccgacaagucucua
ucaccaagccaauaagggaguuagagucgccuacuggauaggcuuugacaccaccccuuuuauguuuaagaacuug
gcuggagcauauccaucauacucuaccaacugggccgacgaaaccguguuaacggcucguaacauaggccuaugca
gcucugacguuauggagcggucacguagagggauguccauucuuagaaagaaguauuugaaaccauccaacaaugu
ucuauucucuguuggcucgaccaucuaccacgagaagagggacuuacugaggagcuggcaccugccgucuguauuu
cacuuacguggcaagcaaaauuacacaugucggugugagacuauaguuaguugcgacggguacgucguuaaaagaa
uagcuaucaguccaggccuguaugggaagccuucaggcuaugcugcuacgaugcaccgcgagggauucuugugcug
caaagugacagacacauugaacggggagagggucucuuuucccgugugcacguaugugccagcuacauugugugac
caaaugacuggcauacuggcaacagaugucagugcggacgacgcgcaaaaacugcugguugggcucaaccagcgua
uagucgucaacggucgcacccagagaaacaccaauaccaugaaaaauuaccuuuugcccguaguggcccaggcauu
ugcuaggugggcaaaggaauauaaggaagaucaagaagaugaaaggccacuaggacuacgagauagacaguuaguc
augggguguuguugggcuuuuagaaggcacaagauaacaucuauuuauaagcgcccggauacccaaaccaucauca
aagugaacagcgauuuccacucauucgugcugcccaggauaggcaguaacacauuggagaucgggcugagaacaag
aaucaggaaaauguuagaggagcacaaggagccgucaccucucauuaccgccgaggacguacaagaagcuaagugc
gcagccgaugaggcuaaggaggugcgugaagccgaggaguugcgcgcagcucuaccaccuuuggcagcugauguug
aggagcccacucuggaggcagacgucgacuugauguuacaagaggcuggggccggcucaguggagacaccucgugg
cuugauaaagguuaccagcuacgauggcgaggacaagaucggcucuuacgcugugcuuucuccgcaggcuguacuc
aagagugaaaaauuaucuugcauccacccucucgcugaacaagucauagugauaacacacucuggccgaaaagggc
guuaugccguggaaccauaccaugguaaaguaguggugccagagggacaugcaauacccguccaggacuuucaagc
ucugagugaaagugccaccauuguguacaacgaacgugaguucguaaacagguaccugcaccauauugccacacau
ggaggagcgcugaacacugaugaagaauauuacaaaacugucaagcccagcgagcacgacggcgaauaccuguacg
acaucgacaggaaacagugcgucaagaaagaacuagucacugggcuagggcucacaggcgagcugguggauccucc
cuuccaugaauucgccuacgagagucugagaacacgaccagccgcuccuuaccaaguaccaaccauagggguguau
ggcgugccaggaucaggcaagucuggcaucauuaaaagcgcagucaccaaaaaagaucuaguggugagcgccaaga
aagaaaacugugcagaaauuauaagggacgucaagaaaaugaaagggcuggacgucaaugccagaacuguggacuc
agugcucuugaauggaugcaaacaccccguagagacccuguauauugacgaagcuuuugcuugucaugcagguacu
cucagagcgcucauagccauuauaagaccuaaaaaggcagugcucugcggggaucccaaacagugcgguuuuuuua
acaugaugugccugaaagugcauuuuaaccacgagauuugcacacaagucuuccacaaaagcaucucucgccguug
cacuaaaucugugacuucggucgucucaaccuuguuuuacgacaaaaaaaugagaacgacgaauccgaaagagacu
aagauugugauugacacuacoggcaguaccaaaccuaagcaggacgaucucauucucacuuguuucagaggguggg
ugaagcaguugcaaauagauuacaaaggcaacgaaauaaugacggcagcugccucucaagggcugacccguaaagg
uguguaugccguucgguacaaggugaaugaaaauccucuguacgcacccaccucagaacaugugaacguccuacug
acccgcacggaggaccgcaucguguggaaaacacuagccggcgacccauggauaaaaacacugacugccaaguacc
cugggaauuucacugccacgauagaggaguggcaagcagagcaugaugccaucaugaggcacaucuuggagagacc
ggacccuaccgacgucuuccagaauaaggcaaacguguguugggccaaggcuuuagugccggugcugaagaccgcu
ggcauagacaugaccacugaacaauggaacacuguggauuauuuugaaacggacaaagcucacucagcagagauag
uauugaaccaacuaugcgugagguucuuuggacucgaucuggacuccggucuauuuucugcacccacuguuccguu
auccauuaggaauaaucacugggauaacuccccgucgccuaacauguacgggcugaauaaagaagugguccgucag
cucucucgcagguacccacaacugccucgggcaguugccacuggaagagucuaugacaugaacacugguacacugc
gcaauuaugauccgcgcauaaaccuaguaccuguaaacagaagacugccucaugcuuuaguccuccaccauaauga
acacccacagagugacuuuucuucauucgucagcaaauugaagggcagaacuguccugguggucggggaaaaguug
uccgucccaggcaaaaugguugacugguugucagaccggccugaggcuaccuucagagcucggcuggauuuaggca
ucccaggugaugugcccaaauaugacauaauauuuguuaaugugaggaccccauauaaauaccaucacuaucagca
gugugaagaccaugccauuaagcuuagcauguugaccaagaaagcuugucugcaucugaaucccggcggaaccugu
gucagcauagguuaugguuacgcugacagggccagcgaaagcaucauuggugcuauagcgcggcaguucaaguuuu
cccggguaugcaaaccgaaauccucacuugaagagacggaaguucuguuuguauucauuggguacgaucgcaaggc
ccguacgcacaauccuuacaagcuuucaucaaccuugaccaacauuuauacagguuccagacuccacgaagccgga
ugugcacccucauaucauguggugcgaggggauauugccacggccaccgaaggagugauuauaaaugcugcuaaca
gcaaaggacaaccuggcggaggggugugcggagcgcuguauaagaaauucccggaaagcuucgauuuacagccgau
cgaaguaggaaaagcgcgacuggucaaaggugcagcuaaacauaucauucaugccguaggaccaaacuucaacaaa
guuucggagguugaaggugacaaacaguuggcagaggcuuaugaguccaucgcuaagauugucaacgauaacaauu
acaagucaguagcgauuccacuguuguccaccggcaucuuuuccgggaacaaagaucgacuaacccaaucauugaa
ccauuugcugacagcuuuagacaccacugaugcagauguagccauauacugcagggacaagaaaugggaaaugacu
cucaaggaagcaguggcuaggagagaagcaguggaggagauaugcauauccgacgacucuucagugacagaaccug
augcagagcuggugagggugcauccgaagaguucuuuggcuggaaggaagggcuacagcacaagcgauggcaaaac
uuucucauauuuggaagggaccaaguuucaccaggcggccaaggauauagcagaaauuaaugccauguggcccguu
gcaacggaggccaaugagcagguaugcauguauauccucggagaaagcaugagcaguauuaggucgaaaugccccg
ucgaagagucggaagccuccacaccaccuagcacgcugccuugcuugugcauccaugccaugacuccagaaagagu
acagcgccuaaaagccucacguccagaacaaauuacugugugcucauccuuuccauugccgaaguauagaaucacu
ggugugcagaagauccaaugcucccagccuauauuguucucaccgaaagugccugcguauauucauccaaggaagu
aucucguggaaacaccaccgguagacgagacuccggagccaucggcagagaaccaauccacagaggggacaccuga
acaaccaccacuuauaaccgaggaugagaccaggacuagaacgccugagccgaucaucaucgaagaggaagaagag
gauagcauaaguuugcugucagauggcccgacccaccaggugcugcaagucgaggcagacauucacgggccgcccu
cuguaucuagcucauccugguccauuccucaugcauccgacuuugauguggacaguuuauccauacuugacacccu
ggagggagcuagcgugaccagcggggcaacgucagccgagacuaacucuuacuucgcaaagaguauggaguuucug
gcgcgaccggugccugcgccucgaacaguauucaggaacccuccacaucccgcuccgcgcacaagaacaccgucac
uugcacccagcagggccugcucgagaaccagccuaguuuccaccccgccaggcgugaauagggugaucacuagaga
ggagcucgaggcgcuuaccccgucacgcacuccuagcaggucggucucgagaaccagccuggucuccaacccgcca
ggcguaaauagggugauuacaagagaggaguuugaggcguucguagcacaacaacaaugacgguuugaugcgggug
cauacaucuuuuccuccgacaccggucaagggcauuuacaacaaaaaucaguaaggcaaacggugcuauccgaagu
gguguuggagaggaccgaauuggagauuucguaugccccgcgccucgaccaagaaaaagaagaauuacuacgcaag
aaauuacaguuaaaucccacaccugcuaacagaagcagauaccaguccaggaagguggagaacaugaaagccauaa
cagcuagacguauucugcaaggccuagggcauuauuugaaggcagaaggaaaaguggagugcuaccgaacccugca
uccuguuccuuuguauucaucuagugugaaccgugccuuuucaagccccaaggucgcaguggaagccuguaacgcc
auguugaaagagaacuuuccgacuguggcuucuuacuguauuauuccagaguacgaugccuauuuggacaugguug
acggagcuucaugcugcuuagacacugccaguuuuugcccugcaaagcugcgcagcuuuccaaagaaacacuccua
uuuggaacccacaauacgaucggcagugccuucagcgauccagaacacgcuccagaacguccuggcagcugccaca
aaaagaaauugcaaugucacgcaaaugagagaauugcccguauuggauucggcggccuuuaauguggaaugcuuca
agaaauaugcguguaauaaugaauauugggaaacguuuaaagaaaaccccaucaggcuuacugaagaaaacguggu
aaauuacauuaccaaauuaaaaggaccaaaagcugcugcucuuuuugcgaagacacauaauuugaauauguugcag
gacauaccaauggacagguuuguaauggacuuaaagagagacgugaaagugacuccaggaacaaaacauacugaag
aacggcccaagguacaggugauccaggcugccgauccgcuagcaacagcguaucugugcggaauccaccgagagcu
gguuaggagauuaaaugcgguccugcuuccgaacauucauacacuguuugauaugucggcugaagacuuugacgcu
auuauagccgagcacuuccagccuggggauuguguucuggaaacugacaucgcgucguuugauaaaagugaggacg
acgccauggcucugaccgcguuaaugauucuggaagacuuagguguggacgcagagcuguugacgcugauugaggc
ggcuuucggcgaaauuucaucaauacauuugcccacuaaaacuaaauuuaaauucggagccaugaugaaaucugga
auguuccucacacuguuugugaacacagucauuaacauuguaaucgcaagcagaguguugagagaacggcuaaccg
gaucaccaugugcagcauucauuggagaugacaauaucgugaaaggagucaaaucggacaaauuaauggcagacag
gugcgccaccugguugaauauggaagucaagauuauagaugcuguggugggcgagaaagcgccuuauuucugugga
ggguuuauuuugugugacuccgugaccggcacagcgugccguguggcagacccccuaaaaaggcuguuuaagcuug
gcaaaccucuggcagcagacgaugaacaugaugaugacaggagaagggcauugcaugaagagucaacacgcuggaa
ccgaguggguauucuuucagagcugugcaaggcaguagaaucaagguaugaaaccguaggaacuuccaucauaguu
auggccaugacuacucuagcuagcaguguuaaaucauucagcuaccugagaggggccccuauaacucucuacggcu
aaccugaauggacuacgacauagucuaguccgcCGCCACCAUGUUUCUGUUGACGACCAAGCGAACGAUGUUCGUU
UUCUUGGUGCUUUUGCCACUUGUCAGUUCCCAGUGCGUCAAUCUGACGACACGAACACAGCUGCCUCCUGCGUACA
CUAACAGUUUUACGCGAGGAGUGUAUUACCCUGACAAAGUUUUCCGCUCUAGUGUCCUCCAUAGCACACAGGACUU
GUUUCUCCCCUUCUUUUCCAACGUUACGUGGUUCCAUGUGAUUAGUGGAACUAACGGUACUAAAAGAUUCGACAAU
CCAGUAUUGCCUUUCAACGAUGGGGUCUAUUUCGCGUCCAUCGAGAAAUCAAAUAUCAUUCGCGGUUGGAUUUUUG
GAACGACACUCGAUUCAAAGACGCAAUCCCUCCUUAUUGUCAACAACGCCACUAACGUAGUCAUUAAGGUUUGUGA
GUUCCAGUUUUGUAAUGAUCCCUUUUUUGACCACAAGAAUAACAAGAGCUGGAUGGAAAGCGAGUUCAGAGUGUAU
AGCUCUGCAAACAACUGUACUUUUGAAUACGUGAGUCAACCUUUCCUUAUGGACCUUGAAGGUAAACAGGGUAACU
UUAAGAAUUUGCGCGAAUUUGUUUUCAAAAACAUUGAUGGUUACUUUAAAAUCUAUAGUAAGCACACUCCUAUCAU
UGUAAGAGAGCCGGAGGACCUUCCACAGGGUUUUAGUGCGCUCGAGCCCCUCGUUGACCUGCCCAUUGGGAUCAAC
AUAACUCGAUUCCAAACAUUGCUCGCCCUUCAUCGGUCCUAUCUGACUCCCGGUGACUCCUCUAGCGGAUGGACGG
CAGGUGCCGCCGCAUACUACGUGGGGUACCUUCAACCUCGGACAUUUUUGUUGAAAUACAAUGAGAAUGGCACUAU
AACUGACGCGGUUGAUUGCGCGCUCGACCCAUUGUCCGAAACUAAGUGUACUUUGAAGUCAUUUACAGUGGAGAAA
GGAAUAUAUCAGACUAGCAAUUUUCGGGUACAGCCCACGGAGUCUAUCGUACGGUUUCCUAACAUCACGAAUCUGU
GCCCUUUUGAUGAGGUCUUUAAUGCAACACGGUUCGCCUCCGUCUAUGCGUGGAAUAGAAAGCGCAUCUCAAAUUG
UGUAGCUGAUUAUUCCGUACUUUACAACUUGGCCCCGUUCUUCACAUUUAAGUGCUACGGUGUAAGCCCUACUAAA
CUGAACGAUUUGUGUUUCACCAACGUCUAUGCAGAUAGCUUUGUUAUUCGAGGCGAUGAGGUACGCCAGAUUGCGC
CUGGUCAAACGGGUAAUAUCGCCGACUACAAUUAUAAAUUGCCAGACGAUUUUACUGGUUGUGUCAUCGCUUGGAA
UAGUAAUAAGUUGGACAGUAAGGUAUCCGGCAAUUACAACUAUCUCUACCGAUUGUUCCGGAAGUCUAACCUCAAG
CCGUUUGAAAGAGACAUAUCCACUGAGAUAUACCAAGCAGGCAAUAAACCAUGCAACGGAGUUGCUGGUUUCAACU
GCUAUUUCCCGUUGCGGUCUUAUUCCUUCAGACCUACUUACGGAGUCGGACACCAACCCUACAGGGUCGUCGUUUU
GAGUUUUGAAUUGUUGCAUGCUCCAGCAACCGUGUGUGGACCUAAAAAGUCCACGAAUCUCGUGAAGAAUAAGUGC
GUAAACUUCAAUUUCAACGGUCUGAAAGGGACUGGUGUAUUGACAGAAAGCAACAAGAAGUUUCUGCCAUUCCAGC
AAUUUGGUAGGGAUAUAGCGGAUACAACUGAUGCCGUUCGGGAUCCUCAAACAUUGGAGAUCUUGGACAUCACACC
GUGUUCUUUUGGGGGUGUCUCCGUUAUCACACCGGGUACAAAUACGAGCAAUCAGGUUGCGGUCCUUUACCAAGGC
GUUAAUUGUACCGAGGUUCCAGUAGCAAUACACGCGGAUCAACUCACGCCCACAUGGAGGGUUUACAGUACAGGCA
GUAAUGUUUUCCAAACGAGAGCGGGAUGCCUCAUCGGGGCAGAAUACGUAAAUAAUUCUUACGAAUGCGACAUCCC
UAUUGGCGCAGGAAUUUGCGCAAGUUACCAAACCCAGACCAAGUCUCAUAGGCGGGCGCGGUCUGUUGCAAGCCAA
UCUAUAAUAGCGUACACUAUGUCCCUCGGCGCGGAGAACAGUGUCGCAUAUUCCAACAACUCUAUUGCGAUACCUA
CUAAUUUCACUAUUAGCGUCACAACUGAGAUCCUUCCCGUCAGUAUGACCAAAACGUCUGUCGACUGUACUAUGUA
UAUUUGCGGCGACAGUACCGAAUGCUCUAAUCUUUUGUUGCAGUAUGGUUCUUUUUGCACGCAACUUAAGAGAGCU
UUGACGGGGAUAGCUGUGGAACAAGAUAAAAACACACAGGAGGUAUUUGCACAAGUGAAACAGAUCUAUAAAACUC
CACCGAUCAAGUACUUUGGCGGCUUUAACUUCUCCCAGAUCUUGCCCGACCCGUCUAAACCAAGUAAACGGAGUUU
UAUAGAGGACCUUCUCUUCAAUAAGGUAACAUUGGCAGACGCCGGCUUCAUUAAACAAUACGGAGAUUGCCUUGGA
GACAUCGCUGCGCGCGACUUGAUCUGCGCACAAAAAUUUAAAGGCUUGACGGUCCUCCCUCCUUUGCUCACAGACG
AGAUGAUAGCACAAUACACUUCCGCACUGCUUGCUGGAACCAUCACCUCUGGUUGGACAUUCGGUGCGGGAGCGGC
UUUGCAGAUUCCGUUUGCGAUGCAAAUGGCUUAUCGGUUUAACGGCAUUGGAGUAACACAGAAUGUGCUCUACGAG
AAUCAAAAGCUUAUUGCGAAUCAAUUCAACUCUGCGAUUGGCAAAAUUCAAGAUUCAUUGAGUAGCACCGCCAGUG
CUCUUGGCAAGCUUCAGGAUGUCGUAAACCACAAUGCACAAGCUCUGAAUACACUGGUUAAACAAUUGUCCAGUAA
AUUUGGGGCAAUCUCUUCAGUGCUGAACGACAUUUUCUCAAGAUUGGAUCCACCCGAAGCGGAGGUACAGAUUGAC
CGCCUGAUAACCGGGAGGUUGCAAAGCCUUCAGACUUAUGUUACACAACAGUUGAUUCGGGCAGCAGAGAUAAGAG
CCUCAGCAAACCUCGCAGCUACGAAGAUGUCAGAGUGUGUCCUUGGGCAAUCUAAGCGGGUAGAUUUCUGCGGCAA
AGGAUAUCAUUUGAUGAGCUUUCCCCAAUCAGCCCCACAUGGAGUAGUUUUUCUUCAUGUCACUUACGUUCCGGCG
CAGGAAAAGAACUUCACCACAGCGCCAGCCAUUUGUCAUGAUGGGAAGGCGCAUUUCCCAAGAGAAGGUGUUUUCG
UGUCUAACGGUACCCACUGGUUCGUUACGCAGCGGAAUUUCUACGAACCACAGAUCAUCACUACCGACAACACGUU
UGUCUCUGGAAAUUGUGACGUUGUCAUAGGGAUAGUGAACAAUACAGUAUAUGAUCCACUUCAGCCUGAACUUGAC
UCUUUUAAGGAGGAGCUGGACAAAUAUUUCAAAAAUCAUACAAGCCCGGACGUCGAUCUUGGAGAUAUUUCAGGUA
UCAACGCAAGUGUCGUAAAUAUUCAGAAGGAGAUCGAUCGAUUGAACGAGGUUGCAAAAAACCUUAAUGAGAGCCU
UAUAGAUCUUCAAGAGCUGGGGAAGUAUGAACAAUAUAUCAAGUGGCCUUGGUACAUUUGGCUCGGGUUCAUUGCC
GGACUUAUCGCGAUCGUAAUGGUAACAAUCAUGCUCUGCUGUAUGACUUCUUGCUGUUCAUGCCUCAAAGGUUGUU
GCUCCUGUGGGUCUUGCUGUAAAUUUGACGAGGAUGAUUCUGAACCAGUGCUUAAAGGCGUGAAGCUCCACUAUAC
CUGAuaaccgcggugucaaaaaccgcguggacgugguuaacaucccugcugggaggaucagccguaauuauuauaa
uuggcuuggugcuggcuacuauuguggccauguacgugcugaccaaccagaaacauaauugaauacagcagcaauu
ggcaagcugcuuacauagaacucgcggcgauuggcaugccgccuuaaaauuuuuauuuuauuuuuucuuuucuuuu
ccgaaucggauuuuguuuuuaauauuucaaaaaaaaaaaaaaaaaaaaaaaaaaaaaaaaaaaaaaaaaaaaa
Vector Backbone RNA Sequence 1
SEQ ID NO: 32
auaggcggcgcaugagagaagcccagaccaauuaccuacccaaaauggagaaaguucacguugacaucgaggaagacag
cccauuccucagagcuuugcagcggagcuucccgcaguuugagguagaagccaagcaggucacugauaaugaccaugcu
aaugccagagcguuuucgcaucuggcuucaaaacugaucgaaacggagguggacccauccgacacgauccuugacauug
gaagugcgcccgcccgcagaauguauucuaagcacaaguaucauuguaucuguccgaugagaugugcggaagauccgga
cagauuguauaaguaugcaacuaagcugaagaaaaacuguaaggaaauaacugauaaggaauuggacaagaaaaugaag
gagcuggccgccgucaugagcgacccugaccuggaaacugagacuaugugccuccacgacgacgagucgugucgcuacg
aagggcaagucgcuguuuaccaggauguauacgcgguugacggaccgacaagucucuaucaccaagccaauaagggagu
uagagucgccuacuggauaggcuuugacaccaccccuuuuauguuuaagaacuuggcuggagcauauccaucauacucu
accaacugggccgacgaaaccguguuaacggcucguaacauaggccuaugcagcucugacguuauggagcggucacgua
gagggauguccauucuuagaaagaaguauuugaaaccauccaacaauguucuauucucuguuggcucgaccaucuacca
cgagaagagggacuuacugaggagcuggcaccugccgucuguauuucacuuacguggcaagcaaaauuacacaugucgg
ugugagacuauaguuaguugcgacggguacgucguuaaaagaauagcuaucaguccaggccuguaugggaagccuucag
gcuaugcugcuacgaugcaccgcgagggauucuugugcugcaaagugacagacacauugaacggggagagggucucuuu
ucccgugugcacguaugugccagcuacauugugugaccaaaugacuggcauacuggcaacagaugucagugcggacgac
gcgcaaaaacugcugguugggcucaaccagcguauagucgucaacggucgcacccagagaaacaccaauaccaugaaaa
auuaccuuuugcccguaguggcccaggcauuugcuaggugggcaaaggaauauaaggaagaucaagaagaugaaaggcc
acuaggacuacgagauagacaguuagucaugggguguuguugggcuuuuagaaggcacaagauaacaucuauuuauaag
cgcccggauacccaaaccaucaucaaagugaacagcgauuuccacucauucgugcugcccaggauaggcaguaacacau
uggagaucgggcugagaacaagaaucaggaaaauguuagaggagcacaaggagccgucaccucucauuaccgccgagga
cguacaagaagcuaagugcgcagccgaugaggcuaaggaggugcgugaagccgaggaguugcgcgcagcucuaccaccu
uuggcagcugauguugaggagcccacucuggaggcagacgucgacuugauguuacaagaggcuggggccggcucagugg
agacaccucguggcuugauaaagguuaccagcuacgauggcgaggacaagaucggcucuuacgcugugcuuucuccgca
ggcuguacucaagagugaaaaauuaucuugcauccacccucucgcugaacaagucauagugauaacacacucuggccga
aaagggcguuaugccguggaaccauaccaugguaaaguaguggugccagagggacaugcaauacccguccaggacuuuc
aagcucugagugaaagugccaccauuguguacaacgaacgugaguucguaaacagguaccugcaccauauugccacaca
uggaggagcgcugaacacugaugaagaauauuacaaaacugucaagcccagcgagcacgacggcgaauaccuguacgac
aucgacaggaaacagugcgucaagaaagaacuagucacugggcuagggcucacaggcgagcugguggauccucccuucc
augaauucgccuacgagagucugagaacacgaccagccgcuccuuaccaaguaccaaccauagggguguauggcgugcc
aggaucaggcaagucuggcaucauuaaaagcgcagucaccaaaaaagaucuaguggugagcgccaagaaagaaaacugu
gcagaaauuauaagggacgucaagaaaaugaaagggcuggacgucaaugccagaacuguggacucagugcucuugaaug
gaugcaaacaccccguagagacccuguauauugacgaagcuuuugcuugucaugcagguacucucagagcgcucauagc
cauuauaagaccuaaaaaggcagugcucugcggggaucccaaacagugcgguuuuuuuaacaugaugugccugaaagug
cauuuuaaccacgagauuugcacacaagucuuccacaaaagcaucucucgccguugcacuaaaucugugacuucggucg
ucucaaccuuguuuuacgacaaaaaaaugagaacgacgaauccgaaagagacuaagauugugauugacacuaccggcag
uaccaaaccuaagcaggacgaucucauucucacuuguuucagagggugggugaagcaguugcaaauagauuacaaaggc
aacgaaauaaugacggcagcugccucucaagggcugacccguaaagguguguaugccguucgguacaaggugaaugaaa
auccucuguacgcacccaccucagaacaugugaacguccuacugacccgcacggaggaccgcaucguguggaaaacacu
agccggcgacccauggauaaaaacacugacugccaaguacccugggaauuucacugccacgauagaggaguggcaagca
gagcaugaugccaucaugaggcacaucuuggagagaccggacccuaccgacgucuuccagaauaaggcaaacguguguu
gggccaaggcuuuagugccggugcugaagaccgcuggcauagacaugaccacugaacaauggaacacuguggauuauuu
ugaaacggacaaagcucacucagcagagauaguauugaaccaacuaugcgugagguucuuuggacucgaucuggacucc
ggucuauuuucugcacccacuguuccguuauccauuaggaauaaucacugggauaacuccccgucgccuaacauguacg
ggcugaauaaagaagugguccgucagcucucucgcagguacccacaacugccucgggcaguugccacuggaagagucua
ugacaugaacacugguacacugcgcaauuaugauccgcgcauaaaccuaguaccuguaaacagaagacugccucaugcu
uuaguccuccaccauaaugaacacccacagagugacuuuucuucauucgucagcaaauugaagggcagaacuguccugg
uggucggggaaaaguuguccgucccaggcaaaaugguugacugguugucagaccggccugaggcuaccuucagagcucg
gcuggauuuaggcaucccaggugaugugcccaaauaugacauaauauuuguuaaugugaggaccccauauaaauaccau
cacuaucagcagugugaagaccaugccauuaagcuuagcauguugaccaagaaagcuugucugcaucugaaucccggcg
gaaccugugucagcauagguuaugguuacgcugacagggccagcgaaagcaucauuggugcuauagcgcggcaguucaa
guuuucccggguaugcaaaccgaaauccucacuugaagagacggaaguucuguuuguauucauuggguacgaucgcaag
gcccguacgcacaauccuuacaagcuuucaucaaccuugaccaacauuuauacagguuccagacuccacgaagccggau
gugcacccucauaucauguggugcgaggggauauugccacggccaccgaaggagugauuauaaaugcugcuaacagcaa
aggacaaccuggcggaggggugugcggagcgcuguauaagaaauucccggaaagcuucgauuuacagccgaucgaagua
ggaaaagcgcgacuggucaaaggugcagcuaaacauaucauucaugccguaggaccaaacuucaacaaaguuucggagg
uugaaggugacaaacaguuggcagaggcuuaugaguccaucgcuaagauugucaacgauaacaauuacaagucaguagc
gauuccacuguuguccaccggcaucuuuuccgggaacaaagaucgacuaacccaaucauugaaccauuugcugacagcu
uuagacaccacugaugcagauguagccauauacugcagggacaagaaaugggaaaugacucucaaggaagcaguggcua
ggagagaagcaguggaggagauaugcauauccgacgacucuucagugacagaaccugaugcagagcuggugagggugca
uccgaagaguucuuuggcuggaaggaagggcuacagcacaagcgauggcaaaacuuucucauauuuggaagggaccaag
uuucaccaggcggccaaggauauagcagaaauuaaugccauguggcccguugcaacggaggccaaugagcagguaugca
uguauauccucggagaaagcaugagcaguauuaggucgaaaugccccgucgaagagucggaagccuccacaccaccuag
cacgcugccuugcuugugcauccaugccaugacuccagaaagaguacagcgccuaaaagccucacguccagaacaaauu
acugugugcucauccuuuccauugccgaaguauagaaucacuggugugcagaagauccaaugcucccagccuauauugu
ucucaccgaaagugccugcguauauucauccaaggaaguaucucguggaaacaccaccgguagacgagacuccggagcc
aucggcagagaaccaauccacagaggggacaccugaacaaccaccacuuauaaccgaggaugagaccaggacuagaacg
ccugagccgaucaucaucgaagaggaagaagaggauagcauaaguuugcugucagauggcccgacccaccaggugcugc
aagucgaggcagacauucacgggccgcccucuguaucuagcucauccugguccauuccucaugcauccgacuuugaugu
ggacaguuuauccauacuugacacccuggagggagcuagcgugaccagcggggcaacgucagccgagacuaacucuuac
uucgcaaagaguauggaguuucuggcgcgaccggugccugcgccucgaacaguauucaggaacccuccacaucccgcuc
cgcgcacaagaacaccgucacuugcacccagcagggccugcucgagaaccagccuaguuuccaccccgccaggcgugaa
uagggugaucacuagagaggagcucgaggcgcuuaccccgucacgcacuccuagcaggucggucucgagaaccagccug
gucuccaacccgccaggcguaaauagggugauuacaagagaggaguuugaggcguucguagcacaacaacaaugacggu
uugaugcgggugcauacaucuuuuccuccgacaccggucaagggcauuuacaacaaaaaucaguaaggcaaacggugcu
auccgaagugguguuggagaggaccgaauuggagauuucguaugccccgcgccucgaccaagaaaaagaagaauuacua
cgcaagaaauuacaguuaaaucccacaccugcuaacagaagcagauaccaguccaggaagguggagaacaugaaagcca
uaacagcuagacguauucugcaaggccuagggcauuauuugaaggcagaaggaaaaguggagugcuaccgaacccugca
uccuguuccuuuguauucaucuagugugaaccgugccuuuucaagccccaaggucgcaguggaagccuguaacgccaug
uugaaagagaacuuuccgacuguggcuucuuacuguauuauuccagaguacgaugccuauuuggacaugguugacggag
cuucaugcugcuuagacacugccaguuuuugcccugcaaagcugcgcagcuuuccaaagaaacacuccuauuuggaacc
cacaauacgaucggcagugccuucagcgauccagaacacgcuccagaacguccuggcagcugccacaaaaagaaauugc
aaugucacgcaaaugagagaauugcccguauuggauucggcggccuuuaauguggaaugcuucaagaaauaugcgugua
auaaugaauauugggaaacguuuaaagaaaaccccaucaggcuuacugaagaaaacgugguaaauuacauuaccaaauu
aaaaggaccaaaagcugcugcucuuuuugcgaagacacauaauuugaauauguugcaggacauaccaauggacagguuu
guaauggacuuaaagagagacgugaaagugacuccaggaacaaaacauacugaagaacggcccaagguacaggugaucc
aggcugccgauccgcuagcaacagcguaucugugcggaauccaccgagagcugguuaggagauuaaaugcgguccugcu
uccgaacauucauacacuguuugauaugucggcugaagacuuugacgcuauuauagccgagcacuuccagccuggggau
uguguucuggaaacugacaucgcgucguuugauaaaagugaggacgacgccauggcucugaccgcguuaaugauucugg
aagacuuagguguggacgcagagcuguugacgcugauugaggcggcuuucggcgaaauuucaucaauacauuugcccac
uaaaacuaaauuuaaauucggagccaugaugaaaucuggaauguuccucacacuguuugugaacacagucauuaacauu
guaaucgcaagcagaguguugagagaacggcuaaccggaucaccaugugcagcauucauuggagaugacaauaucguga
aaggagucaaaucggacaaauuaauggcagacaggugcgccaccugguugaauauggaagucaagauuauagaugcugu
ggugggcgagaaagcgccuuauuucuguggaggguuuauuuugugugacuccgugaccggcacagcgugccguguggca
gacccccuaaaaaggcuguuuaagcuuggcaaaccucuggcagcagacgaugaacaugaugaugacaggagaagggcau
ugcaugaagagucaacacgcuggaaccgaguggguauucuuucagagcugugcaaggcaguagaaucaagguaugaaac
cguaggaacuuccaucauaguuauggccaugacuacucuagcuagcaguguuaaaucauucagcuaccugagaggggcc
ccuauaacucucuacggcuaaccugaauggacuacgacauagucuaguccgccaagnugauaaccgcggugucaaaaac
gccauguacgugcugaccaaccagaaacauaauugaauacagcagcaauuggcaagcugcuuacauagaacucgcggcg
auuggcaugccgccuuaaaauuuuuauuuuauuuuuucuuuucuuuuccgaaucggauuuuguuuuuaauauuucaaaa
aaaaaaaaaaaaaaaaaaaaaaaaaaaaaaaaaaaaaaaaa
Vector Backbone RNA Sequence 2
SEQ ID NO: 33
auaggcggcgcaugagagaagcccagaccaauuaccuacccaaaauggagaaaguucacguugacaucgaggaagacag
cccauuccucagagcuuugcagcggagcuucccgcaguuugagguagaagccaagcaggucacugauaaugaccaugcu
aaugccagagcguuuucgcaucuggcuucaaaacugaucgaaacggagguggacccauccgacacgauccuugacauug
gaagugcgcccgcccgcagaauguauucuaagcacaaguaucauuguaucuguccgaugagaugugcggaagauccgga
cagauuguauaaguaugcaacuaagcugaagaaaaacuguaaggaaauaacugauaaggaauuggacaagaaaaugaag
gagcuggccgccgucaugagcgacccugaccuggaaacugagacuaugugccuccacgacgacgagucgugucgcuacg
aagggcaagucgcuguuuaccaggauguauacgcgguugacggaccgacaagucucuaucaccaagccaauaagggagu
uagagucgccuacuggauaggcuuugacaccaccccuuuuauguuuaagaacuuggcuggagcauauccaucauacucu
accaacugggccgacgaaaccguguuaacggcucguaacauaggccuaugcagcucugacguuauggagcggucacgua
gagggauguccauucuuagaaagaaguauuugaaaccauccaacaauguucuauucucuguuggcucgaccaucuacca
cgagaagagggacuuacugaggagcuggcaccugccgucuguauuucacuuacguggcaagcaaaauuacacaugucgg
ugugagacuauaguuaguugcgacggguacgucguuaaaagaauagcuaucaguccaggccuguaugggaagccuucag
gcuaugcugcuacgaugcaccgcgagggauucuugugcugcaaagugacagacacauugaacggggagagggucucuuu
ucccgugugcacguaugugccagcuacauugugugaccaaaugacuggcauacuggcaacagaugucagugcggacgac
gcgcaaaaacugcugguugggcucaaccagcguauagucgucaacggucgcacccagagaaacaccaauaccaugaaaa
auuaccuuuugcccguaguggcccaggcauuugcuaggugggcaaaggaauauaaggaagaucaagaagaugaaaggcc
acuaggacuacgagauagacaguuagucaugggguguuguugggcuuuuagaaggcacaagauaacaucuauuuauaag
cgcccggauacccaaaccaucaucaaagugaacagcgauuuccacucauucgugcugcccaggauaggcaguaacacau
uggagaucgggcugagaacaagaaucaggaaaauguuagaggagcacaaggagccgucaccucucauuaccgccgagga
cguacaagaagcuaagugcgcagccgaugaggcuaaggaggugcgugaagccgaggaguugcgcgcagcucuaccaccu
uuggcagcugauguugaggagcccacucuggaggcagacgucgacuugauguuacaagaggcuggggccggcucagugg
agacaccucguggcuugauaaagguuaccagcuacgauggcgaggacaagaucggcucuuacgcugugcuuucuccgca
ggcuguacucaagagugaaaaauuaucuugcauccacccucucgcugaacaagucauagugauaacacacucuggccga
aaagggcguuaugccguggaaccauaccaugguaaaguaguggugccagagggacaugcaauacccguccaggacuuuc
aagcucugagugaaagugccaccauuguguacaacgaacgugaguucguaaacagguaccugcaccauauugccacaca
uggaggagcgcugaacacugaugaagaauauuacaaaacugucaagcccagcgagcacgacggcgaauaccuguacgac
aucgacaggaaacagugcgucaagaaagaacuagucacugggcuagggcucacaggcgagcugguggauccucccuucc
augaauucgccuacgagagucugagaacacgaccagccgcuccuuaccaaguaccaaccauagggguguauggcgugcc
aggaucaggcaagucuggcaucauuaaaagcgcagucaccaaaaaagaucuaguggugagcgccaagaaagaaaacugu
gcagaaauuauaagggacgucaagaaaaugaaagggcuggacgucaaugccagaacuguggacucagugcucuugaaug
gaugcaaacaccccguagagacccuguauauugacgaagcuuuugcuugucaugcagguacucucagagcgcucauagc
cauuauaagaccuaaaaaggcagugcucugcggggaucccaaacagugcgguuuuuuuaacaugaugugccugaaagug
cauuuuaaccacgagauuugcacacaagucuuccacaaaagcaucucucgccguugcacuaaaucugugacuucggucg
ucucaaccuuguuuuacgacaaaaaaaugagaacgacgaauccgaaagagacuaagauugugauugacacuaccggcag
uaccaaaccuaagcaggacgaucucauucucacuuguuucagagggugggugaagcaguugcaaauagauuacaaaggc
aacgaaauaaugacggcagcugccucucaagggcugacccguaaagguguguaugccguucgguacaaggugaaugaaa
auccucuguacgcacccaccucagaacaugugaacguccuacugacccgcacggaggaccgcaucguguggaaaacacu
agccggcgacccauggauaaaaacacugacugccaaguacccugggaauuucacugccacgauagaggaguggcaagca
gagcaugaugccaucaugaggcacaucuuggagagaccggacccuaccgacgucuuccagaauaaggcaaacguguguu
gggccaaggcuuuagugccggugcugaagaccgcuggcauagacaugaccacugaacaauggaacacuguggauuauuu
ugaaacggacaaagcucacucagcagagauaguauugaaccaacuaugcgugagguucuuuggacucgaucuggacucc
ggucuauuuucugcacccacuguuccguuauccauuaggaauaaucacugggauaacuccccgucgccuaacauguacg
ggcugaauaaagaagugguccgucagcucucucgcagguacccacaacugccucgggcaguugccacuggaagagucua
ugacaugaacacugguacacugcgcaauuaugauccgcgcauaaaccuaguaccuguaaacagaagacugccucaugcu
uuaguccuccaccauaaugaacacccacagagugacuuuucuucauucgucagcaaauugaagggcagaacuguccugg
uggucggggaaaaguuguccgucccaggcaaaaugguugacugguugucagaccggccugaggcuaccuucagagcucg
gcuggauuuaggcaucccaggugaugugcccaaauaugacauaauauuuguuaaugugaggaccccauauaaauaccau
cacuaucagcagugugaagaccaugccauuaagcuuagcauguugaccaagaaagcuugucugcaucugaaucccggcg
gaaccugugucagcauagguuaugguuacgcugacagggccagcgaaagcaucauuggugcuauagcgcggcaguucaa
guuuucccggguaugcaaaccgaaauccucacuugaagagacggaaguucuguuuguauucauuggguacgaucgcaag
gcccguacgcacaauccuuacaagcuuucaucaaccuugaccaacauuuauacagguuccagacuccacgaagccggau
gugcacccucauaucauguggugcgaggggauauugccacggccaccgaaggagugauuauaaaugcugcuaacagcaa
aggacaaccuggcggaggggugugcggagcgcuguauaagaaauucccggaaagcuucgauuuacagccgaucgaagua
ggaaaagcgcgacuggucaaaggugcagcuaaacauaucauucaugccguaggaccaaacuucaacaaaguuucggagg
uugaaggugacaaacaguuggcagaggcuuaugaguccaucgcuaagauugucaacgauaacaauuacaagucaguagc
gauuccacuguuguccaccggcaucuuuuccgggaacaaagaucgacuaacccaaucauugaaccauuugcugacagcu
uuagacaccacugaugcagauguagccauauacugcagggacaagaaaugggaaaugacucucaaggaagcaguggcua
ggagagaagcaguggaggagauaugcauauccgacgacucuucagugacagaaccugaugcagagcuggugagggugca
uccgaagaguucuuuggcuggaaggaagggcuacagcacaagcgauggcaaaacuuucucauauuuggaagggaccaag
uuucaccaggcggccaaggauauagcagaaauuaaugccauguggcccguugcaacggaggccaaugagcagguaugca
uguauauccucggagaaagcaugagcaguauuaggucgaaaugccccgucgaagagucggaagccuccacaccaccuag
cacgcugccuugcuugugcauccaugccaugacuccagaaagaguacagcgccuaaaagccucacguccagaacaaauu
acugugugcucauccuuuccauugccgaaguauagaaucacuggugugcagaagauccaaugcucccagccuauauugu
ucucaccgaaagugccugcguauauucauccaaggaaguaucucguggaaacaccaccgguagacgagacuccggagcc
aucggcagagaaccaauccacagaggggacaccugaacaaccaccacuuauaaccgaggaugagaccaggacuagaacg
ccugagccgaucaucaucgaagaggaagaagaggauagcauaaguuugcugucagauggcccgacccaccaggugcugc
aagucgaggcagacauucacgggccgcccucuguaucuagcucauccugguccauuccucaugcauccgacuuugaugu
ggacaguuuauccauacuugacacccuggagggagcuagcgugaccagcggggcaacgucagccgagacuaacucuuac
uucgcaaagaguauggaguuucuggcgcgaccggugccugcgccucgaacaguauucaggaacccuccacaucccgcuc
cgcgcacaagaacaccgucacuugcacccagcagggccugcucgagaaccagccuaguuuccaccccgccaggcgugaa
uagggugaucacuagagaggagcucgaggcgcuuaccccgucacgcacuccuagcaggucggucucgagaaccagccug
gucuccaacccgccaggcguaaauagggugauuacaagagaggaguuugaggcguucguagcacaacaacaaugacggu
uugaugcgggugcauacaucuuuuccuccgacaccggucaagggcauuuacaacaaaaaucaguaaggcaaacggugcu
auccgaagugguguuggagaggaccgaauuggagauuucguaugccccgcgccucgaccaagaaaaagaagaauuacua
cgcaagaaauuacaguuaaaucccacaccugcuaacagaagcagauaccaguccaggaagguggagaacaugaaagcca
uaacagcuagacguauucugcaaggccuagggcauuauuugaaggcagaaggaaaaguggagugcuaccgaacccugca
uccuguuccuuuguauucaucuagugugaaccgugccuuuucaagccccaaggucgcaguggaagccuguaacgccaug
uugaaagagaacuuuccgacuguggcuucuuacuguauuauuccagaguacgaugccuauuuggacaugguugacggag
cuucaugcugcuuagacacugccaguuuuugcccugcaaagcugcgcagcuuuccaaagaaacacuccuauuuggaacc
cacaauacgaucggcagugccuucagcgauccagaacacgcuccagaacguccuggcagcugccacaaaaagaaauugc
aaugucacgcaaaugagagaauugcccguauuggauucggcggccuuuaauguggaaugcuucaagaaauaugcgugua
auaaugaauauugggaaacguuuaaagaaaaccccaucaggcuuacugaagaaaacgugguaaauuacauuaccaaauu
aaaaggaccaaaagcugcugcucuuuuugcgaagacacauaauuugaauauguugcaggacauaccaauggacagguuu
guaauggacuuaaagagagacgugaaagugacuccaggaacaaaacauacugaagaacggcccaagguacaggugaucc
aggcugccgauccgcuagcaacagcguaucugugcggaauccaccgagagcugguuaggagauuaaaugcgguccugcu
uccgaacauucauacacuguuugauaugucggcugaagacuuugacgcuauuauagccgagcacuuccagccuggggau
uguguucuggaaacugacaucgcgucguuugauaaaagugaggacgacgccauggcucugaccgcguuaaugauucugg
aagacuuagguguggacgcagagcuguugacgcugauugaggcggcuuucggcgaaauuucaucaauacauuugcccac
uaaaacuaaauuuaaauucggagccaugaugaaaucuggaauguuccucacacuguuugugaacacagucauuaacauu
guaaucgcaagcagaguguugagagaacggcuaaccggaucaccaugugcagcauucauuggagaugacaauaucguga
aaggagucaaaucggacaaauuaauggcagacaggugcgccaccugguugaauauggaagucaagauuauagaugcugu
ggugggcgagaaagcgccuuauuucuguggaggguuuauuuugugugacuccgugaccggcacagcgugccguguggca
gacccccuaaaaaggcuguuuaagcuuggcaaaccucuggcagcagacgaugaacaugaugaugacaggagaagggcau
ugcaugaagagucaacacgcuggaaccgaguggguauucuuucagagcugugcaaggcaguagaaucaagguaugaaac
cguaggaacuuccaucauaguuauggccaugacuacucuagcuagcaguguuaaaucauucagcuaccugagaggggcc
ccuauaacucucuacggcuaaccugaauggacuacgacauagucuaguccgccaagnugauaaccgcggugucaaaaac
gccauguacgugcugaccaaccagaaacauaauugaauacagcagcaauuggcaagcugcuuacauagaacucgcggcg
auuggcaugccgccuuaaaauuuuuauuuuauuuuuucuuuucuuuuccgaaucggauuuuguuuuuaauauuucaaaa
aaaaaaaaaaaaaaaaaaaaaaaaaaaaaaaaaaaaaaaaa
Vector Backbone RNA Sequence 3
SEQ ID NO: 34
auaggcggcgcaugagagaagcccagaccaauuaccuacccaaaauggagaaaguucacguugacaucgaggaagacag
cccauuccucagagcuuugcagcggagcuucccgcaguuugagguagaagccaagcaggucacugauaaugaccaugcu
aaugccagagcguuuucgcaucuggcuucaaaacugaucgaaacggagguggacccauccgacacgauccuugacauug
gaagugcgcccgcccgcagaauguauucuaagcacaaguaucauuguaucuguccgaugagaugugcggaagauccgga
cagauuguauaaguaugcaacuaagcugaagaaaaacuguaaggaaauaacugauaaggaauuggacaagaaaaugaag
gagcuggccgccgucaugagcgacccugaccuggaaacugagacuaugugccuccacgacgacgagucgugucgcuacg
aagggcaagucgcuguuuaccaggauguauacgcgguugacggaccgacaagucucuaucaccaagccaauaagggagu
uagagucgccuacuggauaggcuuugacaccaccccuuuuauguuuaagaacuuggcuggagcauauccaucauacucu
accaacugggccgacgaaaccguguuaacggcucguaacauaggccuaugcagcucugacguuauggagcggucacgua
gagggauguccauucuuagaaagaaguauuugaaaccauccaacaauguucuauucucuguuggcucgaccaucuacca
cgagaagagggacuuacugaggagcuggcaccugccgucuguauuucacuuacguggcaagcaaaauuacacaugucgg
ugugagacuauaguuaguugcgacggguacgucguuaaaagaauagcuaucaguccaggccuguaugggaagccuucag
gcgcaaaaacugcugguugggcucaaccagcguauagucgucaacggucgcacccagagaaacaccaauaccaugaaaa
ucccgugugcacguaugugccagcuacauugugugaccaaaugacuggcauacuggcaacagaugucagugcggacgac
gcgcaaaaacugcugguugggcucaaccagcguauagucgucaacggucgcacccagagaaacaccaauaccaugaaaa
auuaccuuuugcccguaguggcccaggcauuugcuaggugggcaaaggaauauaaggaagaucaagaagaugaaaggcc
auuaccuuuugcccguaguggcccaggcauuugcuaggugggcaaaggaauauaaggaagaucaagaagaugaaaggcc
cgcccggauacccaaaccaucaucaaagugaacagcgauuuccacucauucgugcugcccaggauaggcaguaacacau
uggagaucgggcugagaacaagaaucaggaaaauguuagaggagcacaaggagccgucaccucucauuaccgccgagga
cguacaagaagcuaagugcgcagccgaugaggcuaaggaggugcgugaagccgaggaguugcgcgcagcucuaccaccu
uuggcagcugauguugaggagcccacucuggaggcagacgucgacuugauguuacaagaggcuggggccggcucagugg
agacaccucguggcuugauaaagguuaccagcuacgauggcgaggacaagaucggcucuuacgcugugcuuucuccgca
ggcuguacucaagagugaaaaauuaucuugcauccacccucucgcugaacaagucauagugauaacacacucuggccga
aaagggcguuaugccguggaaccauaccaugguaaaguaguggugccagagggacaugcaauacccguccaggacuuuc
aagcucugagugaaagugccaccauuguguacaacgaacgugaguucguaaacagguaccugcaccauauugccacaca
uggaggagcgcugaacacugaugaagaauauuacaaaacugucaagcccagcgagcacgacggcgaauaccuguacgac
aucgacaggaaacagugcgucaagaaagaacuagucacugggcuagggcucacaggcgagcugguggauccucccuucc
augaauucgccuacgagagucugagaacacgaccagccgcuccuuaccaaguaccaaccauagggguguauggcgugcc
aggaucaggcaagucuggcaucauuaaaagcgcagucaccaaaaaagaucuaguggugagcgccaagaaagaaaacugu
gcagaaauuauaagggacgucaagaaaaugaaagggcuggacgucaaugccagaacuguggacucagugcucuugaaug
gaugcaaacaccccguagagacccuguauauugacgaagcuuuugcuugucaugcagguacucucagagcgcucauagc
cauuauaagaccuaaaaaggcagugcucugcggggaucccaaacagugcgguuuuuuuaacaugaugugccugaaagug
cauuuuaaccacgagauuugcacacaagucuuccacaaaagcaucucucgccguugcacuaaaucugugacuucggucg
ucucaaccuuguuuuacgacaaaaaaaugagaacgacgaauccgaaagagacuaagauugugauugacacuaccggcag
uaccaaaccuaagcaggacgaucucauucucacuuguuucagagggugggugaagcaguugcaaauagauuacaaaggc
aacgaaauaaugacggcagcugccucucaagggcugacccguaaagguguguaugccguucgguacaaggugaaugaaa
auccucuguacgcacccaccucagaacaugugaacguccuacugacccgcacggaggaccgcaucguguggaaaacacu
agccggcgacccauggauaaaaacacugacugccaaguacccugggaauuucacugccacgauagaggaguggcaagca
gagcaugaugccaucaugaggcacaucuuggagagaccggacccuaccgacgucuuccagaauaaggcaaacguguguu
gggccaaggcuuuagugccggugcugaagaccgcuggcauagacaugaccacugaacaauggaacacuguggauuauuu
ugaaacggacaaagcucacucagcagagauaguauugaaccaacuaugcgugagguucuuuggacucgaucuggacucc
ggucuauuuucugcacccacuguuccguuauccauuaggaauaaucacugggauaacuccccgucgccuaacauguacg
ggcugaauaaagaagugguccgucagcucucucgcagguacccacaacugccucgggcaguugccacuggaagagucua
ugacaugaacacugguacacugcgcaauuaugauccgcgcauaaaccuaguaccuguaaacagaagacugccucaugcu
uuaguccuccaccauaaugaacacccacagagugacuuuucuucauucgucagcaaauugaagggcagaacuguccugg
uggucggggaaaaguuguccgucccaggcaaaaugguugacugguugucagaccggccugaggcuaccuucagagcucg
gcuggauuuaggcaucccaggugaugugcccaaauaugacauaauauuuguuaaugugaggaccccauauaaauaccau
cacuaucagcagugugaagaccaugccauuaagcuuagcauguugaccaagaaagcuugucugcaucugaaucccggcg
gaaccugugucagcauagguuaugguuacgcugacagggccagcgaaagcaucauuggugcuauagcgcggcaguucaa
guuuucccggguaugcaaaccgaaauccucacuugaagagacggaaguucuguuuguauucauuggguacgaucgcaag
gcccguacgcacaauccuuacaagcuuucaucaaccuugaccaacauuuauacagguuccagacuccacgaagccggau
gugcacccucauaucauguggugcgaggggauauugccacggccaccgaaggagugauuauaaaugcugcuaacagcaa
aggacaaccuggcggaggggugugcggagcgcuguauaagaaauucccggaaagcuucgauuuacagccgaucgaagua
ggaaaagcgcgacuggucaaaggugcagcuaaacauaucauucaugccguaggaccaaacuucaacaaaguuucggagg
uugaaggugacaaacaguuggcagaggcuuaugaguccaucgcuaagauugucaacgauaacaauuacaagucaguagc
gauuccacuguuguccaccggcaucuuuuccgggaacaaagaucgacuaacccaaucauugaaccauuugcugacagcu
uuagacaccacugaugcagauguagccauauacugcagggacaagaaaugggaaaugacucucaaggaagcaguggcua
ggagagaagcaguggaggagauaugcauauccgacgacucuucagugacagaaccugaugcagagcuggugagggugca
uccgaagaguucuuuggcuggaaggaagggcuacagcacaagcgauggcaaaacuuucucauauuuggaagggaccaag
uuucaccaggcggccaaggauauagcagaaauuaaugccauguggcccguugcaacggaggccaaugagcagguaugca
uguauauccucggagaaagcaugagcaguauuaggucgaaaugccccgucgaagagucggaagccuccacaccaccuag
cacgcugccuugcuugugcauccaugccaugacuccagaaagaguacagcgccuaaaagccucacguccagaacaaauu
acugugugcucauccuuuccauugccgaaguauagaaucacuggugugcagaagauccaaugcucccagccuauauugu
ucucaccgaaagugccugcguauauucauccaaggaaguaucucguggaaacaccaccgguagacgagacuccggagcc
aucggcagagaaccaauccacagaggggacaccugaacaaccaccacuuauaaccgaggaugagaccaggacuagaacg
ccugagccgaucaucaucgaagaggaagaagaggauagcauaaguuugcugucagauggcccgacccaccaggugcugc
aagucgaggcagacauucacgggccgcccucuguaucuagcucauccugguccauuccucaugcauccgacuuugaugu
ggacaguuuauccauacuugacacccuggagggagcuagcgugaccagcggggcaacgucagccgagacuaacucuuac
uucgcaaagaguauggaguuucuggcgcgaccggugccugcgccucgaacaguauucaggaacccuccacaucccgcuc
cgcgcacaagaacaccgucacuugcacccagcagggccugcucgagaaccagccuaguuuccaccccgccaggcgugaa
uagggugaucacuagagaggagcucgaggcgcuuaccccgucacgcacuccuagcaggucggucucgagaaccagccug
gucuccaacccgccaggcguaaauagggugauuacaagagaggaguuugaggcguucguagcacaacaacaaugacggu
uugaugcgggugcauacaucuuuuccuccgacaccggucaagggcauuuacaacaaaaaucaguaaggcaaacggugcu
auccgaagugguguuggagaggaccgaauuggagauuucguaugccccgcgccucgaccaagaaaaagaagaauuacua
cgcaagaaauuacaguuaaaucccacaccugcuaacagaagcagauaccaguccaggaagguggagaacaugaaagcca
uaacagcuagacguauucugcaaggccuagggcauuauuugaaggcagaaggaaaaguggagugcuaccgaacccugca
uccuguuccuuuguauucaucuagugugaaccgugccuuuucaagccccaaggucgcaguggaagccuguaacgccaug
uugaaagagaacuuuccgacuguggcuucuuacuguauuauuccagaguacgaugccuauuuggacaugguugacggag
cuucaugcugcuuagacacugccaguuuuugcccugcaaagcugcgcagcuuuccaaagaaacacuccuauuuggaacc
cacaauacgaucggcagugccuucagcgauccagaacacgcuccagaacguccuggcagcugccacaaaaagaaauugc
aaugucacgcaaaugagagaauugcccguauuggauucggcggccuuuaauguggaaugcuucaagaaauaugcgugua
auaaugaauauugggaaacguuuaaagaaaaccccaucaggcuuacugaagaaaacgugguaaauuacauuaccaaauu
aaaaggaccaaaagcugcugcucuuuuugcgaagacacauaauuugaauauguugcaggacauaccaauggacagguuu
guaauggacuuaaagagagacgugaaagugacuccaggaacaaaacauacugaagaacggcccaagguacaggugaucc
aggcugccgauccgcuagcaacagcguaucugugcggaauccaccgagagcugguuaggagauuaaaugcgguccugcu
uccgaacauucauacacuguuugauaugucggcugaagacuuugacgcuauuauagccgagcacuuccagccuggggau
uguguucuggaaacugacaucgcgucguuugauaaaagugaggacgacgccauggcucugaccgcguuaaugauucugg
aagacuuagguguggacgcagagcuguugacgcugauugaggcggcuuucggcgaaauuucaucaauacauuugcccac
uaaaacuaaauuuaaauucggagccaugaugaaaucuggaauguuccucacacuguuugugaacacagucauuaacauu
guaaucgcaagcagaguguugagagaacggcuaaccggaucaccaugugcagcauucauuggagaugacaauaucguga
aaggagucaaaucggacaaauuaauggcagacaggugcgccaccugguugaauauggaagucaagauuauagaugcugu
ggugggcgagaaagcgccuuauuucuguggaggguuuauuuugugugacuccgugaccggcacagcgugccguguggca
gacccccuaaaaaggcuguuuaagcuuggcaaaccucuggcagcagacgaugaacaugaugaugacaggagaagggcau
ugcaugaagagucaacacgcuggaaccgaguggguauucuuucagagcugugcaaggcaguagaaucaagguaugaaac
cguaggaacuuccaucauaguuauggccaugacuacucuagcuagcaguguuaaaucauucagcuaccugagaggggcc
ccuauaacucucuacggcuaaccugaauggacuacgacauagucuaguccgccaagnuaguaaccgcggugucaaaaac
gccauguacgugcugaccaaccagaaacauaauugaauacagcagcaauuggcaagcugcuuacauagaacucgcggcg
auuggcaugccgccuuaaaauuuuuauuuuauuuuuucuuuucuuuuccgaaucggauuuuguuuuuaauauuucaaaa
aaaaaaaaaaaaaaaaaaaaaaaaaaaaaaaaaaaaaaaaa
Vector Backbone RNA Sequence 4
SEQ ID NO: 35
auaggcggcgcaugagagaagcccagaccaauuaccuacccaaaauggagaaaguucacguugacaucgaggaagacag
cccauuccucagagcuuugcagcggagcuucccgcaguuugagguagaagccaagcaggucacugauaaugaccaugcu
aaugccagagcguuuucgcaucuggcuucaaaacugaucgaaacggagguggacccauccgacacgauccuugacauug
gaagugcgcccgcccgcagaauguauucuaagcacaaguaucauuguaucuguccgaugagaugugcggaagauccgga
cagauuguauaaguaugcaacuaagcugaagaaaaacuguaaggaaauaacugauaaggaauuggacaagaaaaugaag
gagcuggccgccgucaugagcgacccugaccuggaaacugagacuaugugccuccacgacgacgagucgugucgcuacg
aagggcaagucgcuguuuaccaggauguauacgcgguugacggaccgacaagucucuaucaccaagccaauaagggagu
uagagucgccuacuggauaggcuuugacaccaccccuuuuauguuuaagaacuuggcuggagcauauccaucauacucu
accaacugggccgacgaaaccguguuaacggcucguaacauaggccuaugcagcucugacguuauggagcggucacgua
gagggauguccauucuuagaaagaaguauuugaaaccauccaacaauguucuauucucuguuggcucgaccaucuacca
cgagaagagggacuuacugaggagcuggcaccugccgucuguauuucacuuacguggcaagcaaaauuacacaugucgg
ugugagacuauaguuaguugcgacggguacgucguuaaaagaauagcuaucaguccaggccuguaugggaagccuucag
gcuaugcugcuacgaugcaccgcgagggauucuugugcugcaaagugacagacacauugaacggggagagggucucuuu
ucccgugugcacguaugugccagcuacauugugugaccaaaugacuggcauacuggcaacagaugucagugcggacgac
gcgcaaaaacugcugguugggcucaaccagcguauagucgucaacggucgcacccagagaaacaccaauaccaugaaaa
auuaccuuuugcccguaguggcccaggcauuugcuaggugggcaaaggaauauaaggaagaucaagaagaugaaaggcc
acuaggacuacgagauagacaguuagucaugggguguuguugggcuuuuagaaggcacaagauaacaucuauuuauaag
cgcccggauacccaaaccaucaucaaagugaacagcgauuuccacucauucgugcugcccaggauaggcaguaacacau
uggagaucgggcugagaacaagaaucaggaaaauguuagaggagcacaaggagccgucaccucucauuaccgccgagga
cguacaagaagcuaagugcgcagccgaugaggcuaaggaggugcgugaagccgaggaguugcgcgcagcucuaccaccu
uuggcagcugauguugaggagcccacucuggaggcagacgucgacuugauguuacaagaggcuggggccggcucagugg
agacaccucguggcuugauaaagguuaccagcuacgauggcgaggacaagaucggcucuuacgcugugcuuucuccgca
ggcuguacucaagagugaaaaauuaucuugcauccacccucucgcugaacaagucauagugauaacacacucuggccga
aaagggcguuaugccguggaaccauaccaugguaaaguaguggugccagagggacaugcaauacccguccaggacuuuc
aagcucugagugaaagugccaccauuguguacaacgaacgugaguucguaaacagguaccugcaccauauugccacaca
uggaggagcgcugaacacugaugaagaauauuacaaaacugucaagcccagcgagcacgacggcgaauaccuguacgac
aucgacaggaaacagugcgucaagaaagaacuagucacugggcuagggcucacaggcgagcugguggauccucccuucc
augaauucgccuacgagagucugagaacacgaccagccgcuccuuaccaaguaccaaccauagggguguauggcgugcc
aggaucaggcaagucuggcaucauuaaaagcgcagucaccaaaaaagaucuaguggugagcgccaagaaagaaaacugu
gcagaaauuauaagggacgucaagaaaaugaaagggcuggacgucaaugccagaacuguggacucagugcucuugaaug
gaugcaaacaccccguagagacccuguauauugacgaagcuuuugcuugucaugcagguacucucagagcgcucauagc
cauuauaagaccuaaaaaggcagugcucugcggggaucccaaacagugcgguuuuuuuaacaugaugugccugaaagug
cauuuuaaccacgagauuugcacacaagucuuccacaaaagcaucucucgccguugcacuaaaucugugacuucggucg
ucucaaccuuguuuuacgacaaaaaaaugagaacgacgaauccgaaagagacuaagauugugauugacacuaccggcag
uaccaaaccuaagcaggacgaucucauucucacuuguuucagagggugggugaagcaguugcaaauagauuacaaaggc
aacgaaauaaugacggcagcugccucucaagggcugacccguaaagguguguaugccguucgguacaaggugaaugaaa
auccucuguacgcacccaccucagaacaugugaacguccuacugacccgcacggaggaccgcaucguguggaaaacacu
agccggcgacccauggauaaaaacacugacugccaaguacccugggaauuucacugccacgauagaggaguggcaagca
gagcaugaugccaucaugaggcacaucuuggagagaccggacccuaccgacgucuuccagaauaaggcaaacguguguu
gggccaaggcuuuagugccggugcugaagaccgcuggcauagacaugaccacugaacaauggaacacuguggauuauuu
ugaaacggacaaagcucacucagcagagauaguauugaaccaacuaugcgugagguucuuuggacucgaucuggacucc
ggucuauuuucugcacccacuguuccguuauccauuaggaauaaucacugggauaacuccccgucgccuaacauguacg
ggcugaauaaagaagugguccgucagcucucucgcagguacccacaacugccucgggcaguugccacuggaagagucua
ugacaugaacacugguacacugcgcaauuaugauccgcgcauaaaccuaguaccuguaaacagaagacugccucaugcu
uuaguccuccaccauaaugaacacccacagagugacuuuucuucauucgucagcaaauugaagggcagaacuguccugg
uggucggggaaaaguuguccgucccaggcaaaaugguugacugguugucagaccggccugaggcuaccuucagagcucg
gcuggauuuaggcaucccaggugaugugcccaaauaugacauaauauuuguuaaugugaggaccccauauaaauaccau
cacuaucagcagugugaagaccaugccauuaagcuuagcauguugaccaagaaagcuugucugcaucugaaucccggcg
gaaccugugucagcauagguuaugguuacgcugacagggccagcgaaagcaucauuggugcuauagcgcggcaguucaa
guuuucccggguaugcaaaccgaaauccucacuugaagagacggaaguucuguuuguauucauuggguacgaucgcaag
gcccguacgcacaauccuuacaagcuuucaucaaccuugaccaacauuuauacagguuccagacuccacgaagccggau
gugcacccucauaucauguggugcgaggggauauugccacggccaccgaaggagugauuauaaaugcugcuaacagcaa
aggacaaccuggcggaggggugugcggagcgcuguauaagaaauucccggaaagcuucgauuuacagccgaucgaagua
ggaaaagcgcgacuggucaaaggugcagcuaaacauaucauucaugccguaggaccaaacuucaacaaaguuucggagg
uugaaggugacaaacaguuggcagaggcuuaugaguccaucgcuaagauugucaacgauaacaauuacaagucaguagc
gauuccacuguuguccaccggcaucuuuuccgggaacaaagaucgacuaacccaaucauugaaccauuugcugacagcu
uuagacaccacugaugcagauguagccauauacugcagggacaagaaaugggaaaugacucucaaggaagcaguggcua
ggagagaagcaguggaggagauaugcauauccgacgacucuucagugacagaaccugaugcagagcuggugagggugca
uccgaagaguucuuuggcuggaaggaagggcuacagcacaagcgauggcaaaacuuucucauauuuggaagggaccaag
uuucaccaggcggccaaggauauagcagaaauuaaugccauguggcccguugcaacggaggccaaugagcagguaugca
uguauauccucggagaaagcaugagcaguauuaggucgaaaugccccgucgaagagucggaagccuccacaccaccuag
cacgcugccuugcuugugcauccaugccaugacuccagaaagaguacagcgccuaaaagccucacguccagaacaaauu
acugugugcucauccuuuccauugccgaaguauagaaucacuggugugcagaagauccaaugcucccagccuauauugu
ucucaccgaaagugccugcguauauucauccaaggaaguaucucguggaaacaccaccgguagacgagacuccggagcc
aucggcagagaaccaauccacagaggggacaccugaacaaccaccacuuauaaccgaggaugagaccaggacuagaacg
ccugagccgaucaucaucgaagaggaagaagaggauagcauaaguuugcugucagauggcccgacccaccaggugcugc
aagucgaggcagacauucacgggccgcccucuguaucuagcucauccugguccauuccucaugcauccgacuuugaugu
ggacaguuuauccauacuugacacccuggagggagcuagcgugaccagcggggcaacgucagccgagacuaacucuuac
uucgcaaagaguauggaguuucuggcgcgaccggugccugcgccucgaacaguauucaggaacccuccacaucccgcuc
cgcgcacaagaacaccgucacuugcacccagcagggccugcucgagaaccagccuaguuuccaccccgccaggcgugaa
uagggugaucacuagagaggagcucgaggcgcuuaccccgucacgcacuccuagcaggucggucucgagaaccagccug
gucuccaacccgccaggcguaaauagggugauuacaagagaggaguuugaggcguucguagcacaacaacaaugacggu
uugaugcgggugcauacaucuuuuccuccgacaccggucaagggcauuuacaacaaaaaucaguaaggcaaacggugcu
auccgaagugguguuggagaggaccgaauuggagauuucguaugccccgcgccucgaccaagaaaaagaagaauuacua
cgcaagaaauuacaguuaaaucccacaccugcuaacagaagcagauaccaguccaggaagguggagaacaugaaagcca
uaacagcuagacguauucugcaaggccuagggcauuauuugaaggcagaaggaaaaguggagugcuaccgaacccugca
uccuguuccuuuguauucaucuagugugaaccgugccuuuucaagccccaaggucgcaguggaagccuguaacgccaug
uugaaagagaacuuuccgacuguggcuucuuacuguauuauuccagaguacgaugccuauuuggacaugguugacggag
cuucaugcugcuuagacacugccaguuuuugcccugcaaagcugcgcagcuuuccaaagaaacacuccuauuuggaacc
cacaauacgaucggcagugccuucagcgauccagaacacgcuccagaacguccuggcagcugccacaaaaagaaauugc
aaugucacgcaaaugagagaauugcccguauuggauucggcggccuuuaauguggaaugcuucaagaaauaugcgugua
auaaugaauauugggaaacguuuaaagaaaaccccaucaggcuuacugaagaaaacgugguaaauuacauuaccaaauu
aaaaggaccaaaagcugcugcucuuuuugcgaagacacauaauuugaauauguugcaggacauaccaauggacagguuu
guaauggacuuaaagagagacgugaaagugacuccaggaacaaaacauacugaagaacggcccaagguacaggugaucc
aggcugccgauccgcuagcaacagcguaucugugcggaauccaccgagagcugguuaggagauuaaaugcgguccugcu
uccgaacauucauacacuguuugauaugucggcugaagacuuugacgcuauuauagccgagcacuuccagccuggggau
uguguucuggaaacugacaucgcgucguuugauaaaagugaggacgacgccauggcucugaccgcguuaaugauucugg
aagacuuagguguggacgcagagcuguugacgcugauugaggcggcuuucggcgaaauuucaucaauacauuugcccac
uaaaacuaaauuuaaauucggagccaugaugaaaucuggaauguuccucacacuguuugugaacacagucauuaacauu
guaaucgcaagcagaguguugagagaacggcuaaccggaucaccaugugcagcauucauuggagaugacaauaucguga
aaggagucaaaucggacaaauuaauggcagacaggugcgccaccugguugaauauggaagucaagauuauagaugcugu
gacccccuaaaaaggcuguuuaagcuuggcaaaccucuggcagcagacgaugaacaugaugaugacaggagaagggcau
ugcaugaagagucaacacgcuggaaccgaguggguauucuuucagagcugugcaaggcaguagaaucaagguaugaaac
cguaggaacuuccaucauaguuauggccaugacuacucuagcuagcaguguuaaaucauucagcuaccugagaggggcc
ccuauaacucucuacggcuaaccugaauggacuacgacauagucuaguccgccaagnugauaaccgcggugucaaaaac
gccauguacgugcugaccaaccagaaacauaauugaauacagcagcaauuggcaagcugcuuacauagaacucgcggcg
auuggcaugccgccuuaaaauuuuuauuuuauuuuuucuuuucuuuuccgaaucggauuuuguuuuuaauauuucaaaa
aaaaaaaaaaaaaaaaaaaaaaaaaaaaaaaaaaaaaaaaa
Vector Backbone RNA Sequence 5
SEQ ID NO: 36
auaggcggcgcaugagagaagcccagaccaauuaccuacccaaaauggagaaaguucacguugacaucgaggaagacag
cccauuccucagagcuuugcagcggagcuucccgcaguuugagguagaagccaagcaggucacugauaaugaccaugcu
aaugccagagcguuuucgcaucuggcuucaaaacugaucgaaacggagguggacccauccgacacgauccuugacauug
gaagugcgcccgcccgcagaauguauucuaagcacaaguaucauuguaucuguccgaugagaugugcggaagauccgga
cagauuguauaaguaugcaacuaagcugaagaaaaacuguaaggaaauaacugauaaggaauuggacaagaaaaugaag
gagcuggccgccgucaugagcgacccugaccuggaaacugagacuaugugccuccacgacgacgagucgugucgcuacg
aagggcaagucgcuguuuaccaggauguauacgcgguugacggaccgacaagucucuaucaccaagccaauaagggagu
uagagucgccuacuggauaggcuuugacaccaccccuuuuauguuuaagaacuuggcuggagcauauccaucauacucu
accaacugggccgacgaaaccguguuaacggcucguaacauaggccuaugcagcucugacguuauggagcggucacgua
gagggauguccauucuuagaaagaaguauuugaaaccauccaacaauguucuauucucuguuggcucgaccaucuacca
cgagaagagggacuuacugaggagcuggcaccugccgucuguauuucacuuacguggcaagcaaaauuacacaugucgg
ugugagacuauaguuaguugcgacggguacgucguuaaaagaauagcuaucaguccaggccuguaugggaagccuucag
gcuaugcugcuacgaugcaccgcgagggauucuugugcugcaaagugacagacacauugaacggggagagggucucuuu
ucccgugugcacguaugugccagcuacauugugugaccaaaugacuggcauacuggcaacagaugucagugcggacgac
gcgcaaaaacugcugguugggcucaaccagcguauagucgucaacggucgcacccagagaaacaccaauaccaugaaaa
auuaccuuuugcccguaguggcccaggcauuugcuaggugggcaaaggaauauaaggaagaucaagaagaugaaaggcc
acuaggacuacgagauagacaguuagucaugggguguuguugggcuuuuagaaggcacaagauaacaucuauuuauaag
cgcccggauacccaaaccaucaucaaagugaacagcgauuuccacucauucgugcugcccaggauaggcaguaacacau
uggagaucgggcugagaacaagaaucaggaaaauguuagaggagcacaaggagccgucaccucucauuaccgccgagga
cguacaagaagcuaagugcgcagccgaugaggcuaaggaggugcgugaagccgaggaguugcgcgcagcucuaccaccu
uuggcagcugauguugaggagcccacucuggaggcagacgucgacuugauguuacaagaggcuggggccggcucagugg
agacaccucguggcuugauaaagguuaccagcuacgauggcgaggacaagaucggcucuuacgcugugcuuucuccgca
ggcuguacucaagagugaaaaauuaucuugcauccacccucucgcugaacaagucauagugauaacacacucuggccga
aaagggcguuaugccguggaaccauaccaugguaaaguaguggugccagagggacaugcaauacccguccaggacuuuc
aagcucugagugaaagugccaccauuguguacaacgaacgugaguucguaaacagguaccugcaccauauugccacaca
uggaggagcgcugaacacugaugaagaauauuacaaaacugucaagcccagcgagcacgacggcgaauaccuguacgac
aucgacaggaaacagugcgucaagaaagaacuagucacugggcuagggcucacaggcgagcugguggauccucccuucc
augaauucgccuacgagagucugagaacacgaccagccgcuccuuaccaaguaccaaccauagggguguauggcgugcc
aggaucaggcaagucuggcaucauuaaaagcgcagucaccaaaaaagaucuaguggugagcgccaagaaagaaaacugu
gcagaaauuauaagggacgucaagaaaaugaaagggcuggacgucaaugccagaacuguggacucagugcucuugaaug
gaugcaaacaccccguagagacccuguauauugacgaagcuuuugcuugucaugcagguacucucagagcgcucauagc
cauuauaagaccuaaaaaggcagugcucugcggggaucccaaacagugcgguuuuuuuaacaugaugugccugaaagug
cauuuuaaccacgagauuugcacacaagucuuccacaaaagcaucucucgccguugcacuaaaucugugacuucggucg
ucucaaccuuguuuuacgacaaaaaaaugagaacgacgaauccgaaagagacuaagauugugauugacacuaccggcag
uaccaaaccuaagcaggacgaucucauucucacuuguuucagagggugggugaagcaguugcaaauagauuacaaaggc
aacgaaauaaugacggcagcugccucucaagggcugacccguaaagguguguaugccguucgguacaaggugaaugaaa
auccucuguacgcacccaccucagaacaugugaacguccuacugacccgcacggaggaccgcaucguguggaaaacacu
agccggcgacccauggauaaaaacacugacugccaaguacccugggaauuucacugccacgauagaggaguggcaagca
gagcaugaugccaucaugaggcacaucuuggagagaccggacccuaccgacgucuuccagaauaaggcaaacguguguu
gggccaaggcuuuagugccggugcugaagaccgcuggcauagacaugaccacugaacaauggaacacuguggauuauuu
ugaaacggacaaagcucacucagcagagauaguauugaaccaacuaugcgugagguucuuuggacucgaucuggacucc
ggucuauuuucugcacccacuguuccguuauccauuaggaauaaucacugggauaacuccccgucgccuaacauguacg
ggcugaauaaagaagugguccgucagcucucucgcagguacccacaacugccucgggcaguugccacuggaagagucua
ugacaugaacacugguacacugcgcaauuaugauccgcgcauaaaccuaguaccuguaaacagaagacugccucaugcu
uuaguccuccaccauaaugaacacccacagagugacuuuucuucauucgucagcaaauugaagggcagaacuguccugg
uggucggggaaaaguuguccgucccaggcaaaaugguugacugguugucagaccggccugaggcuaccuucagagcucg
gcuggauuuaggcaucccaggugaugugcccaaauaugacauaauauuuguuaaugugaggaccccauauaaauaccau
cacuaucagcagugugaagaccaugccauuaagcuuagcauguugaccaagaaagcuugucugcaucugaaucccggcg
gaaccugugucagcauagguuaugguuacgcugacagggccagcgaaagcaucauuggugcuauagcgcggcaguucaa
guuuucccggguaugcaaaccgaaauccucacuugaagagacggaaguucuguuuguauucauuggguacgaucgcaag
gcccguacgcacaauccuuacaagcuuucaucaaccuugaccaacauuuauacagguuccagacuccacgaagccggau
gugcacccucauaucauguggugcgaggggauauugccacggccaccgaaggagugauuauaaaugcugcuaacagcaa
aggacaaccuggcggaggggugugcggagcgcuguauaagaaauucccggaaagcuucgauuuacagccgaucgaagua
ggaaaagcgcgacuggucaaaggugcagcuaaacauaucauucaugccguaggaccaaacuucaacaaaguuucggagg
uugaaggugacaaacaguuggcagaggcuuaugaguccaucgcuaagauugucaacgauaacaauuacaagucaguagc
gauuccacuguuguccaccggcaucuuuuccgggaacaaagaucgacuaacccaaucauugaaccauuugcugacagcu
uuagacaccacugaugcagauguagccauauacugcagggacaagaaaugggaaaugacucucaaggaagcaguggcua
ggagagaagcaguggaggagauaugcauauccgacgacucuucagugacagaaccugaugcagagcuggugagggugca
uccgaagaguucuuuggcuggaaggaagggcuacagcacaagcgauggcaaaacuuucucauauuuggaagggaccaag
uuucaccaggcggccaaggauauagcagaaauuaaugccauguggcccguugcaacggaggccaaugagcagguaugca
uguauauccucggagaaagcaugagcaguauuaggucgaaaugccccgucgaagagucggaagccuccacaccaccuag
cacgcugccuugcuugugcauccaugccaugacuccagaaagaguacagcgccuaaaagccucacguccagaacaaauu
acugugugcucauccuuuccauugccgaaguauagaaucacuggugugcagaagauccaaugcucccagccuauauugu
ucucaccgaaagugccugcguauauucauccaaggaaguaucucguggaaacaccaccgguagacgagacuccggagcc
aucggcagagaaccaauccacagaggggacaccugaacaaccaccacuuauaaccgaggaugagaccaggacuagaacg
ccugagccgaucaucaucgaagaggaagaagaggauagcauaaguuugcugucagauggcccgacccaccaggugcugc
aagucgaggcagacauucacgggccgcccucuguaucuagcucauccugguccauuccucaugcauccgacuuugaugu
ggacaguuuauccauacuugacacccuggagggagcuagcgugaccagcggggcaacgucagccgagacuaacucuuac
uucgcaaagaguauggaguuucuggcgcgaccggugccugcgccucgaacaguauucaggaacccuccacaucccgcuc
cgcgcacaagaacaccgucacuugcacccagcagggccugcucgagaaccagccuaguuuccaccccgccaggcgugaa
uagggugaucacuagagaggagcucgaggcgcuuaccccgucacgcacuccuagcaggucggucucgagaaccagccug
gucuccaacccgccaggcguaaauagggugauuacaagagaggaguuugaggcguucguagcacaacaacaaugacggu
uugaugcgggugcauacaucuuuuccuccgacaccggucaagggcauuuacaacaaaaaucaguaaggcaaacggugcu
auccgaagugguguuggagaggaccgaauuggagauuucguaugccccgcgccucgaccaagaaaaagaagaauuacua
cgcaagaaauuacaguuaaaucccacaccugcuaacagaagcagauaccaguccaggaagguggagaacaugaaagcca
uaacagcuagacguauucugcaaggccuagggcauuauuugaaggcagaaggaaaaguggagugcuaccgaacccugca
uccuguuccuuuguauucaucuagugugaaccgugccuuuucaagccccaaggucgcaguggaagccuguaacgccaug
uugaaagagaacuuuccgacuguggcuucuuacuguauuauuccagaguacgaugccuauuuggacaugguugacggag
cuucaugcugcuuagacacugccaguuuuugcccugcaaagcugcgcagcuuuccaaagaaacacuccuauuuggaacc
cacaauacgaucggcagugccuucagcgauccagaacacgcuccagaacguccuggcagcugccacaaaaagaaauugc
aaugucacgcaaaugagagaauugcccguauuggauucggcggccuuuaauguggaaugcuucaagaaauaugcgugua
auaaugaauauugggaaacguuuaaagaaaaccccaucaggcuuacugaagaaaacgugguaaauuacauuaccaaauu
aaaaggaccaaaagcugcugcucuuuuugcgaagacacauaauuugaauauguugcaggacauaccaauggacagguuu
guaauggacuuaaagagagacgugaaagugacuccaggaacaaaacauacugaagaacggcccaagguacaggugaucc
aggcugccgauccgcuagcaacagcguaucugugcggaauccaccgagagcugguuaggagauuaaaugcgguccugcu
uccgaacauucauacacuguuugauaugucggcugaagacuuugacgcuauuauagccgagcacuuccagccuggggau
uguguucuggaaacugacaucgcgucguuugauaaaagugaggacgacgccauggcucugaccgcguuaaugauucugg
aagacuuagguguggacgcagagcuguugacgcugauugaggcggcuuucggcgaaauuucaucaauacauuugcccac
uaaaacuaaauuuaaauucggagccaugaugaaaucuggaauguuccucacacuguuugugaacacagucauuaacauu
guaaucgcaagcagaguguugagagaacggcuaaccggaucaccaugugcagcauucauuggagaugacaauaucguga
aaggagucaaaucggacaaauuaauggcagacaggugcgccaccugguugaauauggaagucaagauuauagaugcugu
ggugggcgagaaagcgccuuauuucuguggaggguuuauuuugugugacuccgugaccggcacagcgugccguguggca
gacccccuaaaaaggcuguuuaagcuuggcaaaccucuggcagcagacgaugaacaugaugaugacaggagaagggcau
ugcaugaagagucaacacgcuggaaccgaguggguauucuuucagagcugugcaaggcaguagaaucaagguaugaaac
cguaggaacuuccaucauaguuauggccaugacuacucuagcuagcaguguuaaaucauucagcuaccugagaggggcc
ccuauaacucucuacggcuaaccugaauggacuacgacauagucuaguccgccaagnugauaaccgcggugucaaaaac
gccauguacgugcugaccaaccagaaacauaauugaauacagcagcaauuggcaagcugcuuacauagaacucgcggcg
auuggcaugccgccuuaaaauuuuuauuuuauuuuuucuuuucuuuuccgaaucggauuuuguuuuuaauauuucaaaa
aaaaaaaaaaaaaaaaaaaaaaaaaaaaaaaaaaaaaaaaa
Vector Backbone RNA Sequence 6
SEQ ID NO: 37
auaggcggcgcaugagagaagcccagaccaauuaccuacccaaaauggagaaaguucacguugacaucgaggaagacag
cccauuccucagagcuuugcagcggagcuucccgcaguuugagguagaagccaagcaggucacugauaaugaccaugcu
aaugccagagcguuuucgcaucuggcuucaaaacugaucgaaacggagguggacccauccgacacgauccuugacauug
gaagugcgcccgcccgcagaauguauucuaagcacaaguaucauuguaucuguccgaugagaugugcggaagauccgga
cagauuguauaaguaugcaacuaagcugaagaaaaacuguaaggaaauaacugauaaggaauuggacaagaaaaugaag
gagcuggccgccgucaugagcgacccugaccuggaaacugagacuaugugccuccacgacgacgagucgugucgcuacg
aagggcaagucgcuguuuaccaggauguauacgcgguugacggaccgacaagucucuaucaccaagccaauaagggagu
uagagucgccuacuggauaggcuuugacaccaccccuuuuauguuuaagaacuuggcuggagcauauccaucauacucu
accaacugggccgacgaaaccguguuaacggcucguaacauaggccuaugcagcucugacguuauggagcggucacgua
gagggauguccauucuuagaaagaaguauuugaaaccauccaacaauguucuauucucuguuggcucgaccaucuacca
cgagaagagggacuuacugaggagcuggcaccugccgucuguauuucacuuacguggcaagcaaaauuacacaugucgg
ugugagacuauaguuaguugcgacggguacgucguuaaaagaauagcuaucaguccaggccuguaugggaagccuucag
gcuaugcugcuacgaugcaccgcgagggauucuugugcugcaaagugacagacacauugaacggggagagggucucuuu
ucccgugugcacguaugugccagcuacauugugugaccaaaugacuggcauacuggcaacagaugucagugcggacgac
gcgcaaaaacugcugguugggcucaaccagcguauagucgucaacggucgcacccagagaaacaccaauaccaugaaaa
auuaccuuuugcccguaguggcccaggcauuugcuaggugggcaaaggaauauaaggaagaucaagaagaugaaaggcc
acuaggacuacgagauagacaguuagucaugggguguuguugggcuuuuagaaggcacaagauaacaucuauuuauaag
cgcccggauacccaaaccaucaucaaagugaacagcgauuuccacucauucgugcugcccaggauaggcaguaacacau
uggagaucgggcugagaacaagaaucaggaaaauguuagaggagcacaaggagccgucaccucucauuaccgccgagga
cguacaagaagcuaagugcgcagccgaugaggcuaaggaggugcgugaagccgaggaguugcgcgcagcucuaccaccu
uuggcagcugauguugaggagcccacucuggaggcagacgucgacuugauguuacaagaggcuggggccggcucagugg
agacaccucguggcuugauaaagguuaccagcuacgauggcgaggacaagaucggcucuuacgcugugcuuucuccgca
ggcuguacucaagagugaaaaauuaucuugcauccacccucucgcugaacaagucauagugauaacacacucuggccga
aaagggcguuaugccguggaaccauaccaugguaaaguaguggugccagagggacaugcaauacccguccaggacuuuc
aagcucugagugaaagugccaccauuguguacaacgaacgugaguucguaaacagguaccugcaccauauugccacaca
uggaggagcgcugaacacugaugaagaauauuacaaaacugucaagcccagcgagcacgacggcgaauaccuguacgac
aucgacaggaaacagugcgucaagaaagaacuagucacugggcuagggcucacaggcgagcugguggauccucccuucc
augaauucgccuacgagagucugagaacacgaccagccgcuccuuaccaaguaccaaccauagggguguauggcgugcc
aggaucaggcaagucuggcaucauuaaaagcgcagucaccaaaaaagaucuaguggugagcgccaagaaagaaaacugu
gcagaaauuauaagggacgucaagaaaaugaaagggcuggacgucaaugccagaacuguggacucagugcucuugaaug
gaugcaaacaccccguagagacccuguauauugacgaagcuuuugcuugucaugcagguacucucagagcgcucauagc
cauuauaagaccuaaaaaggcagugcucugcggggaucccaaacagugcgguuuuuuuaacaugaugugccugaaagug
cauuuuaaccacgagauuugcacacaagucuuccacaaaagcaucucucgccguugcacuaaaucugugacuucggucg
ucucaaccuuguuuuacgacaaaaaaaugagaacgacgaauccgaaagagacuaagauugugauugacacuaccggcag
uaccaaaccuaagcaggacgaucucauucucacuuguuucagagggugggugaagcaguugcaaauagauuacaaaggc
aacgaaauaaugacggcagcugccucucaagggcugacccguaaagguguguaugccguucgguacaaggugaaugaaa
auccucuguacgcacccaccucagaacaugugaacguccuacugacccgcacggaggaccgcaucguguggaaaacacu
agccggcgacccauggauaaaaacacugacugccaaguacccugggaauuucacugccacgauagaggaguggcaagca
gagcaugaugccaucaugaggcacaucuuggagagaccggacccuaccgacgucuuccagaauaaggcaaacguguguu
gggccaaggcuuuagugccggugcugaagaccgcuggcauagacaugaccacugaacaauggaacacuguggauuauuu
ugaaacggacaaagcucacucagcagagauaguauugaaccaacuaugcgugagguucuuuggacucgaucuggacucc
ggucuauuuucugcacccacuguuccguuauccauuaggaauaaucacugggauaacuccccgucgccuaacauguacg
ggcugaauaaagaagugguccgucagcucucucgcagguacccacaacugccucgggcaguugccacuggaagagucua
ugacaugaacacugguacacugcgcaauuaugauccgcgcauaaaccuaguaccuguaaacagaagacugccucaugcu
uuaguccuccaccauaaugaacacccacagagugacuuuucuucauucgucagcaaauugaagggcagaacuguccugg
uggucggggaaaaguuguccgucccaggcaaaaugguugacugguugucagaccggccugaggcuaccuucagagcucg
gcuggauuuaggcaucccaggugaugugcccaaauaugacauaauauuuguuaaugugaggaccccauauaaauaccau
cacuaucagcagugugaagaccaugccauuaagcuuagcauguugaccaagaaagcuugucugcaucugaaucccggcg
gaaccugugucagcauagguuaugguuacgcugacagggccagcgaaagcaucauuggugcuauagcgcggcaguucaa
guuuucccggguaugcaaaccgaaauccucacuugaagagacggaaguucuguuuguauucauuggguacgaucgcaag
gcccguacgcacaauccuuacaagcuuucaucaaccuugaccaacauuuauacagguuccagacuccacgaagccggau
gugcacccucauaucauguggugcgaggggauauugccacggccaccgaaggagugauuauaaaugcugcuaacagcaa
aggacaaccuggcggaggggugugcggagcgcuguauaagaaauucccggaaagcuucgauuuacagccgaucgaagua
ggaaaagcgcgacuggucaaaggugcagcuaaacauaucauucaugccguaggaccaaacuucaacaaaguuucggagg
uugaaggugacaaacaguuggcagaggcuuaugaguccaucgcuaagauugucaacgauaacaauuacaagucaguagc
gauuccacuguuguccaccggcaucuuuuccgggaacaaagaucgacuaacccaaucauugaaccauuugcugacagcu
uuagacaccacugaugcagauguagccauauacugcagggacaagaaaugggaaaugacucucaaggaagcaguggcua
ggagagaagcaguggaggagauaugcauauccgacgacucuucagugacagaaccugaugcagagcuggugagggugca
uccgaagaguucuuuggcuggaaggaagggcuacagcacaagcgauggcaaaacuuucucauauuuggaagggaccaag
uuucaccaggcggccaaggauauagcagaaauuaaugccauguggcccguugcaacggaggccaaugagcagguaugca
uguauauccucggagaaagcaugagcaguauuaggucgaaaugccccgucgaagagucggaagccuccacaccaccuag
cacgcugccuugcuugugcauccaugccaugacuccagaaagaguacagcgccuaaaagccucacguccagaacaaauu
acugugugcucauccuuuccauugccgaaguauagaaucacuggugugcagaagauccaaugcucccagccuauauugu
ucucaccgaaagugccugcguauauucauccaaggaaguaucucguggaaacaccaccgguagacgagacuccggagcc
aucggcagagaaccaauccacagaggggacaccugaacaaccaccacuuauaaccgaggaugagaccaggacuagaacg
ccugagccgaucaucaucgaagaggaagaagaggauagcauaaguuugcugucagauggcccgacccaccaggugcugc
aagucgaggcagacauucacgggccgcccucuguaucuagcucauccugguccauuccucaugcauccgacuuugaugu
ggacaguuuauccauacuugacacccuggagggagcuagcgugaccagcggggcaacgucagccgagacuaacucuuac
uucgcaaagaguauggaguuucuggcgcgaccggugccugcgccucgaacaguauucaggaacccuccacaucccgcuc
cgcgcacaagaacaccgucacuugcacccagcagggccugcucgagaaccagccuaguuuccaccccgccaggcgugaa
uagggugaucacuagagaggagcucgaggcgcuuaccccgucacgcacuccuagcaggucggucucgagaaccagccug
gucuccaacccgccaggcguaaauagggugauuacaagagaggaguuugaggcguucguagcacaacaacaaugacggu
uugaugcgggugcauacaucuuuuccuccgacaccggucaagggcauuuacaacaaaaaucaguaaggcaaacggugcu
auccgaagugguguuggagaggaccgaauuggagauuucguaugccccgcgccucgaccaagaaaaagaagaauuacua
cgcaagaaauuacaguuaaaucccacaccugcuaacagaagcagauaccaguccaggaagguggagaacaugaaagcca
uaacagcuagacguauucugcaaggccuagggcauuauuugaaggcagaaggaaaaguggagugcuaccgaacccugca
uccuguuccuuuguauucaucuagugugaaccgugccuuuucaagccccaaggucgcaguggaagccuguaacgccaug
uugaaagagaacuuuccgacuguggcuucuuacuguauuauuccagaguacgaugccuauuuggacaugguugacggag
cuucaugcugcuuagacacugccaguuuuugcccugcaaagcugcgcagcuuuccaaagaaacacuccuauuuggaacc
cacaauacgaucggcagugccuucagcgauccagaacacgcuccagaacguccuggcagcugccacaaaaagaaauugc
aaugucacgcaaaugagagaauugcccguauuggauucggcggccuuuaauguggaaugcuucaagaaauaugcgugua
auaaugaauauugggaaacguuuaaagaaaaccccaucaggcuuacugaagaaaacgugguaaauuacauuaccaaauu
aaaaggaccaaaagcugcugcucuuuuugcgaagacacauaauuugaauauguugcaggacauaccaauggacagguuu
guaauggacuuaaagagagacgugaaagugacuccaggaacaaaacauacugaagaacggcccaagguacaggugaucc
aggcugccgauccgcuagcaacagcguaucugugcggaauccaccgagagcugguuaggagauuaaaugcgguccugcu
uccgaacauucauacacuguuugauaugucggcugaagacuuugacgcuauuauagccgagcacuuccagccuggggau
uguguucuggaaacugacaucgcgucguuugauaaaagugaggacgacgccauggcucugaccgcguuaaugauucugg
aagacuuagguguggacgcagagcuguugacgcugauugaggcggcuuucggcgaaauuucaucaauacauuugcccac
uaaaacuaaauuuaaauucggagccaugaugaaaucuggaauguuccucacacuguuugugaacacagucauuaacauu
guaaucgcaagcagaguguugagagaacggcuaaccggaucaccaugugcagcauucauuggagaugacaauaucguga
aaggagucaaaucggacaaauuaauggcagacaggugcgccaccugguugaauauggaagucaagauuauagaugcugu
ggugggcgagaaagcgccuuauuucuguggaggguuuauuuugugugacuccgugaccggcacagcgugccguguggca
gacccccuaaaaaggcuguuuaagcuuggcaaaccucuggcagcagacgaugaacaugaugaugacaggagaagggcau
ugcaugaagagucaacacgcuggaaccgaguggguauucuuucagagcugugcaaggcaguagaaucaagguaugaaac
cguaggaacuuccaucauaguuauggccaugacuacucuagcuagcaguguuaaaucauucagcuaccugagaggggcc
ccuauaacucucuacggcuaaccugaauggacuacgacauagucuaguccgccaagAUGUUUCUGCUCACAACCAAACG
CACUAUGUUUGUUUUCCUCGUGCUGCUCCCUUUGGUAAGUUCUCAGUGUGUAAACCUGagaACACGAACCCAGUUGCCU
CCAGCUUAUACCAACUCAUUUACUCGCGGAGUAUAUUAUCCCGAUAAGGUCUUUAGAAGUAGCGUGUUGCACUCUACAC
AGGAUCUGUUCUUGCCCUUCUUUAGUAACGUUACCUGGUUUCAUGCAAUACAUGUGAGCGGAACAAAUGGAACAAAAAG
AUUUGACAAUCCAGUGCUUCCAUUUAAUGAUGGGGUUUACUUUGCCAGUaucGAAAAGUCAAACAUAAUCCGGGGGUGG
AUCUUUGGAACCACUUUGGACUCUAAGACACAGUCUCUCCUCAUAGUAAACAACGCCACCAAUGUUGUCAUAAAAGUAU
GCGAAUUUCAGUUUUGCAACGAUCCCUUUCUCgacGUGUAUUACCAUAAGAAUAAUAAAUCCUGGAUGGAGUCUgggGU
UUAUAGUAGUGCUAAUAAUUGCACUUUCGAAUACGUGUCCCAACCAUUCCUCAUGGACCUUGAGGGCAAACAGGGGAAU
UUUAAAAACUUGCGCGAAUUUGUCUUUAAGAAUAUCGACGGAUACUUUAAGAUCUAUAGUAAACACACUCCUAUCAACC
UCGUUCGGGAUCUUCCCCAAGGCUUUUCUGCUCUCGAACCCCUCGUAGACUUGCCAAUUGGGAUAAAUAUCACUCGCUU
UCAAACUUUGCUUGCCCUCCACAGGAGCUACCUGACACCCGGCGACUCUUCUUCUGGUuugACCGCCGGCGCCGCUGCC
UAUUAUGUUGGUUACCUUCAGCCACGAACAUUCUUGCUCAAGUAUAACGAGAAUGGCACCAUUACCGACGCCGUCGAUU
GUGCAUUGGAUCCCUUGUCUGAAACAAAAUGUACCUUGAAGUCCUUUACCGUAGAGAAAGGCAUAUACCAGACUUCCAA
CUUCCGAGUUCAGCCUACAGAAUCCAUCGUACGAUUUCCCAACAUCACAAACCUCUGCCCUUUCGGUGAAGUAUUUAAU
GCUACACGCUUCGCUUCAGUCUAUGCCUGGAAUAGGAAGCGCAUAUCAAAUUGCGUGGCCGAUUAUUCAGUCCUCUAUA
AUAGCGCAUCCUUCAGUACUUUCAAGUGCUACGGCGUUUCCCCCACCAAACUCAAUGAUCUUUGCUUCACCAACGUCUA
UGCUGACAGUUUUGUCAUACGAGGCGACGAAGUACGCCAGAUUGCCCCCGGGCAGACAGGUaauAUUGCUGAUUAUAAU
UAUAAACUCCCAGAUGACUUUACUGGAUGCGUCAUAGCCUGGAAUUCCAACAAUCUAGAUUCCAAGGUUGGUGGGAAUU
AUAAUUACcguUAUCGACUGUUCAGAAAGAGUAACUUGAAACCAUUUGAGAGAGACAUAUCCACCGAGAUUUACCAGGC
AGGCAGUaagCCUUGUAACGGCGUUGAGGGAUUUAACUGCUAUUUUCCUUUGCAAUCCUAUGGCUUUCAACCAACAAAC
GGGGUUGGCUAUCAACCCUAUCGAGUGGUUGUCCUCAGCUUUGAACUUUUGCACGCUCCCGCCACAGUCUGCGGACCAA
AAAAGAGUACAAAUCUUGUCAAGAAUAAGUGCGUAAAUUUCAAUUUCAAUGGCCUUACAGGAACAGGCGUGCUGACUGA
GUCAAACAAGaauUUCCUGCCAUUUCAGCAGUUUGGGCGGGAUAUAGCAGACACAACUGACGCUGUACGCGAUCCUCAG
ACUUUGGAGAUCUUGGACAUCACUCCCUGUUCUUUCGGAGGGGUAUCUGUCAUCACCCCCGGAACUAAUACAUCAAAUC
AGGUCGCUGUGUUGUACCAAgguGUCAACUGCACAGAAGUCCCCGUUGCUAUACACGCAGACCAGCUCACCCCCACAUG
GCGGGUGUACUCAACUGGCUCAAACGUAUUCCAGACCAGAGCUGGGUGCUUGAUCGGUGCUGAACACGUGAACAAUAGC
UAUGAAUGCGAUAUUCCCAUCGGUGCCGGGAUCUGCGCUAGCUAUCAGACACAGACCAAUUCCcgcAGGCGGGCUCGCU
CUGUAGCAUCCCAGUCUAUUAUUGCCUACACUAUGUCAUUGGGCGCCGAGAAUAGCGUCGCAUAUUCAAAUAAUUCUAU
UGCAAUACCCACCAACUUCACAAUCUCCGUAACUACAGAAAUACUUCCAGUUUCCAUGACAAAGACAUCAGUGGAUUGU
ACAAUGUAUAUAUGCGGAGAUUCCACAGAAUGUUCAAAUUUGCUCUUGCAGUACGGCUCCUUCUGCACCCAGCUCAACA
GGGCACUUACAGGUAUUGCUGUCGAACAGGACAAGAACACACAAGAAGUCUUCGCCCAAGUCAAACAGAUAUACAAAAC
UCCUCCCAUAAAGGAUUUUGGCGGCUUCAACUUUAGUCAGAUCCUCCCAGACCCUUCAAAACCAUCUAAACGAUCAUUU
AUUGAAGAUCUGCUGUUCAACAAGGUCACUCUUGCCGAUGCUGGAUUCAUUAAGCAAUACGGUGACUGCCUUGGUGAUA
UUGCUGCCCGAGAUCUGAUCUGUGCCCAGAAAUUCAACGGGCUCACUGUACUCCCUCCACUGCUCACAGACGAAAUGAU
UGCACAGUACACAAGUGCCCUGUUGGCAGGCACAAUCACUAGCGGCUGGACCUUUGGCGCAGGUGCAGCACUCCAAAUA
CCUUUUGCCAUGCAGAUGGCCUAUCGGUUUAAUGGGAUAGGCGUGACUCAAAAUGUCCUCUACGAAAACCAAAAGUUGA
UAGCUAACCAAUUCAAUUCAGCAAUCGGGAAGAUACAGGAUUCACUGUCUAGUACUGCUAGUGCCCUUGGUAAGCUGCA
GaacGUUGUCAACCAGAAUGCUCAAGCUCUGAAUACAUUGGUUAAGCAGCUCUCUAGUAAUUUUGGGGCCAUCUCUUCA
GUACUUAAUGAUAUUUUGAGCCGAUUGGACccaccuGAAGCUGAAGUACAGAUCGACAGGCUGAUAACAGGCCGGCUCC
AAUCCCUCCAAACAUACGUGACACAACAACUCAUACGCGCAGCCGAAAUCCGAGCCAGCGCUAACCUGGCAGCUACCAA
GAUGUCAGAAUGCGUUCUGGGCCAGAGUAAACGCGUAGAUUUCUGCGGGAAAGGGUACCACCUGAUGUCCUUUCCACAA
UCUGCACCUCACGGGGUCGUCUUUUUGCAUGUAACAUACGUACCCGCACAAGAGAAGAAUUUUACUACCGCUCCUGCCA
UCUGUCAUGACGGGAAAGCUCAUUUUCCUCGCGAAGGUGUGUUUGUAUCUAAUGGUACACAUUGGUUUGUCACACAGCG
GAAUUUCUAUGAACCCCAGAUCAUUACAACUGACAACACUUUUGUUUCCGGGAAUUGUGACGUGGUCAUAGGAAUCGUA
AAUAACACUGUAUAUGAUCCCCUCCAACCAGAGCUGGACUCUUUUAAAGAAGAACUGGAUAAAUAUUUCAAGAACCACA
CAAGUCCCGACGUGGACCUUGGGGACAUAAGUGGUAUUAACGCAUCUGUGGUUAACAUUCAAAAGGAAAUCGACAGACU
CAACGAGGUGGCCAAAAACCUGAACGAAAGCUUGAUAGAUCUCCAGGAGUUGGGCAAGUAUGAACAGUACAUUAAAUGG
CCAUGGUACAUAUGGCUUGGCUUUAUCGCUGGCCUUAUCGCCAUCGUAAUGGUUACAAUCAUGCUGUGCUGCAUGACCU
CCUGCUGUUCUUGUUUGAAAGGGUGUUGUUCUUGUGGUAGUUGUUGCAAGUUUGACGAAGAUGAUUCCGAACCUGUUCU
UAAGGGGGUAAAGCUUCACUAUACAUGAuaaccgcggugucaaaaaccgcguggacgugguuaacaucccugcugggag
gaucagccguaauuauuauaauuggcuuggugcuggcuacuauuguggccauguacgugcugaccaaccagaaacauaa
uugaauacagcagcaauuggcaagcugcuuacauagaacucgcggcgauuggcaugccgccuuaaaauuuuuauuuuau
uuuuucuuuucuuuuccgaaucggauuuuguuuuuaauauuucaaaaaaaaaaaaaaaaaaaaaaaaaaaaaaaaaaaa
aaaaaaaaaaaa
Vector Backbone RNA Sequence 7
SEQ ID NO: 38
auaggcggcgcaugagagaagcccagaccaauuaccuacccaaaauggagaaaguucacguugacaucgaggaagacagc
ccauuccucagagcuuugcagcggagcuucccgcaguuugagguagaagccaagcaggucacugauaaugaccaugcuaa
ugccagagcguuuucgcaucuggcuucaaaacugaucgaaacggagguggacccauccgacacgauccuugacauuggaa
gugcgcccgcccgcagaauguauucuaagcacaaguaucauuguaucuguccgaugagaugugcggaagauccggacaga
uuguauaaguaugcaacuaagcugaagaaaaacuguaaggaaauaacugauaaggaauuggacaagaaaaugaaggagcu
ggccgccgucaugagcgacccugaccuggaaacugagacuaugugccuccacgacgacgagucgugucgcuacgaagggc
aagucgcuguuuaccaggauguauacgcgguugacggaccgacaagucucuaucaccaagccaauaagggaguuagaguc
gccuacuggauaggcuuugacaccaccccuuuuauguuuaagaacuuggcuggagcauauccaucauacucuaccaacug
ggccgacgaaaccguguuaacggcucguaacauaggccuaugcagcucugacguuauggagcggucacguagagggaugu
ccauucuuagaaagaaguauuugaaaccauccaacaauguucuauucucuguuggcucgaccaucuaccacgagaagagg
gacuuacugaggagcuggcaccugccgucuguauuucacuuacguggcaagcaaaauuacacaugucggugugagacuau
aguuaguugcgacggguacgucguuaaaagaauagcuaucaguccaggccuguaugggaagccuucaggcuaugcugcua
cgaugcaccgcgagggauucuugugcugcaaagugacagacacauugaacggggagagggucucuuuucccgugugcacg
uaugugccagcuacauugugugaccaaaugacuggcauacuggcaacagaugucagugcggacgacgcgcaaaaacugcu
gguugggcucaaccagcguauagucgucaacggucgcacccagagaaacaccaauaccaugaaaaauuaccuuuugcccg
uaguggcccaggcauuugcuaggugggcaaaggaauauaaggaagaucaagaagaugaaaggccacuaggacuacgagau
agacaguuagucaugggguguuguugggcuuuuagaaggcacaagauaacaucuauuuauaagcgcccggauacccaaac
caucaucaaagugaacagcgauuuccacucauucgugcugcccaggauaggcaguaacacauuggagaucgggcugagaa
caagaaucaggaaaauguuagaggagcacaaggagccgucaccucucauuaccgccgaggacguacaagaagcuaagugc
gcagccgaugaggcuaaggaggugcgugaagccgaggaguugcgcgcagcucuaccaccuuuggcagcugauguugagga
gcccacucuggaggcagacgucgacuugauguuacaagaggcuggggccggcucaguggagacaccucguggcuugauaa
agguuaccagcuacgauggcgaggacaagaucggcucuuacgcugugcuuucuccgcaggcuguacucaagagugaaaaa
uuaucuugcauccacccucucgcugaacaagucauagugauaacacacucuggccgaaaagggcguuaugccguggaacc
auaccaugguaaaguaguggugccagagggacaugcaauacccguccaggacuuucaagcucugagugaaagugccacca
uuguguacaacgaacgugaguucguaaacagguaccugcaccauauugccacacauggaggagcgcugaacacugaugaa
gaauauuacaaaacugucaagcccagcgagcacgacggcgaauaccuguacgacaucgacaggaaacagugcgucaagaa
agaacuagucacugggcuagggcucacaggcgagcugguggauccucccuuccaugaauucgccuacgagagucugagaa
cacgaccagccgcuccuuaccaaguaccaaccauagggguguauggcgugccaggaucaggcaagucuggcaucauuaaa
agcgcagucaccaaaaaagaucuaguggugagcgccaagaaagaaaacugugcagaaauuauaagggacgucaagaaaau
gaaagggcuggacgucaaugccagaacuguggacucagugcucuugaauggaugcaaacaccccguagagacccuguaua
uugacgaagcuuuugcuugucaugcagguacucucagagcgcucauagccauuauaagaccuaaaaaggcagugcucugc
ggggaucccaaacagugcgguuuuuuuaacaugaugugccugaaagugcauuuuaaccacgagauuugcacacaagucuu
ccacaaaagcaucucucgccguugcacuaaaucugugacuucggucgucucaaccuuguuuuacgacaaaaaaaugagaa
cgacgaauccgaaagagacuaagauugugauugacacuacoggcaguaccaaaccuaagcaggacgaucucauucucacu
uguuucagagggugggugaagcaguugcaaauagauuacaaaggcaacgaaauaaugacggcagcugccucucaagggcu
gacccguaaagguguguaugccguucgguacaaggugaaugaaaauccucuguacgcacccaccucagaacaugugaacg
uccuacugacccgcacggaggaccgcaucguguggaaaacacuagccggcgacccauggauaaaaacacugacugccaag
uacccugggaauuucacugccacgauagaggaguggcaagcagagcaugaugccaucaugaggcacaucuuggagagacc
ggacccuaccgacgucuuccagaauaaggcaaacguguguugggccaaggcuuuagugccggugcugaagaccgcuggca
uagacaugaccacugaacaauggaacacuguggauuauuuugaaacggacaaagcucacucagcagagauaguauugaac
caacuaugcgugagguucuuuggacucgaucuggacuccggucuauuuucugcacccacuguuccguuauccauuaggaa
uaaucacugggauaacuccccgucgccuaacauguacgggcugaauaaagaagugguccgucagcucucucgcagguacc
cacaacugccucgggcaguugccacuggaagagucuaugacaugaacacugguacacugcgcaauuaugauccgcgcaua
aaccuaguaccuguaaacagaagacugccucaugcuuuaguccuccaccauaaugaacacccacagagugacuuuucuuc
auucgucagcaaauugaagggcagaacuguccugguggucggggaaaaguuguccgucccaggcaaaaugguugacuggu
ugucagaccggccugaggcuaccuucagagcucggcuggauuuaggcaucccaggugaugugcccaaauaugacauaaua
uuuguuaaugugaggaccccauauaaauaccaucacuaucagcagugugaagaccaugccauuaagcuuagcauguugac
caagaaagcuugucugcaucugaaucccggcggaaccugugucagcauagguuaugguuacgcugacagggccagcgaaa
gcaucauuggugcuauagcgcggcaguucaaguuuucccggguaugcaaaccgaaauccucacuugaagagacggaaguu
cuguuuguauucauuggguacgaucgcaaggcccguacgcacaauccuuacaagcuuucaucaaccuugaccaacauuua
uacagguuccagacuccacgaagccggaugugcacccucauaucauguggugcgaggggauauugccacggccaccgaag
gagugauuauaaaugcugcuaacagcaaaggacaaccuggcggaggggugugcggagcgcuguauaagaaauucccggaa
agcuucgauuuacagccgaucgaaguaggaaaagcgcgacuggucaaaggugcagcuaaacauaucauucaugccguagg
accaaacuucaacaaaguuucggagguugaaggugacaaacaguuggcagaggcuuaugaguccaucgcuaagauuguca
acgauaacaauuacaagucaguagcgauuccacuguuguccaccggcaucuuuuccgggaacaaagaucgacuaacccaa
ucauugaaccauuugcugacagcuuuagacaccacugaugcagauguagccauauacugcagggacaagaaaugggaaau
gacucucaaggaagcaguggcuaggagagaagcaguggaggagauaugcauauccgacgacucuucagugacagaaccug
augcagagcuggugagggugcauccgaagaguucuuuggcuggaaggaagggcuacagcacaagcgauggcaaaacuuuc
ucauauuuggaagggaccaaguuucaccaggcggccaaggauauagcagaaauuaaugccauguggcccguugcaacgga
ggccaaugagcagguaugcauguauauccucggagaaagcaugagcaguauuaggucgaaaugccccgucgaagagucgg
aagccuccacaccaccuagcacgcugccuugcuugugcauccaugccaugacuccagaaagaguacagcgccuaaaagcc
ucacguccagaacaaauuacugugugcucauccuuuccauugccgaaguauagaaucacuggugugcagaagauccaaug
cucccagccuauauuguucucaccgaaagugccugcguauauucauccaaggaaguaucucguggaaacaccaccgguag
acgagacuccggagccaucggcagagaaccaauccacagaggggacaccugaacaaccaccacuuauaaccgaggaugag
accaggacuagaacgccugagccgaucaucaucgaagaggaagaagaggauagcauaaguuugcugucagauggcccgac
ccaccaggugcugcaagucgaggcagacauucacgggccgcccucuguaucuagcucauccugguccauuccucaugcau
ccgacuuugauguggacaguuuauccauacuugacacccuggagggagcuagcgugaccagcggggcaacgucagccgag
acuaacucuuacuucgcaaagaguauggaguuucuggcgcgaccggugccugcgccucgaacaguauucaggaacccucc
acaucccgcuccgcgcacaagaacaccgucacuugcacccagcagggccugcucgagaaccagccuaguuuccaccccgc
caggcgugaauagggugaucacuagagaggagcucgaggcgcuuaccccgucacgcacuccuagcaggucggucucgaga
accagccuggucuccaacccgccaggcguaaauagggugauuacaagagaggaguuugaggcguucguagcacaacaaca
augacgguuugaugcgggugcauacaucuuuuccuccgacaccggucaagggcauuuacaacaaaaaucaguaaggcaaa
cggugcuauccgaagugguguuggagaggaccgaauuggagauuucguaugccccgcgccucgaccaagaaaaagaagaa
uuacuacgcaagaaauuacaguuaaaucccacaccugcuaacagaagcagauaccaguccaggaagguggagaacaugaa
agccauaacagcuagacguauucugcaaggccuagggcauuauuugaaggcagaaggaaaaguggagugcuaccgaaccc
ugcauccuguuccuuuguauucaucuagugugaaccgugccuuuucaagccccaaggucgcaguggaagccuguaacgcc
auguugaaagagaacuuuccgacuguggcuucuuacuguauuauuccagaguacgaugccuauuuggacaugguugacgg
agcuucaugcugcuuagacacugccaguuuuugcccugcaaagcugcgcagcuuuccaaagaaacacuccuauuuggaac
ccacaauacgaucggcagugccuucagcgauccagaacacgcuccagaacguccuggcagcugccacaaaaagaaauugc
aaugucacgcaaaugagagaauugcccguauuggauucggcggccuuuaauguggaaugcuucaagaaauaugcguguaa
uaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuugua
auggacuuaaagagagacgugaaagugacuccaggaacaaaacauacugaagaacggcccaagguacaggugauccaggc
ugccgauccgcuagcaacagcguaucugugcggaauccaccgagagcugguuaggagauuaaaugcgguccugcuuccga
acauucauacacuguuugauaugucggcugaagacuuugacgcuauuauagccgagcacuuccagccuggggauuguguu
cuggaaacugacaucgcgucguuugauaaaagugaggacgacgccauggcucugaccgcguuaaugauucuggaagacuu
agguguggacgcagagcuguugacgcugauugaggcggcuuucggcgaaauuucaucaauacauuugcccacuaaaacua
aauuuaaauucggagccaugaugaaaucuggaauguuccucacacuguuugugaacacagucauuaacauuguaaucgca
agcagaguguugagagaacggcuaaccggaucaccaugugcagcauucauuggagaugacaauaucgugaaaggagucaa
aucggacaaauuaauggcagacaggugcgccaccugguugaauauggaagucaagauuauagaugcuguggugggcgaga
aagcgccuuauuucuguggaggguuuauuuugugugacuccgugaccggcacagcgugccguguggcagacccccuaaaa
aggcuguuuaagcuuggcaaaccucuggcagcagacgaugaacaugaugaugacaggagaagggcauugcaugaagaguc
aacacgcuggaaccgaguggguauucuuucagagcugugcaaggcaguagaaucaagguaugaaaccguaggaacuucca
ucauaguuauggccaugacuacucuagcuagcaguguuaaaucauucagcuaccugagaggggccccuauaacucucuac
ggcuaaccugaauggacuacgacauagucuaguccgccgccaccnuaaccgcggugucaaaaaccgcguggacgugguua
caaccagaaacauaauugaauacagcagcaauuggcaagcugcuuacauagaacucgcggcgauuggcaugccgccuuaa
aauuuuuauuuuauuuuuucuuuucuuuccgaaucgaaucggauuuuguuuuuaauauuucaaaaaaaaaaaaaaaaaaa
aaaaaaaaaaaaaaaaaaaaaa
DNA Sequence Encoding SEAP
SEQ ID NO: 39
tcgcgcgtttcggtgatgacggtgaaaacctctgacacatgcagctcccggagacggtcacagcttgtctgtaagcggat
gccgggagcagacaagcccgtcagggcgcgtcagcgggtgttggcgggtgtcggggctggcttaactatgcggcatcaga
gcagattgtactgagagtgcaccatatgcggtgtgaaataccgcacagatgcgtaaggagaaaataccgcatcagattgg
ctattggccattgcatacgttgtatccatatcataatatgtacatttatattggctcatgtccaacattaccgccatgtt
gacattgattattgactagttattaatagtaatcaattacggggtcattagttcatagcccatatatggagttccgcgtt
acataacttacggtaaatggcccgcctggctgaccgcccaacgacccccgcccattgacgtcaataatgacgtatgttcc
catagtaacgccaatagggactttccattgacgtcaatgggtggagtatttacggtaaactgcccacttggcagtacatc
aagtgtatcatatgccaagtacgccccctattgacgtcaatgacggtaaatggcccgcctggcattatgcccagtacatg
accttatgggactttcctacttggcagtacatctacgtattagtcatcgctattaccatggtgatgcggttttggcagta
catcaatgggcgtggatagcggtttgactcacggggatttccaagtctccaccccattgacgtcaatgggagtttgtttt
ggcaccaaaatcaacgggactttccaaaatgtcgtaacaactccgccccattgacgcaaatgggcggtaggcgtgtacgg
tgggaggtctatataagcagagctcgtttagtgaaccgtcagatcgcctggagacgccatccacgctgttttgacctcca
tagaagacaccgggaccgatccagcctccgcggccgggaacggtgcattggaacgcggattccccgtgccaagagtgacg
taagtaccgcctatagactctataggcacacccctttggctcttatgcatgctatactgtttttggcttggggcctatac
acccccgcttccttatgctataggtgatggtatagcttagcctataggtgtgggttattgaccattattgaccactcccc
tattggtgacgatactttccattactaatccataacatggctctttgccacaactatctctattggctatatgccaatac
tctgtccttcagagactgacacggactctgtatttttacaggatggggtcccatttattatttacaaattcacatataca
acaacgccgtcccccgtgcccgcagtttttattaaacatagcgtgggatctccacgcgaatctcgggtacgtgttccgga
catgggctcttctccggtagcggcggagcttccacatccgagccctggtcccatgcctccagcggctcatggtcgctcgg
cagctccttgctcctaacagtggaggccagacttaggcacagcacaatgcccaccaccaccagtgtgccgcacaaggccg
tggcggtagggtatgtgtctgaaaatgagcgtggagattgggctcgcacggctgacgcagatggaagacttaaggcagcg
gcagaagaagatgcaggcagctgagttgttgtattctgataagagtcagaggtaactcccgttgcggtgctgttaacggt
ggagggcagtgtagtctgagcagtactcgttgctgccgcgcgcgccaccagacataatagctgacagactaacagactgt
tcctttccatgggtcttttctgcagtcaccgtcgtcgacaagcttcctgcatgctgctgctgctgctgctgctgggcctg
aggctacagctctccctgggcatcatcccagttgaggaggagaacccggacttctggaaccgcgaggcagccgaggccct
gggtgccgccaagaagctgcagcctgcacagacagccgccaagaacctcatcatcttcctgggcgatgggatgggggtgt
ctacggtgacagctgccaggatcctaaaagggcagaagaaggacaaactggggcctgagatacccctggccatggaccgc
ttcccatatgtggctctgtccaagacatacaatgtagacaaacatgtgccagacagtggagccacagccacggcctacct
gtgcggggtcaagggcaacttccagaccattggcttgagtgcagccgcccgctttaaccagtgcaacacgacacgcggca
acgaggtcatctccgtgatgaatcgggccaagaaagcagggaagtcagtgggagtggtaaccaccacacgagtgcagcac
gcctcgccagccggcacctacgcccacacggtgaaccgcaactggtactcggacgccgacgtgcctgcctcggcccgcca
ggaggggtgccaggacatcgctacgcagctcatctccaacatggacattgacgtgatcctaggtggaggccgaaagtaca
tgtttcgcatgggaaccccagaccctgagtacccagatgactacagccaaggtgggaccaggctggacgggaagaatctg
gtgcaggaatggctggcgaagcgccagggtgcccggtatgtgtggaaccgcactgagctcatgcaggcttccctggaccc
gtctgtgacccatctcatgggtctctttgagcctggagacatgaaatacgagatccaccgagactccacactggacccct
ccctgatggagatgacagaggctgccctgcgcctgctgagcaggaacccccgcggcttcttcctcttcgtggagggtggt
cgcatcgaccatggtcatcatgaaagcagggcttaccgggcactgactgagacgatcatgttcgacgacgccattgagag
ggcgggccagctcaccagcgaggaggacacgctgagcctcgtcactgccgaccactcccacgtcttctccttcggaggct
accccctgcgagggagctccatcttcgggctggcccctggcaaggcccgggacaggaaggcctacacggtcctcctatac
ggaaacggtccaggctatgtgctcaaggacggcgcccggccggatgttaccgagagcgagagcgggagccccgagtatcg
gcagcagtcagcagtgcccctggacgaagagacccacgcaggcgaggacgtggcggtgttcgcgcgcggcccgcaggcgc
acctggttcacggcgtgcaggagcagaccttcatagcgcacgtcatggccttcgccgcctgcctggagccctacaccgcc
tgcgacctggcgccccccgccggcaccaccgacgccgcgcacccgggttaacccgtggtcagatccagatccagatcact
tctggctaataaaagatcagagctctagagatctgtgtgttggttttttgtggatctgctgtgccttctagttgccagcc
atctgttgtttgcccctcccccgtgccttccttgaccctggaaggtgccactcccactgtcctttcctaataaaatgagg
aaattgcatcgcattgtctgagtaggtgtcattctattctggggggtggggtggggcagcacagcaagggggaggattgg
gaagacaatagcaggcatgctggggatgcggtgggctctatgggtacctctctctctctctctctctctctctctctctc
tctctctcggtacctctctctctctctctctctctctctctctctctctctctcggtaccaggtgctgaagaattgaccc
ggttcctcctgggccagaaagaagcaggcacatccccttctctgtgacacaccctgtccacgcccctggttcttagttcc
agccccactcataggacactcatagctcaggagggctccgccttcaatcccacccgctaaagtacttggagcggtctctc
cctccctcatcagcccaccaaaccaaacctagcctccaagagtgggaagaaattaaagcaagataggctattaagtgcag
agggagagaaaatgcctccaacatgtgaggaagtaatgagagaaatcatagaatttcttccgcttcctcgctcactgact
cgctgcgctcggtcgttcggctgcggcgagcggtatcagctcactcaaaggcggtaatacggttatccacagaatcaggg
gataacgcaggaaagaacatgtgagcaaaaggccagcaaaaggccaggaaccgtaaaaaggccgcgttgctggcgttttt
ccataggctccgcccccctgacgagcatcacaaaaatcgacgctcaagtcagaggtggcgaaacccgacaggactataaa
gataccaggcgtttccccctggaagctccctcgtgcgctctcctgttccgaccctgccgcttaccggatacctgtccgcc
tttctcccttcgggaagcgtggcgctttctcaatgctcacgctgtaggtatctcagttcggtgtaggtcgttcgctccaa
gctgggctgtgtgcacgaaccccccgttcagcccgaccgctgcgccttatccggtaactatcgtcttgagtccaacccgg
taagacacgacttatcgccactggcagcagccactggtaacaggattagcagagcgaggtatgtaggcggtgctacagag
ttcttgaagtggtggcctaactacggctacactagaaggacagtatttggtatctgcgctctgctgaagccagttacctt
cggaaaaagagttggtagctcttgatccggcaaacaaaccaccgctggtagcggtggtttttttgtttgcaagcagcaga
ttacgcgcagaaaaaaaggatctcaagaagatcctttgatcttttctacggggtctgacgctcagtggaacgaaaactca
cgttaagggattttggtcatgagattatcaaaaaggatcttcacctagatccttttaaattaaaaatgaagttttaaatc
aatctaaagtatatatgagtaaacttggtctgacagttaccaatgcttaatcagtgaggcacctatctcagcgatctgtc
tatttcgttcatccatagttgcctgactccggggggggggggcgctgaggtctgcctcgtgaagaaggtgttgctgactc
ataccaggcctgaatcgccccatcatccagccagaaagtgagggagccacggttgatgagagctttgttgtaggtggacc
agttggtgattttgaacttttgctttgccacggaacggtctgcgttgtcgggaagatgcgtgatctgatccttcaactca
gcaaaagttcgatttattcaacaaagccgccgtcccgtcaagtcagcgtaatgctctgccagtgttacaaccaattaacc
aattctgattagaaaaactcatcgagcatcaaatgaaactgcaatttattcatatcaggattatcaataccatatttttg
aaaaagccgtttctgtaatgaaggagaaaactcaccgaggcagttccataggatggcaagatcctggtatcggtctgcga
ttccgactcgtccaacatcaatacaacctattaatttcccctcgtcaaaaataaggttatcaagtgagaaatcaccatga
gtgacgactgaatccggtgagaatggcaaaagcttatgcatttctttccagacttgttcaacaggccagccattacgctc
gtcatcaaaatcactcgcatcaaccaaaccgttattcattcgtgattgcgcctgagcgagacgaaatacgcgatcgctgt
taaaaggacaattacaaacaggaatcgaatgcaaccggcgcaggaacactgccagcgcatcaacaatattttcacctgaa
tcaggatattcttctaatacctggaatgctgttttcccggggatcgcagtggtgagtaaccatgcatcatcaggagtacg
gataaaatgcttgatggtcggaagaggcataaattccgtcagccagtttagtctgaccatctcatctgtaacatcattgg
caacgctacctttgccatgtttcagaaacaactctggcgcatcgggcttcccatacaatcgatagattgtcgcacctgat
tgcccgacattatcgcgagcccatttatacccatataaatcagcatccatgttggaatttaatcgcggcctcgagcaaga
cgtttcccgttgaatatggctcataacaccccttgtattactgtttatgtaagcagacagttttattgttcatgatgata
tatttttatcttgtgcaatgtaacatcagagattttgagacacaacgtggctttccccccccccccattattgaagcatt
tatcagggttattgtctcatgagcggatacatatttgaatgtatttagaaaaataaacaaataggggttccgcgcacatt
tccccgaaaagtgccacctgacgtctaagaaaccattattatcatgacattaacctataaaaataggcgtatcacgagg
ccctttcgtc

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A dried composition comprising: a) a lipid carrier, wherein the lipid carrier is a nanoemulsion comprising a hydrophobic core, and one or more lipids; b) at least one nucleic acid that comprises a sequence which encodes a sequence capable of expressing an antigen, optionally wherein the antigen is a SARS-CoV-2 spike protein or a functional variant thereof; and c) at least one sugar present in amount of at least about 50% by weight of the dried composition.

2. The composition of claim 1, wherein the composition is lyophilized.

3. The composition of claim 1, wherein the composition is thermally stable:
at about −20 degrees Celsius,
at about 25 degrees Celsius,
at about 45 degrees Celsius,
at about 2 degrees Celsius to about 8 degrees Celsius, or
a combination thereof.

4. The composition of claim 1, wherein the composition is thermally stable for at least 1 week.

5. The composition of claim 1, wherein the hydrophobic core comprises an oil.

6. The composition of claim 5, wherein the oil comprises at least one of a-tocopherol, lauroyl polyoxylglyceride, monoacylglycerol, propolis, squalene, mineral oil, grapeseed oil, olive oil, paraffin oil, peanut oil, soybean oil, sunflower oil, soy lecithin, triglyceride, vitamin E, a caprylic/capric triglyceride, a triglyceride ester of saturated coconut/palmkernel oil derived caprylic and capric fatty acids and plant derived glycerol, dihydroisosqualene (DHIS), farnesene and squalane.

7. The composition of claim 1, wherein the one or more lipids is selected from the group consisting of cationic lipids, anionic lipids, neutral lipids, and any combinations thereof.

8. The composition of claim 7, wherein the one or more lipids comprises a cationic lipid.

9. The composition of claim 8, wherein the cationic lipid is selected from the group consisting of 1,2-dioleoyloxy-3-(trimethylammonium)propane (DOTAP); 30-[N—(N′,N′-dimethylaminoethane)-carbamoyl]cholesterol (DC Cholesterol); dimethyldioctadecylammonium (DDA); 1,2-dimyristoyl-3-trimethylammoniumpropane (DMTAP); dipalmitoyl(C16:0)trimethyl ammonium propane (DPTAP); distearoyltrimethylammonium propane (DSTAP); N-[l-(2,3-dioleyloxy)propyl]-N,N,Ntrimethylammonium chloride (DOTMA); N,N-dioleoyl-N,N-dimethylammonium chloride (DODAC); 1,2-dioleoyl-sn-glycero-3-ethylphosphocholine (DOEPC); 1,2-dioleoyl-3-dimethylammonium-propane (DODAP); 1,2-dilinoleyloxy-3-dimethylaminopropane (DLin-DMA); 1, 1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200); 1,2-dioleoyl-sw-glycero-3-phosphoethanolamine (DOPE); 7V-decyl-7V,7V-dimethyldecan-1-aminium bromide (DDAB); 2,3-dioleyloxy-7V-[2-(sperminecarboxamido)ethyl]-7V,7V-dimethyl-1-propanaminium trifluoroacetate (DOSPA); ethylphosphatidylcholine (ePC); and any combinations thereof.

10. The composition of claim 1, wherein the lipid carrier comprises at least one surfactant selected from the group consisting of a hydrophobic surfactant, a hydrophilic surfactant, and any combinations thereof.

11. The composition of claim 10, wherein the hydrophobic surfactant comprises a sorbitan ester selected from the group consisting of sorbitan monolaurate, sorbitan monopalmitate, sorbitan monostearate, sorbitan tristearate, sorbitan monooleate, and sorbitan trioleate; and the hydrophilic surfactant comprises a polysorbate.

12. The composition of claim 1, wherein the lipid carrier has a z-average hydrodynamic diameter ranging from about 40 nm to about 150 nm, with an average poly dispersity index ranging from about 0.1 to about 0.4.

13. The composition of claim 1, wherein the one or more nucleic acid is a DNA or a RNA.

14. The composition of claim 13, wherein the RNA is a self-replicating RNA.

15. The composition of claim 1, wherein a molar ratio of the lipid carrier to the one or more nucleic acids is characterized by a nitrogen-to-phosphate (N:P) molar ratio that ranges from about 1:1 to about 150:1.

16. The composition of claim 1, wherein the at least one sugar is selected from the group consisting of sucrose, maltose, trehalose, mannitol, glucose, and any combinations thereof.

17. The composition of claim 1, wherein the at least one nucleic acid comprises a sequence at least 85% identical to SEQ ID NOS: 1-8.

18. The composition of claim 1, wherein the at least one sugar present in an amount of least about 50 mg.

19. A composition comprising: a lipid carrier, wherein the lipid carrier comprises: liquid oil; and surfactants, wherein the surfactants comprise: a cationic lipid; a hydrophilic surfactant; and a hydrophobic surfactant; and at least one nucleic acid, wherein the at least one nucleic acid comprises a sequence at least 85% identical to SEQ ID NOS: 1 to 7.

20. The composition of claim 19, wherein the nucleic acid comprises a sequence that is at least 85% identical to SEQ ID NO: 4 or SEQ ID NO: 27.

21. The composition of claim 19, wherein the nucleic acid further encodes for an RNA polymerase.

22. The composition of claim 21, wherein the RNA polymerase is a Venezuelan equine encephalitis virus (VEEV) RNA polymerase.

23. The composition of claim 22, wherein is the nucleic acid coding the RNA polymerase comprises a nucleic acid sequence of SEQ ID NO: 20.

24. The composition of claim 19, wherein the liquid oil is a-tocopherol, coconut oil, grapeseed oil, lauroyl polyoxylglyceride, mineral oil, monoacylglycerol, palmkemal oil, olive oil, paraffin oil, peanut oil, propolis, squalene, squalane, soy lecithin, soybean oil, sunflower oil, a triglyceride, or vitamin E.

25. The composition of claim 24, wherein the triglyceride is capric triglyceride, caprylic triglyceride, a caprylic and capric triglyceride, a triglyceride ester, or myristic acid triglycerin.

26. The composition of claim 19, wherein the cationic lipid is 1,2-dioleoyloxy-3 (trimethylammonium)propane (DOTAP); 3β-[N—(N′,N′-dimethylaminoethane) carbamoyl]cholesterol (DC Cholesterol); dimethyldioctadecylammonium (DDA); 1,2-dimyristoyl 3-trimethylammoniumpropane(DMTAP); dipalmitoyl(C16:0)trimethyl ammonium propane (DPTAP); distearoyltrimethylammonium propane (DSTAP); N-[l-(2,3-dioleyloxy)propyl]N,N,Ntrimethylammonium chloride (DOTMA); N,N-dioleoyl-N,N-dimethylammonium chloride (DODAC); 1,2-dioleoyl-sn-glycero-3-ethylphosphocholine (DOEPC); 1,2-dioleoyl-3-dimethylammonium-propane (DODAP); 1,2-dilinoleyloxy-3-dimethylaminopropane (DLinDMA); 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200); 306Oᵢ₁₀, tetrakis(8-methylnonyl) 3,3′,3″,3″′-(((methylazanediyl) bis(propane-3,1 diyl))bis (azanetriyl))tetrapropionate; 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-TH-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 30-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″″,9″″′-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9″′Z,12Z,12′,12″Z,12″′Z)-tetrakis (octadeca-9,12-dienoate); PEG2000-DMG, (R)-2,3-bis(myristoyloxy)propyl-1-(methoxy poly(ethylene glycol)2000) carbamate; or TT3, N1,N3,N5-tris(3-(didodecylamino)propyl)benzene-1,3,5-tricarboxamide.

27. The composition of claim 19, wherein the lipid carrier comprises a hydrophobic core, an inorganic particle, or both.

28. The composition of claim 27, wherein the inorganic particle comprises a metal salt, a metal oxide, a metal hydroxide, or a metal phosphate.

29. The composition of claim 28, wherein the metal oxide comprises aluminum oxide, aluminum oxyhydroxide, iron oxide, titanium dioxide, or silicon dioxide.

30. The composition of claim 19, wherein the hydrophobic surfactant is sorbitan monolaurate, sorbitan monopalmitate, sorbitan monostearate, sorbitan monooleate, or sorbitan trioleate.

31. The composition of claim 19, wherein the hydrophilic surfactant is a polysorbate.


SEQUENCE LISTING (39 sequences)

SEQ ID NO: 1 - RNA, 3846 nt, Severe acute respiratory syndrome coronavirus 2
  source 1..3846: mol_type = other RNA
atgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacaaagtggaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactataca

SEQ ID NO: 2 - RNA, 3846 nt, Severe acute respiratory syndrome coronavirus 2
  source 1..3846: mol_type = other RNA
atgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactataca

SEQ ID NO: 3 - RNA, 3846 nt, Severe acute respiratory syndrome coronavirus 2
  source 1..3846: mol_type = other RNA
atgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcaggccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacaaagtggaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactataca

SEQ ID NO: 4 - RNA, 3837 nt, Severe acute respiratory syndrome coronavirus 2
  source 1..3837: mol_type = other RNA
atgtttctgctcacaaccaaacgcactatgtttgttttcctcgtgctgctccctttggtaagttctcagtgtgtaaacttcacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgccaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcggggccttccccaaggcttttctgctctcgaacccctcgtagacttgccaattgggataaatatcactcgctttcaaactttgcacatcagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaacattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttaagggatttaactgctattttcctttgcaatcctatggctttcaaccaacatacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggcgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgtggagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactataca

SEQ ID NO: 5 - RNA, 3837 nt, Severe acute respiratory syndrome coronavirus 2
  source 1..3837: mol_type = other RNA
atgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaataagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacatacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagacgacacaactgacgctgtacgcgatcctcagactttggagatcttggacatcactccctgttctttcggaggggtatctgtcatcacccccggaactaatacatcaaatcaggtcgctgtgttgtaccaaggcgtcaactgcacagaagtccccgttgctatacacgcagatcagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattcccatcggcgagcacgatctgtagcatcccagtctattattgcctacactatgtcattgggcgccgagaatagcgtcgcatattcaaataattctattgcaatacccatcaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttggcccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactcacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactataca

SEQ ID NO: 6 - RNA, 3843 nt, Severe acute respiratory syndrome coronavirus 2
  source 1..3843: mol_type = other RNA
atgtttctgctcacaaccaaacgcactatgtttgttttcctcgtgctgctccctttggtaagttctcagtgtgtaaacctgagaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtatcgaaaagtcaaacataatccgggggtggatctttggaaccactttggactctaagacacagtctctcctcatagtaaacaacgccaccaatgttgtcataaaagtatgcgaatttcagttttgcaacgatccctttctcgacgtgtattaccataagaataataaatcctggatggagtctggggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggtttgaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccatcgtacgatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaatattgctgattataattataaactcccagatgactttactggatgcgtcatagcctggaattccaacaatctagattccaaggttggtgggaattataattaccgttatcgactgttcagaaagagtaacttgaaaccatttgagagagacatatccaccgagatttaccaggcaggcagtaagccttgtaacggcgttgagggatttaactgctattttcctttgcaatcctatggctttcaaccaacaaacggggttggctatcaaccctatcgagtggttgtcctcagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaatttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggtgtcaactgcacagaagtccccgttgctatacacgcagaccagctcacccccacatggcgggtgtactcaactggctcaaacgtattccagaccagagctgggtgcttgatcggtgctgaacacgtgaacaatagctatgaatgcgatattcccatcggtgccgggatctgcgctagctatcagacacagaccaattcccgcaggcgggctcgctctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcacttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcagaacgttgtcaaccagaatgctcaagctctgaatacattggttaagcagctctctagtaattttggggccatctcttcagtacttaatgatattttgagccgattggacccacctgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatacgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaagggggtaaagcttcactatacatga

SEQ ID NO: 7 - RNA, 3843 nt, Severe acute respiratory syndrome coronavirus 2; identical to SEQ ID NO: 6
  source 1..3843: mol_type = other RNA

SEQ ID NO: 8 - RNA, 3847 nt, Severe acute respiratory syndrome coronavirus 2
  source 1..3847: mol_type = other RNA
cgccaccatgtttctgttgacgaccaagcgaacgatgttcgttttcttggtgcttttgccacttgtcagttcccagtgcgtcaatctgacgacacgaacacagctgcctcctgcgtacactaacagttttacgcgaggagtgtattaccctgacaaagttttccgctctagtgtcctccatagcacacaggacttgtttctccccttcttttccaacgttacgtggttccatgtgattagtggaactaacggtactaaaagattcgacaatccagtattgcctttcaacgatggggtctatttcgcgtccatcgagaaatcaaatatcattcgcggttggatttttggaacgacactcgattcaaagacgcaatccctccttattgtcaacaacgccactaacgtagtcattaaggtttgtgagttccagttttgtaatgatcccttttttgaccacaagaataacaagagctggatggaaagcgagttcagagtgtatagctctgcaaacaactgtacttttgaatacgtgagtcaacctttccttatggaccttgaaggtaaacagggtaactttaagaatttgcgcgaatttgttttcaaaaacattgatggttactttaaaatctatagtaagcacactcctatcattgtaagagagccggaggaccttccacagggttttagtgcgctcgagcccctcgttgacctgcccattgggatcaacataactcgattccaaacattgctcgcccttcatcggtcctatctgactcccggtgactcctctagcggatggacggcaggtgccgccgcatactacgtggggtaccttcaacctcggacatttttgttgaaatacaatgagaatggcactataactgacgcggttgattgcgcgctcgacccattgtccgaaactaagtgtactttgaagtcatttacagtggagaaaggaatatatcagactagcaattttcgggtacagcccacggagtctatcgtacggtttcctaacatcacgaatctgtgcccttttgatgaggtctttaatgcaacacggttcgcctccgtctatgcgtggaatagaaagcgcatctcaaattgtgtagctgattattccgtactttacaacttggccccgttcttcacatttaagtgctacggtgtaagccctactaaactgaacgatttgtgtttcaccaacgtctatgcagatagctttgttattcgaggcgatgaggtacgccagattgcgcctggtcaaacgggtaatatcgccgactacaattataaattgccagacgattttactggttgtgtcatcgcttggaatagtaataagttggacagtaaggtatccggcaattacaactatctctaccgattgttccggaagtctaacctcaagccgtttgaaagagacatatccactgagatataccaagcaggcaataaaccatgcaacggagttgctggtttcaactgctatttcccgttgcggtcttattccttcagacctacttacggagtcggacaccaaccctacagggtcgtcgttttgagttttgaattgttgcatgctccagcaaccgtgtgtggacctaaaaagtccacgaatctcgtgaagaataagtgcgtaaacttcaatttcaacggtctgaaagggactggtgtattgacagaaagcaacaagaagtttctgccattccagcaatttggtagggatatagcggatacaactgatgccgttcgggatcctcaaacattggagatcttggacatcacaccgtgttcttttgggggtgtctccgttatcacaccgggtacaaatacgagcaatcaggttgcggtcctttaccaaggcgttaattgtaccgaggttccagtagcaatacacgcggatcaactcacgcccacatggagggtttacagtacaggcagtaatgttttccaaacgagagcgggatgcctcatcggggcagaatacgtaaataattcttacgaatgcgacatccctattggcgcaggaatttgcgcaagttaccaaacccagaccaagtctcataggcgggcgcggtctgttgcaagccaatctataatagcgtacactatgtccctcggcgcggagaacagtgtcgcatattccaacaactctattgcgatacctactaatttcactattagcgtcacaactgagatccttcccgtcagtatgaccaaaacgtctgtcgactgtactatgtatatttgcggcgacagtaccgaatgctctaatcttttgttgcagtatggttctttttgcacgcaacttaagagagctttgacggggatagctgtggaacaagataaaaacacacaggaggtatttgcacaagtgaaacagatctataaaactccaccgatcaagtactttggcggctttaacttctcccagatcttgcccgacccgtctaaaccaagtaaacggagttttatagaggaccttctcttcaataaggtaacattggcagacgccggcttcattaaacaatacggagattgccttggagacatcgctgcgcgcgacttgatctgcgcacaaaaatttaaaggcttgacggtcctccctcctttgctcacagacgagatgatagcacaatacacttccgcactgcttgctggaaccatcacctctggttggacattcggtgcgggagcggctttgcagattccgtttgcgatgcaaatggcttatcggtttaacggcattggagtaacacagaatgtgctctacgagaatcaaaagcttattgcgaatcaattcaactctgcgattggcaaaattcaagattcattgagtagcaccgccagtgctcttggcaagcttcaggatgtcgtaaaccacaatgcacaagctctgaatacactggttaaacaattgtccagtaaatttggggcaatctcttcagtgctgaacgacattttctcaagattggatccacccgaagcggaggtacagattgaccgcctgataaccgggaggttgcaaagccttcagacttatgttacacaacagttgattcgggcagcagagataagagcctcagcaaacctcgcagctacgaagatgtcagagtgtgtccttgggcaatctaagcgggtagatttctgcggcaaaggatatcatttgatgagctttccccaatcagccccacatggagtagtttttcttcatgtcacttacgttccggcgcaggaaaagaacttcaccacagcgccagccatttgtcatgatgggaaggcgcatttcccaagagaaggtgttttcgtgtctaacggtacccactggttcgttacgcagcggaatttctacgaaccacagatcatcactaccgacaacacgtttgtctctggaaattgtgacgttgtcatagggatagtgaacaatacagtatatgatccacttcagcctgaacttgactcttttaaggaggagctggacaaatatttcaaaaatcatacaagcccggacgtcgatcttggagatatttcaggtatcaacgcaagtgtcgtaaatattcagaaggagatcgatcgattgaacgaggttgcaaaaaaccttaatgagagccttatagatcttcaagagctggggaagtatgaacaatatatcaagtggccttggtacatttggctcgggttcattgccggacttatcgcgatcgtaatggtaacaatcatgctctgctgtatgacttcttgctgttcatgcctcaaaggttgttgctcctgtgggtcttgctgtaaatttgacgaggatgattctgaaccagtgcttaaaggcgtgaagctccactatacctga

SEQ ID NO: 9 - protein, 1280 aa, Severe acute respiratory syndrome coronavirus 2
MFLLTTKRTMFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGLTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKNFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 1280 aa, Severe acute respiratory syndrome coronavirus 2; identical to SEQ ID NO: 9

SEQ ID NO: 11 - protein, 1280 aa, Severe acute respiratory syndrome coronavirus 2; identical to SEQ ID NO: 9

SEQ ID NO: 12 - protein, 1279 aa, Severe acute respiratory syndrome coronavirus 2
MFLLTTKRTMFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - protein, 1282 aa, Severe acute respiratory syndrome coronavirus 2
MFLLTTKRTMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHAGQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 14 - protein, 1282 aa, Severe acute respiratory syndrome coronavirus 2
MFLLTTKRTMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - protein, 1260 aa, Severe acute respiratory syndrome coronavirus 2
MFLLTTKRTMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCG

SEQ ID NO: 16 - protein, 1279 aa, Severe acute respiratory syndrome coronavirus 2
MFLLTTKRTMFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFFDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 18 - protein, 302 aa, Severe acute respiratory syndrome coronavirus 2
QNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPW

SEQ ID NO: 19 - protein, 302 aa, Severe acute respiratory syndrome coronavirus 2
QNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPW

SEQ ID NO: 20 - RNA, 7561 nt, Venezuelan equine encephalitis virus
  source 1..7561: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaag

SEQ ID NO: 21 - protein, 51 aa, Venezuelan equine encephalitis virus
RELPVLDSAAFNVECFKKYACNNEYWETFKENPIRLTEENVVNYITKLKGP

SEQ ID NO: 22 - protein, 41 aa, Venezuelan equine encephalitis virus
TQMRELPVLDSAAFNVECFKKYACNNEYWETFKENPIRLTE

SEQ ID NO: 23 - protein, 536 aa, Venezuelan equine encephalitis virus
MKAITARRILQGLGHYLKAEGKVECYRTLHPVPLYSSSVNRAFSSPKVAVEACNAMLKENFPTVASYCIIPEYDAYLDMIDGASCCLDTASFCPAKLRSFPKKHSYLEPTIRSAVPSAIQNTLQNVLAAATKRNCNVTQMRELPVLDSAAFNVECFKKYACNNEYWKTFKENPIRLTEENVINYITKLKGPKAAALYAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTAMMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILPELCKAVESRYETVGTSVIVMAMATLASSVKSFSYLRGAPITLYG

SEQ ID NO: 24 - RNA, 11707 nt, synthetic construct
  misc_feature 1..11707: note = Description of Artificial Sequence: Synthetic polynucleotide
  source 1..11707: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacaaagtggaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 25 - RNA, 11707 nt, synthetic construct
  misc_feature 1..11707: note = Description of Artificial Sequence: Synthetic polynucleotide
  source 1..11707: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 26 - RNA, 11707 nt, synthetic construct
  misc_feature 1..11707: note = Description of Artificial Sequence: Synthetic polynucleotide
  source 1..11707: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcaggccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacaaagtggaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactatacatagtaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 27 - RNA, 11698 nt, synthetic construct
  misc_feature 1..11698: note = Description of Artificial Sequence: Synthetic polynucleotide
  source 1..11698: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacttcacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgccaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcggggccttccccaaggcttttctgctctcgaacccctcgtagacttgccaattgggataaatatcactcgctttcaaactttgcacatcagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaacattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttaagggatttaactgctattttcctttgcaatcctatggctttcaaccaacatacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggcgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgtggagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 28 - RNA, 11698 nt, synthetic construct
  misc_feature 1..11698: note = Description of Artificial Sequence: Synthetic polynucleotide
  source 1..11698: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaataagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacatacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagacgacacaactgacgctgtacgcgatcctcagactttggagatcttggacatcactccctgttctttcggaggggtatctgtcatcacccccggaactaatacatcaaatcaggtcgctgtgttgtaccaaggcgtcaactgcacagaagtccccgttgctatacacgcagatcagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattcccatcggcgagcacgatctgtagcatcccagtctattattgcctacactatgtcattgggcgccgagaatagcgtcgcatattcaaataattctattgcaatacccatcaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttggcccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactcacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 29 - RNA, 11701 nt, synthetic construct
  misc_feature 1..11701: note = Description of Artificial Sequence: Synthetic polynucleotide
  source 1..11701: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgagaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtatcgaaaagtcaaacataatccgggggtggatctttggaaccactttggactctaagacacagtctctcctcatagtaaacaacgccaccaatgttgtcataaaagtatgcgaatttcagttttgcaacgatccctttctcgacgtgtattaccataagaataataaatcctggatggagtctggggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggtttgaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccatcgtacgatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaatattgctgattataattataaactcccagatgactttactggatgcgtcatagcctggaattccaacaatctagattccaaggttggtgggaattataattaccgttatcgactgttcagaaagagtaacttgaaaccatttgagagagacatatccaccgagatttaccaggcaggcagtaagccttgtaacggcgttgagggatttaactgctattttcctttgcaatcctatggctttcaaccaacaaacggggttggctatcaaccctatcgagtggttgtcctcagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaatttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggtgtcaactgcacagaagtccccgttgctatacacgcagaccagctcacccccacatggcgggtgtactcaactggctcaaacgtattccagaccagagctgggtgcttgatcggtgctgaacacgtgaacaatagctatgaatgcgatattcccatcggtgccgggatctgcgctagctatcagacacagaccaattcccgcaggcgggctcgctctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcacttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcagaacgttgtcaaccagaatgctcaagctctgaatacattggttaagcagctctctagtaattttggggccatctcttcagtacttaatgatattttgagccgattggacccacctgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatacgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaagggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 30 - RNA, 11703 nt, synthetic construct
  misc_feature 1..11703: note = Description of Artificial Sequence: Synthetic polynucleotide
  source 1..11703: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatgtttctgctcacaaccaaacgcactatgtttgttttcctcgtgctgctccctttggtaagttctcagtgtgtaaacctgagaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtatcgaaaagtcaaacataatccgggggtggatctttggaaccactttggactctaagacacagtctctcctcatagtaaacaacgccaccaatgttgtcataaaagtatgcgaatttcagttttgcaacgatccctttctcgacgtgtattaccataagaataataaatcctggatggagtctggggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggtttgaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccatcgtacgatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaatattgctgattataattataaactcccagatgactttactggatgcgtcatagcctggaattccaacaatctagattccaaggttggtgggaattataattaccgttatcgactgttcagaaagagtaacttgaaaccatttgagagagacatatccaccgagatttaccaggcaggcagtaagccttgtaacggcgttgagggatttaactgctattttcctttgcaatcctatggctttcaaccaacaaacggggttggctatcaaccctatcgagtggttgtcctcagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaatttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggtgtcaactgcacagaagtccccgttgctatacacgcagaccagctcacccccacatggcgggtgtactcaactggctcaaacgtattccagaccagagctgggtgcttgatcggtgctgaacacgtgaacaatagctatgaatgcgatattcccatcggtgccgggatctgcgctagctatcagacacagaccaattcccgcaggcgggctcgctctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcacttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcagaacgttgtcaaccagaatgctcaagctctgaatacattggttaagcagctctctagtaattttggggccatctcttcagtacttaatgatattttgagccgattggacccacctgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatacgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaagggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 31 - RNA, 11701 nt, synthetic construct
  misc_feature 1..11701: note = Description of Artificial Sequence: Synthetic polynucleotide
  source 1..11701: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatgtttctgttgacgaccaagcgaacgatgttcgttttcttggtgcttttgccacttgtcagttcccagtgcgtcaatctgacgacacgaacacagctgcctcctgcgtacactaacagttttacgcgaggagtgtattaccctgacaaagttttccgctctagtgtcctccatagcacacaggacttgtttctccccttcttttccaacgttacgtggttccatgtgattagtggaactaacggtactaaaagattcgacaatccagtattgcctttcaacgatggggtctatttcgcgtccatcgagaaatcaaatatcattcgcggttggatttttggaacgacactcgattcaaagacgcaatccctccttattgtcaacaacgccactaacgtagtcattaaggtttgtgagttccagttttgtaatgatcccttttttgaccacaagaataacaagagctggatggaaagcgagttcagagtgtatagctctgcaaacaactgtacttttgaatacgtgagtcaacctttccttatggaccttgaaggtaaacagggtaactttaagaatttgcgcgaatttgttttcaaaaacattgatggttactttaaaatctatagtaagcacactcctatcattgtaagagagccggaggaccttccacagggttttagtgcgctcgagcccctcgttgacctgcccattgggatcaacataactcgattccaaacattgctcgcccttcatcggtcctatctgactcccggtgactcctctagcggatggacggcaggtgccgccgcatactacgtggggtaccttcaacctcggacatttttgttgaaatacaatgagaatggcactataactgacgcggttgattgcgcgctcgacccattgtccgaaactaagtgtactttgaagtcatttacagtggagaaaggaatatatcagactagcaattttcgggtacagcccacggagtctatcgtacggtttcctaacatcacgaatctgtgcccttttgatgaggtctttaatgcaacacggttcgcctccgtctatgcgtggaatagaaagcgcatctcaaattgtgtagctgattattccgtactttacaacttggccccgttcttcacatttaagtgctacggtgtaagccctactaaactgaacgatttgtgtttcaccaacgtctatgcagatagctttgttattcgaggcgatgaggtacgccagattgcgcctggtcaaacgggtaatatcgccgactacaattataaattgccagacgattttactggttgtgtcatcgcttggaatagtaataagttggacagtaaggtatccggcaattacaactatctctaccgattgttccggaagtctaacctcaagccgtttgaaagagacatatccactgagatataccaagcaggcaataaaccatgcaacggagttgctggtttcaactgctatttcccgttgcggtcttattccttcagacctacttacggagtcggacaccaaccctacagggtcgtcgttttgagttttgaattgttgcatgctccagcaaccgtgtgtggacctaaaaagtccacgaatctcgtgaagaataagtgcgtaaacttcaatttcaacggtctgaaagggactggtgtattgacagaaagcaacaagaagtttctgccattccagcaatttggtagggatatagcggatacaactgatgccgttcgggatcctcaaacattggagatcttggacatcacaccgtgttcttttgggggtgtctccgttatcacaccgggtacaaatacgagcaatcaggttgcggtcctttaccaaggcgttaattgtaccgaggttccagtagcaatacacgcggatcaactcacgcccacatggagggtttacagtacaggcagtaatgttttccaaacgagagcgggatgcctcatcggggcagaatacgtaaataattcttacgaatgcgacatccctattggcgcaggaatttgcgcaagttaccaaacccagaccaagtctcataggcgggcgcggtctgttgcaagccaatctataatagcgtacactatgtccctcggcgcggagaacagtgtcgcatattccaacaactctattgcgatacctactaatttcactattagcgtcacaactgagatccttcccgtcagtatgaccaaaacgtctgtcgactgtactatgtatatttgcggcgacagtaccgaatgctctaatcttttgttgcagtatggttctttttgcacgcaacttaagagagctttgacggggatagctgtggaacaagataaaaacacacaggaggtatttgcacaagtgaaacagatctataaaactccaccgatcaagtactttggcggctttaacttctcccagatcttgcccgacccgtctaaaccaagtaaacggagttttatagaggaccttctcttcaataaggtaacattggcagacgccggcttcattaaacaatacggagattgccttggagacatcgctgcgcgcgacttgatctgcgcacaaaaatttaaaggcttgacggtcctccctcctttgctcacagacgagatgatagcacaatacacttccgcactgcttgctggaaccatcacctctggttggacattcggtgcgggagcggctttgcagattccgtttgcgatgcaaatggcttatcggtttaacggcattggagtaacacagaatgtgctctacgagaatcaaaagcttattgcgaatcaattcaactctgcgattggcaaaattcaagattcattgagtagcaccgccagtgctcttggcaagcttcaggatgtcgtaaaccacaatgcacaagctctgaatacactggttaaacaattgtccagtaaatttggggcaatctcttcagtgctgaacgacattttctcaagattggatccacccgaagcggaggtacagattgaccgcctgataaccgggaggttgcaaagccttcagacttatgttacacaacagttgattcgggcagcagagataagagcctcagcaaacctcgcagctacgaagatgtcagagtgtgtccttgggcaatctaagcgggtagatttctgcggcaaaggatatcatttgatgagctttccccaatcagccccacatggagtagtttttcttcatgtcacttacgttccggcgcaggaaaagaacttcaccacagcgccagccatttgtcatgatgggaaggcgcatttcccaagagaaggtgttttcgtgtctaacggtacccactggttcgttacgcagcggaatttctacgaaccacagatcatcactaccgacaacacgtttgtctctggaaattgtgacgttgtcatagggatagtgaacaatacagtatatgatccacttcagcctgaacttgactcttttaaggaggagctggacaaatatttcaaaaatcatacaagcccggacgtcgatcttggagatatttcaggtatcaacgcaagtgtcgtaaatattcagaaggagatcgatcgattgaacgaggttgcaaaaaaccttaatgagagccttatagatcttcaagagctggggaagtatgaacaatatatcaagtggccttggtacatttggctcgggttcattgccggacttatcgcgatcgtaatggtaacaatcatgctctgctgtatgacttcttgctgttcatgcctcaaaggttgttgctcctgtgggtcttgctgtaaatttgacgaggatgattctgaaccagtgcttaaaggcgtgaagctccactatacctgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 32 - RNA, 7862 nt, synthetic construct
  misc_feature 1..7862: note = Description of Artificial Sequence: Synthetic polynucleotide
  misc_difference 7562: note = a, c, g, or u
  source 1..7862: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagntgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 33 - RNA, 7862 nt, synthetic construct; identical to SEQ ID NO: 32
  misc_feature 1..7862: note = Description of Artificial Sequence: Synthetic polynucleotide
  misc_difference 7562: note = a, c, g, or u
  source 1..7862: mol_type = other RNA

SEQ ID NO: 34 - RNA, 7862 nt, synthetic construct
  misc_feature 1..7862: note = Description of Artificial Sequence: Synthetic polynucleotide
  misc_difference 7562: note = a, c, g, or u
  source 1..7862: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagntagtaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 35 - RNA, 7862 nt, synthetic construct; identical to SEQ ID NO: 32
  misc_feature 1..7862: note = Description of Artificial Sequence: Synthetic polynucleotide
  misc_difference 7562: note = a, c, g, or u
  source 1..7862: mol_type = other RNA

SEQ ID NO: 36 - RNA, 7862 nt, synthetic construct; identical to SEQ ID NO: 32
  misc_feature 1..7862: note = Description of Artificial Sequence: Synthetic polynucleotide
  misc_difference 7562: note = a, c, g, or u
  source 1..7862: mol_type = other RNA

SEQ ID NO: 37 - RNA, 11701 nt, synthetic construct; identical to SEQ ID NO: 29
  misc_feature 1..11701: note = Description of Artificial Sequence: Synthetic polynucleotide
  source 1..11701: mol_type = other RNA

SEQ ID NO: 38 - RNA, 7862 nt, synthetic construct
  misc_feature 1..7862: note = Description of Artificial Sequence: Synthetic polynucleotide
  misc_difference 7565: note = a, c, g, or u
  source 1..7862: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccntaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 39 - DNA, 6569 bp, synthetic construct
  misc_feature 1..6569: note = Description of Artificial Sequence: Synthetic polynucleotide
  source 1..6569: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacaagcttcctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaacccgtggtcagatccagatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc